(19) United States
(12) Patent Application Publication
(10) Pub. No.: US 2008/0207608 A1
(43) Pub. Date: Aug. 28, 2008

(54) 2-Propene-1-Ones As Hsp 70 Inducers

(76) Inventors: Prabhat Kumar, Gujarat (IN); Uttam Rajaram Mane, Gujarat (IN); Ramesh Chandra Gupta, Gujarat (IN); Sunil Sadanand Nadkarni, Gujarat (IN); Anookh Mohanan, Gujarat (IN); Ruchi Tandon, Gujarat (IN); Siralee Munshi, Gujarat (IN)

Correspondence Address: JACOBSON HOLMAN PLLC, 400 SEVENTH STREET N.W., SUITE 600, WASHINGTON, DC, 20004, US

(21) Appl. No.: 11/578,354

(22) PCT Filed: Apr. 11, 2005
(86) PCT No.: PCT/IN05/00112

§ 371 Date: Jul. 25, 2007

(30) Foreign Application Priority Data
Apr. 12, 2004 (IN) 178/KOL/2004

(51) Int. Cl. A61K 31/5377; A61K 31/44; A61K 31/445; A61K 31/47; A61P 29/00; A61P 9/00; C07D 215/00; C07D 239/72; C07D 241/36; C07D 295/00; C07D 241/00; C07D 239/02; C07D 213/00; A61P 43/00; A61P 25/00; A61K 31/495; A61K 31/517

(52) U.S. Cl. 514/232.2; 514/234.8; 514/235.2; 514/235.5; 514/235.8; 514/249; 514/253.06; 514/312; 514/314; 514/341; 544/120; 544/121; 544/124; 544/128; 544/354; 544/363; 544/82; 546/152; 546/155; 546/275.4

(57) ABSTRACT

The present invention relates to novel compounds of 2-propene-1-one series, of general formula (I), their derivatives, analogs, tautomeric forms, stereoisomers, polymorphs, pharmaceutically acceptable salts, pharmaceutically acceptable solvates and pharmaceutically acceptable compositions containing them, wherein R₅, R₆, Q and Y are as defined in the specification. The present invention also relates to a process for preparing such compounds, compositions containing such compounds, and use of such compound and composition in medicine. The compounds of the general formula (I) induce HSP-70 and are useful for the treatment of diseases accompanying pathological stress in a living mammalian organism, including a human being, such as stroke, myocardial infarction, inflammatory disorder, hepatotoxicity, sepsis, diseases of viral origin, allograft rejection, tumourous diseases, gastric mucosal damage, brain haemorrhage, endothelial dysfunctions, diabetic complications, neuro-degenerative diseases, post-traumatic neuronal damage, acute renal failure, glaucoma and aging related skin degeneration.

### FIELD OF THE INVENTION

The present invention relates to novel compounds of 2-propene-1-one series, of general formula (I), their derivatives, their analogs, their tautomeric forms, their stereoisomers, their polymorphs, their pharmaceutically acceptable salts, their pharmaceutically acceptable solvates and pharmaceutically acceptable compositions containing them wherein R₅, R₆, Q and Y have the meanings as defined hereinafter.

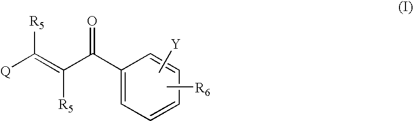

The present invention also relates to a process for the preparation of the above said novel compounds, their derivatives, their analogs, their tautomeric forms, their stereoisomers, their polymorphs, their pharmaceutically acceptable salts, their pharmaceutically acceptable solvates, and pharmaceutically acceptable compositions containing them.

The compounds of the general formula (I) are useful for the treatment and/or prophylaxis of ischaemia related injuries such as stroke, myocardial infarction, inflammatory disorder, hepatotoxicity, sepsis, diseases of viral origin, allograft rejection, tumourous diseases, gastric mucosal damage, brain haemorrhage, endothelial dysfunctions, diabetic complications, neuro-degenerative diseases, post-traumatic neuronal damage, acute renal failure, glaucoma and aging related skin degeneration, wherein the underlying mechanism is Heat Shock Protein (HSP) induction.

### BACKGROUND OF THE INVENTION

Heat shock proteins (HSPs) have been well documented to play a cytoprotective role in almost all living cells under various pathological stresses through a mechanism known as thermotolerance or cross tolerance. Heat shock proteins function as molecular chaperones or proteases that, under physiological conditions, have a number of intracellular functions. Chaperones are involved in the assembly and folding of misfolded or denatured oligomeric proteins, whereas proteases mediate the degradation of damaged proteins.

Heat shock proteins are categorized into several families that are named on the basis of their approximate molecular mass (e.g. the 70 kDa HSP-70, ubiquitin, HSP-10, HSP-27, HSP-32, HSP-60, HSP-90 etc). HSP-70 is the most abundant HSP found in normal cells. HSP-70, and its inducible form, called HSP-72, is found in all living cells. Following heat shock, its synthesis increases to a point to where it becomes the most abundant single protein in the cell.

Although some proteins refold spontaneously, in vitro, when diluted at low concentrations from denaturants, larger, multidomain proteins often have a propensity to misfold and aggregate. Consequently, the challenge within the densely packed cellular environment is to ensure that non-native intermediates are efficiently captured, maintained in intermediate folded states, and subsequently either refolded or degraded. Molecular chaperones such as HSP-90, HSP-70 and HSP-60 accomplish this by capturing non-native intermediates and, together with co-chaperones and ATP.

The HSP-70 chaperones, for example, recognize stretches of hydrophobic residues in polypeptide chains that are transiently exposed in early folding intermediates and typically confined to the hydrophobic core in the native state. The consequence of chaperone interactions, therefore, is to shift the equilibrium of protein folding and refolding reactions toward productive on-pathway events and to minimize the appearance of non-productive intermediates that have a propensity to aggregate as misfolded species.

Over the past years, a number of studies have shown that the major heat-inducible protein, HSP-72, is critical for protection of cells and tissues from heat shock and other stresses. HSP-72 functions as molecular chaperone in refolding and degradation of damaged proteins. This has led to the common assumption that chaperoning activities of HSP-72 determine its role in ability of a cell to protect itself against stresses. Upon exposure to stresses that lead to a massive protein damage and necrotic death, the anti-aggregating and protein refolding activities of HSP-72 may indeed become critical for cell protection. On the other hand, upon exposure to stresses that lead to apoptosis, the protective function of HSP-72 could be fully accounted for by its distinct role in cell signaling. Under these conditions, protein damage on its own is not sufficient for cell death because suppression of the apoptotic signaling pathway restores cell viability.

The term heat shock protein is somewhat of a misnomer, as they are not induced solely by heat shock. Indeed, in addition to being constitutively expressed (making up 5-10% of the total protein content under normal growth conditions), these proteins can be markedly induced (up to 15% of the total cellular protein content) by a range of stimuli including various pathological stresses.

Pathological stresses inducing heat shock protein expression include a wide variety of conditions associated with many diseases. The synthesis of heat shock proteins in cells exposed to such stresses indicates the first line of defense of the cell against the pathological stresses.

Stroke

One such pathological condition wherein protective role of HSP-70 has been implicated is cerebral ischemic injury (stroke). Cerebral ischaemia causes severe depletion of blood supply to the brain tissues, as a result of which the cells gradually proceed to death due to lack of oxygen. In such a situation, there is increased expression of heat shock protein in the brain tissue. Transient ischemia induces HSPs in the brain and the ability of neuronal population to survive an ischemic trauma is correlated with increased expression of HSP-70. HSP-70 mRNA was induced in neurons at the periphery of ischemia. It is proposed that the peripheral zone of ischemia, penumbra can be rescued by pharmacological agents. It was in this zone that HSP-70 protein was found to be localized primarily in neurons. [Dienel G. A. et al., J. Cereb. Blood Flow Metab., 1986, Vol. 6, pp. 505-510; Kinouchi H. et al., Brain Research, 1993, Vol. 619, pp. 334-338]. The direct assessment of the protective role of HSP-70 is shown by using transgenic mice overexpressing the rat HSP (HSP-70tg mice). In contrast to wild-type littermates, high levels of HSP messenger RNA and protein were detected in brains of HSP-70tg mice under normal conditions, immunohistochemical analysis revealed primarily neuronal expression of HSP-70. Heterozygous HSP-70tg mice and their wild type littermates were subjected to permanent focal cerebral ischemia by intraluminal blockade of middle cerebral artery. Cerebral infarction after 6 hours of ischemia, as evaluated by nissl staining, was significantly less in HSP-70tg mice compared with wild type littermate mice. The HSP-70tg mice were still protected against cerebral infarction 24 hours after permanent focal ischemia. The data suggest that HSP-70 can markedly protect the brain against ischemic damage. [Rajdev S., Hara K, et al., Ann. Neurol., 2000 June, Vol. 47 (6), pp. 782-791] The 72-kD inducible heat shock protein (HSP-72) plays a very important role in attenuating cerebral ischemic injury. Striatal neuronal survival was significantly improved when HSP-72 vectors was delivered after ischemia onset into each striatum. [Hoehn B. et al., J. Cereb. Blood Flow Metab., 2001 November, Vol. 21(11), pp. 1303-1309].

Experiments have proved that neurological deficits induced by ischemia were found to be reduced on treatment with HSP-inducers like lithium. These neuroprotective effects were associated with an up-regulation of cytoprotective heat shock protein −70 in the ischemic hemisphere [Ren M. et al., Proc. Natl. Acad. Sci. USA., 2003 May 13; Vol. 100(10), pp. 6210-6215]. Thus induction of HSP-70 would confer a protective effect in cerebral ischaemic injury (stroke).

Myocardial Infarction

Another pathological condition analogous to cerebral ischaemia is myocardial infarction, in which case, severe ischemia even for relatively short periods of time, lead to extensive death of cardiomyocytes. Induction of HSP-70 has been shown to confer protection against subsequent ischemia as is evident by a direct correlation to post-ischemic myocardial preservation, reduction in infarct size and improved metabolic and functional recovery. Overexpression of inducible HSP-70 in adult cardiomyocytes were associated with a 34% decrease in lactate dehydrogenase in response to ischemic injury. [Hutter M. M. et al., Circulation, 1994, Vol. 89, pp. 355-360; Liu X. et al., Circulation, 1992, Vol. 86, pp. II358-II363; Martin J. L., Circulation, 1997, Vol. 96, pp. 4343-4348].

Experiments have shown that oral pretreatment of rats with an HSP inducer Bimoclomol elevated myocardial HSP-70 and reduced infarct size in a rat model of ischemia [Lubbers N. L. et al., Eur. J. Pharmacol., 2002 Jan. 18, Vol. 435(1), pp. 79-83]. There was a significant correlation between HSP-70 induction and infarct size reduction after oral administration of Bimoclomol. Further, Bimoclomol also improved cell survival in rat neonatal cardiomyocytes by increasing the levels of HSP-70 [Polakowski J. S. et al., Eur. J. Pharmacol, 2002 Jan. 18, Vol. 435 (1), pp. 73-77].

In further experiments, transgenic mice were engineered to express high levels of the rat-inducible HSP-70 [Marber M. S. et al., J. Clin. Invest, 1995 April, Vol. 95, pp. 1446-1456]. It was observed that there was a significant reduction in infarct size by about 40% after 20 minutes of global ischemia in the heart of the transgenic mice, and contractile function doubled during reperfusion period compared to wild type.

Moreover, evidence indicate that myocardial stress protein HSP-70 is directly protective is provided by the observation that transfected myocyte lines overexpressing HSP-70 have enhanced resistance to hypoxic stress [Mestril R. et al., J. Clin. Invest., 1994 February, Vol. 93, pp. 759-767].

Further investigations into the role of HSP-70 overexpression through gene therapy on mitochondrial function and ventricular recovery has shown that, HSP-70 upregulation protects mitochondrial function after ischemia-reperfusion injury and was associated with improved preservation of myocardial function. Post ischemic mitochondrial respiratory control indices linked to NAD and FAD were better preserved and recovery of mechanical function was greater in HSP transfected than control hearts. [Jayakumar J. et al., Circulation, 2001 Sep. 18, Vol. 104 (12 Suppl 1), pp. 1303-1307]. Thus, the foregoing evidence indicates that induction of HSP-70 would be useful for treating myocardial infarction.

Inflammatory Disorders

Yet another example of pathological stress on tissues and organs, causing HSP-70 induction is provided by inflammatory diseases.

Inflammation is caused by activation of phagocytic cells like leucocytes, primarily by monocytes-macrophages, which generate high levels of reactive oxygen species (ROS) as well as cytokines. Both ROS and cytokines upregulate the expression of heat shock proteins (HSP), while HSPs in turn protect cells and tissues from the deleterious effects of inflammation. In an in vivo model for adult respiratory distress syndrome, an acute pulmonary inflammatory condition which caused HSP induction, it HSP completely prevented mortality. [Jacquier-Salin M. R. et al., Experientia, 1994 Nov. 30, Vol. 50 (11-12), pp. 1031-1038].

HSP exert multiple protective effects in inflammation, including self/non-self discrimination, enhancement of immune responses, immune protection, thermotolerance and protection against the cytotoxicity of inflammatory mediators [Polla B. S. et al., EXS., 1996, Vol. 77, pp. 375-91].

Heat shock proteins (HSPs) have been repeatedly implicated in the control of the progression of rheumatoid arthritis. An up-regulation of HSP-70 expression in synovial tissue is consistently observed in patients with rheumatoid arthritis. Recent investigations have shown that, pro-inflammatory cytokines induced activation of HSF 1-DNA binding and HSP-70 expression in cultivated synovial fibroblast-like cells [Georg Schett et. al, J. Clin. Invest., 1998 July, Vol. 102 (2), pp. 302-311]. Since HSP-70 is critically involved in protein folding and may prevent apoptotic cell death, facilitating synovial growth and pannus formation, their elevated levels would, play a crucial role in controlling the progression of the disease state.

Anti-inflammatory agents such as NSAIDS activate HSF-1 DNA binding and glucocortcoids at high dose activate HSF-1 as well as induce HSP expression [Georg Schett et. al., J. Clin. Invest., 1998 July, Vol. 102 (2), pp. 302-311].

HSP-70 has a role in controlling inflammation. The induction of HSP-70 before the onset of inflammation can reduce organ damage [Hayashi Y. et al, Circulation, 2002 Nov. 12, Vol. 106(20), pp. 2601-2607]. Preoperative administration of HSP-70 inducers seem to be useful in attenuating cardiopulmonary bypass (CPB)-induced inflammatory response.

Investigations into the anti-inflammatory property of 2-cyclopentene-1-one demonstrated that the heat shock factor 1 (HSF 1) activation, subsequent induction of HSP-72 expression occurs in inflamed tissue and this effect is associated with the remission of the inflammatory reaction. [Ianaro A. et al., Mol Pharmacol, 2003 July, Vol. 64(1), pp. 85-93]. The anti-inflammatory properties of 2-cyclopenten-1-one were associated with HSF-1 induced HSP-72 expression in vivo.

The HSP co-inducer BRX-220 has been examined for effects on the Cholecystokinin-octapeptide (CCK)-induced acute pancreatitis in rats [Rakonczay Z. Jr. et al., Free Radio. Biol. Med., 2002 Jun. 15, Vol. 32 (12), pp. 1283-1292]. The pancreatic levels of HSP-60 and HSP-72 were significantly increased in the animals treated with BRX-220. Further, pancreatic total protein content, amylase and trypginogen activities were higher with increased- glutathione peroxidase activity. A decrease in plasma trypsinogen activation peptide concentration, pancreatic lipid peroxidation, protein oxidation, and the activity of Cu/Zn-Superoxide dismutase were also observed. The protective action of BRX-220 on pancreatitis was ascribed directly to its HSP-70 inducing action.

Whole body hyperthermia in rats leading to induction of HSP-70 has been shown to protect against subsequent caerulein-induced acute pancreatitis. More specifically the degradation and disorganization of the actin cytoskeleton, an important early component of pancreatitis was prevented [Tashiro M. et al., Digestion, 2002, Vol. 65 (2), pp. 118-126], hence, reducing damage in pancreatitis secondary to inflammation. Thus induction of HSP-70 would be beneficial in treating inflammatory disorders.

Hepatotoxicity

Another example of a pathological stress wherein protective role of HSP-70 has been implicated is hepatotoxicity. Overproduction of heat shock protein 70 (HSP-70) in the liver protects hepatocytes; under various pathologic conditions. Studies aimed at examining the effects of HSP-70 inducers, on acute hepatic failure after 95% hepatectomy have shown significantly suppressed release of aspartate or alanine aminotransferase and elevation of the serum interleukin-6 level [Oda H. et al, J. Gastrointest. Surg., 2002 May-June, Vol. 6(3), pp. 464-472].

The effect of HSP Inducer gadolinium chloride was studied in relation to its effect on metallothionein and heat shock protein expression in an in-vivo model of liver necrosis induced by thioacetamide [Andrés D. et al., Biochem. Pharmacol, 2003 Sep. 15, Vol. 66 (6), pp. 917-926]. Gadolinium significantly reduced serum myeloperoxidase activity and serum concentration of TNF-alpha and IL-6, increased by thioacetamide. The extent of necrosis, the degree of oxidative stress and lipoperoxidation and microsomal FAD monoxygenase activity were significantly diminished. These beneficial effects are attributed to enhanced expression of HSP-70 following Gadolinium administration.

Thus induction of HSP-70 would exert a protective effect in case of hepatotoxicity.

Sepsis

Yet another pathological condition wherein induction of HSP-70 has been found to be beneficial is sepsis. Sepsis is a severe illness caused by overwheming infection of the bloodstream by toxin-producing bacteria. Induction of HSPs by heat shock treatment significantly decreased the mortality rate of late sepsis. The involvement of HSPs during the progression of sepsis could add to a first line of host defense against invasive pathogens.

Expression of HSP-72 and their protective role has been studied using a rat model of cecal ligation and puncture [Yang R. C. et al., Kaohsiung J. Med. Sci., 1998 Nov., Vol. 14 (11), pp. 664-672]. Induction of HSP-70 expression by Geranylgeranyl acetone has shown to protect against cecal ligation and perforation induced diaphragmatic dysfunction. It showed a time dependant induction of HSP-70 in the diaphragm, which attenuated septic diaphragm impairment. [Masuda Y. et al., Crit. Care Med., 2003 Nov., Vol. 31(11), pp. 2585-2591]. GGA has found to induce HSP-70 expression in the diaphragm, which was attributed to be the underlying mechanism for the protective action of GGA

Further experiments indicate that induction of HSP-70 by the administration of sodium arsenite conferred significant protection against cecal, ligation and perforation-induced mortality [Ribeiro S. P. et al., Crit. Care Med, 1994 Jun., Vol, 22(6), pp. 922-929]. In-vivo Sodium arsenite injection in the absence of an increase in body temperature induced expression of HSP-72 in the lungs and protected against experimental sepsis. Protection conferred resulting in reduced mortality correlated directly with the expression of heat shock protein 72 in the lungs at 18 and 24 hours after perforation.

It was observed that induction of heat shock proteins by thermal stress reduced organ injury and death in a rat model of intra-abdominal sepsis and sepsis-induced acute lung injury [Villar J. et al., Crit. Care Med., 1994 June, Vol. 22 (6), pp. 914-921].

Acute respiratory distress syndrome (ARDS) provokes three pathologic processes: unchecked inflammation, interstitial/alveolar protein accumulation and destruction of pulmonary epithelial cells. Heat shock protein HSP-70 can limit all three responses, only if expressed adequately. Restoring expression of HSP-70 using adenovirus-mediated gene therapy has shown to be beneficial [Yoram G. W. et al., J. Clin. Invest, 2002, Vol. 110, pp. 801-806]. HSP-70 administration significantly attenuated interstitial and alveolar edema along with protein exudation and dramatically decreased neutrophil accumulation. Approximately 2-fold higher expression of HSP-70 conferred 68% survival at 48 hours as opposed to only 25% in untreated animals. Modulation of HSP-70 production reduced the pathological changes and improved outcome in experimental acute respiratory distress syndrome. Thus, inducers of HSP-70 would confer protective effect in sepsis.

Viral Diseases

Another pathological condition in which induction of HSP-70 occurs is in case of viral diseases. Heat shock proteins (HSPs) and molecular chaperones have been known for several years to protect cells against virus infection [Lindquist S. et al., Annu. Rev. Genet., 1988, Vol. 22, pp. 631-637]. It has been demonstrated that induction of HSP-70 is associated with inhibition of infectious virus production and viral protein synthesis in monkey kidney epithelial cells infected with vesicular stomatitis virus (VSV) [Antonio R. et al., J. of Biol. Chem., 1996 Issue of December 13, Vol. 271 (50), pp. 32196-32196]. The pathogenic activity of Viral protein R (Vpr) of human immunodeficiency virus type 1 (HIV-1) is related in part to its capacity to induce cell cycle G2 arrest and apoptosis of target T cells. Overexpression of HSP-70 reduced the Vpr-dependent G2 arrest and apoptosis and also reduced replication of the Vpr-positive, but not Vpr-deficient, HIV-1. [Iordanskiy S. et al., J. Virol, 2004 September, Vol. 78 (18), pp. 9697-9704]. Induction of HSP-70 by prostaglandin A1 (PGA1) caused the suppression of influenza virus production. [Hirayama E., Yakugaku Zasshi, 2004 July, Vol. 124 (7), pp. 437-442].

The antiviral activity of Cyclopentenone prostaglandins is mediated by induction of HSP-70. It has been shown that increased synthesis of HSP-70 exerts potent antiviral activity in several DNA and RNA virus models—vesicular stomatitis virus, sindbis virus, sendai virus, polio virus etc. [Santoro M. G., Experientia, 1994 Nov. 30, Vol. 50 (11-12), pp. 1039-1047; Amici C. et al., J. Gen. Virol, 1991 August, Vol. 72, pp. 1877-1885; Amici C. et al., J. Virol., 1994 November, Vol. 68(11), pp. 6890-6899; Conti C. et al, Antimicrob. Agents Chemother., 1996 February, Vol. 40(2), pp. 367-372; Conti C. et al., Antimicrob. Agents Chemother., 1999 April, Vol. 43 (4), pp. 822-829]. Therefore, induction of HSP-70 would exert antiviral effect.

Allograft Rejection

Allograft (transplant of an organ or tissue from one individual to another of the same species with a different genotype) rejection is a pathological condition causing induction of HSP-70. HSP-70 induction has a protective effect, which preserves organ function after transplantation. Kidneys can be preserved only for a limited time without jeopardizing graft function and survival. Induction of heat shock proteins (HSPs) has been found to improve the outcome following isotransplantation after an extended period of cold storage. Heat precondition induced the expression of HSP-70 and the grafts were protected against structural ischemia-reperfusion injuries when assessed histologically. [Wagner M. et al., Kidney Int., 2003 April, Vol. 63 (4), pp. 1564-1573]. There was inhibition of apoptosis and activation of caspase-3 was found to be inhibited.

Geranylgeranyl acetone, a non-toxic heat shock protein inducer has been studied in a rat orthotopic liver transplantation model to study the beneficial effects in warm ischemia-reperfusion injury [Fudaba Y. et al., Transplantation, 2001 Jul. 27, Vol. 72(2), pp. 184-189]. GGA administration accumulated mRNA for both HSP-72 and HSP 90 in the livers even before warm ischemia and facilitated the syntheses of HSP-72 and HSP 90 after warm ischemia. Further, GGA pretreatment also significantly reduced the serum levels of tumor necrosis factor-alpha after reperfusion. The findings indicate that both the enhanced induction of HSPs and the downstream events would be involved in the beneficial effects of GGA on ischemia-reperfusion injury. Besides, compared to donors treated with vehicle were all recipients died of primary non-function, when donors were treated with Geranylgeranyl acetone (GGA) the 7-day survival of the recipients was closed to 90%.

Investigations revealed an inverse relationship between HSP expression and rejection with the possibility that elevated levels of HSP in the myocardium results in low rejection of heart transplants. [Baba H. A. et al., Transplantation, 1998 Mar. 27, Vol. 65 (6), pp. 799-804]. Significant improvement of post-ischemic recovery of mechanical function in HSP-70 gene transfected hearts compared to controls were observed following a protocol mimicking conditions of preservation for heart transplantation. These results confirmed the findings observed previously in cell culture models and extended then to show the role of HSP-70 in protecting against ischemia-reperfusion injury in a whole-heart model, which parallels more closely the clinical situation. [Jayakumar J. et al., Circulation, 2000, Vol. 102 [suppl III], pp. 111-302 to 111-306].

The heat shock response also exerts a protective effect on skin flap ischemia. Heat shock protein (HSP) expression is augmented in-vivo with the administration of high dose aspirin before heat treatment [Ghavami A. et al., Ann. Plast. Surg., 2002 January, Vol. 48(1), pp. 60-67]. Immunohistochemistry confirmed HSP expression, and skin flap survival was improved significantly. Thus, HSP-70 induction would be beneficial in preserving organ function after transplantation.

Tumorous Diseases

I Induction of HSP-70 has also been shown to be advantageous in treating neoplasms. Enhanced expression of HSP-70 has been found to help in causing tumor regression in various animal models. Heat shock proteins (HSPs) are involved in the development of resistance (thermotolerance) to subsequent hyperthermic stresses as well as enhancement of the clinical response of certain chemotherapeutic agents in cancers such as the prostate. Colony formation assays revealed sensitizing effect of hyperthermia when simultaneously combined with each chemotherapeutic agent, resulting in a potentiated localized cytotoxicity [Roigas J. et al, Prostate, 1998 Feb. 15, Vol. 34 (3), pp. 195-202]. Synchronous application of chemotherapeutic agents and hyperthermia has been shown to have synergistic cytotoxic effect on Dunning rat adenocarcinoma of the prostate. Furthermore it is demonstrated that the induction of HSPs in thermotolerant cells, as measured by HSP-70 induction, results in a modulation of the chemotherapeutic-mediated cytotoxicity.

Direct induction of heat shock proteins are recognized to contribute significantly in cancer immunity. Anti-tumor immunity is induced by hyperthermia and further enhanced by administration of recombinant HSP-70 protein into the tumor in-situ. [Ito A. et al., Cancer Immunol. Immunother., 2004 January, Vol 53(1), pp. 26-32]. The induction of hyperthermia using a 500 KHz alternating magnetic field combined with magnetite cationic liposomes, which have a positive charge and generate heat in an alternating magnetic field along with administration of recombinant HSP-70 protein into the subcutaneous murine melanoma inhibited tumor growth over a 30-day period and complete regression of tumors was observed in 20% of mice. It was also found that systemic anti-tumor immunity was induced in cured mice. In another study carried out to determine whether anti-tumor immunity induced by hyperthermia is enhanced by HSP-70 gene transfer [Ito A. et al., Cancer Gene Ther., 2003 December, Vol. 10(12), pp. 918-925] showed that the combined treatment strongly arrested tumor growth over a 30-day period and complete regression of tumors was observed in 30% mice. Thus, induction of HSP-70 would be useful for the treatment of tumorous diseases.

Gastric Mucosal Damage

Gastric mucosal damage caused by insults derived from ingested foods and Helicobacter pylori infection constitute another pathological condition causing induction of HSP-70. Gastric surface mucous cells are the first line of defense against such insults. Primary cultures of gastric surface mucous cells from guinea-pig fundic glands exhibited a typical heat shock response after exposure to elevated temperature or metabolic insults, such as ethanol and hydrogen peroxide, and they were able to acquire resistance to these stressors. HSP-70 mRNA protein has been induced in rat gastric mucosa following stress and the extent of induction inversely correlated with the severity of mucosal, lesions suggesting protective role of HSP-70 in gastric mucosal defense. [Rokutan K., J. Gastroenterol. Hepatol, 2000 March, Vol. 15 Suppl, pp. D12-9].

Brain Haemorrhage

Another pathological condition causing induction of HSP-70 is in case of brain haemorrhage. Studies with Bimoclomol showed an ability to reduce the pathological increase in the permeability of blood brain barrier during cerebrovascular injury, particularly if the vascular insult is evoked by sub-arachnoidal autologous blood [Erdo F. et al., Brain Research Bulletin, 1998, Vol. 45(2), pp. 163-166]. Bimoclomol strongly reduced the size of cerebral tissue stained with Evans blue leakage by 39%. Bimoclomol confers beneficial influences in experimental sub-arachnoid haemorrhage through its co-inducer effect on HSP-72 expression.

Endothelial Dysfunctions

Various endothelial dysfunctions constitute pathological conditions which results in induction of HSP-70 in the body cells. The effect of a co-inducer of heat shock proteins, Bimoclomol treatment on endothelial function and expression of 72 Kd heat shock protein was investigated in spontaneously hypertensive rats [Jednakovits A. et. al., Life Sci., 2000 Aug. 25, Vol. 67(14), pp. 1791-1797]. Significant age-dependant decline in relaxation to acetylcholine and vascular HSP-72 mRNA levels were observed in SHR animals. These changes were found to be prevented by application of Bimoclomol suggesting the relationship between preservation of endothelial function with sustained levels of HSP-72.

Diabetic Complications

Complications arising in diabetic patients such as neuropathy, nephropathy and delayed wound healing constitute pathological conditions wherein protective role of HSP-70 has been implicated.

(a) Diabetic Neuropathy

Endoneurial microangiopathy causing nerve infarctions is considered to be involved in the pathogenesis of diabetic neuropathy [Malik R. A. et al, Diabetic Neuropathy: New Concepts and Insights, 1995, pp 131-135]. Experimental evidence is suggestive of a protective effect of HSP-72 induction on diabetic neuropathy [Biro K. et. al, Brain Research Bulletin, 1997, Vol. 44(3), pp. 259-263 ]. Treatment with Bimoclomol, by virtue of its HSP-70 inducing property significantly reduced nerve conduction slowing, motor by 38% and sensory by 42%, which show a dose dependant response. It also retarded the typical elevated ischemic resistance due to streptozotocin-induced neuropathy by 71%. These effects were observed at doses known to induce transcription of HSP-72 in other tissues like heart and kidney in response to ischemia.

(b) Diabetic Retinopathy

Diabetic retinopathy is associated with the breakdown of the blood-retinal barrier (BRB) and results in macular edema, the leading cause of visual loss in diabetes. The HSP co-inducer Bimoclomol (BRLP-42) has shown efficacy in diabetes-induced retinopathy [Hegedius S. et al., Diabetologia, 1994, Vol. 37, p. 138]. The protection reflected in lower degree of edema in and beneath the photoreceptor zone, almost normal arrangement of retinal pigment epithelial microvilli and a more compact and even retinal capillary basement membrane. [Biro K. et al, Neuro Report, 1998 Jun. 22, Vol. 9(9), pp. 2029-2033]. Improvements are attributed to the cytoprotective effect of Bimoclomol on retinal glia and/or neurons against diabetes related ischemic cell damages. Further, overexpression of HSP-70 has shown protective effect on retinal photic injuries [Kim J. H. et al., Korean J. Ophthalmol: 2003 June, Vol. 17(1), pp. 7-13].

(c) Chronic Wound Healing

HSPs are involved in regulation of cell proliferation. Impaired expression of HSP-70 has been associated with delayed wound healing in diabetic animals [McMurtry A. L. et al., J. Surg. Res., 1999, Vol. 86, pp. 36-41]. Faster and stronger healing is achieved by activation of HSP-70 in a wound by laser [Capon A. et al., Lasers Surg. Med., 2001, Vol. 28, pp. 168-175].

Thus, induction of HSP-70 would be beneficial in treating various diabetic complications.

Neuro-Degenerative Diseases

Neurodegenerative diseases such as Alzheimer's disease, Amyotrophic lateral sclerosis and Parkinson's disease constitute a set of pathological conditions wherein HSP-70 has been implicated to exert a protective affect and delay the progression of these diseases.

(a) Alzheimer's disease, is a neurodegenerative disorder characterized by beta-amyloid and tau protein aggregates (neurofibrillary tangles) Increased levels of HSP (8-10 fold increase) in various cellular models have shown to promote tau solubility and tau binding to microtubules, reduce insoluble tau and cause reduced tau phosphorylation. Hence upregulation of HSP will suppress formation of neurofibrillary tangles. [Dou F. et al., Proc. Natl. Acad. Sci. USA, 2003 Jan. 21, Vol. 100 (2), pp. 721-726]. Studies have shown that virally mediated HSP-70 overexpression rescued neurons from the toxic effects of intracellular beta-amyloid accumulation. [Magrané J. et al., J. Neurosci., 2004 Feb. 18, Vol. 24 (7), pp. 1700-1706].

(b) Amyotrophic lateral sclerosis (ALS) is a fatal neurodegenerative condition in which motor-neurons of the spinal cord and motor cortex die, resulting in progressive paralysis. Etiology of ALS involves mutation in the gene encoding Cu/Zn superoxide dismutase-1 (SOD1). Treatment with arimoclomol, an inducer of heat shock proteins (HSPs), significantly delays disease progression in transgenic mice overexpressing human mutant SOD1 that shows a phenotype and pathology that is very similar to that seen in human ALS patients. [Kieran D. et al., Nat. Med., 2004 April, Vol 10 (4), pp. 402-405; Susanna C. B. et al, Nat. Med., 2004, Vol. 10, pp. 345-347].

(c) Parkinson's disease is a common neurodegenerative disease characterized by the loss of dopaminergic neurons in the substantia nigra pars compacta and the accumulation of the misfolded protein alpha-synuclein into aggregates called Lewy bodies and Lewy neuritis, which are very cytotoxic. Mitochondrial dysfunction, oxidative stress, protein misfolding, aggregation, and failure in the proteasomal degradation of specific neuronal proteins have been implicated in pathogenesis of Parkinson disease (PD). Upregulation of HSP-70 by HSP-70 gene transfer to dopamine neurons by a recombinant adeno-associated virus significantly protects the mouse dopaminergic system against MPTP-induced dopamine neuron loss and the associated decline in striatal dopamine levels. [Dong Z. et al., Mol. Ther., 2005 January, Vol. 11(1), pp. 80-88]. Recent experimental evidences show that deprenyl and other propargylamines which are used clinically in treating Parkinson's disease increase, neuronal survivability by increasing synthesis of HSP-70 and other anti-apoptotic proteins. [Tatton W. et al., J. Neural. Transm., 2003 May, Vol. 110(5), pp. 509-515]. Introducing HSP-70 in alpha-synuclein transgenic mice by breeding with HSP-70 overexpressing mice led to significant reduction in misfolded and aggregated alpha-synuclein in the progeny. [Klucken J. et al., J. Biol. Chem., 2004 Jun. 11, Vol. 279 (24), pp. 5497-5502]. Recent evidences show that Geldanamycin protects neurons against alpha-synuclein toxicity by enhancing the HSP-70 mediated chaperonic activity. [Auluck P. K. et al., J. Biol. Chem., 2005 Jan. 28, Vol. 280 (4), pp. 2873-2878].

Thus, HSP-70 inducers would be useful in the treatment and delaying the progression of the above neurodegenerative disease conditions.

Post-Traumatic Neuronal Damage

Pathological stress associated with post-traumatic neuronal damage cause induction of HSP-70 in the neuronal tissues. The expression of HSP-70 following traumatic injury to the neuronal tissue has been speculated to be part of a cellular response, which is involved in the repair of damaged proteins [Dutcher S. A et al, J. Neurotrauma, 1998, Vol. 15 (6), pp. 411-420]. BRX-220, an inducer of HSP-70 has been examined for its effect on the survival of injured motoneurones following rat pup sciatic nerve crush [Kalmar B. et al., Exp. Neurol, 2002 July, Vol. 176 (1), pp. 87-97]. It has been found that significantly more number of neurons survived with BRX-220 treatment and there was no further loss of motoneurones. 14 days after injury, 39% of motoneurones survived in BRX220 treated group compared to 21% in vehicle group. Moreover in BRX 220 treated group no further loss of motoneurones occurred, at 10 weeks 42% of motoneurons survived compared to 15% in untreated group. There were also more functional motor units in the hind limb muscles of the treated group compared to that of the control. These observations were correlated to elevated levels of HSP-70 and this compound protects motoneurones from axotomy-induced cell death through a HSP-70 mediated mechanism. Therefore, induction of HSP-70 would be beneficial in post-traumatic neuronal damage.

Acute Renal Failure

Another pathological condition causing induction of HSP-70 is acute renal failure. Acute renal failure is the sudden loss of the ability of the kidneys to excrete wastes, concentrate urine and conserve the electrolytes. Induction of heat shock proteins (HSPs) plays a protective role in ischaemic acute renal failure. Administration of Sodium arsenite or Uranyl acetate in cisplatin-induced acute renal failure resulted in significant increase in HSP-72 expression. Both Sodium arsenite and Uranyl acetate attenuated the cisplatin-induced increase in serum creatinine and tubular damage scores [Zhou H. et al., Pflugers Arch., 2003 April, Vol. 446 (1), pp. 116-124]. Findings suggest that HSP-72 attenuates CDDP-induced nephrotoxicity. The protective effects of HSP-72 are associated with an increased Bcl-2/Bax ratio and reduced apoptosis.

Glaucoma

Still another pathological condition which causes induction of HSP-70 is glaucoma. Glaucoma is characterized by rising intra intraocular pressure and subsequent damage to the optic nerve with selective loss of retinal ganglion cells (RGCs). It has been postulated that apoptosis, a highly regulated process of cell death, is the final common pathway for RGC death in glaucoma. Studies suggest that the induced expression of HSP-72 enhances RGC survival in harmful conditions and ameliorates glaucomatous damage in a rat model [Ishii Y. et al, Invest. Ophthalmol. Vis. Sci., 2003 May, Vol. 44(5), pp. 1982-1992]. The study revealed that HSP-72 expression was increased in retinal ganglion cells after administration of HSP inducer geranylgeranyl acetone. The treatment further reduced the loss of retinal ganglion cells, reduced optic nerve damage and decreased the number of TUNEL positive cells in retinal ganglion cell layer.

Aging Related Skin Degeneration

There is an attenuation of induction of HSP-70 in human keratocytes with aging [Verbeke P. et al, Cell Biol. Int., 2001, Vol. 25 (9), pp. 845-857]. Furthermore, human skin cells have been shown to maintain several characteristics of young cells until late in life, when exposed to repetitive mild heat shocks [Rattan S. I. et al., Biochem. Mol. Biol. Int., 1998, Vol. 45(4), pp. 753-759].

Over expression of heat shock protein gene is sufficient to protect against otherwise lethal exposures to heat, ischemia, cytotoxic drugs, and toxins. The above examples illustrate the ability of HSP-70 to protect cells against various pathological stresses contributing towards different diseases.

U.S. Pat. No. 5,348,945 describes methods for enhancing the survivality of cells and tissues by treating the same with exogenous HSP-70.

A number of compounds have been reported to be useful for increasing levels of HSPs thereby treating a range of disorders.

U.S. Pat. No. 6,096,711 discloses methods for inducing HSP-72 production in an aged cell by contacting the aged cell with a proteasome inhibitor, and treating stress-induced pathologies associated with apoptosis and inflammation in aged individuals.

U.S. Pat. No. 6,174,875 discloses methods for inducing HSP-70 and treating neurological injuries resulting from cardiac arrest and stroke by inhibiting cell death induced by oxidative stress, with benzoquinoid ansamycins.

U.S. Pat. No. 6,653,326 describes methods for increasing expression of molecular chaperones, including HSP-70 using hydroxylamine derivatives, and thereby treating stress related diseases like stroke, cerebrovascular ischaemia, coronarial diseaseas, allergic diseases, immune diseases, autoimmune diseases, diseases of viral or bacterial origin, tumourous, skin and/or mucous diseases, epithelial disease of renal tubules, atherosclerosis, pulmonary hypertonia and traumatic head injury.

In view of the advantages associated with increased expression of HSP-70 in cells, a method, which increases such expression or increases activity of HSP-70 would be highly advantageous for prevention and treatment of various diseases. Small molecules that either enhances the expression or function of heat shock proteins could have promise in chronic or acute treatment of certain human diseases.

Compounds of the present invention have been categorically shown to induce HSP-70. Therefore, these compounds would be beneficial in the prevention and treatment of conditions where HSP induction has been shown to protect in various diseased states, for example in stroke, myocardial infarction, inflammatory diseases, diseases of viral origin, tumourous diseases, brain haemorrhage, endothelial dysfunctions, diabetic neuropathy, hepatotoxicity, acute renal failure, glaucoma, sepsis, gastric mucosal damage, allograft rejection, chronic wounds in diabetics, neurodegenerative diseases, post-traumatic neuronal damage and aging-related skin degeneration.

None of the compounds as disclosed in this application have been identified in any published patents, patent applications or journal articles. A few structurally similar propen-1-one derivatives and their analogs that have been reported are discussed hereinbelow.

PCT publications WO 03/097575 and WO 03/097576 disclose certain amino and diamino functional chalcones that are useful against bacterial as well as parasitic infections of general formula (I)
(Y¹)ₘ—Ar¹(X¹)—C(=O)VAr²(X²)—(Y²)ₚ (I)
wherein Ar¹ and Ar² independently designate aryl or heteroaryl; V designates —CH=CH—; m is 0, 1 or 2; p is 0, 1 or 2; wherein the sum of m and p is at least 1; Y¹ and Y² independently designate amino or diamino substituent of the formula -Z—N(R¹)R² or —NR³-Z—N(R¹)R²; wherein Z is a biradical —(C(R^{H})₂)ₙ—, wherein n is an integer in the range of 1-6, and each R^{H} is independently selected from hydrogen or C₁₋₆alkyl, or two R_{H} on the same carbon atom may designate =O. Examples of these compounds are 1-{3-[(2-Dimethylamino-ethyl)-methyl-amino]-phenyl}-3-phenyl-propenone and 3-(4-Dibutylamino-phenyl)-1-(3-dimethylaminomethyl-phenyl)-propenone.

PCT Publication WO 95/06628 describes the preparation of chalcones that are useful against bacterial as well as parasitic infections.

PCT Publication WO 93/17671 also describes the preparation of chalcones of general formula (I) useful against bacterial as well as parasitic infections.
Xₘ—Ar¹—CO—W—Ar²—Yₙ (I)
In the above chalcones of formula (I), W is —CR=CR— or —C≡C—, wherein each R independently designates hydrogen, C₁₋₃alkyl, or halogen; Ar¹ amd Ar² independently designate phenyl and 5- or 6-membered unsaturated heterocyclic ring; Y and X independently designate AR_{H} or AZ, wherein A is —O—, —S—, —NH— or —N(C₁₋₆alkyl)—; R_{H} designates C₁₋₆alkyl, C₁₋₆alkylene or C₁₋₆alkylyne; Z designates H; m designates an integer from 0 to 2 and n designates an integer from 0 to 3.

JP 05025115 describes the preparation of phenyl acetamide derivatives of formula (I) as antilipidemic agents, inhibiting the enzymes acyl-coenzyme A and cholesterol acryltransferase. 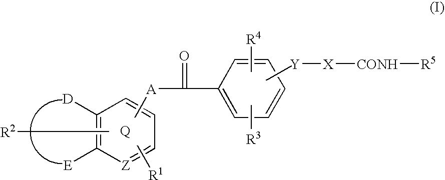
In the said amide compounds of formula (I), R¹ and R² are H, halogen, nitro, amino, OH, alkyl, alkoxy, aryl; R³ and R⁴ are H, halogen, amino, alkyl, alkoxy, R⁵ is aryl or arylalkyl; A is alkylene or alkenylene; X is single bond or N (R⁷) (R⁷ is H, alkyl, cycloalkyl); Y is alkylene, thia-alkylene or a single bond; Z is CH or N; D and E are H, R¹, R², H. An example of these compounds is N-(2,6-diethylphenyl)-3-[4-[2-(3,5-di-tertbutyl-4-hydroxyphenylthio)ethoxy]cinnamoyl]phenylacetamide.

JP 2003040888 describes the preparation of imidazoles of formula (I) having inhibitory activity against adhesion of synoviocyte to collagen and production of cytokine. 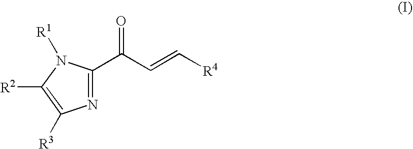
In the said imidazole derivative of general formula (I), R¹ is a substituted aryl; R² and R³ are each H, a substituted heteroaryl or R² and R³ together form a substituted heteroaryl; and R⁴ is a substituted heteroaryl.

WO 02/098875 discusses about carboline derivatives of general formula (I) as phosphodiesterase 5 (PDE5) inhibitors.

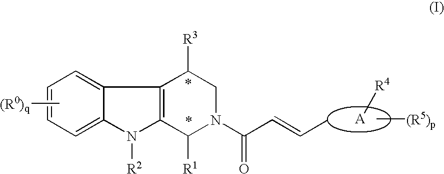

Some structurally similar propen-1-one derivatives and their analogs have also been reported to be useful as Heat Shock Protein (HSP) inducers. PCT Publication WO 00/18390 discloses several chalcone derivatives of general formula (I)

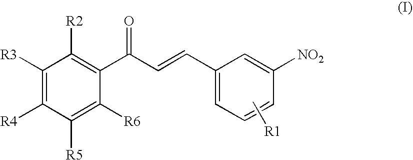

As it is evident from the general formula, the —NO₂ group present on the aryl ring is essential for activity in this series of molecules. Moreover, the general formula of WO 00/18390 consists of substituted or unsubstituted phenyl ring on the carbonyl side of the enone chain, while the substituted or unsubstituted 3-nitrophenyl ring is present on the olefinic side of the enone chain.

### SUMMARY OF THE INVENTION

The object of the invention is to provide novel compounds of the general formula (I), their derivatives, their analogs, their tautomeric forms, their stereoisomers, their polymorphs, their pharmaceutically acceptable solvates, their pharmaceutically acceptable salts, esters or prodrugs and pharmaceutically acceptable compositions containing them, which are useful in the treatment and/or prophylaxis of diseases accompanying pathological stress selected from stroke, myocardial infarction, inflammation, diseases of viral origin, tumourous diseases, brain haemorrhage, endothelial dysfunctions, diabetic neuropathy, hepatotoxicity, acute renal failure, glaucoma, sepsis, gastric mucosal damage, allograft rejection, chronic wounds in diabetics, neurodegenerative diseases, post-traumatic neuronal damage and aging-related skin degeneration wherein the underlying mechanism is Heat Shock Protein (HSP) induction.

Another object of the present invention is to provide a process for the preparation of the compounds of the general formula (I).

A further object of the invention is to provide the pharmaceutically acceptable compositions containing compounds of the general formula (I).

Yet another object of the invention is to use compounds of general formula (I) in the manufacture of medicaments useful for treatment of disease conditions in a mammal by induction of HSP.

Still further object of the invention is to provide a method of treatment of disease conditions that can be treated by induction of HSP through administration to a patient in need an effective amount of compounds of general formula (I).

### DETAILED DESCRIPTION OF THE INVENTION

Accordingly, the present invention provides for novel compounds of 2-propene-1-one series, of general formula (I), 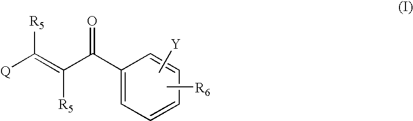
their derivatives, their analogs, their tautomeric forms, their stereoisomers, their polymorphs, their pharmaceutically acceptable solvates, their pharmaceutically acceptable salts, esters or prodrugs, and pharmaceutically acceptable compositions containing them, wherein Q represents a heteroaryl ring, said heteroaryl ring containing upto 2 nitrogen atoms and is selected from : 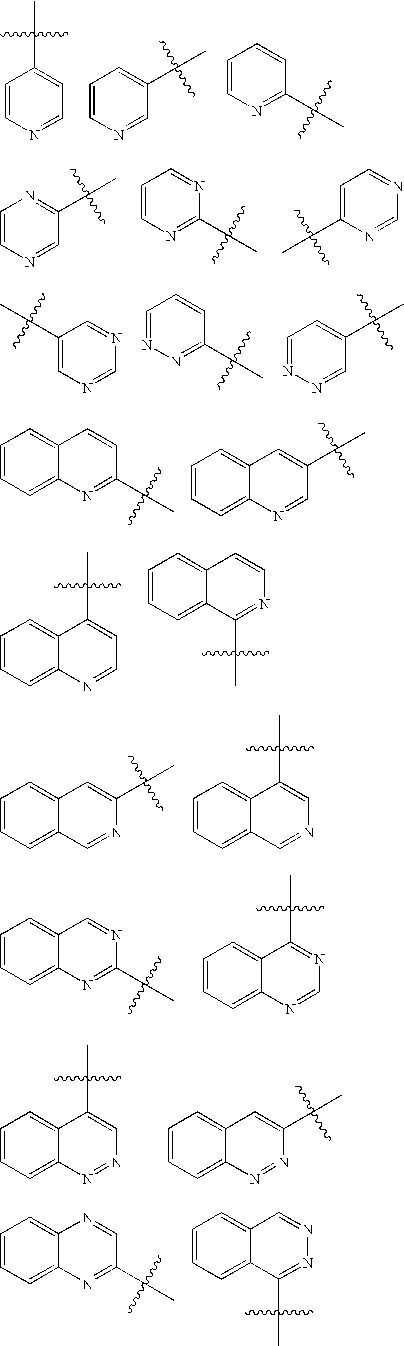 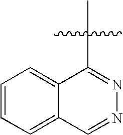
wherein, Q is optionally substituted by R₁ and/or R₂, and the number of substituents are selected from one to six;

R₁ is independently selected at each occurrence from —SO₂OR₇, —SO₂O(C₁₋₈alkyl), —NHNH₂, —NHNHSO₂R₇, —NH(CH₂)ₙR₄, —NHCO₂R₇, —NHCO₂(C₁₋₈alkyl), —NHSO₂O(C₁₋₈alkyl), —NHSO₂OR₇, —NHSO₂NH₂, —NH(CH₂)ₙCOR₄, —NH(CH₂)ₙOR₄, —NH(CH₂)ₙSR₇, —NH(CH₂)ₙSO₂R₇, —NH(CH₂)ₙNHCOR₄, —NH(CH₂)ₙN(C₁₋₈alkyl)COR₄, —N(C₁₋₈alkyl)(CH₂)ₙNHCOR₄, —NH(CH)₂)ₙNHNHSO₂R₇, —NH(CH₂)ₙNHSO₂R₄, —NH(CH₂)ₙN(C₁₋₈alkyl)SO₂R₄, —NH(CH₂)ₙN(NH₂)R₇, —NH(CH₂)ₙN[N(C₁₋₈alkyl)₂]R₇, —N(C₁₋₈alkyl)CO₂R₇, —N(C₁₋₈alkyl)CO₂(C₁₋₈alkyl), —N(C₁₋₈alkyl)SO₂O(C₁₋₈alkyl), —N(C₁₋₈alkyl)SO₂OR₇, —N(C₁₋₈alkyl)SO₂NH₂, —N(C₁₋₈alkyl)N(C₁₋₈alkyl)₂, —N(C₁₋₈alkyl)NH₂, —NHNHCO(C₁₋₈alkyl), —N(C₁₋₈alkyl)NHCO(C₁₋₈alkyl), —NHNHCOR₇, —N(C₁₋₈alkyl)NHCOR₇, —N(C₁₋₈alkyl)—(CH₂)ₙR₄, —N(C₁₋₈alkyl)(CH₂)ₙCOR₄, —(CH₂)ₙSO₂R₇, —(CH₂)ₙCOR₄, —(CH₂)ₙR₄, —(CH₂)ₙNHSO₂R₄, —(CH₂)ₙN(C₁₋₈alkyl)SO₂R₄, —(CH₂)ₙNHCOR₇, —CH₂)ₙN(C₁₋₈alkyl)COR₇, —CH₂)ₙOR₄, —(CH₂)ₙSR₄, —(CH₂)ₙSR₃, —(CH₂)ₙSO₂R₇, —(CH₂)ₙNHNHSO₂R₇, —(CH₂)ₙN(NH₂)R₇, or —(CH₂)ₙN[N(C₁₋₈alkyl)₂]R₇;

R₂ is independently selected at each occurrence from hydrogen, hydroxy, halo, amino, C₁₋₈alkyl, —O(C₁₋₈alkyl), —S(C₁₋₈alkyl), —SO₂(C₁₋₈alkyl), oxo, thioxo, mono(C₁₋₈alkyl)amino, di(C₁₋₈alkyl)amino, —NHCO(C₁₋₈alkyl), —N(C₁₋₈alkyl)CO(C₁₋₈alkyl), —NHSO₂(C₁₋₈alkyl), —NHSO₂CF₃, —N(C₁₋₈alkyl)SO₂CF₃, —NHSO₂O(C₁₋₈alkyl), —N(C₁₋₈alkyl)SO₂(C₁₋₈alkyl), —N(C₁₋₈alkyl)SO₂O(C₁₋₈alkyl), —COOH, —CO₂(C₁₋₈alkyl), —NHCO₂(C₁₋₈alkyl), —N(C₁₋₈alkyl)CO₂(C₁₋₈alkyl), —CONH₂, —CONH(C₁₋₈alkyl), —CON(C₁₋₈alkyl)₂, formyl, CF₃, CN, —(CH₂)ₙOH, —(CH₂)ₙNH₂, —(CH₂)ₙNH(C₁₋₈alkyl), —(CH₂)ₙN(C₁₋₈alkyl)₂, —(CH₂)ₙO(C₁₋₈alkyl), —SO₃H, —SO₂O(C₁₋₈alkyl), —SO₂NH₂, —SO₂N(C₁₋₈alkyl)₂, —SO₂NH(C₁₋₈alkyl), —OSO₂(C₁₋₈alkyl), —N(C₁₋₈alkyl)SO₂NH₂, —NHSO₂NH(C₁₋₈alkyl), —NHSO₂N(C₁₋₈alkyl)₂, —N(C₁₋₈alkyl)SO₂N(C₁₋₈alkyl)₂, —NHSO₂NH₂, —NHC(NH)NH₂, —NHCONH₂, —NHC(O)NH(C₁₋₈alkyl), —NHC(O)N(C₁₋₈alkyl)₂, —N(C₁₋₈alkyl)C(O)N(C₁₋₈alkyl)₂, —NHNH₂, —N(C₁₋₈alkyl)N(C₁₋₈alkyl)₂, —N(C₁₋₈alkyl)NH₂, tetrazolyl, or three- to seven-membered heterocyclyl or heteroaryl ring having upto three heteroatoms independently selected from N, O, or S, wherein said three- to seven- membered heterocyclyl or heteroaryl ring is optionally substituted with 1, 2 or 3 substituents independently selected from the group consisting of halo, hydroxy, C₁₋₈alkyl, —O(C₁₋₈alkyl), nitro, amino, mono(C₁₋₈alkyl)amino, di(C₁₋₈alkyl)amino, —NHCO(C₁₋₈alkyl), —N(C₁₋₈alkyl)CO(C₁₋₈alkyl), —NHSO₂(C₁₋₈alkyl), —N(C₁₋₈alkyl)SO₂(C₁₋₈alkyl), —NHSO₂CF₃, —N(C₁₋₈alkyl)SO₂CF₃, —COOH, —CONH₂, —CONH(C₁₋₈alkyl), —CON(C₁₋₈alkyl)₂, —CO₂(C₁₋₈alkyl), —NHCO₂(C₁₋₈alkyl), —N(C₁₋₈alkyl)CO₂(C₁₋₈alkyl), CF₃, CN, —(CH₂)ₙOH, —(CH₂)ₙNH₂, —(CH₂)ₙNH(C₁₋₈alkyl), —(CH₂)ₙN(C₁₋₈alkyl)₂, —CH₂O(C₁₋₈alkyl), —NHSO₂NH₂, —N(C₁₋₈alkyl)SO₂NH₂, —NHSO₂NH(C₁₋₈alkyl), —NHSO₂N(C₁₋₈alkyl)₂, —N(C₁₋₈alkyl)SO₂N(C₁₋₈alkyl)₂, —NHCONH₂, —NHCONH(C₁₋₈alkyl), —NHCON(C₁₋₈alkyl)₂, —N(C₁₋₈alkyl)CON(C₁₋₈alkyl)₂, —S(C₁₋₈alkyl), —SO₂(C₁₋₈alkyl), —SO₃H, —SO₂O(C₁₋₈alkyl), —SO₂NH₂, —SO₂N(C₁₋₈alkyl)₂, —SO₂NH(C₁₋₈alkyl), or —NHC(NH)NH₂;

‘Y’ is selected from the group consisting of:
(a) —C(O)NRₐR_{b},
(b) —NR_{c}C(X)NRₐR_{b},
(C) —NR_{c}C(X)NR_{d}Rₑ,
(d) —NR_{c}C(O)OR_{f},
(e) —NR_{c}C(O)C(O)R_{g};

X is selected from O or S;

Rₐ and R_{b} together with the atoms with which they are attached form a three- to ten-membered monocyclic or bicyclic heterocyclyl or heteroaryl ring selected from the group consisting of aziridinyl, azepanyl, azetindinyl, azocanyl, azepinyl, diazepanyl, diazocanyl, hexahydropyridazinyl, hexahydropyrimidinyl, isothiazolidinyl, isoxazolidonyl, imidazolyl, imidazolidinyl, morpholinyl, oxazolidonyl, oxazolanyl, oxazetanyl, piperazinyl, piperazinonyl, piperidinyl, piperidonyl, pyrrolidinyl, pyrrolinyl, pyrroyl, pyrrolonyl, pyrrolidonyl, pyrazolyl, pyrazolonyl, thiomorpholinyl, thiomorpholin-1,1-dioxide, thiazolidinyl, thiazepanyl, thiazinyl, thiazocanyl, thiazetanyl, triazolyl, indolyl, indolinyl, indazolyl, tetrahydroquinolyl, tetrahydroisoquinolyl, or benzimidazolyl, wherein, said three- to ten-membered monocyclic or bicyclic heterocyclyl or heteroaryl ring is optionally substituted with 1, 2 or 3 substituents independently selected from the group consisting of

(1) halo, (2) hydroxy, (3) optionally substituted C₁₋₈alkyl, where the substituents are amino, C₁₋₃ alkoxy, mono(C₁₋₃alkyl)amino, di(C₁₋₃alkyl)amino, and hydroxy, (4) —O(C₁₋₈alkyl), (5) nitro, (6) amino, (7) mono(C₁₋₈alkyl)amino, (8) di(C₁₋₈alkyl)amino, (9) —COOH, (10) —CO(C₁₋₈alkyl), (11) —CONH₂, (12) —CONH(C₁₋₈alkyl), (13) —CON(C₁₋₈alkyl)₂, (14) —CO₂(C₁₋₈alkyl), (15) formyl, (16) =NOH, (17) CF₃, (18) CN, (19) —NHSO₂NH₂, (20) —NHCO(C₁₋₈alkyl), (21) —N(C₁₋₈alkyl)CO(C₁₋₈alkyl), (22) —NHSO₂(C₁₋₈alkyl), (23) —N(C₁₋₈alkyl)SO₂(C₁₋₈alkyl), (24) —NHSO₂CF₃, (25) —N(C₁₋₈alkyl)CO₂(C₁₋₈alkyl), (26) —N(C₁₋₈alkyl)SO₂CF₃, (27) —N(C₁₋₈alkyl)SO₂NH₂, (28) —NHSO₂NH(C₁₋₈alkyl), (29) —NHSO₂N(C₁₋₈alkyl)₂, (30) —N(C₁₋₈alkyl)SO₂N(C₁₋₈alkyl)₂, (31) —NHCONH₂, (32) —NHCONH(C₁₋₈alkyl), (33) —NHCON(C₁₋₈alkyl)₂, (34) —N(C₁₋₈alkyl)CO(C₁₋₈alkyl), (35) —N(C₁₋₈alkyl)CO₂(C₁₋₈alkyl), (36) —N(C₁₋₈alkyl)CON(C₁₋₈alkyl)₂, (37) —S(C₁₋₈alkyl), (38) —SO₂(C₁₋₈alkyl), (39) —SO₃H, (40) —SO₂O(C₁₋₈alkyl), (41) —SO₂NH₂, (42) —SO₂N(C₁₋₈alkyl)₂, (43) —SO₂NH(C₁₋₈alkyl), (44) —NHC(NH)NH₂, (45) phenyl, unsubstituted or substituted with one to two substituents selected from halo, nitro, C₁₋₃alkyl, C₁₋₃alkoxy, hydroxy, amino, mono(C₁₋₈alkyl)amino, di(C₁₋₈alkyl)amino, —NHCO(C₁₋₈alkyl), —N(C₁₋₈alkyl)CO(C₁₋₈alkyl), —NHCO₂(C₁₋₈alkyl), —N(C₁₋₈alkyl)CO₂(C₁₋₈alkyl), —NHNH₂, —N(C₁₋₈alkyl)N(C₁₋₈alkyl)₂, and —N(C₁₋₈alkyl)NH₂, (46) pyridyl, unsubstituted or substituted with one to two substituents selected from halo, C₁₋₃alkyl and C₁₋₃alkoxy, (47) —CO—(optionally substituted heteroaryl), (48) —CO—(optionally substituted heterocyclyl), (49) —O— (optionally substituted heteroaryl), (50) —O—(optionally substituted heterocyclyl), (51) optionally substituted heterocyclyl, (52) —NH—(optionally substituted heterocyclyl),

wherein the substituents on the optionally substituted heteroaryl and heterocyclyl are one to two groups independently selected from hydroxy, C₁₋₈alkyl, —O(C₁₋₈alkyl), oxo, thioxo, amino, mono(C₁₋₈alkyl)amino, di(C₁₋₈alkyl)amino, —NHCO(C₁₋₈alkyl), —N(C₁₋₈alkyl)CO(C₁₋₈alkyl), —NHCO₂(C₁₋₈alkyl), —N(C₁₋₈alkyl)CO₂(C₁₋₈alkyl), —NHNH₂, —N(C₁₋₈alkyl)N(C₁₋₈alkyl)₂, —NHSO₂(C₁₋₈alkyl), —NHSO₂NH₂ or —N(C₁₋₈alkyl)NH₂;

R_{c} and R_{d} are independently selected from hydrogen or C₁₋₆alkyl;

Rₑ is selected from R₇, —SO₂R₇, —SO₂R₃, —SO₂R₄, _—COR₇, —(CH₂)ₙR₇, —(CH₂)ₙR₇, —CH₂)ₙCOR₇, —(CH₂)ₙSR₇, —(CH₂)ₙSO₂R₇, —(CH₂)ₙNHCOR₇, —(CH₂)ₙNHSO₂R₇, —CH₂)ₙN(C₁₋₈alkyl)COR₇, —(CH₂)ₙNHNHSO₂R₇, —(CH₂)ₙNHSO₂R₄, —(CH₂)ₙN(C₁₋₈alkyl)SO₂R₄, —(CH₂)ₙN(NH₂)R₇, —(CH₂)ₙN[N(C₁₋₈alkyl)₂]R₇, —NHSO₂R₇, optionally substituted C₁₋₈alkyl, wherein the substitutents are C₁₋₃ alkoxy, amino, mono(C₁₋₃alkyl)amino, di(C₁₋₃alkyl)amino, or hydroxy;

R_{f} is selected from the group consisting of (1) optionally substituted C₁₋₈alkyl, wherein the substituents are selected from C₁₋₃alkoxy, amino, mono(C₁₋₃alkyl)amino, di(C₁₋₃alkyl)amino, C₁₋₃alkyl, phenyl, or hydroxy, (2) —R₃, (3) —R₄, (4) phenyl, unsubstituted or substituted with R₂, (5) —(CH₂)ₙR₇, (6) —(CH₂)ₙCOR₇, (7) —(CH₂)ₙNR_{c}R₇, (8) —(CH₂)ₙNHSO₂R₇, (9) —(CH₂)ₙN(C₁₋₈alkyl)SO₂R₇, (10) —(CH₂)ₙNHCOR₇, (11) —(CH₂)ₙN(C₁₋₈alkyl)COR₇, (12) —(CH₂)ₙOR₇, (13) —(CH₂)ₙSR₇, (14) —(CH₂)ₙSO₂R₇, (15) —(CH₂)ₙNHNHSO₂R₇, (16) —(CH₂)ₙN(NH₂)R₇, (17) —(CH₂)ₙN{N(C₁₋₈alkyl)₂}R₇ or, (18) CCl₃;

R_{g} is selected from the group consisting of (1) mono(C₁₋₈alkyl)amino (2) di(C₁₋₈alkyl)amino, (3) NH₂, (4) —NHR₇, (5) —NR_{c}(CH₂)ₙR₇, (6) —NR_{c}(CH₂)ₙCOR₇, (7) —NH(CH₂)ₙO(C₁₋₈alkyl), (8) —NR_{c}(CH₂)ₙOR₇, (9) —NR_{c}(CH₂)ₙNHSO₂R₇, (10) —NR_{c}(CH₂)ₙN(C₁₋₈alkyl)SO₂R₇, (11) —NR_{c}(CH₂)ₙSO₂R₇, (12) —NR_{c}SO₂R₇, (13) —NR_{c}(CH₂)ₙSR₇, (14) —N(NH₂)R₇, (15) —N[N(C₁₋₈alkyl)₂]R₇, (16) —NR_{c}(CH₂)ₙNHNHSO₂R₇, (17) —NR_{c}(CH₂)ₙN(NH₂)R₇, (18) —NR_{c}(CH₂)ₙN[N(C₁₋₈alkyl)₂]R₇, (19) —NR_{c}(CH₂)ₙNHCOR₇, (20) —NHNHSO₂R₇, (21) optionally substituted three- to ten-membered monocyclic or bicyclic heterocyclyl or heteroaryl ring attached through the ring nitrogen atom and selected from the group consisting of aziridinyl, azepanyl, azetindinyl, azocanyl, azepinyl, diazepanyl, diazocanyl, hexahydropyridazinyl, hexahydropyrimidinyl, isothiazolidinyl, isoxazolidonyl, imidazolyl, imidazolidinyl, morpholinyl, oxazolidonyl, oxazolanyl, oxazetanyl, piperazinyl, piperazinonyl, piperidinyl, piperidonyl, pyrrolidinyl, pyrrolinyl, pyrroyl, pyrrolonyl, pyrrolidonyl, pyrazolyl, pyrazolonyl, thiomorpholinyl, thiomorpholin-1,1-dioxide, thiazolidinyl, thiazepanyl, thiazinyl, thiazocanyl, thiazetanyl, triazolyl, indolyl, indolinyl, indazolyl, tetrahydroquinolyl, tetrahydroisoquinolyl, or benzimidazolyl,

wherein, the substituents on said optionally substituted three- to ten-membered monocyclic or bicyclic heterocyclyl or heteroaryl ring are 1, 2 or 3 groups independently selected from (1) halo, (2) hydroxy, (3) C₁₋₈alkyl, unsubstituted or substituted with C₁₋₃alkoxy, amino, mono(C₁₋₃alkyl)amino, di(C₁₋₃alkyl)amino, C₁₋₃alkyl, and hydroxy, (4) —O(C₁₋₈alkyl), (5) nitro, (6) amino, (7) mono(C₁₋₈alkyl)amino, (8) di(C₁₋₈alkyl)amino, (9) —COOH, (10) —CO(C₁₋₈alkyl), (11) —CONH₂, (12) —CONH(C₁₋₈alkyl), (13) —CON(C₁₋₈alkyl)₂, (14) —CO₂(C₁₋₈alkyl), (15) formyl, (16) =NOH, (17) CF₃, (18) CN, (19) —NHSO₂NH₂, (20) —NHCO(C₁₋₈alkyl), (21) —N(C₁₋₈alkyl)CO(C₁₋₈alkyl), (22) —NHSO₂(C₁₋₈alkyl), (23) —N(C₁₋₈alkyl)SO₂(C₁₋₈alkyl), (24) —NHSO₂CF₃, (25) —N(C₁₋₈alkyl)CO₂(C₁₋₈alkyl), (26) —N(C₁₋₈alkyl)SO₂CF₃, (27) —N(C₁₋₈alkyl)SO₂NH₂, (28) —NHSO₂NH(C₁₋₈alkyl), (29) —NHSO₂N(C₁₋₈alkyl)₂, (30) —N(C₁₋₈alkyl)SO₂N(C₁₋₈alkyl)₂, (31) —NHCONH₂, (32) —NHCONH(C₁₋₈alkyl), (33) —NHCON(C₁₋₈alkyl)₂, (34) —N(C₁₋₈alkyl)CO (C₁₋₈alkyl), (35) —N(C₁₋₈alkyl)CO₂(C₁₋₈alkyl), (36) —N(C₁₋₈alkyl)CON(C₁₋₈alkyl)₂, (37) —S(C₁₋₈alkyl), (38) —SO₂(C₁₋₈alkyl), (39) —SO₃H, (40) —SO₂O(C₁₋₈alkyl), (41) —SO₂NH₂, (42) —SO₂N(C₁₋₈alkyl)₂, (43) —SO₂NH(C₁₋₈alkyl), (44) —NHC(NH)NH₂,

n is independently selected at each occurrence, from 1, 2 or 3;

R₃ at each occurrence is optionally substituted monocyclic three- to seven-membered heteroaryl ring having one to three heteroatoms independently selected from N, O, or S, wherein the substitution is by 1,2 or 3 substituents represented by R₂;

R₄ at each occurrence is optionally substituted monocyclic three- to seven-membered heterocyclyl ring having one to three heteroatoms independently selected from N, O or S, wherein the substitution is by 1,2 or 3 substituents represented by R₂;

R₅ at each occurrence is independently selected from hydrogen, C₁₋₆alkyl or CF₃;

R₆ at each occurrence are 1 or 2 groups independently selected from hydrogen, —O(C₁₋₈alkyl), halo, C₁₋₆alkyl, mono(C₁₋₆alkyl)amino or di(C₁₋₆alkyl)amino;

R₇ at each occurrence is
1. optionally substituted monocyclic three- to seven-membered aryl;
2. optionally substituted monocyclic three- to seven-membered heteroaryl or heterocyclyl having one to three heteroatoms independently selected from N, O or S,
   wherein the substitution on R₇ is by 1, 2 or 3 substituents represented by R₂.

A family of specific compounds of particular interest within the above formula (I) consists of compound and pharmaceutically acceptable salts thereof as follows:
1-[4-(Morpholine-4-carbonyl)-phenyl]-3-quinolin-2-yl-propenone (Compound No. 1);
1-[4-(3-Quinolin-2-yl-acryloyl)-benzoyl]-piperidin-4-one (Compound No. 2);
1-[4-(4-Methyl-piperazine-1-carbonyl)-phenyl]-3-quinolin-2-yl-propenone (Compound No. 3);
1-[4-(3-Quinolin-2-yl-acryloyl)-benzoyl]-pyrrolidine-2-carboxylic acid amide (Compound No. 4);
1-[4-(3-Quinolin-2-yl-acryloyl)-benzoyl]-piperidine-4-carboxylic acid isopropyl ester (Compound No. 5);
1-[4-(Piperazine-1-carbonyl)-phenyl]-3-quinolin-2-yl-propenone (Compound No. 6);
1-[4-(4-Acetyl-piperazine-1-carbonyl)-phenyl]-3-quinolin-2-yl-propenone (Compound No. 7);
1-(4-Nitro-phenyl)-4-[4-(3-quinolin-2-yl-acryloyl)-benzoyl]-piperazin-2-one (Compound No. 8);
1-[4-(3-Quinolin-2-yl-acryloyl)-benzoyl]-piperidine-4-carboxylic acid (Compound No. 9);
3-Quinolin-2-yl-1-[4-(thiomorpholine-4-carbonyl)-phenyl]-propenone (Compound No. 10);
1-[4-(Pyrrolidine-1-carbonyl)-phenyl]-3-quinolin-2-yl-propenone (Compound No. 11);
1-[4-(Piperidine-1-carbonyl)-phenyl]-3-quinolin-2-yl-propenone (Compound No. 12);
4-Morpholin-4-yl-2-{3-oxo-3-[4-(pyrrolidine-1-carbonyl)-phenyl]-propenyl}-quinoline-6-carboxylic acid methyl ester (Compound No. 13);
1-{4-[4-(3-Methyl-butyl)-piperazine-1-carbonyl]-phenyl}-3-quinolin-2-yl-propenone (Compound No. 14);
1-{4-[4-(3-Chloro-phenyl)-piperazine-1-carbonyl]-phenyl}-3-quinolin-2-yl-propenone (Compound No. 15);
1-{4-[4-(2,3-Dichloro-phenyl)-piperazine-1-carbonyl]-phenyl}-3-quinolin-2-yl-propenone (Compound No. 16);
N-(4-{2-Oxo-4-[4-(3-quinolin-2-yl-acryloyl)-benzoyl]-piperazin-1-yl}-phenyl)-acetamide (Compound No. 17);
4-Imidazol-1-yl-2-[3-oxo-3-(4-trichloromethoxycarbonylamino-phenyl)-propenyl]-quinoline-6-carboxylic acid methyl ester (Compound No. 18);
Pyrrolidine-1-carboxylic acid {4-[3-(4-morpholin-4-yl-quinolin-2-yl)-acryloyl] -phenyl}-amide (Compound No. 19);
3-Quinolin-2-yl-1-{4-[4-(tetrahydro-furan-2-carbonyl)-piperazine-1-carbonyl]-phenyl}-propenone (Compound No. 20);
1-{4-[4-(Furan-2-carbonyl)-piperazine-1-carbonyl]-phenyl}-3-quinolin-2-yl-propenone (Compound No. 21);
1-[4-(4-Pyridin-4-yl-piperazine-1-carbonyl)-phenyl]-3-quinolin-2-yl-propenone (Compound No. 22);
{4-[3-(4-Morpholin-4-yl-quinolin-2-yl)-acryloyl]-phenyl}-carbamic acid ethyl ester (Compound No: 23);
{4-[3-(4-Morpholin-4-yl-quinolin-2-yl)-acryloyl]-phenyl}-carbamic acid 2,2-dimethyl-propyl ester (Compound No. 24);
1-{4-[3-(4-Morpholin-4-yl-quinolin-2-yl)-acryloyl]-phenyl}-3-(2-trifluoromethyl-phenyl)urea (Compound No. 25);
1-Benzenesulfonyl-3-{4-[3-(4-morpholin-4-yl-quinolin-2-yl)-acryloyl]-phenyl}-urea (Compound No. 26);
4-(3-{4-[3-(4-Morpholin-4-yl-quinolin-2-yl)-acryloyl]-phenyl}-ureido)-benzoic acid ethyl ester (Compound No. 27);
1-[4-(4-Ethyl-piperazine-1-carbonyl)-phenyl]-3-quinolin-2-yl-propenone (Compound No. 28);
{4-[3-(4-Piperidin-1-yl-quinolin-2-yl)-acryloyl]-phenyl}-carbamic acid ethyl ester (Compound No. 29);
{4-[3-(4-Piperidin-1-yl-quinolin-2-yl)-acryloyl]-phenyl}-carbamic acid isobutyl ester (Compound No. 30);
{4-[3-(2-Pyrrolidin-1-yl-quinolin-3-yl)-acryloyl]-phenyl}-carbamic acid ethyl ester (Compound No. 31);
1-[4-(4-Methyl-piperazine-1-carbonyl)-phenyl]-3-(6-trifluoromethyl-quinolin-2-yl)-propenone (Compound No. 32);
1-Pyridin-2-yl-3-{4-[3-(2-pyrrolidin-1-yl-quinolin-3-yl)-acryloyl]-phenyl}-urea (Compound No. 33);
1-[4-(4-Methyl-piperazine-1-carbonyl)-phenyl]-3-(6-methylsulfanyl-quinolin-2-yl)-propenone (Compound No. 34);
1-{4-[3-(5,6,7-Trimethoxy-6,7-dihydro-quinolin-2-yl)-acryloyl]-benzoyl}-piperidin-4-one (Compound No. 35);
1-[4-(Thiomorpholine-4-carbonyl)-phenyl]-3-(5,6,7-trimethoxy-quinolin-2-yl)-propenone (Compound No. 36);
3-(3-Hydroxy-quinoxalin-2-yl)-1-[4-(4-methyl-piperazine-1-carbonyl)-phenyl]-propenone (Compound No. 37);
1-{4-[3-(3-Hydroxy-quinoxalin-2-yl)-acryloyl]-benzoyl}-piperidin-4-one (Compound No. 38);
1-[4-(4-Methyl-piperazine-1-carbonyl)-phenyl]-3-quinoxalin-2-yl-propenone (Compound No. 39);
1-[4-(Pyrrolidine-1-carbonyl)-phenyl]-3-quinoxalin-2-yl-propenone (Compound No. 40);
1-[4-(3-Quinoxalin-2-yl-acryloyl)-benzoyl]-piperidin-4-one (Compound No. 41);
3-(3-Hydroxy-quinoxalin-2-yl)-1-[4-(pyrazole-1-carbonyl)-phenyl]-propenone (Compound No. 42);
1-[4-(2,3-Dihydro-indole-1-carbonyl)-phenyl]-3-(3-hydroxy-quinoxalin-2-yl)-propenone (Compound No. 43);
1-[4-(3-Dimethylamino-pyrazole-1-carbonyl)-phenyl]-3-quinoxalin-2-yl-propenone (Compound No. 44);
1-[4-(3-Quinoxalin-2-yl-acryloyl)-benzoyl]-piperidin-4-one oxime (Compound No. 45);
2-{3-[4-(3-Dimethylamino-pyrazole-1-carbonyl)-phenyl]-3-oxo-propenyl}-quinoline-6-sulfonic acid amide (Compound No. 46);
2-{3-[4-(3,5-Dimethyl-pyrazole-1-carbonyl)-phenyl]-3-oxo-propenyl}-quinoline-6-sulfonic acid amide (Compound No. 47);
1-[4-(Pyrazole-1-carbonyl)-phenyl]-3-(5,6,7-trimethoxy-quinolin-2-yl)-propenone (Compound No. 48);
1-[4-(3,5-Dimethyl-pyrazole-1-carbonyl)-phenyl]-3-quinolin-2-yl-propenone (Compound No. 49);
1-{4-[4-(2,3-Dichloro-phenyl)-piperazine-1-carbonyl]-phenyl}-3-(2-pyrrolidin-1-yl-quinolin-3-yl)-propenone (Compound No. 50);
1-[4-(4-Methyl-piperazine-1-carbonyl)-phenyl]-3-(5,6,7-trimethoxy-quinolin-2-yl)-propenone (Compound No. 51);
1-[4-(3,5-Dimethyl-pyrazole-1-carbonyl)-phenyl]-3-(5,6,7-trimethoxy-quinolin-2-yl)-propenone (Compound No. 52);
1-{4-[3-(4-Piperidin-1-yl-6-trifluoromethyl-quinolin-2-yl)-acryloyl]-phenyl}-3-(2,3,4-trimethoxy-phenyl)-urea (Compound No. 53);
1-[4-(4-Pyrrolidin-1-yl-piperidine-1-carbonyl)-phenyl]-3-quinolin-2-yl-propenone (Compound No. 54);
1-[4-(Pyrazole-1-carbonyl)-phenyl]-3-quinolin-2-yl-propenone (Compound No. 55);
3-[6-(1-Methyl-5-trifluoromethyl-1H-pyrazol-3-yl)-quinolin-2-yl]-1-[4-(pyrrolidine-1-carbonyl)-phenyl]-propenone (Compound No. 56);
1-Cyclohexyl-3-{4-[3-(4-morpholin-4-yl-quinolin-2-yl)-acryloyl]-phenyl}-urea (Compound No. 57);
2,2-Dimethyl-N-(2-{3-oxo-3-[4-(pyrazole-1-carbonyl)-phenyl]-propenyl}-quinolin-6-yl)propionamide (Compound No. 58);
1-Benzenesulfonyl-3-{4-[3-(6-methyl-4-piperidin-1-yl-quinolin-2-yl)-acryloyl]-phenyl}-urea (Compound No. 59);
1-{4-[3-(5,6,7-Trimethoxy-quinolin-2-yl)-acryloyl]-benzoyl}-piperidine-4-carboxylic acid isopropyl ester (Compound No. 60);
Piperidine-1-carboxylic acid {4-[3-(2-pyrrolidin-1-yl-quinolin-3-yl)-acryloyl]-phenyl}-amide (Compound No. 61);
3-(6-Methylsulfanyl-4-morpholin-4-yl-quinolin-2-yl)-1-[4-(pyrrolidine-1-carbonyl)phenyl]-propenone (Compound No. 62);
3-(6-Methanesulfonyl-4-morpholin-4-yl-quinolin-2-yl)-1-[4-(pyrrolidine-1-carbonyl)phenyl]-propenone (Compound No. 63);
{4-[3-(6-[1, 2, 3]Thiadiazol-4-yl-quinolin-2-yl)-acryloyl]-phenyl}-carbamic acid ethyl ester (Compound No. 64);
1-Benzoyl-3-{4-[3-(6-[1,2,3]thiadiazol-4-yl-quinolin-2-yl)-acryloyl]-phenyl}-urea (Compound No: 65);
{4-[3-(6-[1, 2, 3]Thiadiazol-4-yl-quinolin-2-yl)-acryloyl]-phenyl}-carbamic acid phenyl ester (Compound No. 66);
{4-[3-(6-Morpholin-4-yl-pyridin-2-yl)-acryloyl]-phenyl}-carbamic acid 2-morpholin-4-yl-ethyl ester (Compound No. 67);
{4-[3-(4-Morpholin-4-yl-quinolin-2-yl)-acryloyl]-phenyl}-carbamic acid thiophen-2-ylmethyl ester (Compound No. 68);
{5-Methoxy-2-[3-(4-morpholin-4-yl-quinolin-2-yl)-acryloyl]-phenyl}-carbamic acid methyl ester (Compound No. 69);
Propyl-{4-[3-(2-pyrrolidin-1-yl-quinolin-3-yl)-acryloyl]-phenyl}-carbamic acid ethyl ester (Compound No. 70);
{4-[3-(4-Morpholin-4-yl-quinolin-2-yl)-acryloyl]-phenyl}-carbamic acid phenyl ester (Compound No. 71);
2,2-Dimethyl-N-{1-[4-(3-quinoxalin-2-yl-acryloyl)-benzoyl]-piperidin-4-yl}-propionamide (Compound No. 72);
1-[4-(3-Quinoxalin-2-yl-acryloyl)-benzoyl]-piperidine-4-carboxylic acid dimethylamide (Compound No. 73);
{4-[3-(6-[1, 2, 3]Thiadiazol-4-yl-quinolin-2-yl)-acryloyl]-phenyl}-carbamic acid methyl ester (Compound No. 74);
1-(4-Methyl-benzenesulfonyl)-3-{4-[3-(3,4,5,6-tetrahydro-2H-[1,2′]bipyridinyl-6′-yl)-acryloyl-]-phenyl}-urea (Compound No. 75);
{4-[3-(3,4,5,6-Tetrahydro-2H-[1,2′]bipyridinyl-6′-yl)-acryloyl]-phenyl}-carbamic acid ethyl ester (Compound No. 76);
N-{4-[3-(2-Morpholin-4-yl-quinolin-3-yl)-acryloyl]-phenyl}-oxalamide (Compound No. 77);
2-Morpholin-4-yl-N-{4-[3-(4-morpholin-4-yl-quinolin-2-yl)-acryloyl]-phenyl}-2-oxo-acetamide (Compound No. 78);
2-Morpholin-4-yl-N-{4-[3-(2-morpholin-4-yl-quinolin-3-yl)-acryloyl]-phenyl}-2-oxo-acetamide (Compound No. 79);
N-{4-[3-(2-Piperidin-1-yl-quinolin-3-yl)-acryloyl]-phenyl}-oxalamide (Compound No. 80);
2-Morpholin-4-yl-2-oxo-N-{4-[3-(2-piperidin-1-yl-quinolin-3-yl)-acryloyl]-phenyl}-acetamide (Compound No. 81);
N-{4-[3-(4-Morpholin-4-yl-quinolin-2-yl)-acryloyl]-phenyl}-N′-propyl-oxalamide (Compound No. 82);
2-Morpholin-4-yl-N-{4-[3-(6-morpholin-4-yl-pyridin-2-yl)-acryloyl]-phenyl}-2-oxo-acetamide (Compound No. 83);
(4-{3-[6-(3,5-Dimethyl-morpholin-4-yl)-pyridin-3-yl]-acryloyl}-phenyl)-carbamic acid phenyl ester (Compound No. 84);
N-{4-[3-(4-Morpholin-4-yl-quinolin-2-yl)-acryloyl]-phenyl}-2-oxo-2-piperidin-1-yl-acetamide (Compound No. 85);
5′-[3-Oxo-3-(4-phenoxycarbonylamino-phenyl)-propenyl]-3,4,5,6-tetrahydro-2H-[1,2′]bipyridinyl-4-carboxylic acid (Compound No. 86);
2-Oxo-2-piperidin-1-yl-N-{4-[3-(4-pyrrol-1-yl-quinolin-2-yl)-acryloyl]-phenyl}-acetamide (Compound No. 87);
2-Morpholin-4-yl-2-oxo-N-{4-[3-(4-pyrrol-1-yl-quinolin-2-yl)-acryloyl]-phenyl}-acetamide (Compound No. 88);
C,C,C-Trifluoro-N-{1-[4-(3-quinoxalin-2-yl-acryloyl)-benzoyl]-piperidin-4-yl}-methanesulfonamide (Compound No. 89);
{4-[3-(6-Pyrrolidin-1-yl-pyridin-2-yl)-acryloyl]-phenyl}-carbamic acid pyridin-2-ylmethyl ester (Compound No. 90);
{4-[3-(6-Pyrrolidin-1-yl-pyridin-2-yl)-acryloyl]-phenyl}-carbamic acid 4-fluoro-benzyl ester (Compound No. 91);
{4-[3-(3,4,5,6-Tetrahydro-2H-[1,2′]bipyridinyl-6′-yl)-acryloyl]-phenyl}-carbamic acid 2-(3,5-dimethyl-pyrazol-1-yl)-ethyl ester (Compound No. 92);
{4-[3-(3,4,5,6-Tetrahydro-2H-[1,2′]bipyridinyl-6′-yl)-acryloyl]-phenyl}-carbamic acid furan-2-ylmethyl ester (Compound No. 93);
{4-[3-(3,4,5,6-Tetrahydro-2H-[1,2′]bipyridinyl-6′-yl)-acryloyl]-phenyl}-carbamic acid 3-phenyl-allyl ester (Compound No. 94);
{4-[3-(3,4,5,6-Tetrahydro-2H-[1,2′]bipyridinyl-6′-yl)-acryloyl]-phenyl}-carbamic acid 2-piperidin-1-yl-ethyl ester (Compound No. 95);
{4-[3-(4-Morpholin-4-yl-quinolin-2-yl)-acryloyl]-phenyl}-carbamic acid methyl ester (Compound No. 96);
3-(3-Hydroxy-quinoxalin-2-yl)-1-[4-(morpholine-4-carbonyl)-phenyl]-propenone (Compound No. 97);

3-(3-Hydroxy-quinoxalin-2-yl)-1-[4-(thiomorpholine-4-carbonyl)-phenyl]-propenone (Compound No. 98);
1-[4-(3,5-Dimethyl-pyrazole-1-carbonyl)-phenyl]-3-(3-hydroxy-quinoxalin-2-yl)-propenone (Compound No. 99);
1-{4-[4-(2,3-Dichloro-phenyl)-piperazine-1-carbonyl]-phenyl}-3-(3-hydroxy-quinoxalin-2-yl)-propenone (Compound No. 100);
1-{4-[4-(3-Chloro-phenyl)-piperazine-1-carbonyl]-phenyl}-3-(3-hydroxy-quinoxalin-2-yl)-propenone (Compound No. 101);
1-{4-[3-(3-Hydroxy-quinoxalin-2-yl)-acryloyl]-benzoyl}-piperidine-4-carboxylic acid dimethylamide (Compound No. 102);
N-(1-{4-[3-(3-Hydroxy-quinoxalin-2-yl)-acryloyl]-benzoyl}-piperidin-4-y l)-2,2-dimethyl-propionamide (Compound No. 103);
1-{4-[3-(3-Hydroxy-quinoxalin-2-yl)-acryloyl]-benzoyl}-piperidine-4-carboxylic acid isopropyl ester (Compound No. 104);
1-{4-[3-(3-Hydroxy-quinoxalin-2-yl)-acryloyl]-benzoyl}-pyrrolidine-2-carboxylic acid amide (Compound No. 105);
3-(3-Hydroxy-quinoxalin-2-yl)-1-[4-(4-pyridin-4-yl-piperazine-1-carbonyl)-phenyl]-propenone (Compound No. 106);
2-{3-[4-(Morpholine-4-carbonyl)-phenyl]-3-oxo-propenyl}-3H-quinazolin-4-one (Compound No. 107);
2-{3-Oxo-3-[4-(pyrazole-1-carbonyl)-phenyl]-propenyl}-3H-quinazolin-4-one (Compound No. 108);
2-{3-[4-(4-Methyl-piperazine-1-carbonyl)-phenyl]-3-oxo-propenyl}-3H-quinazolin-4-one (Compound No. 109);
(4-{3-[2-(4-Hydroxy-piperidin-1-yl)-quinolin-3-yl]-acryloyl}-phenyl)-carbamic acid phenyl ester (Compound No. 110);
(4-{3-[2-(4-Hydroxy-piperidin-1-yl)-quinolin-3-yl]-acryloyl}-phenyl)-carbamic acid methyl ester (Compound No. 111);
(4-{3-[2-(4-Hydroxy-piperidin-1-yl)-quinolin-3-yl]-acryloyl}-phenyl)-carbamic acid ethyl ester (Compound No. 112);
1-Benzenesulfonyl-3-(4-{3-[6-(3,5-dimethyl-morpholin-4-yl)-pyridin-3-yl]-acryloyl}-phenyl)-urea (Compound No. 113);
N-{4-[3-(6-Morpholin-4-yl-pyridin-2-yl)-acryloyl]-phenyl}-2-oxo-2-piperidin-1-yl-acetamide ( Compound No. 114);
N-{4-[3-(6-Morpholin-4-yl-pyridin-2-yl)-acryloyl]-phenyl}-oxalamide (Compound No. 115);
2-Oxo-2-piperidin-1-yl-N-{4-[3-(2-piperidin-1-yl-quinolin-3-yl)-acryloyl]-phenyl}-acetamide (Compound No. 116);
2-Oxo-2-piperidin-1-yl-N-[4-(3-quinoxalin-2-yl-acryloyl)-phenyl]-acetamide (Compound No. 117);
2-Morpholin-4-yl-2-oxo-N-[4-(3-quinoxalin-2-yl-acryloyl)-phenyl]-acetamide (Compound No. 118);
N-Propyl-N′-[4-(3-quinoxalin-2-yl-acryloyl)-phenyl]-oxalamide (Compound No. 119);
N-[4-(3-Quinoxalin-2-yl-acryloyl)-phenyl]-oxalamide (Compound No. 120);
N-(2-Methoxy-ethyl)-N′-[4-(3-quinoxalin-2-yl-acryloyl)-phenyl]-oxalamide (Compound No. 121);
1-{4-[4-(3-Chloro-phenyl)-piperazine-1-carbonyl]-phenyl}-3-quinoxalin-2-yl-propenone (Compound No. 122);
3-Quinoxalin-2-yl-1-[4-(thiomorpholine-4-carbonyl)-phenyl]-propenone (Compound No. 123);
1-[4-(3-Quinoxalin-2-yl-acryloyl)-benzoyl]-piperidine-4-carboxylic acid isopropyl ester (Compound No. 124);
1-[4-(4-Pyridin-4-yl-piperazine-1-carbonyl)-phenyl]-3-quinoxalin-2-yl-propenone (Compound No. 125);
1-{4-[3-(6-Morpholin-4-yl-pyridin-2-yl)-acryloyl]-phenyl}-3-(2-oxo-2-piperidin-1-yl-ethyl)-urea (Compound No. 126);
1-(2-Morpholin-4-yl-ethyl)-3-{4-[3-(6-morpholin-4-yl-pyridin-2-yl)-acryloyl]-phenyl}-urea (Compound No. 127);
2,2-Dimethyl-N-{1-[4-(3-quinoxalin-2-yl-acryloyl)-benzoyl]-piperidin-4-yl}-propionamide (Compound No. 128);
{4-[3-(6-Morpholin-4-yl-pyridin-2-yl)-acryloyl]-phenyl}-carbamic acid 2-piperazin-1-yl-ethyl ester (Compound No. 129);
N-(2-Morpholin-4-yl-ethyl)-N′-[4-(3-quinoxalin-2-yl-acryloyl)-phenyl]-oxalamide (Compound No. 130);
1-{4-[3-(6-Morpholin-4-yl-pyridin-2-yl)-acryloyl]-phenyl}-3-[2-(pyridine-2-sulfonyl)-ethyl]-urea (Compound No. 131);
1-{4-[3-(6-Morpholin-4-yl-pyridin-2-yl)-acryloyl]-phenyl}-3-[2-(pyridin-2-ylsulfanyl)-ethyl]-urea (Compound No. 132);
1-[2-(4-Methyl-piperazin-1-yl)-ethyl]-3-{4-[3-(6-morpholin-4-yl-pyridin-2-yl)-acryloyl]-phenyl}-urea (Compound No. 133);
N-(2-Morpholin-4-yl-ethyl)-N′-{4-[3-(6-morpholin-4-yl-pyridin-2-yl)-acryloyl]-phenyl}-oxalamide (Compound No. 134);
1-(2-Morpholin-4-yl-ethyl)-3-[4-(3-quinoxalin-2-yl-acryloyl)-phenyl]-urea (Compound No. 135);
N-(2-{3-[4-(3-Quinoxalin-2-yl-acryloyl)-phenyl]-ureido}-ethyl)-benzenesulfonamide (Compound No. 136);
N-[4-(3-Quinolin-2-yl-acryloyl)-phenyl]-oxalamide (Compound No. 137);
2-Morpholin-4-yl-2-oxo-N-[4-(3-quinolin-2-yl-acryloyl)-phenyl]-acetamide (Compound No. 138);
1-Benzenesulfonyi-hydrazino-3-[4-(3-quinoxalin-2-yl-acryloyl)-phenyl]-urea (Compound No. 139);
1-(2-Morpholin-4-yl-2-oxo-ethyl)-3-[4-(3-quinolin-2-yl-acryloyl)-phenyl]-urea (Compound No. 140);
1-[2-(Pyridin-4-yloxy)-ethyl]-3-[4-(3-quinolin-2-yl-acryloyl)-phenyl]-urea (Compound No. 141);
1-(Morpholine-4-sulfonyl)-3-[4-(3-quinolin-2-yl-acryloyl)-phenyl]-urea (Compound No. 142);
{4-[3-(6-Morpholin-4-yl-pyridin-2-yl)-acryloyl]-phenyl}-carbamic acid piperidin-4-yl ester (Compound No. 143);
4-(3-{4-[3-(4-Morpholin-4-yl-quinolin-2-yl)-acryloyl]-phenyl}-ureido)-benzoic acid (Compound No. 144);
4-(3-{4-[3-(4-Piperidin-1-yl-quinolin-2-yl)-acryloyl]-phenyl}-ureido)-benzoic acid (Compound No. 145);
1-(4-Methyl-thiophen-2-yl)-3-{4-[3-(4-piperidin-1-yl-6-trifluoromethyl-quinolin-2-yl)-acryloyl]-phenyl}-urea (Compound No. 146);
N-(2-{3-[4-(3-Dimethylamino-pyrazole-1-carbonyl)-phenyl]-3-oxo-propenyl}-quinolin-6-yl)-2,2-dimethyl-propionamide (Compound No. 147);
1-[4-(Morpholine-4-carbonyl)-phenyl]-3-(5,6,7-trimethoxy-quinolin-2-yl)-propenone (Compound No. 148);
1-{4-[3-(4-Piperidin-1-yl-6-trifluoromethyl-quinolin-2-yl)-acryloyl]-phenyl}-3-pyridin-2-yl-urea (Compound No. 149);
1-Cyclohexyl-3-{4-[3-(4-piperidin-1-yl-6-trifluoromethyl-quinolin-2-yl)-acryloyl]-phenyl}-urea (Compound No. 150);
1-[4-(4-Methoxy-piperidine-1-carbonyl)-phenyl]-3-quinolin-2-yl-propenone (Compound No. 151);
1-[4-(Pyrazole-1-carbonyl)-phenyl]-3-(3,4,5,6-tetrahydro-2H-[1,2′]bipyridinyl-6′-yl)-propenone ( Compound No. 152);
1-[4-(3,5-Dimethyl-pyrazole-1-carbonyl)-phenyl]-3-(3,4,5,6-tetrahydro-2H-[1,2′]bipyridinyl-6′-yl)-propenone (Compound No. 153);
1-{4-[3-(3,4,5,6-Tetrahydro-2H-[1,2′]bipyridinyl-6′-yl)-acryloyl]-benzoyl}-piperidine-4-carboxylic acid isopropyl ester (Compound No. 154);
1-[2-(Pyridin-2-ylsulfanyl)-ethyl]-3-[4-(3-quinoxalin-2-yl-acryloyl)-phenyl]-urea (Compound No. 155);
{4-[3-(6-Morpholin-4-yl-pyridin-2-yl)-acryloyl]-phenyl}-carbamic acid 2-(pyridine-2-sulfonyl)-ethyl ester (Compound No. 156);
{4-[3-(6-Morpholin-4-yl-pyridin-2-yl)-acryloyl]-phenyl}-carbamic acid 2-(pyridin-2-ylsulfanyl)-ethyl ester (Compound No. 157);
[4-(3-Quinoxalin-2-yl-acryloyl)-phenyl]-carbamic acid 2-benzenesulfonylamino-ethyl ester (Compound No. 158);
{4-[3-(6-Morpholin-4-yl-pyridin-2-yl)-acryloyl]-phenyl}-carbamic acid 2-(N-pyridin-2-yl-hydrazino)-ethyl ester (Compound No. 159);
1-{2-[N-(6-Methyl-pyridin-2-yl)-hydrazino]-ethyl}-3-[4-(3-quinoxalin-2-yl-acryloyl)-phenyl]-urea (Compound No. 160);
1-[4-(Pyrazole-1-carbonyl)-phenyl]-3-(3,4,5,6-tetrahydro-2H-[1,2′]bipyridinyl-5′-yl)-but-2-en-1-one (Compound No. 161);
4,4,4-Trifluoro-1-[4-(morpholine-4-carbonyl)-phenyl]-3-quinolin-3-yl-but-2-en-1-one (Compound No. 62);
N-{4-[3-(6-Morpholin-4-yl-pyridin-3-yl)-but-2-enoyl]-phenyl}-2-oxo-2-piperidin-1-yl-acetamide (Compound No. 163);
2-Morpholin-4-yl-2-oxo-N-[4-(4,4,4-trifluoro-3-quinolin-3-yl-but-2-enoyl)-phenyl]-acetamide (Compound No. 164);
Morpholine-4-carboxylic acid {4-[4,4,4-trifluoro-3-(4-morpholin-4-yl-quinolin-3-yl)-but-2-enoyl]-phenyl}-amide (Compound No. 165);
Morpholine-4-carboxylic acid {4-[3-(6-morpholin-4-yl-pyridin-3-yl)-but-2-enoyl]-phenyl}-amide (Compound No. 166);
N-[2-(3-{4-[3-(6-Morpholin-4-yl-pyridin-2-yl)-acryloyl]-phenyl}-ureido)-ethyl]-nicotinamide (Compound No. 167);
1-[2-(Piperidin-4-yloxy)-ethyl]-3-[4-(3-quinolin-2-yl-acryloyl)-phenyl]-urea (Compound No. 168);
(4-{3-[6-(4-Methyl-piperazin-1-yl)-pyridin-2-yl]-acryloyl}-phenyl)-carbamic acid furan-2-ylmethyl ester (Compound No. 169);
(4-{3-[6-(4-Methyl-piperazin-1-yl)-pyridin-2-yl]-acryloyl}-phenyl)-carbamic acid pyridin-2-ylmethyl ester (Compound No. 170);
(4-{3-[6-(4-Methyl-piperazin-1-yl)-pyridin-2-yl]-acryloyl}-phenyl)-carbamic acid 2-(3,5-dimethyl-pyrazol-1-yl)-ethyl ester (Compound No. 171);
{4-[3-(6-Morpholin-4-yl-pyridin-2-yl)-acryloyl]-phenyl}-carbamic acid 2-[1,2,4]triazol-1-yl-ethyl ester (Compound No. 172);
{4-[3-(6-Piperazin-1-yl-pyridin-2-yl)-acryloyl]-phenyl}-carbamic acid 2-[1,2,4]-triazol-1-yl-ethyl ester (Compound No. 173);
(4-{3-[6-(4-Methyl-piperazin-1-yl)-pyridin-2-yl]-acryloyl}-phenyl)-carbamic acid thiophen-2-ylmethyl ester (Compound No. 174);
2-{3-Oxo-3-[4-(thiophen-2-ylmethoxycarbonylamino)-phenyl]-propenyl}-quinoline-6-carboxylic acid (Compound no. 175);
{4-[3-(6-[1,2,4]Triazol-1-yl-pyridin-2-yl)-acryloyl]-phenyl}-carbamic acid thiophen-2-ylmethyl ester (Compound no. 176);
{4-[3-(6-Tetrazol-1-yl-pyridin-2-yl)-acryloyl]-phenyl}-carbamic acid thiophen-2-yl methyl ester (Compound No. 177);
{4-[3-(6-Morpholin-4-yl-pyridin-2-yl)-acryloyl]-phenyl}-carbamic acid pyridin-2yl methyl ester (Compound No. 178);
4-(3-Quinolin-2-yl-acryloyl)-phenyl]-carbamic acid pyridin-2-ylmethyl ester (Compound No. 179);
(4-{3-[6-(4-Methyl-piperazin-1-yl)-pyridin-2-yl]-acryloyl}-phenyl)-carbamic acid piperidin-4-yl ester (Compound No. 180);
{4-[3-(6-Morpholin-4-yl-pyridin-2-yl)-acryloyl]-phenyl}-carbamic acid 1-methyl- 1H-pyrrol-3-yl ester (Compound No. 181);
(4-{3-[6-(4-Methyl-piperazin-1-yl)-pyridin-2-yl]-acryloyl}-phenyl)-carbamic acid isoxazol-3-yl ester (Compound No. 182);
[4-(3-Quinolin-2-yl-acryloyl)-phenyl]-carbamic acid 1-methyl-1H-imidazol-4-ylmethyl ester (Compound No. 183);
[4-(3-Pyridin-2-yl-acryloyl)-phenyl]-carbamic acid 2-(4-methyl-piperazine-1-sulfonylamino)-ethyl ester (Compound No. 184);
[4-(3-Quinolin-2-yl-acryloyl)-phenyl]-carbamic acid 2-(4-methyl-piperazine-1-sulfonylamino)-ethyl ester (Compound No. 185);
(4-{3-[6-(4-Methyl-piperazin-1-yl)-pyridin-2-yl]-acryloyl}-phenyl)-carbamic acid 2-benzenesulfonylamino-ethyl ester (Compound No. 186);
{4-[3-(6-Morpholin-4-yl-pyridin-2-yl)-acryloyl]-phenyl}-carbamic acid 2-[(4-methyl-piperazine-1-carbonyl)-amino]-ethyl ester (Compound No. 187);
[4-(3-Quinolin-2-yl-acryloyl)-phenyl]-carbamic acid 2-[(4-methyl-piperazine-1-carbonyl)-amino]-ethyl ester (Compound No. 188);
[4-(3-Pyridin-2-yl-acryloyl)-phenyl]-carbamic acid 2-(N-pyridin-2-yl-hydrazino)-ethyl ester (Compound No. 189);
[4-(3-Pyridin-2-yl-acryloyl)-phenyl]-carbamic acid 2-[N-(5-methyl-isoxazol-3-yl)-hydrazino]-ethyl ester (Compound No. 190);
[4-(3-Pyridin-2-yl-acryloyl)-phenyl]-carbamic acid 2-(N′-benzenesulfonyl-hydrazino)-ethyl ester (Compound No. 191);
1-{4-[3-(6-Morpholin-4-yl-pyridin-2-yl)-acryloyl]-phenyl}-3-(2-oxo-2-piperazin-1-yl-ethyl)-urea (Compound No. 192);
1-[2-(4-Methyl-piperazin-1-yl)-2-oxo-ethyl]-3-[4-(3-quinolin-2-yl-acryloyl)-phenyl]-urea (Compound No. 193);
1-(2-Morpholin-4-yl-2-oxo-ethyl)-3-{4-[3-(6-piperazin-1-yl-pyridin-2-yl)-acryloyl]-phenyl}-urea (Compound No. 194);
1-[2-(Pyridine-2-sulfonyl)-ethyl]-3-[4-(3-pyridin-2-yl-acryloyl)-phenyl]-urea (Compound No. 195);
1-{4-[3-(6-Piperazin-1-yl-pyridin-2-yl)-acryloyl]-phenyl}-3-[2-(piperidine-4-sulfonyl)-ethyl]-urea (Compound No. 196);
4-Methyl-piperazine-1-sulfonic acid(2-{3-[4-(3-quinolin-2-yl-acryloyl)-phenyl]-ureido}-ethyl)-amide (Compound No. 197);
1-{4-[3-(6-Morpholin-4-yl-pyridin-2-yl)-acryloyl]-phenyl}-3-[2-(piperidin-4-ylsulfanyl)-ethyl]-urea (Compound No. 198);
1-{2-[N-(1-Methyl-piperidin-4-yl)-hydrazino]-ethyl}-3-[4-(3-pyridin-2-yl-acryloyl)-phenyl]-urea (Compound No. 199);
1-{2-[N-(1-Methyl-piperidin-4-yl)-hydrazino]-ethyl}-3-[4-(3-quinoxalin-2-yl-acryloyl)-phenyl]-urea (Compound No. 200);
1-{2-[N-(6-Methyl-pyridin-2-yl)-hydrazino]-ethyl}-3-{4-[3-(6-morpholin-4-yl-pyridin-2-yl)-acryloyl]-phenyl}-urea (Compound No. 201);
1-(2-Piperazin-1-yl-ethyl)-3-[4-(3-quinoxalin-2-yl-acryloyl)-phenyl]-urea (Compound No. 202);
1-(2-Morpholin-4-yl-ethyl)-3-[4-(3-quinolin-2-yl-acryloyl)-phenyl]urea (Compound No. 203);
1-(2-Piperazin-1-yl-ethyl)-3-[4-(3-quinolin-2-yl-acryloyl)-phenyl]-urea (Compound No. 204);
1-{4-[3-(6-Piperazin-1-yl-pyridin-2-yl)-acryloyl]-phenyl}-3-(2-piperidin-1-yl-ethyl)-urea (Compound No. 205);
1-{4-[3-(6-Piperazin-1-yl-pyridin-2-yl)-acryloyl]-phenyl}-3-(2-thiomorpholin 1,1-dioxide-4-yl-ethyl)-urea (Compound No. 206);
1-(2-Piperazin-1-yl-ethyl)-3-{4-[3-(3,4,5,6-tetrahydro-2H-[1,2′]bipyridinyl-6′-yl)-acryloyl]-phenyl}-urea (Compound No. 207);
Piperidine-4-carboxylic acid [2-(3-{4-[3-(6-morpholin-4-yl-pyridin-2-yl)-acryloyl]-phenyl}-ureido)-ethyl]-amide (Compound No. 208);
N-(2-{3-[4-(3-Pyridin-2-yl-acryloyl)-phenyl]-ureido}-ethyl)-nicotinamide (Compound No. 209);
1-[2-(N′-Benzenesulfonyl-hydrazino)-ethyl]-3-[4-(3-pyridin-2-yl-acryloyl)-phenyl]-urea (Compound No. 210);
1-[2-(N′-Benzenesulfonyl-hydrazino)-ethyl]-3-[4-(3-quinolin-2-yl-acryloyl)-phenyl]-urea (Compound No. 211);
1-(Piperazine-1-sulfonyl)-3-[4-(3-pyridin-2-yl-acryloyl)phenyl]-urea (Compound No. 212);
1-{4-[3-(6-Morpholin-4-yl-pyridin-2-yl)-acryloyl]-phenyl}-3-(piperazine-1-sulfonyl)-urea (Compound No. 213);
1-(Piperazine-1-sulfonyl)-3-[4-(3-quinoxalin-2-yl-acryloyl)-phenyl]-urea (Compound No. 214);
1-(Piperazine-1-sulfonyl)-3-[4-(3-quinolin-2-yl-acryloyl) phenyl]-urea (Compound No. 215);
N-(2-Piperazin-1-yl-ethyl)-N′-[4-(3-pyridin-2-yl-acryloyl)-phenyl]-oxalamide (Compound No. 216);
N-{4-[3-(6-Morpholin-4-yl-pyridin-2-yl)-acryloyl]-phenyl}-N′-(2-piperazin-1-yl-ethyl)-oxalamide (Compound No. 217);
1-[2-({4-[3-(6-Morpholin-4-yl-pyridin-2-yl)-acryloyl]-phenylaminooxalyl}-amino)-acetyl]-piperidine-4-carboxylic acid (Compound No. 218);
N-(2-Oxo-2-piperazin-1-yl-ethyl)-N′-[4-(3-pyridin-2-yl-acryloyl)-phenyl]-oxalamide (Compound No. 219);
N-[4-(3-Pyridin-2-yl-acryloyl)-phenyl]-N′-[2-(pyridine-2-yl-sulfonyl)-ethyl]-oxalamide (Compound No. 220);
N-[2-(Piperidin-4-ylsulfanyl)-ethyl]-N′-[4-(3-pyridin-2-yl-acryloyl)-phenyl]-oxalamide (Compound No. 221);
N-[2-(Pyridine-2-sulfonyl)-ethyl]-N′-[4-(3-quinolin-2-yl-acryloyl)-phenyl]-oxalamide (Compound No. 222);
2-[N-(6-Methyl-pyridin-2-yl)-hydrazino]-2-oxo-N-[4-(3-quinolin-2-yl-acryloyl)-phenyl]-acetamide (Compound No. 223);
2-Oxo-2-(N-phenyl-hydrazine)-N-[4-(3-pyridin-2-yl-acryloyl)-phenyl]-acetamide (Compound No. 224);
2-Oxo-2-(piperazine-1-sulfonylamino)-N-[4-(3-pyridin-2-yl-acryloyl)-phenyl]-acetamide (Compound No. 225);
2-Benzenesulfonylamino-2-oxo-N-[4-(3-pyridin-2-yl-acryloyl)-phenyl]-acetamide (Compound No. 226);
N-[2-(Piperazine-1-sulfonylamino)-ethyl]-N′-[4-(3-pyridin-2-yl-acryloyl)-phenyl]-oxalamide (Compound No. 227);
2-(N′-Benzenesulfonyl-hydrazino)-2-oxo-N-[4-(3-pyridin-2-yl-acryloyl)-phenyl]-acetamide (Compound No. 228);
N-{2-[N-(6-Methyl-pyridin-2-yl)-hydrazino]-ethyl}-N′-[4-(3-pyridin-2-yl-acryloyl)-phenyl]-oxalamide (Compound No. 229);
N-{2-[(Piperazine-1-carbonyl)-amino]-ethyl}-N′-[4-(3-pyridin-2-yl-acryloyl)-phenyl]-oxalamide (Compound No. 230);
N-{2-[(Pyridine-3-carbonyl)-amino]-ethyl}-N′-[4-(3-pyridin-2-yl-acryloyl)-phenyl]-oxalamide (Compound No. 231);
N-{2-[(Piperidine-4-carbonyl)-amino]-ethyl}-N′-[4-(3-pyridin-2-yl-acryloyl)-phenyl]-oxalamide (Compound No. 232);
1-[4-(4-Methyl-piperazine-1-carbonyl)-phenyl]-3-{6-[2-(4-methyl-piperazin-1-yl)-ethylamino]-pyridin-2-yl}-propenone (Compound No. 233);
(2-{3-[4-(4-Methyl-piperazine-1-carbonyl)-phenyl]-3-oxo-propenyl}-quinolin-6-yl)-carbamic acid ethyl ester (Compound No. 234);
(6-{3-[4-(4-Methyl-piperazine-1-carbonyl)-phenyl]-3-oxo-propenyl}-pyridin-2-yl)-carbamic acid phenyl ester (Compound No. 235);
1-[4-(Morpholine-4-carbonyl)-phenyl]-3-{6-[(piperidin-1-ylmethyl)-amino]-pyridin-2-yl}-propenone (Compound No. 236);
1-[4-(3-Dimethylamino-pyrazole-1-carbonyl)-phenyl]-3-[6-(2-oxo-2-piperidin-1-yl-ethylamino)-pyridin-2-yl]-propenone (Compound No. 237);
1-[4-(Piperazine-1-carbonyl)-phenyl]-3-{6-[2-(pyridine-2-sulfonyl)-ethylamino]-pyridin-2-yl}-propenone (Compound No. 238);
Benzenesulfonic acid N′-(6-{3-oxo-3-[4-(piperidine-1-carbonyl)-phenyl]-propenyl}-pyridin-2-yl)-hydrazide (Compound No. 239);
(6-{3-[4-(3-Benzenesulfonyl-ureido)-phenyl]-3-oxo-propenyl}-pyridin-2-yl)-carbamic acid ethyl ester (Compound No. 240);
Morpholine-4-carboxylic acid (4-{3-[6-(2-piperidin-1-yl-ethylamino)-pyridin-2-yl]-acryloyl}-phenyl)-amide (Compound No. 241);
{2-[3-(4-Ethoxycarbonylamino-phenyl)-3-oxo-propenyl]-quinolin-6-yl}-carbamic acid ethyl ester (Compound No. 242);
{2-[3-Oxo-3-(4-phenoxycarbonylamino-phenyl)-propenyl]-quinolin-4-yl}-carbamic acid ethyl ester (Compound No. 243);
[2-(3-{4-[2-(4-Methyl-piperazin-1-yl)-2-oxo-acetylamino]-phenyl}-3-oxo-propenyl)-quinolin-4-yl]-carbamic acid methyl ester (Compound No. 244);
1-(2-Morpholin-4-yl-ethyl)-3-(4-{3-[6-(2-piperidin-1-yl-ethylamino)-pyridin-2-yl]-acryloyl}-phenyl)-urea (Compound No. 245);
1-[4-(3-Dimethylamino-pyrazole-1-carbonyl)-phenyl]-3-{6-[2-(4-methyl-piperazin-1-yl)-2-oxo-ethylamino]-pyridin-2-yl}-propenone (Compound No. 246);
N-(1-{4-[4-(3-Pyridin-2-yl-acryloyl)-benzoyl]-piperazin-1-yl}-piperidin-4-yl)-methanesulfonamide (Compound No. 247);
(1-{4-[4-(3-Pyridin-2-yl-acryloyl)-benzoyl]-piperazin-1-yl}-piperidin-4-yl)-sulfonylurea (Compound No. 248);
1-{4-[4-(4-Cyclohexylamino-piperidin-1-yl)-piperazine-1-carbonyl]-phen yl}-3-pyridin-2-yl-propenone (Compound No. 249);
[6-(3-Oxo-3-{4-[4-(pyrrolidine-1-carbonyl)-piperidine-1-carbonyl]-phenyl}-propenyl)-pyridin-2-yl]-carbamic acid methyl ester (Compound No. 250);
[6-(3-Oxo-3-{4-[4-(pyridin-2-yloxy)-piperidine-1-carbonyl]-phenyl}-propenyl)-pyridin-2-yl]-carbamic acid ethyl ester (Compound No. 251).

A preferred embodiment of the invention consists of those compounds of Formula (I), wherein

Q is as defined hereinabove, which may be unsubstituted or substituted by 1 to 6 substituents represented by R₂;

R₂ is independently selected at each occurrence from hydrogen, hydroxy, halo, amino, C₁₋₈alkyl, —O(C₁₋₈alkyl), —S(C₁₋₈alkyl), —SO₂(C₁₋₈alkyl), oxo, thioxo, mono(C₁₋₈alkyl)amino, di(C₁₋₈alkyl)amino, —NHCO(C₁₋₈alkyl), —N(C₁₋₈alkyl)CO(C₁₋₈alkyl), —NHSO₂(C₁₋₈alkyl), —NHSO₂CF₃, —N(C₁₋₈alkyl)SO₂CF₃, —NHSO₂O(C₁₋₈alkyl), —N(C₁₋₈alkyl)SO₂(C₁₋₈alkyl), —N(C₁₋₈alkyl)SO₂O(C₁₋₈alkyl), —COOH, —CO₂(C₁₋₈alkyl), —NHCO₂(C₁₋₈alkyl), —N(C₁₋₈alkyl)CO_{2(C}₁₋₈alkyl), —CONH₂, —CONH(C₁₋₈alkyl), —CON(C₁₋₈alkyl)₂, formyl, CF₃, CN, —(CH₂)ₙOH, —(CH₂)ₙNH₂, —(CH₂)ₙNH(C₁₋₈alkyl), —(CH₂)ₙN(C₁₋₈alkyl)₂, —(CH₂)ₙO(C₁₋₈alkyl), —SO₃H, —SO₂O(C₁₋₈alkyl), —SO₂NH₂, —SO₂N(C₁₋₈alkyl)₂, —SO₂NH(C₁₋₈alkyl), —OSO₂(C₁₋₈alkyl), —N(C₁₋₈alkyl)SO₂NH₂, —NHSO₂NH(C₁₋₈alkyl), —NHSO₂N(C₁₋₈alkyl)₂, —N(C₁₋₈alkyl)SO₂N(C₁₋₈alkyl)₂, —NHSO₂NH₂, —NHC(NH)NH₂, —NHCONH₂, —NHC(O)NH(C₁₋₈alkyl), —NHC(O)N(C₁₋₈alkyl)₂, —N(C₁₋₈alkyl)C(O)N(C₁₋₈alkyl)₂, —NHNH₂, —N(C₁₋₈alkyl)N(C₁₋₈alkyl)₂, —N(C₁₋₈alkyl)NH₂, tetrazolyl or three- to seven-membered heterocyclyl or heteroaryl ring having upto three heteroatoms independently selected from N, O, or S, wherein said three- to seven-membered heterocyclyl or heteroaryl ring is optionally substituted with 1, 2 or 3 substituents independently selected from the group consisting of halo, hydroxy, C₁₋₈alkyl, —O(C₁₋₈alkyl), nitro, amino, mono(C₁₋₈alkyl)amino, di(C₁₋₈alkyl)amino, —NHCO(C₁₋₈alkyl), —N(C₁₋₈alkyl)CO(C₁₋₈alkyl), —NHSO₂(C₁₋₈alkyl), —N(C₁₋₈alkyl)SO₂(C₁₋₈alkyl), —NHSO₂CF₃, —N(C₁₋₈alkyl)SO₂CF₃, —COOH, —CONH₂, —CONH(C₁₋₈alkyl), —CON(C₁₋₈alkyl)₂, —CO₂(C₁₋₈alkyl), —NHCO₂(C₁₋₈alkyl), —N(C₁₋₈alkyl)CO₂(C₁₋₈alkyl), CF₃, CN, —(CH₂)ₙOH, —(CH₂)ₙNH₂, —(CH₂)ₙNH(C₁₋₈alkyl), —(CH₂)ₙN(C₁₋₈alkyl)₂, —CH₂O(C₁₋₈alkyl), —NHSO₂NH₂, —N(C₁₋₈alkyl)SO₂NH₂, —NHSO₂NH(C₁₋₈alkyl), —NHSO₂N(C₁₋₈alkyl)₂, —N(C₁₋₈alkyl)SO₂N(C₁₋₈alkyl)₂, —NHCONH₂, —NHCONH(C₁₋₈alkyl), —NHCON(C₁₋₈alkyl)₂, —N(C₁₋₈alkyl)CON(C₁₋₈alkyl)₂, —S(C₁₋₈alkyl), —SO₂(C₁₋₈alkyl), —SO₃H, —SO₂O(C₁₋₈alkyl), —SO₂NH₂, —SO₂N(C₁₋₈alkyl)₂, —SO₂NH(C₁₋₈alkyl), or —NHC(NH)NH₂;

‘Y’ is selected from the group consisting of:
(a) —C(O)NRₐR_{b},
(b) —NR_{c}C(X)NRₐR_{b},
(c) —NR_{c}C(X)NR_{d}Rₑ,
(d) —NR_{c}C(O)OR_{f},
(e) —NR_{c}C(O)C(O)R_{g};

X is selected from O or S;

Rₐ and R_{b} together with the atoms with which they are attached form a three- to ten-membered monocyclic or bicyclic heterocyclyl or heteroaryl ring selected from the group consisting of aziridinyl, azepanyl, azetindinyl, azocanyl, azepinyl, diazepanyl, diazocanyl, hexahydropyridazinyl, hexahydropyrimidinyl, isothiazolidinyl, isoxazolidonyl, imidazolyl, imidazolidinyl, morpholinyl, oxazolidonyl, oxazolanyl, oxazetanyl, piperazinyl, piperazinonyl, piperidinyl, piperidonyl, pyrrolidinyl, pyrrolinyl, pyrroyl, pyrrolonyl, pyrrolidonyl, pyrazolyl, pyrazolonyl, thiomorpholinyl, thiomorpholin-1,1-dioxide, thiazolidinyl, thiazepanyl, thiazinyl, thiazocanyl, thiazetanyl, triazolyl, indolyl, indolinyl, indazolyl, tetrahydroquinolyl, tetrahydroisoquinolyl, or benzimidazolyl, wherein, said three- to ten-membered monocyclic or bicyclic heterocyclyl or heteroaryl ring is optionally substituted with 1, 2, or 3 substituents independently selected from the group consisting of

(1) phenyl, unsubstituted or substituted with a single substituent selected from hydroxy, mono(C₁₋₈alkyl)amino, di(C₁₋₈alkyl)amino, —NHCO(C₁₋₈alkyl), —N(C₁₋₈alkyl)CO(C₁₋₈alkyl), —NHCO₂(C₁₋₈alkyl), —N(C₁₋₈alkyl)CO₂(C₁₋₈alkyl), —NHNH₂, —N(C₁₋₈alkyl)N(C₁₋₈alkyl)₂, and —N(C₁₋₈alkyl)NH₂,

(2) =NOH, (3) optionally substituted heterocyclyl, (4) —O-optionally substituted heteroaryl, (5) —O-optionally substituted heterocyclyl, (6) —CO-optionally substituted heteroaryl, or (7) —CO-optionally substituted heterocyclyl,

wherein the substituent on the optionally substituted heteroaryl and heterocyclyl is a single group selected from hydroxy, C₁₋₈alkyl, —O(C₁₋₈alkyl), oxo, thioxo, amino, mono(C₁₋₈alkyl)amino, di(C₁₋₈alkyl)amino, —NHCO(C₁₋₈alkyl), —N(C₁₋₈alkyl)CO(C₁₋₈alkyl), NHCO₂(C₁₋₈alkyl), —N(C₁₋₈alkyl)CO₂(C₁₋₈alkyl), —NHNH₂, —N(C₁₋₈alkyl)N(C₁₋₈alkyl)₂, or —N(C₁₋₈alkyl)NH₂;

R_{c} and R_{d} are independently selected from hydrogen or C₁₋₆alkyl;

Rₑ is independently selected from —SO₂R₃, —SO₂R₄, —(CH₂)ₙR₄, —(CH₂)ₙR₄, —(CH₂)ₙCOR₄, —(CH₂)ₙOR₄, —(CH₂)ₙSR₇, —(CH₂)ₙSO₂R₇, —(CH₂)ₙNHCOR₇, —(CH₂)ₙN(C₁₋₈alkyl)COR₇, —(CH₂)ₙNHNHSO₂R₇, —(CH₂)ₙNHSO₂R₄, —(CH₂)ₙN(C₁₋₈alkyl)SO₂R₄, —(CH₂)ₙN(NH₂ )R₇, —(CH₂)ₙN[N(C₁₋₈alkyl)₂]R₇, or —NHSO₂R₇;

R_{f} is selected from the group consisting of (1) optionally substituted C₁₋₈alkyl, wherein the substituents are selected from oxo, thioxo, amino, C₁₋₃alkoxy, mono(C₁₋₃)alkylamino, di(C₁₋₃alkyl)amino, or hydroxy, (2) —R₃, (3) —R₄, (4) phenyl, unsubstituted or substituted with R₂, (5) —(CH₂)ₙR₇, (6) —(CH₂)ₙCOR₇, (7) —(CH₂)ₙNR_{c}R₇, (8) —(CH₂)ₙNHSO₂R₇, (9) —(CH₂)ₙN(C₁₋₈alkyl)SO₂R₇, (10) —(CH₂)ₙNHCOR₇, (11) —(CH₂)ₙN(C₁₋₈alkyl)COR₇, (12) —(CH₂)ₙOR₇, (13) —(CH₂)ₙSR₇, (14) —(CH₂)ₙSO₂R₇, (15) —CH₂)ₙNHNHSO₂R₇, (16) —(CH₂)ₙN(NH₂)R₇, or (17) —(CH₂)ₙN{N(C₁₋₈alkyl)₂}R₇;

R_{g} is selected from the group consisting of —NR_{c}(CH₂)ₙR₄, —NR_{c}(CH₂)ₙCOR₄, —NR_{c}(CH₂)ₙOR₄, —NR_{c}(CH₂)ₙNHSO₂R₄, —NR_{c}(CH₂)ₙN(C₁₋₈alkyl)SO₂R₄, —NR_{c}(CH₂)ₙSO₂R₇, —NR_{c}SO₂R₇, —NR_{c}(CH₂)ₙSR₇, —N(NH₂)R₇, —N[N(C₁₋₈alkyl)₂]R₇, —NR_{c}(CH₂)ₙNHNHSO₂R₇, —NR_{c}(CH₂ )ₙN(NH₂)R₇, —NR_{c}(CH₂)ₙN[N(C₁₋₈alkyl)₂]R₇, or —NR_{c}(CH₂)ₙNHCOR₇;

n is independently selected at each occurrence, from 1, 2 or 3;

R₃ at each occurrence is optionally substituted monocyclic three to seven membered heteroaryl ring having one to three heteroatoms independently selected from N, O, or S, wherein the substitution is by 1, 2 or 3 substituents represented by R₂;

R₄ at each occurrence is optionally substituted monocyclic three to seven membered heterocyclyl ring having one to three heteroatoms independently selected from N, O or S, wherein the substitution is by 1, 2 or 3 substituents represented by R₂;

R₅ at each occurrence is independently selected from hydrogen, C₁₋₆alkyl or CF₃;

R₆ at each occurrence are 1 or 2 groups independently selected from hydrogen, —O(C₁₋₈alkyl), halo, C₁₋₆alkyl, mono(C₁₋₆alkyl)amino or di(C₁₋₆alkyl)amino;

R₇ at each occurrence is
1. optionally substituted monocyclic three- to seven-membered aryl;
2. optionally substituted monocyclic three- to seven-membered heteroaryl or heterocyclyl having one to three heteroatoms independently selected from N, O or S,
   wherein the substitution on R₇ is by 1, 2 or 3 substituents represented by R₂;
   with the proviso that,
   when R_{f} is C₁₋₈alkyl, aryl, or R₃, then R₂ is an optionally substituted three- to seven-membered heterocyclyl or heteroaryl ring having upto three heteroatoms independently selected from N, O, or S.

A family of specific compounds of particular interest within the above formula (I) consists of compound and pharmaceutically acceptable salts thereof as follows:
N-(4-{2-Oxo-4-[4-(3-quinolin-2-yl-acryloyl)-benzoyl]-piperazin-1-yl}-phenyl)-acetamide (Compound No. 17);
3-Quinolin-2-yl-1-{4-[4-(tetrahydro-furan-2-carbonyl)-piperazine-1-carbonyl]-phenyl}-propenone (Compound No. 20);
1-{4-[4-(Furan-2-carbonyl)-piperazine-1-carbonyl]-phenyl}-3-quinolin-2-yl-propenone (Compound No. 21);
{4-[3-(4-Morpholin-4-yl-quinolin-2-yl)-acryloyl]-phenyl}-carbamic acid ethyl ester (Compound No. 23);
{4-[3-(4-Morpholin-4-yl-quinolin-2-yl)-acryloyl]-phenyl}-carbamic acid 2,2-dimethyl-propyl ester (Compound No. 24);
{4-[3-(4-Piperidin-1-yl-quinolin-2-yl)-acryloyl]-phenyl}-carbamic acid ethyl ester (Compound No. 29);
{4-[3-(4-Piperidin-1-yl-quinolin-2-yl)-acryloyl]-phenyl}-carbamic acid isobutyl ester (Compound No. 30);
{4-[3-(2-Pyrrolidin-1-yl-quinolin-3-yl)-acryloyl]-phenyl}-carbamic acid ethyl ester (Compound No. 31);
1-[4-(2,3-Dihydro-indole-1-carbonyl)-phenyl]-3-(3-hydroxy-quinoxalin-2-yl)-propenone (Compound No. 43);
1-[4-(3-Quinoxalin-2-yl-acryloyl)-benzoyl]-piperidin-4-one oxime (Compound No. 45);
1-Benzenesulfonyl-3-{4-[3-(6-methyl-4-piperidin-1-yl-quinolin-2-yl)-acryloyl]-phenyl}-urea (Compound No. 59);
{4-[3-(6-[1,2,3]Thiadiazol-4-yl-quinolin-2-yl)-acryloyl]-phenyl}-carbamic acid ethyl ester (Compound No. 64);
{4-[3-(6-[1,2,3]Thiadiazol-4-yl-quinolin-2-yl)-acryloyl]-phenyl}-carbamic acid phenyl ester (Compound No. 66);
{4-[3-(6-Morpholin-4-yl-pyridin-2-yl)-acryloyl]-phenyl}-carbamic acid 2-morpholin-4-yl-ethyl ester (Compound No. 67);
{5-Methoxy-2-[3-(4-morpholin-4-yl-quinolin-2-yl)-acryloyl]-phenyl}-carbamic acid methyl ester (Compound No. 69);
Propyl-{4-[3-(2-pyrrolidin-1-yl-quinolin-3-yl)-acryloyl]-phenyl}-carbamic acid ethyl ester (Compound No. 70);
{4-[3-(4-Morpholin-4-yl-quinolin-2-yl)-acryloyl]-phenyl}-carbamic acid phenyl ester (Compound No. 71);
{4-[3-(6-[1, 2, 3]Thiadiazol-4-yl-quinolin-2-yl)-acryloyl]-phenyl}-carbamic acid methyl ester (Compound No. 74);
1-(4-Methyl-benzenesulfonyl)-3-{4-[3-(3,4,5,6-tetrahydro-2H-[1,2′]bipyridinyl-6′-yl)-acryloyl]-phenyl}-urea (Compound No. 75);
{4-[3-(3,4,5,6-Tetrahydro-2H-[1,2′]bipyridinyl-6′-yl)-acryloyl]-phenyl}-carbamic acid ethyl ester (Compound No. 76);
(4-{3-[6-(3,5-Dimethyl-morpholin-4-yl)-pyridin-3-yl]-acryloyl}-phenyl)-carbamic acid phenyl ester (Compound No. 84);
5′-[3-Oxo-3-(4-phenoxycarbonylamino-phenyl)-propenyl]-3,4,5,6-tetrahydro-2H-[1,2′]bipyridinyl-4-carboxylic acid (Compound No. 86);
{4-[3-(6-Pyrrolidin-1-yl-pyridin-2-yl)-acryloyl]-phenyl}-carbamic acid pyridin-2-ylmethyl ester (Compound No. 90);
{4-[3-(6-Pyrrolidin-1-yl-pyridin-2-yl)-acryloyl]-phenyl}-carbamic acid 4-fluoro-benzyl ester (Compound No. 91);
{4-[3-(3,4,5,6-Tetrahydro-2H-[1,2′]bipyridinyl-6′-yl)-acryloyl]-phenyl}-carbamic acid 2-(3,5-dimethyl-pyrazol-1-yl)-ethyl ester (Compound No. 92);
{4-[3-(3,4,5,6-Tetrahydro-2H-[1,2′]bipyridinyl-6′-yl)-acryloyl]-phenyl}-carbamic acid furan-2-ylmethyl ester (Compound No. 93);
{4-[3-(3,4,5,6-Tetrahydro-2H-[1,2′]bipyridinyl-6′-yl)-acryloyl]-phenyl}-carbamic acid 3-phenyl-allyl ester (Compound No. 94);
{4-[3-(3,4,5,6-Tetrahydro-2H-[1,2′]bipyridinyl-6′-yl)-acryloyl]-phenyl}-carbamic acid 2-piperidin-1-yl-ethyl ester (Compound No. 95);
{4-[3-(4-Morpholin-4-yl-quinolin-2-yl)-acryloyl]-phenyl}-carbamic acid methyl ester (Compound No. 96);
(4-{3-[2-(4-Hydroxy-piperidin-1-yl)-quinolin-3-yl]-acryloyl}-phenyl)-carbamic acid phenyl ester (Compound No. 110);
(4-{3-[2-(4-Hydroxy-piperidin-1-yl)-quinolin-3-yl]-acryloyl}-phenyl)-carbamic acid methyl ester (Compound No. 111);
(4-{3-[2-(4-Hydroxy-piperidin-1-yl)-quinolin-3-yl]-acryloyl}-phenyl)-carbamic acid ethyl ester (Compound No. 112);
1-Benzenesulfonyl-3-(4-{3-[6-(3,5-dimethyl-morpholin-4-yl) -pyridin-3-yl]-acryloyl}-phenyl)-urea (Compound No. 113);
1-{4-[3-(6-Morpholin-4-yl-pyridin-2-yl)-acryloyl]-phenyl}-3-(2-oxo-2-piperidin-1-yl-ethyl)-urea (Compound No. 126);
1-(2-Morpholin-4-yl-ethyl)-3-{4-[3-(6-morpholin-4-yl-pyridin-2-yl)-acryloyl]-phenyl}-urea (Compound No. 127);
{4-[3-(6-Morpholin-4-yl-pyridin-2-yl)-acryloyl]-phenyl}-carbamic acid 2-piperazin-1-yl-ethyl ester (Compound No. 129);
N-(2-Morpholin-4-yl-ethyl)-N′-[4-(3-quinoxalin-2-yl-acryloyl)-phenyl]-oxalamide (Compound No. 130);
1-{4-[3-(6-Morpholin-4-yl-pyridin-2-yl)-acryloyl]-phenyl}-3-[2-(pyridine-2-sulfonyl)-ethyl]-urea (Compound No. 131);
1-{4-[3-(6-Morpholin-4-yl-pyridin-2-yl)-acryloyl]-phenyl}-3-[2-(pyridin-2-ylsulfanyl)-ethyl]-urea (Compound No. 132);
1-[2-(4-Methyl-piperazin-1-yl)-ethyl]-3-{4-[3-(6-morpholin-4-yl-pyridin-2-yl)-acryloyl]-phenyl}-urea (Compound No. 133);
N-(2-Morpholin-4-yl-ethyl)-N′-{4-[3-(6-morpholin-4-yl-pyridin-2-yl)-acryloyl]-phenyl}-oxalamide (Compound No. 134);
1-(2-Morpholin-4-yl-ethyl)-3-[4-(3-quinoxalin-2-yl-acryloyl)-phenyl]-urea (Compound No. 135);
1-Benzenesulfonyl-hydrazino-3-[4-(3-quinoxalin-2-yl-acryloyl)-phenyl]-urea (Compound No. 139);
1-(2-Morpholin-4-yl-2-oxo-ethyl)-3-[4-(3-quinolin-2-yl-acryloyl)-phenyl]-urea (Compound No. 140);
1-(Morpholine-4-sulfonyl)-3-[4-(3-quinolin-2-yl-acryloyl)-phenyl]-urea (Compound No. 142);
{4-[3-(6′-Morpholin-4-yl-pyridin-2-yl)-acryloyl]-phenyl}-carbamic acid piperidin-4-yl ester (Compound No. 143);
1-[2-(Pyridin-2-ylsulfanyl)-ethyl]-3-[4-(3-quinoxalin-2-yl-acryloyl)-phenyl]-urea (Compound No. 155);
{4-[3-(6-Morpholin-4-yl-pyridin-2-yl)-acryloyl]-phenyl}-carbamic acid 2-(pyridine-2-sulfonyl)-ethyl ester (Compound No. 156);
{4-[3-(6-Morpholin-4-yl-pyridin-2-yl)-acryloyl]-phenyl}-carbamic acid 2-(pyridin-2-ylsulfanyl)-ethyl ester (Compound No. 157);
[4-(3-Quinoxalin-2-yl-acryloyl)-phenyl]-carbamic acid 2-benzenesulfonylamino-ethyl ester (Compound No. 158);
{4-[3-(6-Morpholin-4-yl-pyridin-2-yl)-acryloyl]-phenyl}-carbamic acid 2-(N-pyridin-2-yl-hydrazino)-ethyl ester (Compound No. 159);
1-{2-[N-(6-Methyl-pyridin-2-yl-hydrazino]-ethyl}-3-[4-(3-quinoxalin-2-yl-acryloyl)-phenyl]-urea (Compound No. 160);
1-[4-(Pyrazole-1-carbonyl)-phenyl]-3-(3,4,5,6-tetrahydro-2H-[l,2′]bipyridinyl-5′-yl)-but-2-en-1-one (Compound No. 161);
4,4,4-Trifluoro-1-[4-(morpholine-4-carbonyl)-phenyl]-3-quinolin-3-yl-but-2-en-1-one (Compound No. 162);
N-{4-[3-(6-Morpholin-4-yl-pyridin-3-yl)-but-2-enoyl]-phenyl}-2-oxo-2-piperidin-1-yl-acetamide (Compound No. 163);
2-Morpholin-4-yl-2-oxo-N-[4-(4,4,4-trifluoro-3-quinolin-3-yl-but-2-enoyl)-phenyl]-acetamide (Compound No. 164);
Morpholine-4-carboxylic acid {4-[4,4,4-trifluoro-3-(4-morpholin-4-yl-quinolin-3-yl)-but-2-enoyl]-phenyl}-amide (Compound No. 165);
Morpholine-4-carboxylic acid {4-[3-(6-morpholin-4-yl-pyridin-3-yl)-but-2-enoyl]-phenyl}-amide (Compound No. 166);
N-[2-(3-{4-[3-(6-Morpholin-4-yl-pyridin-2-yl)-acryloyl]-phenyl}-ureido)-ethyl]-nicotinamide (Compound No. 167);
1-[2-(Piperidin-4-yloxy)-ethyl]-3-[4-(3-quinolin-2-yl-acryloyl)-phenyl]-urea (Compound No. 168);
(4-{3-[6-(4-Methyl-piperazin-1-yl)-pyridin-2-yl]-acryloyl}-phenyl)-carbamic acid furan-2-ylmethyl ester (Compound No. 169);
(4-{3-[6-(4-Methyl-piperazin-1-yl)-pyridin-2-yl]-acryloyl}-phenyl)-carbamic acid pyridin-2-ylmethyl ester (Compound No. 170);
(4-{3-[6-(4-Methyl-piperazin-1-yl)-pyridin-2-yl]-acryloyl}-phenyl)-carbamic acid 2-(3,5-dimethyl-pyrazol-1-yl)-ethyl ester (Compound No. 171);
{4-[3-(6-Morpholin-4-yl-pyridin-2-yl)-acryloyl]-phenyl}-carbamic acid 2-[1,2,4]triazol-1-yl-ethyl ester (Compound No. 172);
{4-[3-(6-Piperazin-1-yl-pyridin-2-yl)-acryloyl]-phenyl}-carbamic acid 2-[1,2,4]-triazol-1-yl-ethyl ester (Compound No. 173);
(4-{3-[6-(4-Methyl-piperazin-1-yl)-pyridin-2-yl]-acryloyl}-phenyl)-carbamic acid thiophen-2-ylmethyl ester (Compound No. 174);
2-{3-Oxo-3-[4-(thiophen-2-ylmethoxycarbonylamino)-phenyl]-propenyl}-quinoline-6-carboxylic acid (Compound no. 175);
{4-[3-(6-[1,2,4]Triazol-1-yl-pyridin-2-yl)-acryloyl]-phenyl}-carbamic acid thiophen-2-ylmethyl ester (Compound no. 176);
{4-[3-(6-Tetrazol-1-yl-pyridin-2-yl)-acryloyl]-phenyl}-carbamic acid thiophen-2-yl methyl ester (Compound No. 177);
{4-[3-(6-Morpholin-4-yl-pyridin-2-yl)-acryloyl]-phenyl}-carbamic acid pyridin-2yl methyl ester (Compound No. 178);
4-(3-Quinolin-2-yl-acryloyl)-phenyl]-carbamic acid pyridin-2-ylmethyl ester (Compound No. 179);
(4-{3-[6-(4-Methyl-piperazin-1-yl)-pyridin-2-yl]-acryloyl}-phenyl)-carbamic acid piperidin-4-yl ester (Compound No. 180);
{4-[3-(6-Morpholin-4-yl-pyridin-2-yl)-acryloyl]-phenyl}-carbamic acid 1-methyl-1H-pyrrol-3-yl ester! (Compound No. 181);
(4-{3-[6-(4-Methyl-piperazin-1-yl)-pyridin-2-yl]-acryloyl}-phenyl)-carbamic acid isoxazol-3-yl ester (Compound No. 182);
[4-(3-Quinolin-2-yl-acryloyl)-phenyl]-carbamic acid 1-methyl-1H-imidazol-4-ylmethyl ester (Compound No. 183);
[4-(3-Pyridin-2-yl-acryloyl)-phenyl]-carbamic acid 2-(4-methyl-piperazine-1-sulfonylamino)-ethyl ester (Compound No. 184);
[4-(3-Quinolin-2-yl-acryloyl)-phenyl]-carbamic acid 2-(4-methyl-piperazine-1-sulfonylamino)-ethyl ester (Compound No. 185);
(4-{3-[6-(4-Methyl-piperazin-1-yl)-pyridin-2-yl]-acryloyl}-phenyl)-carbamic acid 2-benzenesulfonylamino-ethyl ester (Compound No. 186);
{4-[3-(6-Morpholin-4-yl-pyridin-2-yl)-acryloyl]-phenyl}-carbamic acid 2-[(4-methyl-piperazine-1-carbonyl)-amino]-ethyl ester (Compound No. 187);
[4-(3-Quinolin-2-yl-acryloyl)-phenyl]-carbamic acid 2-[(4-methyl-piperazine-1-carbonyl)-amino]-ethyl ester (Compound No. 188);
[4-(3-Pyridin-2-yl-acryloyl)-phenyl]-carbamic acid 2-(N-pyridin-2-yl-hydrazino)-ethyl ester (Compound No. 189);
[4-(3-Pyridin-2-yl-acryloyl)-phenyl]-carbamic acid 2-[N-(5-methyl-isoxazol-3-yl)-hydrazino]-ethyl ester (Compound No. 190);
[4-(3-Pyridin-2-yl-acryloyl)-phenyl]-carbamic acid 2-(N′-benzenesulfonyl- hydrazino)-ethyl ester (Compound No. 191);
1-{4-[3-(6-Morpholin-4-yl-pyridin-2-yl)-acryloyl]-phenyl}-3-(2-oxo-2-piperazin-1-yl-ethyl)-urea (Compound No. 192);
1-[2-(4-Methyl-piperazin-1-yl)-2-oxo-ethyl]-3-[4-(3-quinolin-2-yl-acryloyl)-phenyl]-urea (Compound No. 193);
1-(2-Morpholin-4-yl-2-oxo-ethyl)-3-{4-[3-(6-piperazin-1-yl-pyridin-2-yl)-acryloyl]-phenyl}-urea (Compound No. 194);
1-[2-(Pyridine-2-sulfonyl)-ethyl]-3-[4-(3-pyridin-2-yl-acryloyl)-phenyl]-urea (Compound No. 195);
1-{4-[3-(6-Piperazin-1-yl-pyridin-2-yl)-acryloyl]-phenyl}-3-[2-(piperidine-4-sulfonyl)-ethyl]-urea (Compound No. 196);
4-Methyl-piperazine-1-sulfonic acid(2-{3-[4-(3-quinolin-2-yl-acryloyl)-phenyl]-ureido}-ethyl)-amide (Compound No. 197);
1-{4-[3-(6-Morpholin-4-yl-pyridin-2-yl)-acryloyl]-phenyl}-3-[2-(piperidin-4-ylsulfanyl)-ethyl] -urea (Compound No. 198);
1-{2-[N-(1-Methyl-piperidin-4-yl)-hydrazino]-ethyl}-3-[4-(3-pyridin-2-yl-acryloyl)-phenyl]-urea (Compound No. 199);
1-{2-[N-(1-Methyl-piperidin-4-yl)-hydrazino]-ethyl}-3-[4-(3-quinoxalin-2-yl-acryloyl)-phenyl]-urea (Compound No. 200);
1-{2-[N-(6-Methyl-pyridin-2-yl)-hydrazino]-ethyl}-3-{4-[3-(6-morpholin-4-yl-pyridin-2-yl)-acryloyl]-phenyl}-urea (Compound No. 201);
1-(2-Piperazin-1-yl-ethyl)-3-[4-(3-quinoxalin-2-yl-acryloyl)-phenyl]-urea (Compound No. 202);
1-(2-Morpholin-4-yl-ethyl)-3-[4-(3-quinolin-2-yl-acryloyl)-phenyl]urea (Compound No. 203);
1-(2-Piperazin-1-yl-ethyl)-3-[4-(3-quinolin-2-yl-acryloyl)-phenyl]-urea (Compound No. 204);
1-{4-[3-(6-Piperazin-1-yl-pyridin-2-yl)-acryloyl]-phenyl}-3-(2-piperidin-1-yl-ethyl)-urea (Compound No. 205);
1-{4-[3-(6-Piperazin-1-yl-pyridin-2-yl)-acryloyl]-phenyl}-3-(2-thiomorpholin 1,1-dioxide-4-yl-ethyl)-urea (Compound No. 206);
1-(2-Piperazin-1-yl-ethyl)-3-{4-[3-(3)3,4,5,6-tetrahydro-2H-[1,2′]bipyridinyl-6′-yl)-acryloyl]-phenyl}-urea (Compound No. 207) ;
Piperidine-4-carboxylic acid [2-(3-{4-[3-(6-morpholin-4-yl-pyridin-2-yl)-acryloyl]-phenyl}-ureido)-ethyl]-amide (Compound No. 208);
N-(2-{3-[4-(3-Pyridin-2-yl-acryloyl)-phenyl]-ureido}-ethyl)-nicotinamide (Compound No. 209);
1-[2-(N′-Benzenesulfonyl-hydrazino)-ethyl]-3-[4-(3-pyridin-2-yl-acryloyl)-phenyl]-urea (Compound No. 210);
1-[2-(N′-Benzenesulfonyl-hydrazino)-ethyl]-3-[4-(3-quinolin-2-yl-acryloyl)-phenyl]-urea (Compound No. 211);
1-(Piperazine-1-sulfonyl)-3-[4-(3-pyridin-2-yl-acryloyl)phenyl]-urea (Compound No. 212);
1-{4-[3-(6-Morpholin-4-yl-pyridin-2-yl)-acryloyl]-phenyl}-3-(piperazine-1-sulfonyl)-urea (Compound No. 213);
1-(Piperazine-1-sulfonyl)-3-[4-(3′-quinoxalin-2-yl-acryloyl)-phenyl]-urea (Compound No. 214);
1-(Piperazine-1-sulfonyl)-3-[4-(3-quinolin-2-yl-acryloyl) phenyl]-urea (Compound No. 215);
N-(2-Piperazin-1-yl-ethyl)-N′-[4-(3-pyridin-2-yl-acryloyl)-phenyl]-oxalamide (Compound No. 216);
N-{4-[3-(6-Morpholin-4-yl-pyridin-2-yl)-acryloyl]-phenyl}-N′-(2-piperazin-1-yl-ethyl)-oxalamide (Compound No. 217);
1-[2-({4-[3-(6-Morpholin-4-yl-pyridin-2-yl)-acryloyl]-phenylaminooxalyl}-amino)-acetyl]-piperidine-4-carboxylic acid (Compound No. 218);
N-(2-Oxo-2-piperazin-1-yl-ethyl)-N′-[4-(3-pyridin-2-yl-acryloyl)-phenyl]-oxalamide (Compound No. 219);
N-[4-(3-Pyridin-2-yl-acryloyl)-phenyl]-N′-[2-(pyridine-2-yl-sulfonyl)-ethyl]-oxalamide (Compound No 220);
N-[2-(Piperidin-4-ylsulfanyl)-ethyl]-N′-[4-(3-pyridin-2-yl-acryloyl)-phenyl]-oxalamidea (Compound No. 221);
N-[2-(Pyridine-2-sulfonyl)-ethyl]-N′-[4-(3-quinolin-2-yl-acryloyl)-phenyl]-oxalamide (Compound No. 222);
2-[N-(6-Methyl-pyridin-2-yl)-hydrazino]-2-oxo-N-[4-(3-quinolin-2-yl-acryloyl)-phenyl]-acetamide (Compound No. 223);
2-Oxo-2-(N-phenyl-hydrazino)-N-[4-(3-pyridin-2-yl-acryloyl)-phenyl]-acetamide (Compound No. 224);
2-Oxo-2-(piperazine-1-sulfonylamino)-N-[4-(3-pyridin-2-yl-acryloyl)-phenyl]-acetamide (Compound No. 225);
2-Benzenesulfonylamino-2-oxo-N-[4-(3-pyridin-2-yl-acryloyl)-phenyl]-acetamide (Compound No. 226);
N-[2-(Piperazine-1-sulfonylamino)-ethyl]-N′-[4-(3-pyridin-2-yl-acryloyl)-phenyl]-oxalamide (Compound No. 227);
2-(N′-Benzenesulfonyl-hydrazino)-2-oxo-N-[4-(3-pyridin-2-yl-acryloyl)-phenyl]-acetamide (Compound No. 228);
N-{2-[N-(6-Methyl-pyridin-2-yl)-hydrazino]-ethyl}-N′-[4-(3-pyridin-2-yl-acryloyl)-phenyl]-oxalamide (Compound No. 229);
N-{2-[(Piperazine-1-carbonyl)-amino]-ethyl}-N′-[4-(3-pyridin-2-yl-acryloyl)-phenyl]-oxalamide (Compound No. 230);
N-{2-[(Pyridine-3-carbonyl)-amino]-ethyl}-N′-[4-(3-pyridin-2-yl-acryloyl)-phenyl]-oxalamide (Compound No. 231);
N-{2-[(Piperidine-4-carbonyl)-amino]-ethyl}-N′-[4-(3-pyridin-2-yl-acryloyl)-phenyl]-oxalamide (Compound No. 232);
N-(1-{4-[4-(3-Pyridin-2-yl-acryloyl)-benzoyl]-piperazin-1-yl}-piperidin-4-yl)-methanesulfonamide (Compound No. 247);
(1-{4-[4-(3-Pyridin-2-yl-acryloyl)-benzoyl]-piperazin-1-yl}-piperidin-4-yl)-sulfonylurea (Compound No. 248);
1-{4-[4-(4-Cyclohexylamino-piperidin-1-yl)-piperazine-1-carbonyl]-phenyl}-3-pyridin-2-yl-propenone (Compound No. 249).

Another embodiment of the invention consists of those compounds of Formula (I), wherein

wherein, Q is as defined hereinabove, substituted by either R₁ or both R₁ and R₂, wherein the number of substituents are selected from one to six;

R₁ is independently selected at each occurrence from, —SO₂OR₇, —SO₂O(C₁₋₈alkyl), —NHNH₂, —NHNHSO₂R₇, —NH(CH₂)ₙR₄, —NHCO₂R₇, —NHCO₂(C₁₋₈alkyl), —NHSO₂O(C₁₋₈alkyl), —NHSO₂OR₇, —NHSO₂NH₂, —NH(CH₂)ₙCOR₄, —NH(CH₂)ₙOR₄, —NH(CH₂)ₙSR₇, —NH(CH₂)ₙSO₂R₇, —NH(CH₂ )ₙNHCOR₄, —NH(CH₂)ₙN(C₁₋₈alkyl)COR₄, —N(C₁₋₈alkyl)(CH₂)ₙNHCOR₄, —NH(CH₂)ₙNHNHSO₂R₇, —NH(CH₂)ₙNHSO₂R₄, —NH(CH₂)ₙN(C₁₋₈alkyl)SO₂R₄, —NH(CH₂)ₙN(NH₂)R₇, —NH(CH₂)ₙN[N(C₁₋₈alkyl)₂]R₇, —N(C₁₋₈alkyl)CO₂R₇, —N(C₁₋₈alkyl)CO₂(C₁₋₈alkyl), —N(C₁₋₈alkyl)SO₂O(C₁₋₈alkyl), —N(C₁₋₈alkyl)SO₂OR₇, —N(C₁₋₈alkyl)SO₂NH₂, —N(C₁₋₈alkyl)N(C₁₋₈alkyl)₂, —N(C₁₋₈alkyl)NH₂, —NHNHCO(C₁₋₈alkyl), —N(C₁₋₈alkyl)NHCO(C₁₋₈alkyl), —NHNHCOR₇, —N(C₁₋₈alkyl)NHCOR₇, —N(C₁₋₈alkyl)—CH₂)ₙR₄, —N(C₁₋₈alkyl)(CH₂)ₙCOR₄, —(CH₂)ₙSO₂R₇, —(CH₂)ₙCOR₄, —(CH₂)ₙR₄, —(CH₂)ₙNHSO₂R₄, —(CH₂)ₙN(C₁₋₈alkyl)SO₂R₄, —(CH₂)ₙNHCOR₇, —(CH₂)ₙN(C₁₋₈alkyl)COR₇, —(CH₂)ₙOR₄, —(CH₂)ₙSR₄, —(CH₂)ₙSR₃, —(CH₂)ₙSO₂R₇, —(CH₂)ₙNHNHSO₂R₇, —(CH₂)ₙN(NH₂)R₇, or —(CH₂)ₙN[N(C₁₋₈alkyl)₂]R₇;

R₂ is as defined hereinabove;

‘Y’ is selected from the group consisting of:
(a) —C(O)NRₐR_{b},
(b) —NR_{c}C(X)NRₐR_{b},
(c) —NR_{c}C(X)NR_{d}Rₐ,
(d) —NR_{c}C(O)OR_{f},
(e) —NR_{c}C(O)C(O)R_{g};

X is selected from O or S;

Rₐ and R_{b} together with the atoms with which they are attached form a three- to ten-membered monocyclic or bicyclic heterocyclyl or heteroaryl ring selected from the group consisting of aziridinyl, azepanyl, azetindinyl, azocanyl, azepinyl, diazepanyl, diazocanyl, hexahydropyridazinyl, hexahydropyrimidinyl, isothiazolidinyl, isoxazolidonyl, imidazolyl, imidazolidinyl, morpholinyl, oxazolidonyl, oxazolanyl, oxazetanyl, piperazinyl, piperazinonyl, piperidinyl, piperidonyl, pyrrolidinyl, pyrrolinyl, pyrroyl, pyrrolonyl, pyrrolidonyl, pyrazolyl, pyrazolonyl, thiomorpholinyl, thiomorpholin-1,1-dioxide, thiazolidinyl, thiazepanyl, thiazinyl, thiazocanyl, thiazetanyl, triazolyl, indolyl, indolinyl, indazolyl, tetrahydroquinolyl, tetrahydroisoquinolyl, or benzimidazolyl, wherein, said three- to ten-membered monocyclic or bicyclic heterocyclyl or heteroaryl ring is optionally substituted with 1, 2, or 3 substituents independently selected from the group consisting of

(1) halo, (2) hydroxy, (3) optionally substituted C₁₋₈alkyl, wherein the substituents are amino, C₁₋₃alkoxy, mono(C₁₋₃alkyl)amino, di(C₁₋₃alkyl)amino, and hydroxy, (4) —O(C₁₋₈alkyl), (5) nitro, (6) amino, (7) mono(C₁₋₈alkyl)amino, (8) di(C₁₋₈alkyl)amino, (9) —COOH, (10) —CO(C₁₋₈alkyl), (11) —CONH₂, (12) —CONH(C₁₋₈alkyl), (13) —CON(C₁₋₈alkyl)₂, (14) —CO₂(C₁₋₈alkyl), (15) formyl, (16) =NOH, (17) CF₃, (18) CN, (19) —NHSO₂NH₂ (20) —NHCO(C₁₋₈alkyl), (21) —N(C₁₋₈alkyl)CO(C₁₋₈alkyl), (22) —NHSO₂(C₁₋₈alkyl), (23) —N(C₁₋₈alkyl)SO₂(C₁₋₈alkyl), (24) —NHSO₂CF₃, (25) —N(C₁₋₈alkyl)CO₂(C₁₋₈alkyl), (26) —N(C₁₋₈alkyl)SO₂CF₃, (27) —N(C₁₋₈alkyl)SO₂NH₂, (28) —NHSO₂NH(C₁₋₈alkyl), (29) —NHSO₂N(C₁₋₈alkyl)₂, (30) —N(C₁₋₈alkyl)SO₂N(C₁₋₈alkyl)₂, (31) —NHCONH₂, (32) —NHCONH(C₁₋₈alkyl), (33) —NHCON(C₁₋₈alkyl)₂, (34) —N(C₁₋₈alkyl)CO(C₁₋₈alkyl), (35) —N(C₁₋₈alkyl)CO₂(C₁₋₈alkyl), (36) —N(C₁₋₈alkyl)CON(C₁₋₈alkyl)₂, (37) —S(C₁₋₈alkyl), (38) —SO₂(C₁₋₈alkyl), (39) —SO₃H, (40) —SO₂O(C₁₋₈alkyl), (41) —SO₂NH₂ , (42) —SO₂N(C₁₋₈alkyl)₂, (43) —SO₂NH(C₁₋₈alkyl), (44) —NHC(NH)NH₂, (45) phenyl, unsubstituted or substituted with one to two substitutents selected from halo, nitro, C₁₋₃alkyl, C₁₋₃alkoxy, hydroxy, amino, mono(C₁₋₈alkyl)amino, di(C₁₋₈alkyl)amino, —NHCO(C₁₋₈alkyl), —N(C₁₋₈alkyl)CO(C₁₋₈alkyl), —NHCO₂(C₁₋₈alkyl), —NHCO₂(C₁₋₈alkyl), —N(C₁₋₈alkyl)CO₂(C₁₋₈alkyl), —NHNH₂, —N(C₁₋₈alkyl)N(C₁₋₈alkyl)₂, and —N(C₁₋₈alkyl)NH₂, (46) pyridyl, unsubstituted or substituted with one to two substitutents selected from halo, C₁₋₃alkyl and C₁₋₃alkoxy, (47) —CO—(optionally substituted heteroaryl), (48) —CO—(optionally substituted heterocyclyl), (49) —O— (optionally substituted heteroaryl), (50) —O—(optionally substituted heterocyclyl), (51) optionally substituted heterocyclyl, (52) —NH—(optionally substituted heterocyclyl),

wherein the substituents on the optionally substituted heteroaryl and heterocyclyl are one to two groups independently selected from hydroxy, C₁₋₈alkyl, —O(C₁₋₈alkyl), oxo, thioxo, amino, mono(C₁₋₈alkyl)amino, di(C₁₋₈alkyl)amino, —NHCO(C₁₋₈alkyl), —N(C₁₋₈alkyl)CO(C₁₋₈alkyl), —NHCO₂(C₁₋₈alkyl), —N(C₁₋₈alkyl)CO₂(C₁₋₈alkyl), —NHNH₂, —N(C₁₋₈alkyl)N(C₁₋₈alkyl)₂, _NHSO₂(C₁₋₈alkyl), —NHSO₂NH₂ or —N(C₁₋₈alkyl)NH₂;

R_{c} and R_{d} are independently selected from hydrogen or C₁₋₆alkyl;

R_{c} is selected from R₇, —SO₂R₇, —SO₂R₃, —SO₂R₄, —COR₇, —(CH₂)ₙR₇, —(CH₂)ₙCOR₇, —(CH₂)ₙOR₇, —(CH₂)ₙSR₇, —(CH₂)ₙSO₂R₇, —(CH₂)ₙNHCOR₇, —(CH₂)ₙNHSO₂R₇, —(CH₂)ₙN(C₁₋₈alkyl)COR₇, —(CH₂)ₙNHNHSO₂R₇, —(CH₂)ₙNHSO₂R₄, —(CH₂)ₙN(C₁₋₈alkyl)SO₂R₄, —(CH₂)ₙN(NH₂)R₇, —(CH₂)ₙN[N(C₁₋₈alkyl)₂]R₇, —NHSO₂R₇, optionally substituted C₁₋₈alkyl, wherein the substituents are C₁₋₃ alkoxy, amino, mono(C₁₋₃alkyl)amino, di(C₁₋₃alkyl)amino, or hydroxy;

R_{f} is selected from the group consisting of (1) optionally substituted C₁₋₈alkyl, wherein the substituents are selected from C₁₋₃alkoxy, amino, mono(C₁₋₃alkyl)amino, di(C₁₋₃alkyl)amino, C₁₋₃alkyl, phenyl, or hydroxy, (2) —R₃, (3) —R₄, (4) phenyl, unsubstituted or substituted with R₂, (5) —(CH₂)ₙR₇, (6) —(CH₂)ₙCOR₇, (7) —(CH₂)ₙNR_{c}R₇, (8) —(CH₂)ₙNHSO₂R₇, (9) —(CH₂)ₙN(C₁₋₈alkyl)SO₂R₇, (10) —(CH₂ )ₙNHCOR₇, (11) —CH₂)ₙN(C₁₋₈alkyl)COR₇, (12) —(CH₂)ₙOR₇, (13) —(CH₂)ₙSR₇, (14) —(CH₂)ₙSO₂R₇, (15) —(CH₂)ₙNHNHSO₂R₇, (16) —(CH₂)ₙN(NH₂)R₇, (17) —(CH₂)ₙN{N(C₁₋₈alkyl)₂}R₇ or (18) CCl₃;

R_{g} is selected from the group consisting of (1) mono(C₁₋₈alkyl)amino (2) di(C₁₋₈-alkyl)amino, (3) NH₂, (4) —NHR₇, (5) —NR_{c}(CH₂)ₙR₇, (6) —NR_{c}(CH₂)ₙCOR₇, (7) —NH(CH₂)ₙO(C₁₋₈alkyl), (8) —NR_{c}(CH₂)ₙOR₇, (9) —NR_{c}(CH₂)ₙNHSO₂R₇, (10) —NR_{c}(CH₂)ₙN(C₁₋₈alkyl)SO₂R₇, (11) —NR_{c}(CH₂)ₙSO₂R₇, (12) —NR_{c}SO₂R₇, (13) —NR_{c}(CH₂)ₙSR₇, (14) —N(NH₂)R₇, (15) —N[N(C₁₋₈alkyl)₂]R₇, (16) —NR_{c}(CH₂)ₙNHNHSO₂R₇, (17) —NR_{c}(CH₂)ₙN(NH₂)R₇, (18) —NR_{c}(CH₂)ₙN[N(C₁₋₈alkyl)₂]R₇, (19) —NR_{c}(CH₂)ₙNHCOR₇, (20) —NHNHSO₂R₇, (21) optionally substituted three- to ten-membered monocyclic or bicyclic heterocyclyl or heteroaryl ring attached through the ring nitrogen atom and selected from the group consisting of aziridinyl, azepanyl, azetindinyl, azocanyl, azepinyl, diazepanyl, diazocanyl, hexahydropyridazinyl, hexahydropyrimidinyl, isothiazolidinyl, isoxazolidonyl, imidazolyl, imidazolidinyl, morpholinyl, oxazolidonyl, oxazolanyl, oxazetanyl, piperazinyl, piperazinonyl, piperidinyl, piperidonyl, pyrrolidinyl, pyrrolinyl, pyrroyl, pyrrolonyl, pyrrolidonyl, pyrazolyl, pyrazolonyl, thiomorpholinyl, thiomorpholin-1,1-dioxide, thiazolidinyl, thiazepanyl, thiazinyl, thiazocanyl, thiazetanyl, triazolyl, indolyl, indolinyl, indazolyl, tetrahydroquinolyl, tetrahydroisoquinolyl, or benzimidazolyl,

wherein, the substituents on said optionally substituted three- to ten-membered monocyclic or bicyclic heterocyclyl or heteroaryl ring are 1, 2 or 3 groups independently selected from (1) halo, (2) hydroxy, (3) C₁₋₈alkyl, unsubstituted or substituted with C₁₋₃alkoxy, amino, mono(C₁₋₃alkyl)amino, di(C₁₋₃alkyl)amino, C₁₋₃alkyl, and hydroxy, (4) —O(C₁₋₈alkyl), (5) nitro, (6) amino, (7) mono(C₁₋₈alkyl)amino, (8) di(C₁₋₈alkyl)amino, (9) —COOH, (10) —CO(C₁₋₈alkyl), (11) —CONH₂, (12) —CONH(C₁₋₈alkyl), (13) —CON(C₁₋₈alkyl)₂, (14) —CO₂(C₁₋₈alkyl), (15) formyl, (16) =NOH, (17) CF₃, (18) CN, (19) —NHSO₂NH₂, (20) —NHCO(C₁₋₈alkyl), (21) —N(C₁₋₈alkyl)CO(C₁₋₈alkyl), (22) —NHSO₂(C₁₋₈alkyl), (23) —N(C₁₋₈alkyl)SO₂(C₁₋₈alkyl), (24) —NHSO₂CF₃, (25) —N(C₁₋₈alkyl)CO₂(C₁₋₈alkyl), (26) —N(C₁₋₈alkyl)SO₂CF₃, (27) —N(C₁₋₈alkyl)SO₂NH₂, (28) —NHSO₂NH(C₁₋₈alkyl), (29) —NHSO₂N(C₁₋₈alkyl)₂, (30) —N(C₁₋₈alkyl)SO₂N(C₁₋₈alkyl)₂, (31) —NHCONH₂, (32) —NHCONH(C₁₋₈alkyl), (33) —NHCON(C₁₋₈alkyl)₂, (34) —N(C₁₋₈alkyl)CO (C₁₋₈alkyl), (35) —N(C₁₋₈alkyl)CO₂(C₁₋₈alkyl), (36) —N(C₁₋₈alkyl)CON(C₁₋₈alkyl)₂, (37) —S(C₁₋₈alkyl), (38) —SO₂(C₁₋₈alkyl), (39) —SO₃H, (40) —SO₂O(C₁₋₈alkyl), (41) —SO₂NH₂, (42) —SO₂N(C₁₋₈alkyl)₂, (43) —SO₂NH(C₁₋₈alkyl), or (44) —NHC(NH)NH₂,

n is independently selected at each occurrence, from 1, 2 or 3;

R₃ at each occurrence is optionally substituted monocyclic three to seven membered heteroaryl ring having one to three heteroatoms independently selected from N, O, or S, wherein the substitution is by 1, 2 or 3 substituents represented by R₂;

R₄ at each occurrence is optionally substituted monocyclic three to seven membered heterocyclyl ring having one to three heteroatoms independently selected from N, O or S, wherein the substitution is by 1, 2 or 3 substituents represented by R₂;

R₅ at each occurrence is independently selected from hydrogen, C₁₋₆alkyl or CF₃;

R₆ at each occurrence are 1 or 2 groups independently selected from hydrogen, —O(C₁₋₈alkyl), halo, C₁₋₆alkyl, mono(C₁₋₆alkyl)amino or di(C₁₋₆alkyl)amino;

R₇ at each occurrence is
1. optionally substituted monocyclic three- to seven-membered aryl;
2. optionally substituted monocyclic three- to seven-membered heteroaryl or heterocyclyl having one to three heteroatoms independently selected from N, O or S,
   wherein the substitution on R₇ is by 1, 2 or 3 substituents represented by R₂.

A further family of specific compounds of particular interest within the above formula (I) consists of compound and pharmaceutically acceptable salts thereof as follows:
1-[4-(4-Methyl-piperazine-1-carbonyl)-phenyl]-3-{6-[2-(4-methyl-piperazin-1-yl)-ethylamino]-pyridin-2-yl}-propenone (Compound No. 233);
(2-{3-[4-(4-Methyl-piperazine-1-carbonyl)-phenyl]-3-oxo-propenyl}-quinolin-6-yl)-carbamic acid ethyl ester (Compound No. 234);
(6-{3-[4-(4-Methyl-piperazine-1-carbonyl)-phenyl]-3-oxo-propenyl}-pyridin-2-yl)-carbamic acid phenyl ester (Compound No. 235);
1-[4-(Morpholine-4-carbonyl)-phenyl]-3-{6-[(piperidin-1-ylmethyl)-amino]-pyridin-2-yl}-propenone (Compound No. 236);
1-[4-(3-Dimethylamino-pyrazole-1-carbonyl)-phenyl]-3-[6-(2-oxo-2-piperidin-1-yl-ethylamino)-pyridin-2-yl]-propenone (Compound No. 237);
1-[4-(Piperazine-1-carbonyl)-phenyl]-3-{6-[2-(pyridine-2-sulfonyl)-ethylamino]-pyridin-2-yl}-propenone (Compound No. 238);
Benzenesulfonic acid N-[2-(6-{3-oxo-3-[4-(piperidine-1-carbonyl)-phenyl]-propenyl}-pyridin-2-ylamino)-ethyl]-hydrazide (Compound No. 239);
(6-{3-[4-(3-Benzenesulfonyl-ureido)-phenyl]-3-oxo-propenyl}-pyridin-2-yl)-carbamic acid ethyl ester (Compound No. 240);
Morpholine-4-carboxylic acid (4-{3-[6-(2-piperidin-1-yl-ethylamino)-pyridin-2-yl]-acryloyl}-phenyl)-amide (Compound No. 241);
{2-[3-(4-Ethoxycarbonylamino-phenyl)-3-oxo-propenyl]-quinolin-6-yl}-carbamic acid ethyl ester (Compound No. 242);
{2-[3-Oxo-3-(4-phenoxycarbonylamino-phenyl)-propenyl]-quinolin-4-yl}-carbamic acid ethyl ester (Compound No. 243);
[2-(3-{4-[2-(4-Methyl-piperazin-1-yl)-2-oxo-acetylamino]-phenyl}-3-oxo-propenyl)-quinolin-4-yl]-carbamic acid methyl ester (Compound No. 244);
1-(2-Morpholin-4-yl-ethyl)-3-(4-{3-[6-(2-piperidin-1-yl-ethylamino)-pyridin-2-yl]-acryloyl}-phenyl)-urea (Compound No. 245);
1-[4-(3-Dimethylamino-pyrazole-1-carbonyl)-phenyl]-3-{6-[2-(4-methyl-piperazin-1-yl)-2-oxo-ethylamino]-pyridin-2-yl}-propenone (Compound No. 246);
[6-(3-Oxo-3-{4-[4-(pyrrolidine-1-carbonyl)-piperidine-1-carbonyl]-phenyl}-propenyl)-pyridin-2-yl]-carbamic acid methyl ester (Compound No. 250);
[6-(3-Oxo-3-{4-[4-(pyridin-2-yloxy)-piperidine-1-carbonyl]-phenyl}-propenyl)-pyridin-2-yl]-carbamic acid ethyl ester (Compound No. 251).

In a more preferred embodiment of the invention, R₂ is an optionally substituted three- to seven-membered heterocyclyl or heteroaryl ring having upto three heteroatoms independently selected from N, O, or S, said optionally substituted heterocyclyl or heteroaryl ring is selected from piperazinyl, piperidinyl, piperidonyl, morpholinyl, thiomorpholinyl, thiomorpholin-1,1-dioxide, pyrrolidinyl pyrrolyl, pyrazolyl, oxazolyl, isoxazolyl, imidazolyl, oxadiazolyl, thiadiazolyl, triazolyl, tetrazolyl and thiazolidinyl;

Rₐ and R_{b} are selected from optionally substituted piperazinyl, piperidinyl, piperidonyl, morpholinyl, thiomorpholinyl, thiomorpholin-1,1-dioxide, pyrrolidinyl pyrrolyl, pyrazolyl, triazolyl and imidazolyl;

X is O;

n is independently selected from 1 or 2;

R₅ is independently selected from hydrogen or methyl.

Ddefinitions:

The patents, published applications, and scientific literature referred to herein establish the knowledge of those skilled in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used herein, “compound” refers to any compound encompassed by the generic formulae disclosed herein. The compounds described herein may contain one or more double bonds and therefore, may exist as stereoisomers, such as double-bond isomers (i.e., geometric isomers). Accordingly, the chemical structures depicted herein encompass all possible stereoisomers of the illustrated compounds including the stereoisomerically pure form (e.g., geometrically pure) and stereoisomeric mixtures. The compounds may also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that may be incorporated into the compounds of the invention include, but are not limited to ²H, ³H, ¹³C, ¹⁴C, ¹⁵N, ¹⁸O, ¹⁷O, etc. Compounds may exist in unsolvated forms as well as solvated forms, including hydrated forms. In general, compounds may be hydrated or solvated. Certain compounds may exist in multiple crystalline or amorphous forms. In general, all physical forms are equivalent for the uses contemplated herein and are intended to be within the scope of the present invention. Further, it should be understood, when partial structures of the compounds are illustrated, a dash (“-”) indicate the point of attachment of the partial structure to the rest of the molecule. The nomenclature of the compounds of the present invention as indicated herein is according to MDL ISIS® Draw Version 2.2.1.

The term “substituted”, as used herein, means that any one or more hydrogens on the designated atom is replaced with a selection from the indicated group, provided that the designated atom's normal valence is not exceeded, and that the substitution results in a stable compound. When a substituent is keto, then 2 hydrogens on the atom are replaced. Groups that are “optionally substituted” may be either unsubstituted or substituted with one or more suitable groups.

When any variable occurs more than once in any constituent or formula for a compound, its definition at each occurrence is independent of its definition at every other occurrence. Thus, for example, if a group is shown to be substituted with 0-2 R*, then said group may optionally be substituted with up to two R* groups and each R* is selected independently from the definition of R*. Also, combinations of substituents and/or variables are permissible only if such combinations result in stable compounds.

The term “alkyl”, used either alone or in attachment with another group, refers to a monovalent, saturated aliphatic hydrocarbon radical having the indicated number of carbon atoms and that is unsubstituted or optionally substituted. When a subscript is used with reference to an alkyl or other group, the subscript refers to the number of carbon atoms that the group may contain. For example, a “C₁₋₈ alkyl” would refer to any alkyl group containing one to eight carbons in the structure, Alkyl may be a straight chain (i.e. linear) or a branched chain or cyclic, and may contain one or two double or triple bonds. The radical may be optionally substituted with substituents at positions that do not significantly interfere with the preparation of compounds falling within the scope of this invention. The alkyl is optionally substituted with one to two substituents independently selected from the group consisting of C₁₋₃alkoxy, amino, mono(C₁₋₃alkyl)amino, di(C₁₋₃alkyl)amino, C₁₋₃alkyl and hydroxy.

The term “alkoxy” refers to an alkyl group as defined above attached to the parent molecular moiety through an oxygen bridge. Representative alkoxy radicals include methoxy, ethoxy, n-propoxy, n-butoxy, n-pentyloxy, n-hexyloxy, isopropoxy, isobutoxy, isopentyloxy, amyloxy, sec-butoxy, tert-butoxy, tert-pentyloxy, and the like. The radical may be optionally substituted with substituents at positions that do, not significantly interfere with the preparation of compounds falling within the scope of this invention.

As used herein, a “halo” substituent is a monovalent halogen radical chosen from chloro, bromo, iodo and fluoro.

The term “monoalkylamino” as employed herein refers to an amino group which is substituted with one alkyl group having from 1 to 8 carbon atoms, for example, methylamino group, ethylamino group, propylamine group, isopropylamino group, butylamino group, isobutylamino group, tert-butylamino group, pentylamino group and isopentylamino group.

The term “dialkylamino” as employed herein refers to an amino group which is independently substituted with two alkyl groups, each having from 1 to 8 carbon atoms, for example, dimethylamino group, ethylmethylamino group, diethylamino group, methylpropylamino group and diisopropylamino group.

The term “aryl” refers to an aromatic group for example, which is a 3 to 10 membered monocyclic or bicyclic carbon-containing ring system, which may be unsubstituted or substituted . Representative aryl groups include phenyl, naphthyl and the like.

The term “heteroaryl” refers to an aromatic group for example, which is a 3 to 10 membered monocyclic or bicyclic ring system, which has at least one heteroatom and at least one carbon atom containing ring. The term “heteroatom” as used in the specification and claims shall include oxygen, sulfur and nitrogen. The heteroaryl group may be attached at any available nitrogen or carbon atom of any ring. Exemplary monocyclic heteroaryl groups include pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, triazinyl and the like. Exemplary bicyclic heteroaryl groups include indolyl, benzothiazolyl, benzodioxolyl, benzoxazolyl, benzothienyl, quinolinyl, isoquinolinyl, benzimidazolyl, cinnolinyl, quinoxalinyl, indazolyl, pyrrolopyridyl, furopyridinyl and the like.

The term “heterocyclyl” refers to a stable, fully saturated or unsaturated nonaromatic cyclic group, for example, which is a 3 to 10 membered monocyclic or bicyclic ring system, which has at least one heteroatom in at least one carbon atom containing ring. Each ring of the heterocyclyl group containing a heteroatom may have 1, 2 or 3 heteroatoms independently selected from nitrogen, oxygen or sulfur atoms. The heterocyclyl group may be attached at any heteroatom or carbon atom of the cycle, which results in the creation of a stable structure. Exemplary monocyclic heterocyclyl groups include aziridinyl, azetidinyl, pyrrolidinyl, pyrazolinyl, imidazolinyl, imidazolidinyl, oxazolidinyl, isoxazolinyl, thiazolidinyl, isothiazolidinyl, tetrahydrofuryl, piperidinyl, piperazinyl, 2-oxopiperazinyl, 2-oxopiperidinyl, 2-oxopyrrolidinyl, 4-piperidonyl, hexahydopyrazine, hexahydopyridazine, hexahydopyrmidine, tetrahydropyranyl, morpholinyl, thiomorpholinyl, thiomorpholinyl sulfoxide, thiomorpholinyl sulfone, isothiazolidinyl and the like. Exemplary bicyclic heterocyclyl groups include tetrahydroisoquinolinyl, benzopyranyl, indolizinyl, chromonyl, dihydroisoindolyl, dihydroquinazolinyl (such as 3,4-dihydro-4-oxo-quinazolinyl), benzothiopyranyl, dihydrobenzofiuyl, dihydrobenzothienyl, dihydrobenzothiopyranyl, dihydrobenzothiopyranyl sulfone, dihydrobenzopyranyl, indolinyl, isoindolinyl, tetrahydroquinolinyl, and the like.

As used herein above and throughout this application, “nitrogen” and “sulfur” include any oxidized form of nitrogen and sulfur and the quaternized form of any basic nitrogen.

“Pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

As used herein, “room temperature” refers to a temperature between 25° C. and −35° C.

Yet another embodiment of the present invention is to provide a process for the preparation of the compounds of the present invention. The compounds of formula 1 can generally be prepared, for example in the course of a convergent synthesis, by linkage of two or more fragments which can be derived retro synthetically from the formula 1. It is to be understood by those skilled in the art of organic synthesis that the functionality present on different parts of the fragment structures should be consistent with the chemical transformations proposed. In the preparation of compounds of formula (I), it may be generally necessary in the course of synthesis to temporarily block functional groups which could lead to undesired reactions or side reactions in a synthetic step by protective group (s) suited to the synthetic problem and known to the person skilled in the art. The method of fragment coupling is not restricted to the following examples, but is generally applicable for the synthesis of compounds of formula (I).

The novel compounds of the present invention are not, however, to be construed as forming the only genus that is considered as the invention, and any combination of the compounds or their moieties may itself form a genus. The following examples further illustrate details for the preparation of the compounds of the present invention. It is to be further understood by those skilled in the art that the order of synthetic steps can be changed, or known variations of the conditions and processes of the following preparative procedures can be used to prepare these compounds. All temperatures are in degrees Celsius unless otherwise noted.

Accordingly, compounds of formula (I) of the present invention may be prepared as described in the schemes below.
PART I: Preparation of the Intermediate (V)

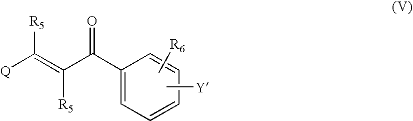

The compounds of general formula (I) can be obtained through the intermediate (V), wherein, R₅ and R₆ are as defined earlier and Y′ represents —COOH or —NHR_{c}. The intermediate compound (V) is obtained by different methods as depicted in the following schemes.

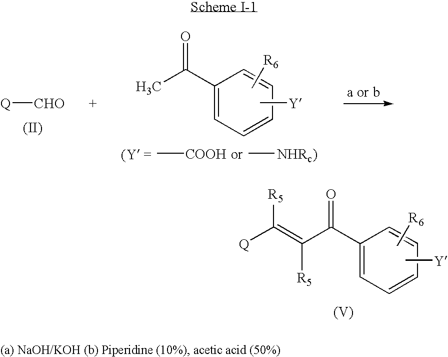

As shown in Scheme I-1, compounds of formula (V) can be prepared by reacting methyl ketones of formula (IV) (Y′ represents —COOH or —NHR_{c}) with a substituted aldehyde of formula (II). The reaction can be carried out in the presence of a base such as aqueous sodium hydroxide or potassium hydroxide in an appropriate alcohol such as methanol, ethanol, n-propanol, isopropanol, n-butanol, iso-butanol or t-butanol as solvent at a temperature of 0° to 100° C. for a period of 2 to 12 hours. Alternately, the compounds of formula (V) can also be prepared by refluxing the methyl ketone (IV) with the substituted aldehyde (II) in an appropriate alcohol such as ethanol containing 10% piperidine and 50% acetic acid with Soxhlet over 4 Å molecular sieves for a period of 24 to 30 hours.

Several alternative routes to obtain the compounds of formula (V) are outlined in Scheme I-2.

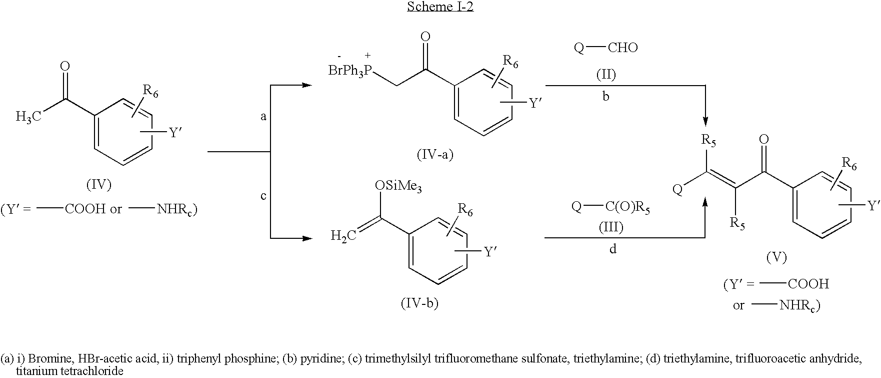

The methyl ketone (IV) (Y′ represents —COOH or —NHR_{c}) is dissolved in an appropriate solvent such as carbon tetrachloride or methanol, containing HBr-acetic acid and treated with an equimolar quantity of bromine at a temperature of 0°-80° C. and the reaction mixture is refluxed for a time period of 2 hours. The crude product obtained is treated with triphenylphosphine in an appropriate solvent such as toluene. The triphenylphosphine salt (IV-a) obtained is treated with the substituted aldehyde (II) in a suitable solvent like pyridine at a temperature in the range of 100° to 115° C. for a period of 4 to 6 hours. In an alternate process, the methyl ketone (IV) can be treated with trimethylsilyl trifluoromethane sulfonate and a base such as triethylamine in an appropriate solvent such as dichloromethane at a temperature of 0° C. for a period of 3 to 4 hours. The silyl enol ether ketone (IV-b) is reacted with a substituted ketone (III) in presence of a base such as triethylamine in an appropriate solvent such as dichloromethane at 0° C. followed by addition of trifluoroacetic anhydride and titanium tetrachloride for a period of 4 to 6 hours from 0° C. to ambient temperature to obtain the compound of formula (V).

PART II: Preparation of Compounds of General Formula (I)

The compound of general formula (I), where Y is —C(O)NRₐR_{b} can be obtained by different methods as shown in the following Scheme II-1.

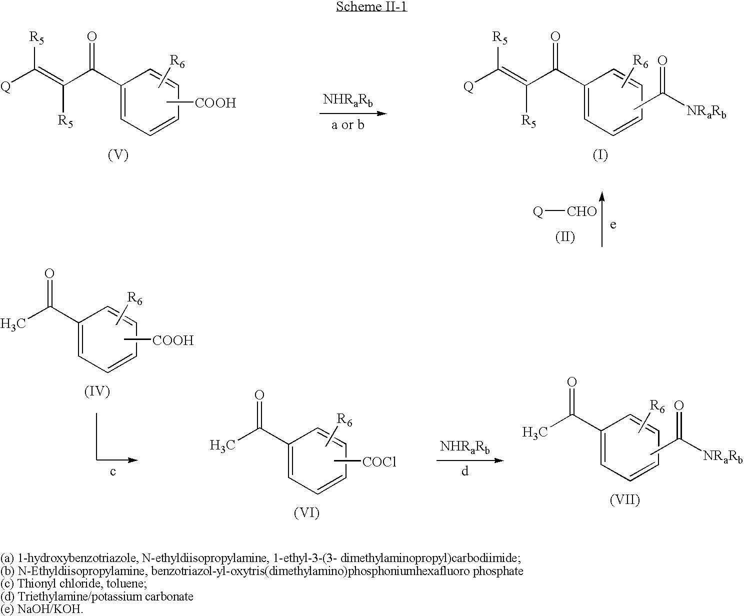

As shown in Scheme II-1, compounds of formula (I) can be prepared by reacting the intermediate (V), wherein Y′ represents —COOH, with 1-hydroxybenzotriazole and 1-ethyl-3-(3-dimethylaminopropyl) carbodiimide (EDCI) in an appropriate solvent such as tetrahydrofuran or dimethylformamide at a temperature of 0° C. to ambient temperature for an hour. The reaction mixture is further treated with an amine NHRₐR_{b} at room temperature for a period of 6 to 20 hours to obtain a compound of formula (I). Alternately, compounds of formula (I) can be prepared by treating the intermediate (V), wherein Y′ represents —COOH, with a base N-ethyldiisopropylamine (DIEA) and benzotriazol-1-yl-oxytris (dimethyl-amino)phosphonium hexafluorophosphate (BOP) in an appropriate solvent such as tetrahydrofuran or dichloromethane at a temperature from 0° C. to ambient temperature for an hour. The solution is further treated with an amine NHRₐR_{b} at room temperature for 6 to 8 hours to obtain a compound of formula (I).

In another alternate process, the acid of formula (IV) is treated with oxalyl chloride or thionyl chloride in an appropriate solvent such as dichloromethane or toluene with a catalytic amount of DMF at a temperature from 0° to 110° C. for 3 to 4 hours to obtain the compound of formula (VI). The said compound (VI) is treated with an amine NHRₐR_{b} in the presence of a base, triethylamine or potassium carbonate in an appropriate solvent such as tetrahydrofuran, toluene, dichloromethane at a temperature from 0° C. to ambient temperature to obtain the compound of formula (VII), which is treated with a substituted aldehyde of formula (II) in the presence of a base such as aqueous NaOH or KOH and an appropriate solvent such as alcohol like methanol, ethanol, n-propanol, isopropanol, n-butanol, iso-butanol or t-butanol at 0° to 100° C. for 2 to 12 hours to obtain the compound of formula (I).

The compound of general formula (I), where Y′ is —NR_{c}C(X)NRₐR_{b} and —NR_{c}C(X)NR_{d}Rₑ, and X is O or S, can be obtained by different methods as shown in the following schemes II-2 (a) to II-2 (i). Scheme II-2 (a) depicts the general procedure for synthesis of compounds of general formula (I), where Y′ represents —NR_{c}C(O)NR_{d}Rₑ or —NR_{c}C(O)NRₐR_{b}.

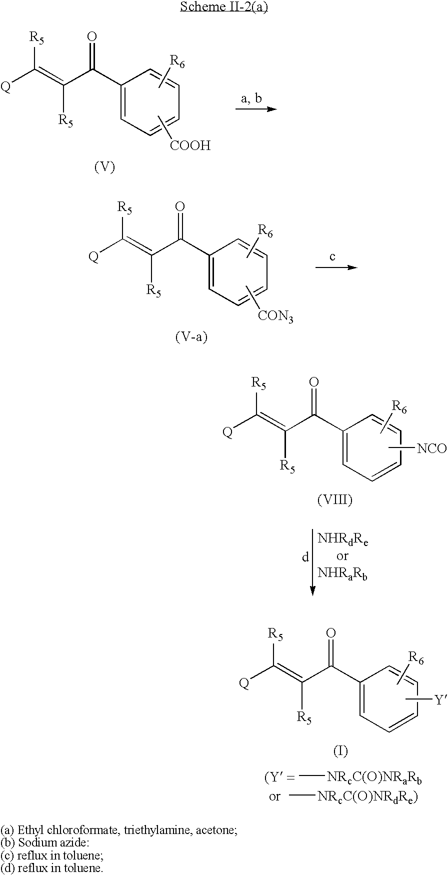

As shown in Scheme II-2 (a), the compound of formula (V), wherein Y′ represents —COOH is treated with a base such as ethyl chloroformate, triethylamine or N-ethyl diisopropylamine in an appropriate solvent such as acetone, dichloromethane, dichloroethane, tetrahydrofuran or toluene at a temperature from 0° to 60° C. for a period of 30 minutes to 3 hours. The crude reaction mixture is treated with sodium azide dissolved in water at a temperature from 25° to 110° C. for a period of 1 to 12 hours. The resulting azide of formula (V-a) was refluxed in toluene or xylene for a period of 1 to 4 hours to obtain the isocyanate of formula (VIII) was treated with NHR_{d}Rₑ or NHRₐR_{b} amine in the solvent such as toluene or xylene at the temperature from 100° to 140° C. for the period of 1 to 12 hours to obtained the compound of formula (I), wherein Y′ represents —NR_{c}C(O)NR_{d}Rₑ or —NR_{c}C(O)NRₐR_{b}.

Scheme II-2 (b) depicts the general procedure for synthesis of compounds of general formula (I), wherein, Y′ represents —NR_{c}C(X)NR_{d}Rₑ or —NR_{c}C(X)NRₐR_{b}.

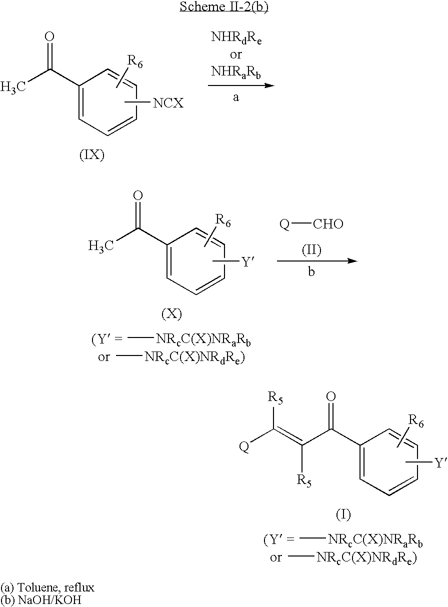

As shown in Scheme II-2 (b), the isocyanate or thioisocyanate of formula (IX) is treated with NHR_{d}Rₑ or NHRₐR_{b} amine in an appropriate solvent such as toluene, xylene or chloroform and refluxed for 6 to 12 hours to obtain the compound of formula (X), which is further treated with a substituted aldehyde of formula (II) in the presence of a base such as aqueous NaOH or KOH in a solvent such as methanol, ethanol, n-propanol, isopropanol, n-butanol, iso-butanol, t-butanol at a temperature of 0° to 100° C. for a period of 2 to 12 hours to obtain the compound of formula (I), wherein Y′ represents NR_{c}C(X)NR_{d}Rₑ or NR_{c}C(X)NRₐR_{b}.

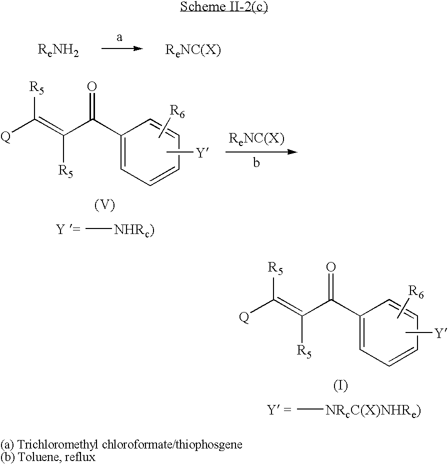

In an alternate embodiment, as shown in Scheme II-2 (c), the compounds of general formula (I), wherein Y′ represents —NR_{c}C(X)NR_{d}Rₑ, can be prepared by reacting the isocyanate or thioisocyanate of Rₑ with a compound of formula (V), wherein Y′ represents —NHR_{c}, in an appropriate solvent such as toluene, xylene or dimethylformamide at a temperature from 80° to 130° C. for a period of 3 to 6 hours. The isocyanate or thioisocyanate of Rₑ can be obtained by reacting Rₑ amine hydrochloride with trichloromethyl chloroformate or thiophosgene in the presence of an acid in a solvent like dioxane at 20° to 100° C. for a period of 2 to 12 hours.

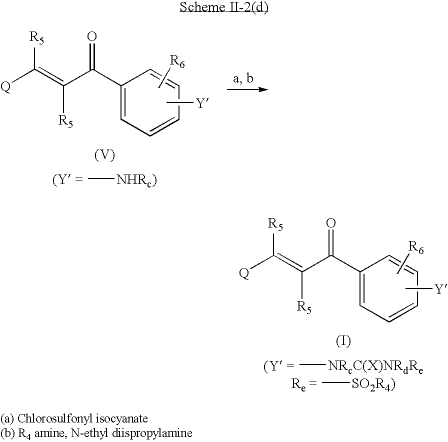

In a specific embodiment, as shown in Scheme II-2 (d), the compounds of general formula (I), wherein Y′ represents —NR_{c}C(X)NR_{d}Rₑ and Rₑ represents —SO₂R₄, can be prepared by reacting a compound of formula (V), wherein Y′ represents —NHR_{c}, with chlorosulphonyl isocyanate in an appropriate solvent such as toluene, xylene or chloroform 60-110° C. for 6 to 12 hours. The resulting intermediate obtained is treated in the presence of base such as N-ethyldiisopropylamine or potassium, carbonate with R₄ amine in a solvent like tetrahydrofuran or dimethylformamide at a temperature from 0-100° C. for a period of 2 to 6 hours to provide the compound of the formula (I).

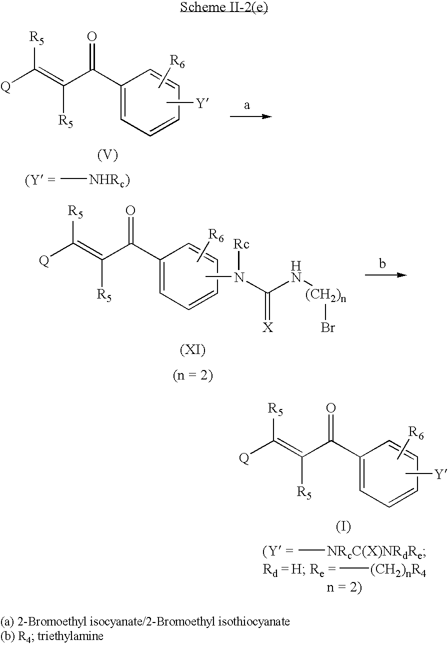

In another specific embodiment, as shown in Scheme II-2 (e), the compounds of general formula (I), wherein Y′ represents —NR_{c}C(X)NR_{d}Rₑ, and R_{d} and Rₑ represent H and —(CH₂)ₙR₄, can be prepared by reacting a compound of formula (V), wherein Y′ represents —NHR_{c}, with 2-bromoethyl isocyanate or 2-bromoethyl isothiocyanate in a solvent such as toluene or chloroform at reflux temperature for 2 to 12 hours to provide the compound of formula (XI) (wherein n=2), which was further treated with R₄ in the presence of a base potassium carbonate or triethylamine in an appropriate solvent such as dimethyl formamide or toluene at 60-100° C. for 2 to 12 hours to give the compound of formula (I). Alternately, the compound of formula (XI) is treated with R₄-boronic acid and tetrakis (triphenylphosphine)palladium(O) in the presence of a base, such as aqueous potassium carbonate or sodium bicarbonate in a solvent such as toluene, ethanol or dimethylformamide at 60-100° C. for a period of 20 to 30 hours to give the compound of formula (I).

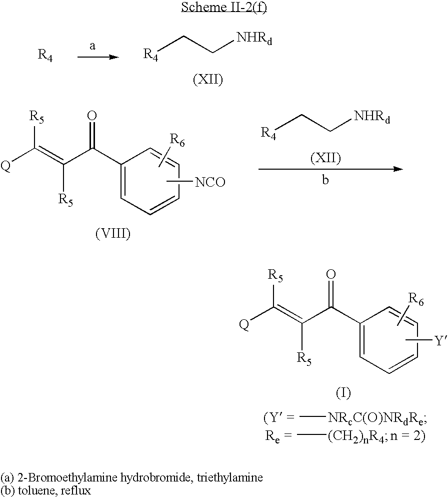

In an alternative embodiment, as shown in Scheme II-2 (f), the compounds of general formula (I), wherein Y′ represents —NR_{c}C(O)NR_{d}Rₑ, and Rₑ represents —(CH₂)ₙR₄, can be prepared by reacting a compound of formula (XII) with the isocyanate of formula (VIII) in a solvent such as toluene or xylene at 100-140° C. for 1 to 12 hours to give the compound of formula (I). The compound of formula (XII) can be obtained by reacting 2-bromoethylamine hydrobromide in the presence of a base such as potassium carbonate or tri-ethylamine in a solvent like tetrahydrofuran, toluene or dimethylformamide at 25-110° C. for 2 to 8 hours.

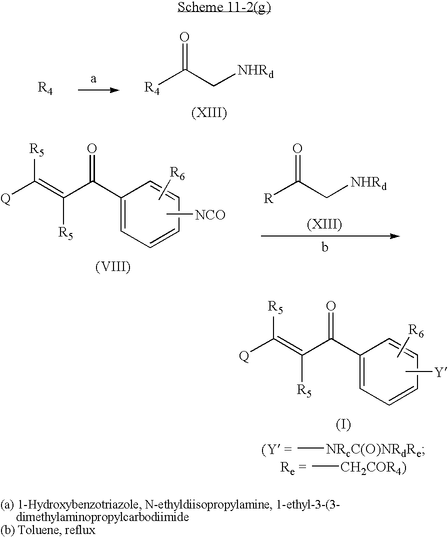

In another specific embodiment, as shown in Scheme II-2 (g), the compounds of general formula (I), wherein Y′ represents —NR_{c}C(O)NR_{d}Rₑ, and Rₑ represents —CH₂COR₄, can be prepared by reacting the compound of formula (XIII) with a compound of formula (VIII) in a solvent such as toluene or xylene at 100-140° C. for 1 to 12 hours. The compound of formula (XIII) can be prepared by treating an N-substituted-Boc-glycine with R₄ in the presence of base such as N-ethyl diisopropylamine, 1-hydroxybenzotriazole and 1-ethyl-3-(3-dimethyl-aminopropyl) carbodiimide (EDCI) in a solvent such as tetrahydrofuran or dimethylformamide at 0° C. to ambient temperature for 6 to 20 hours, followed by removal of the protecting group t-Boc by treatment with trifluoroacetic acid in dichloromethane at 0° to 10° C. for a period of 1 to 6 hours.

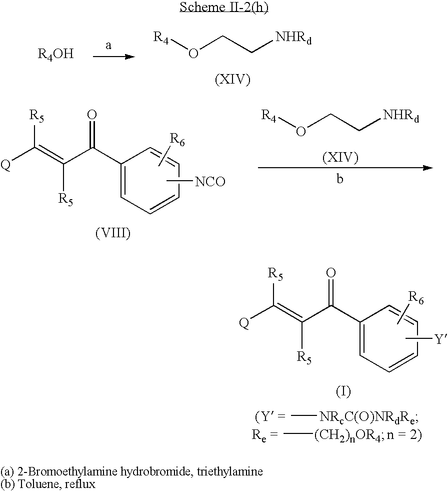

In another specific embodiment, as shown in Scheme II-2 (h), the compounds of general formula (I), wherein Y′ represents —NR_{c}C(O)NR_{d}Rₑ, and Rₑ represents —(CH₂)ₙOR₄, can be prepared by reacting a compound of formula (XIV) with a compound of formula (VIII) in a solvent such as toluene or xylene at 100° to 140° C. for 1 to 12 hours. The compound of formula (XIV) can be prepared by treating 2-bromoethylamine hydrobromide with HO-R₄ in the presence of a base such as triethylamine or potassium carbonate at 20° to 100° C. in a solvent such as tetrahydrofuran, acetonitrile or dimethylformamide for 1 to 6 hours.

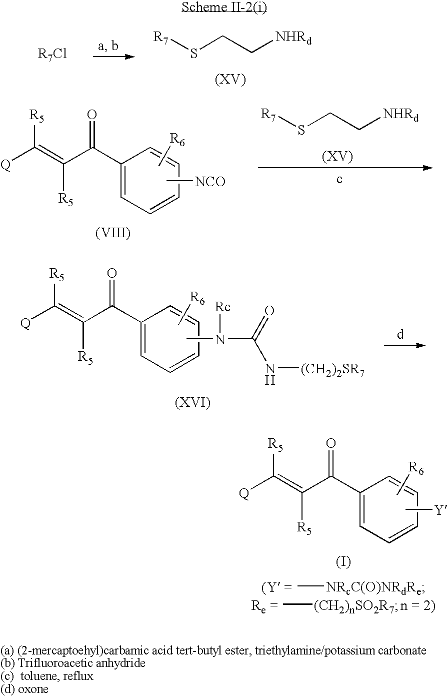

In still another specific embodiment, as shown in Scheme II-2 (i), the compounds of general formula (I), wherein Y′ represents —NR_{c}C(O)NR_{d}Rₑ, and Rₑ represents —(CH₂)ₙSO₂R₇, can be prepared by reacting a compound of formula (XV) with a compound of formula (VIII) in a solvent such as toluene or xylene at 100° to 140° C. for 1 to 12 hours to give the compound (XVI), which on further treatment with oxone in water/methanol at 0° to room temperature gives the compound of formula (I). The compound of formula (XV) can be obtained by treating (2-mercaptoethyl) carbamic acid tert-butyl ester with R₇Cl in the presence of a base such as potassium carbonate or triethylamine at 0° to 100° C. in a solvent like tetrahydrofuran, acetonitrile or dimethylformamide for 1 to 6 hours and the resulting product is treated with trifluoroacetic anhydride in dichloromethane at 0° to ambient temperature for 2 to 6 hours to give the compound of formula (XV).

The compounds of general formula (I), where Y′ is —NR_{c}C(O)OR_{f} can be obtained by different methods as shown in the following schemes II-3 (a) to II-3 (g). Scheme II-3(a) depicts the general procedure for synthesis of compounds of general formula (I), where Y′ represents —NR_{c}C(O)OR_{f}.

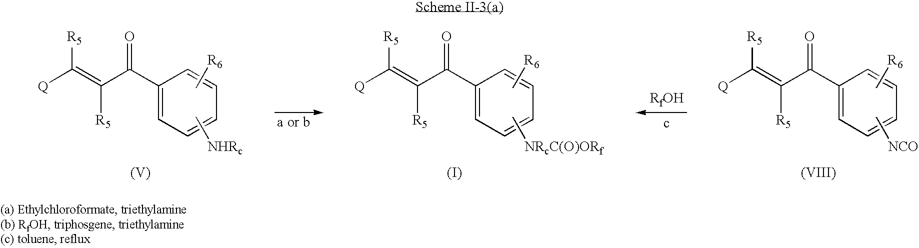

As shown in Scheme II-3(a), the compound of formula (V), wherein Y′ represents —NHR_{c} is treated with ethyl chloroformate or phenyl chloroformate in presence of a base triethylamine or N-ethyldiisopropylamine in a solvent such as dimethylformamide or tetrahydrofuran at 0°to 60° C. for 30 minutes to 8 hours to obtain the compound of formula (I). Alternately, the compound of formula (V), wherein Y′ represents —NHR_{c} is treated with HO—R_{f} and phosgene or triphosgene in the presence of a base such as N-ethyl diisopropylamine, triethylamine, potassium or sodium carbonate at a temperature ranging from 0° to 35° C. for a period of 10 minutes to 3 hours to obtain the compound of formula (I). In another alternate embodiment, the compound of formula (VIII) is treated with HO—R_{f} in a solvent such as toluene or xylene at 100° to 140 ° C. for 1 to 12 hours to obtain the compound of formula (I).

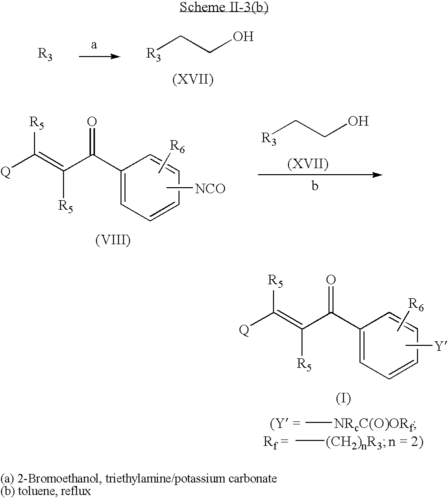

In a specific embodiment, as shown in Scheme II-3 (b), the compounds of general formula (I), wherein Y′ represents —NR_{c}C(O)OR_{f}, and R_{f} represents —(CH₂)ₙR₃, can be prepared by reacting a compound of formula (XVII) with a compound of formula (VIII) in a solvent such as toluene or xylene at 100° to 140° C. for 1 to 12 hours. The compound of formula (XVII) can be obtained by reacting an R₃-amine with 2-bromoethanol or 2-chloroethanol in the presence of a base such as potassium carbonate or triethylamine in a solvent like tetrahydrofuran, toluene or dimethylformamide at 25° to 110° C. for 2 to 8 hours.

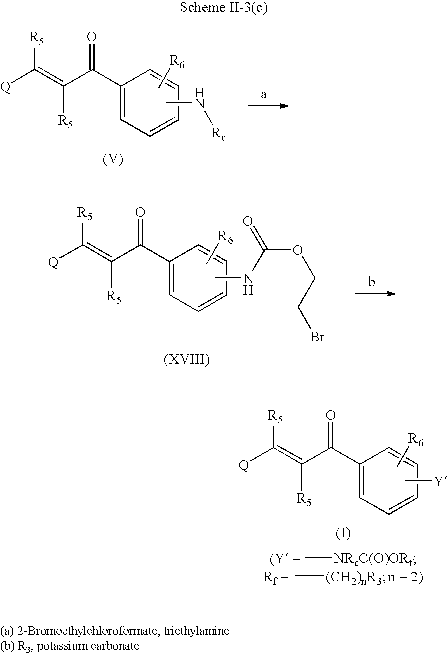

Alternately, as shown in Scheme II-3 (c), the compounds of general formula (I), wherein Y′ represents —NR_{c}C(O)OR_{f}, and R_{f} represents —(CH₂)ₙR₃ can also be prepared by treating the compound of formula (V), wherein Y′ represents —NHR_{c} with 2-bromoethyl chloroformate in the presence of a base such as triethylamine or N-ethyl diisopropyl amine in an appropriate solvent like dichloromethane or tetrahydrofuran at 0° C. to 30° C. for 1 to 6 hours to give the compound (XVIII), which on treatment with R₃ in the presence of base such as potassium carbonate in a solvent like dimethylformamide or acetonitrile at 60° to 100° C. for. 2 to 16 hours gives the compound of formula (I)

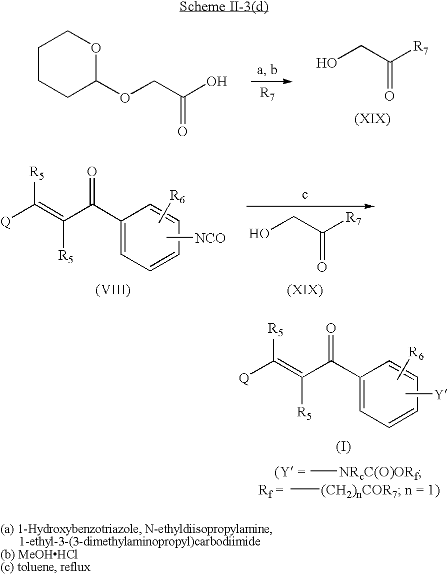

In another specific embodiment, as shown in Scheme II-3 (d), the compounds of general formula (I), wherein Y′ represents —NR_{c}C(O)OR_{f}, and R_{f} represents —(CH₂)ₙCOR₇, can be prepared by reacting the compound of formula (XIX) with compound of formula (VIII) in an appropriate solvent such as toluene or xylene at 100° to 140° C. for 1 to 12 hours. The compound of formula (XIX) can be obtained by treating the R₇ amine with (tetrahydro-pyran-2-yloxy)-acetic acid, 1-hydroxy benzotriazole, N-ethyldiisopropylamine and 1-ethyl-3-[3-dimethylaminopropyl] carbodiimide (EDCI) in an appropriate solvent such as tetrahydrofuran or dimethylformamide at 0° C. to ambient temperature for 6 to 20 hours. The tetrahydropyranyl group was deprotected by refluxing in methanolic hydrochloric acid.

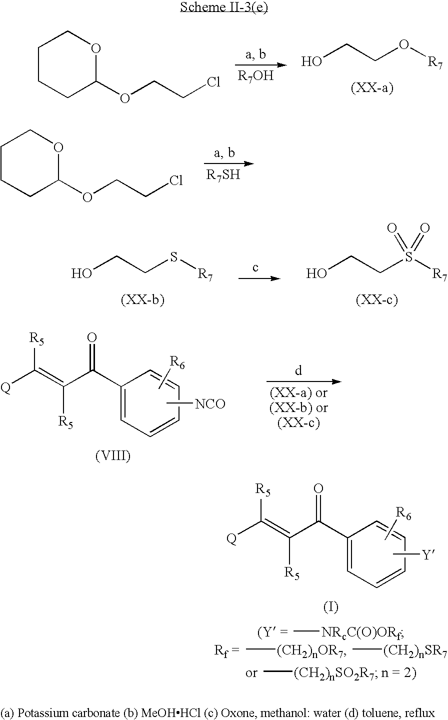

In still another specific embodiment, as shown in Scheme II-3 (e), the compounds of general formula (I), wherein Y′ represents —NR_{c}C(O)OR_{f}, and R_{f} represents —(CH₂)ₙOR₇, —(CH₂)ₙSR₇ or —(CH₂)ₙSO₂R₇ can be prepared by reacting the compound of formula (XX-a), (XX-b) or (XX-c) with a compound of formula (VIII) in an appropriate solvent such as toluene or xylene at 100° to 140° C. for 1 to 12 hours. The compounds of formula (XX-a) and (XX-b) can be obtained by reacting HO—R₇ and HS—R₇ respectively with 2-(2-chloroethoxy) tetrahydropyran in the presence of a base such as potassium carbonate in an appropriate solvent such as dimethylformamide or acetonitrile at 80° to 110° C. for 3 to 18 hours. The tetrahydropyranyl group is deprotected by refluxing in methanolic hydrochloric acid. The compounds of formula (XX-c) can be obtained by reacting the compound of formula (XX-b) with oxone in a methanol: water (2:1) mixture at 0° C. to ambient temperature for 2 to 3 hours.

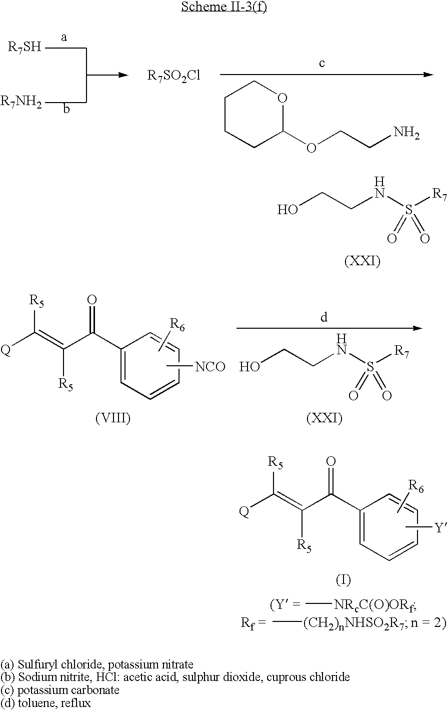

In another specific embodiment, as shown in Scheme II-3 (f), the compounds of general formula (I), wherein Y′ represents —NR_{c}C(O)OR_{f}, and R_{f} represents —(CH₂)ₙNHSO₂R₇ can be prepared by reacting the compound of formula (XXI) with a compound of formula (VIII) in an appropriate solvent such as toluene or xylene at 100° to 140° C. for 1 to 12 hours. The compound of formula (XXI) can be obtained from either HS—R₇ or H₂N—R₇ as follows. HS—R₇ is treated with sulphuryl chloride and potassium nitrate in an appropriate solvent such as acetonitrile or tetrahydrofuran at 0° to 25° C. for 2 to 6 hours. The resulting product is treated with 2-(tetrahydropyran-2-yloxy)ethylamine in the presence of a base such as triethylamine or potassium carbonate in a suitable solvent such as tetrahydrofuran or dichloromethane at 0° to 60° C. for 1 to 6 hours. Alternately, H₂N—R₇is treated with sodium nitrite and a mixture of concentrated HCl:acetic acid (3:1) at −10° to −5° C. for 45 to 90 minutes. The resulting diazonium salt is treated with a solution of sulfur dioxide and cuprous chloride as catalyst in acetic acid at 0° to 10° C. for 30 to 60 minutes to obtain ClO₂S—R₇, which is further treated with 2-(tetrahydropyran-2-yloxy)ethylamine in the presence of a base triethylamine in an appropriate solvent such as tetrahydrofuran or toluene at 0° to 60° C. for 3 to 4 hours. The tetrahydropyranyl group is deprotected by refluxing in methanolic hydrochloric acid to obtain the compound of formula (XXI).

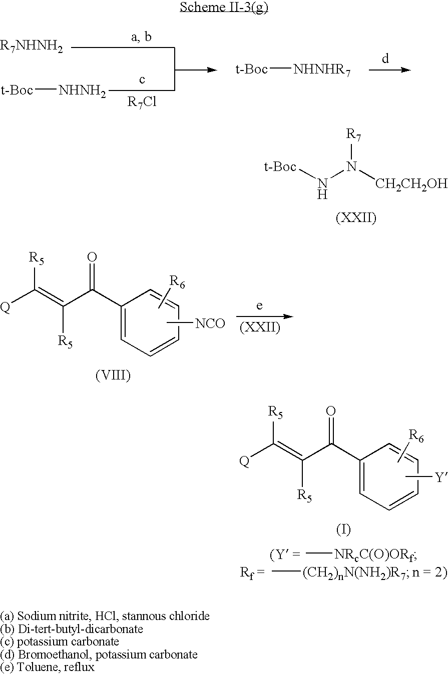

In a still further specific embodiment, as shown in Scheme II-3 (g), the compounds of general formula (I), wherein Y′ represents —NR_{c}C(O)OR_{f}, and R_{f} represents —(CH₂)ₙN(NH_{2)R}₇ can be prepared by reacting the Boc-protected compound of formula (XXII) with a compound of formula (VIII) in an appropriate solvent such as toluene or xylene at 100° to 140° C. for 1 to 12 hours, followed by removal of the Boc-protecting group with trifluoroacetic acid in dichloromethane at 0° C. for the period of 2 to 6 hours. The compound of formula (XXII) can be obtained by treating Boc—NH—NH—R₇ with bromoethanol in the presence of a base such as potassium carbonate or triethylamine in an appropriate solvent such as tetrahydrofuran or dimethylformamide at 20° to 100° C. for 2 to 6 hours. The Boc—NH—NH—R₇ can be obtained either from H₂N—R₇ or Boc-hydrazine as follows. H₂N—R₇ is treated with sodium nitrite, concentrated hydrochloric acid and water at 0° C. for 1 to 2 hours and the diazonium salt thus obtained is reduced with stannous chloride at 0° C. for 3 to 6 hours. The amino function of NH₂NH—R₇ is protected with di-tert-butyl dicarbonate in an appropriate solvent such as ethanol-water for 2 to 4 hours to obtain Boc—NH—NH—R₇. Alternately, the Boc-hydrazine is treated with Hal—R₇ in the presence of a base such as potassium carbonate at the temperature from 20° to 100° C. in an appropriate solvent such as dimethylformamide to provide Boc-NHNHR₇.

The compounds of general formula (I), where Y′ is —NR_{c}C(O)C(O)R_{g} can be obtained by different methods as shown in the following schemes II-4 (a) to II-4 (e). Scheme H-4 (a) depicts the general procedure for synthesis of compounds of general formula (I), where Y′ represents —NR_{c}C(O)C(O)R_{g}.

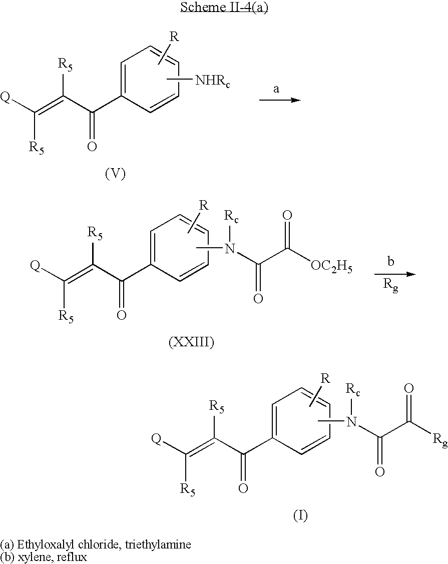

As shown in Scheme II-4 (a), the compound of formula (V), wherein Y′ is —NHR_{c}, is treated with ethyl oxalyl chloride in the presence of a base such as triethylamine or potassium carbonate in an appropriate solvent such as tetrahydrofuran or dichloromethane at 0° C. to ambient temperature for 3 to 6 hours to obtain the compound of formula (XXIII). This is treated with the R_{g} amine in an appropriate solvent such as xylene, dimethylacetamide or N-methyl-2-pyrrolidone at 100°to 160° C. for 2 to 16 hours to give the compound of formula (I).

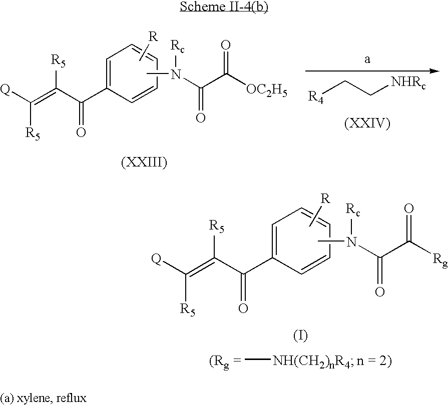

In a specific embodiment, as shown in Scheme II-4 (b), the compounds of general formula (I), wherein Y′ represents —NR_{c}C(O)C(O)R_{g}, and R_{g} represents —NH(CH₂)ₙR₄, can be prepared by treating the compound of formula (XXIII) with the compound of formula (XXIV) in an appropriate solvent such as xylene, dimethylacetamide or N-methyl-2-pyrrolidone at 100°to 160° C. for 2 to 16 hours.

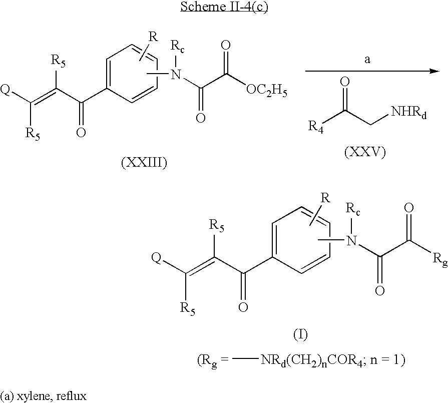

In another specific embodiment, as shown in Scheme II-4 (c), the compounds of general formula (I), wherein Y′ represents —NR_{c}C(O)C(O)R_{g}, and R_{g} represents —NR_{d}(CH₂)ₙCOR₄, can be prepared by reacting the compound of formula (XXIII) with the compound of formula (XXV) in an appropriate solvent such as xylene, dimethylacetamide or N-methyl-2-pyrrolidone at 100° to 160° C. for 2 to 16 hours.

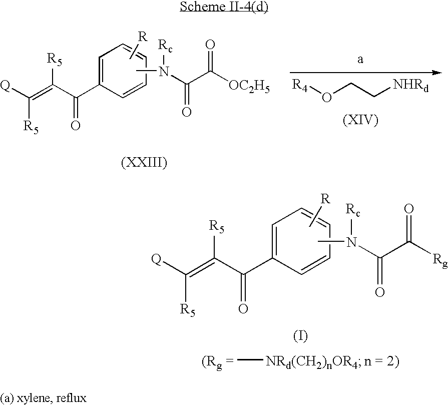

In still another specific embodiment, as shown in Scheme II-4 (d), the compounds of general formula (I), wherein Y′ represents —NR_{c}C(O)C(O)R_{g}, and R_{g} represents —NR_{d}(CH₂)ₙOR₄, can be prepared by reacting the compound of formula (XIV) with the compound of formula (XXIII) in an appropriate solvent such as xylene, dimethylacetamide of N-methyl-2-pyrrolidone at 100° to 160° C. for 2 to 16 hours.

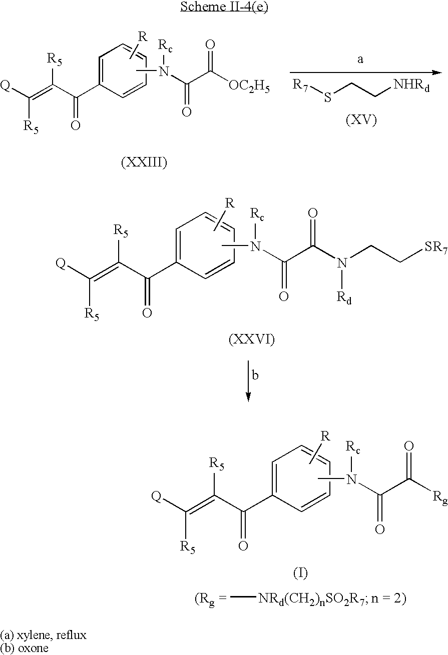

In another specific embodiment, as shown in Scheme II-4 (e), the compounds of general formula (I), wherein Y′ represents —NR_{c}C(O)C(O)R_{g}, and R_{g} represents —NR_{d}(CH₂)ₙSO₂R₇, can be prepared by treating the compound of formula (XXIII) with the compound of formula (XV) in an appropriate solvent such as xylene, dimethylacetamide or N-methyl-2-pyrrolidone at 100°to 160° C. for 2 to 16 hours gives the compound (XXVI), which on treatment with oxone in water/methanol at 0° to room temperature provides the compound of formula (I).

Specific procedures for synthesis of the substituted aldehyde, Q-CHO of formula (II) are described hereinbelow in schemes III-1 to III-5.

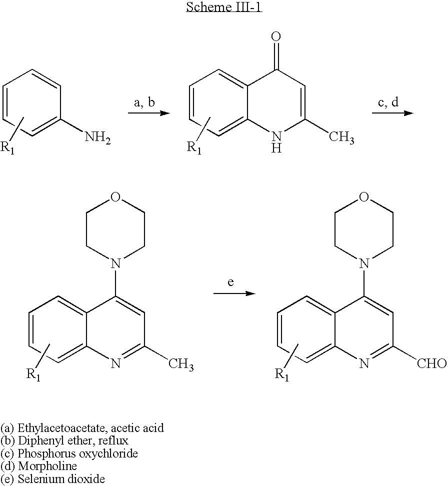

Scheme III-1 depicts the synthesis of the substituted aldehyde Q-CHO, in which Q has a morpholino substituent. The R₁-substituted aniline, acetic acid and ethyl acetoacetate is refluxed using Dean Stark apparatus in an appropriate solvent such as toluene or benzene at 90° to 110° C. for 6 to 18 hours. The crude ester thus obtained is refluxed in a solvent such as diphenyl ether or Dowtherm® for 16 to 24 hours to give a substituted 2-methyl-4-quinolone. The quinolone on treatment with POCl₃ at 0° to 60° C. in an appropriate solvent such as tetrahydrofuran or toluene for 3 to 6 hours gives substituted 2-methyl-4-chloroquinoline, which is further reacted with a nitrogen containing heterocycle R₂ in the presence of a base potassium carbonate in a solvent such as acetonitrile or dimethylformamide at 80° to 100° C. for 3 to 8 hours to give R₂-substituted-2-methyl-quinoline. This on treatment with selenium dioxide in 1,4-dioxane at 100° to 110° C. for 3 to 12 hours gives the aldehyde Q-CHO (II).

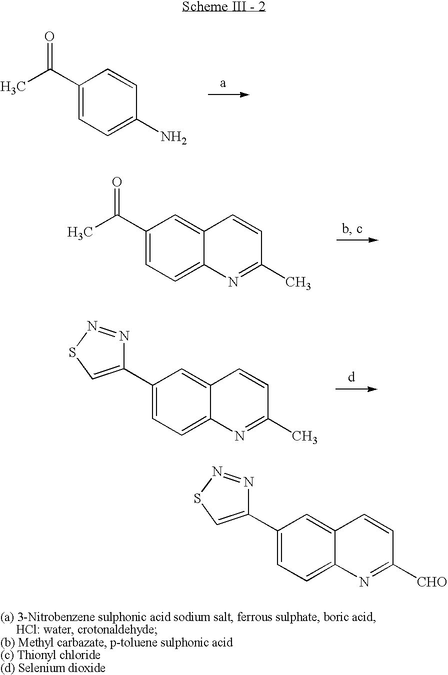

Scheme III-2 depicts the synthesis of the substituted aldehyde Q-CHO, in which Q has a 1, 2, 4-thiadiazole substituent. 6-acetyl-aniline or substituted aniline is treated with 4-aminoacetophenone, 3-nitrobenzene sulphonic acid sodium salt, ferrous sulphate, boric acid in 6N hydrochloric acid at 80° to 100° C. for 1 to 3 hours, followed by addition of crotonaldehyde and heated at 80° to 100° C. for 4 to 12 hours to give 6-acetyl-2-methyl quinoline. This is treated with methylcarbazate and p-toluene sulphonic acid in an appropriate solvent such as toluene or xylene at 100° to 140° C. for 8 to 16 hours. The resulting product is treated with thionyl chloride in a solvent such as toluene or xylene at 60° C. to 100° C. for 1 to 4 hours to give 2-methyl-6-[1,2,3]thiadiazol-4-yl-quinoline. Further, on oxidation of the methyl group with selenium dioxide in dioxane at 60° C. to 100° C. for 3 to 12 hours gives the aldehyde of general formula (II).

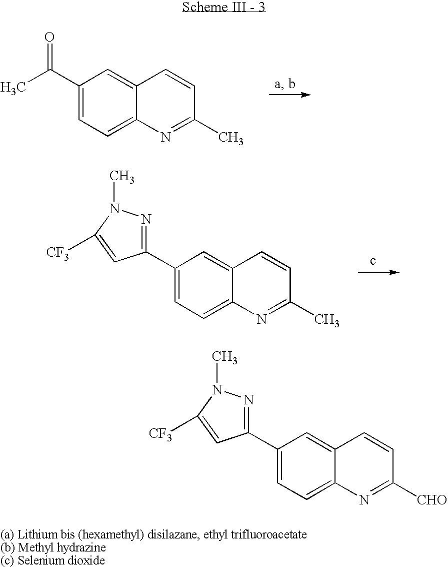

Scheme III-3 depicts the synthesis of the substituted aldehyde Q-CHO, in which Q has a pyrazole substituent. 6-acetyl-2-methyl quinoline is treated with lithium bis(hexamethyl)disilazane in an appropriate solvent such as tetrahydrofuran at −20° C. for an hour, which is then reacted with ethyl trifluoacetate at −20° C. for 2 hours and a further period of 3 hours at ambient temperature to give a diketo compound. The diketo compound is treated with methyl hydrazine to obtain 2-methyl-6-(1-methyl-5-trifluoro-1H-pyrazol-3-yl) quinoline, which on oxidation with selenium dioxide in a solvent such as dioxane at 60° C. to 100° C. for 3 to 12 hours; gives the compound of formula (II).

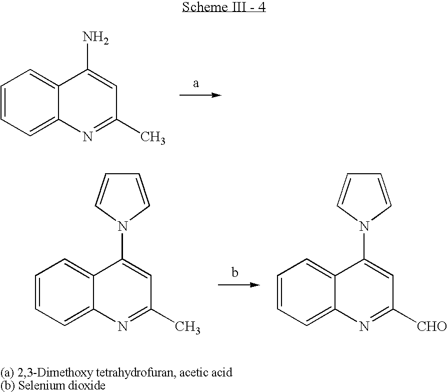

Scheme III-4 depicts the synthesis of the substituted aldehyde Q-CHO, in which Q has a pyrrole substituent. 4-amino-2-methyl quinoline is treated with 2,3-dimethoxy tetrahydrofuran in acetic acid at 120° C. for 2 to 6 hours to give 4-pyrrolo-2-methyl-quinoline. Further, on oxidation of the methyl group with selenium dioxide in dioxane at 60° C. to 100° C. for 3 to 12 hours gives the compound of formula (II).

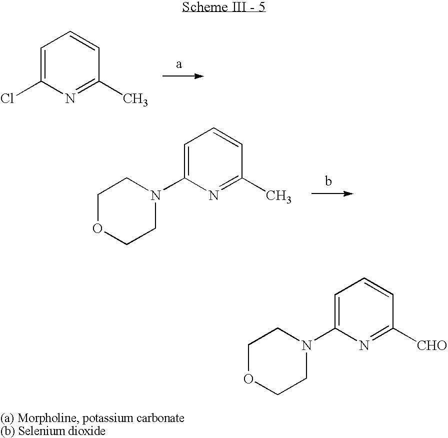

Scheme III-5 depicts the synthesis of the substituted aldehyde Q-CHO, in which Q is pyridine and has a morpholino substituent. 6-chloro-pyridine-2-carboxaldehyde is treated with morpholine in the presence of a base such as potassium carbonate in an appropriate solvent such as dimethylformamide or acetonitrile at 90° to 100° C. for 4 to 24 hours to give the compound of formula (II).

A general synthetic method is provided for each of the disclosed groups of chemical compounds. One of ordinary skill will recognize to substitute appropriately modified starting material containing the various substituents. One of ordinary skill will readily synthesize the disclosed compounds according to the present invention using conventional synthetic organic techniques and microwave techniques from starting material which are either purchased or may be readily prepared using prior art methods.

The compounds of the present invention may have chiral centers and occur as racemates, racemic mixtures and as individual diastereomers, or enantiomers with all isomeric forms being included in the present invention. Therefore, where a compound is chiral, the separate enantiomers, substantially free of the other, are included within the scope of the invention; further included are all mixtures of the two enantiomers. Also included within the scope of the invention are polymorphs as well as hydrates of the compounds of the instant invention.

The representative compounds of the present invention of general formula (I) are useful to raise the levels of HSP-70.

Method of Protecting Cells Against Stress

The present invention relates to a method of inducing the expression of Heat Shock Protein 70 (HSP-70) in cells, by treating the cells with an effective amount of one or more of a 2-propene-1-one derivative, represented by the formula (I), its stereoisomer, tautomer, solvates or its pharmaceutically acceptable salts.

In the present context, “HSP-70” refers to proteins of the HSP family having an approximate molecular mass of 70 kDa, which are induced in response to a pathological stress. “Pathological stress” refers to factors which disturb the homeostasis of the cells thus leading to the increased expression of stress proteins like HSP-70. Such factors are, for example, metabolic, oxidative, stresses caused by hypoxia, ischemia, infections, stresses induced by metals and exogenous substances, immunogenic stresses, cell malignancy, neurodegeneration, trauma, or aging. Other forms of pathological stresses include those causing the formation of free radicals or increase in the quantity of inflammatory cytokines.

In one embodiment of the invention, the diseases accompanying pathological stress are selected from cerebrovascular, cardiovascular diseases, neurodegenerative diseases and immune disorders, such as stroke, myocardial infarction, inflammatory disorder, hepatotoxicity, sepsis, diseases of viral origin, allograft rejection, tumourous diseases, gastric mucosal damage, brain haemorrhage, endothelial dysfunctions, diabetic complications, neuro-degenerative diseases, post-traumatic neuronal damage, acute renal failure, glaucoma and aging related skin degeneration. The compounds of the present invention possess the ability to induce HSP-70 and thereby protect cells against stress-induced damage in the above disease conditions.

The invention also relates to a method of inhibiting TNF-α in cells, by treating the cells with an effective amount of one or more of a 2-propene-1-one derivative, represented by the formula (I), its stereoisomer, tautomer, solvates or its pharmaceutically acceptable salts. Cytokines such as TNF-α produced by activated monocytes/macrophages play an important role in the regulation of the immune response. Studies have shown that TNF-α is involved in the pathogenesis of diabetes, myocardial infarction, liver failure, infectious diseases like sepsis syndrome, autoimmune diseases like rheumatic arthritis, graft rejection, organ transplant rejection, chronic inflammatory disorders such as rheumatoid diseases, arthritic disorders and connective tissue disorders. [Han, H. S: and Yenari, M. A., Current Opinion in Investigational Drugs, 2003, Vol. 4(5), pp. 522-529]. Treatment with 2-propen-1-one derivatives of the instant invention which show TNF-α inhibitory activity exerts a cytoprotective effect in the above disease conditions.

In another embodiment of the invention, a method of increasing HSP-70 expression in cells is provided. HeLa cells, which are well characterized cell lines employed for primary screening is used for this purpose. In this method, the HeLa cells are treated with an effective amount of 2-propene-1-one derivatives. The 2-propene-1-one derivatives substantially increase the expression of HSP-70 in these cells.

In still another embodiment of the invention, a method of inhibition of TNF-α expression is provided. For this purpose, Human monocytic leukaemia cell line, THP-1, differentiated into macrophage-like cells by phorbol merystyl ester treatment was employed. In this method, TNF-α expression was induced in the cell line by treatment with lipopolysaccharide. The cells were subjected to an effective amount of 2-propene-1-one derivatives. The 2-propene-1-one derivatives substantially inhibit the expression of TNF-α in these cells.

An increase in HSP-70 expression and inhibition of TNF-α expression in cells can be detected using well established laboratory procedures such as Real time polymerase chain reaction (Real time PCR), as described in Examples (I) and (II). Real time PCR is a technique that is used for the quantitative measurement of gene expression levels in cells or tissues. The technique is based on the use of a fluorescent reporter dye at 5′end of the probe and a quencher dye at the 3′ end of the probe to monitor the PCR reaction as it occurs. The fluorescence of the reporter molecule increases as products accumulate with each successive round of amplification. The point at which the fluorescence rises appreciably above the background is defined as the threshold cycle and is used for the determination of initial copy number.

For determining the ability of the compounds of the instant invention to afford protection against stress-induced damage in vivo, a pathological stress is applied to an animal, for example, cerebral ischemia, myocardial ischaemia or carrageenan-induced inflammation. Cerebral ischemia can be induced in an animal as described in Example (III), induction of myocardial ischaemia is described in Example (V.) and carrageenan-induced inflammation is described in Example (IV). The compounds of the present invention are administered to the animals and tested for their efficacy against the said disease conditions.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to inhibiting the disease or disorder, either physically, (e.g., stabilization of a discernible symptom), physiologically, (e.g., stabilization of a physical parameter) or both. In yet another embodiment, “treating” or “treatment” refers to delaying the onset of the disease or disorder. As used herein, amelioration of the symptoms of a particular disorder by administration of a particular compound or pharmaceutical composition refers to any lessening, whether permanent or temporary, lasting or transient that can be attributed to or associated with administration of the composition.

The phrase “a therapeutically effective amount” means the amount of a compound that, when administered to a patient for treating a disease, is sufficient to effect such treatment for the disease. The “therapeutically effective amount” will vary depending on the compound, mode of administration, the disease and its severity and the age, weight, etc., of the patient to be treated. Such amount can be readily determined by one skilled in the art, and will not require undue experimentation.

Pharmaceutical Compositions

In another embodiment of the invention is provided a pharmaceutical composition comprising a therapeutically effective amount of one or more of a compound of general formula (I). While it is possible to administer therapeutically effective quantity of compounds of formula (I) either individually or in combination, directly without any formulation, it is common practice to administer the compounds in the form of pharmaceutical dosage forms comprising pharmaceutically acceptable excipient(s) and at least one active ingredient. These dosage forms may be administered by a variety of routes including oral, topical, transdermal, subcutaneous, intramuscular, intravenous, intranasal, pulmonary etc.

Oral compositions may be in the form of solid or liquid dosage form. Solid dosage form may comprise pellets, pouches, sachets or discrete units such as tablets, multi-particulate units, capsules (soft & hard gelatin) etc. Liquid dosage forms may be in the form of elixirs, suspensions, emulsions, solutions, syrups etc. The above pharmaceutical compositions may contain in addition to active ingredients, excipients such as diluents, disintegrating agents, binders, solubilizers, lubricants, glidants, surfactants, suspending agents, emulsifiers, chelating agents, stabilizers, flavours, sweeteners, colours etc. Some example of suitable excipients include lactose, cellulose and its derivatives such as microcrystalline cellulose, methylcelulose, hydroxy propyl methyl cellulose, ethylcellylose, dicalcium phosphate, mannitol, starch, gelatin, polyvinyl pyrolidone, various gums like acadia, tragacanth, xanthan, alginates & its derivatives, sorbitol, dextrose, xylitol, magnesium Stearate, talc, colloidal silicon dioxide, mineral oil, glyceryl mono Stearate, glyceryl behenate, sodium starch glycolate, Cross Povidone, crosslinked carboxymethylcellulose, various emulsifiers such as polyethylene glycol, sorbitol fatty acid, esters, polyethylene glycol alkylethers, sugar esters, polyoxyethylene polyoxypropyl block copolymers, polyethoxylated fatty acid monoesters; diesters and mixtures thereof.

Sterile compositions for injection can be formulated according to conventional pharmaceutical practice by dissolving or suspending the active substance in a vehicle such as water for injection, N-Methyl-2-Pyrrolidone, propylene glycol and other glycols, alcohols, a naturally occurring vegetable oil like sesame oil, coconut oil, peanut oil, cotton sead oil or a synthetic fatty vehicle like ethyl oleate or the like. Buffers, anti-oxidants, preservatives, complexing agents like cellulose derivatives, peptides, polypeptides and cyclodextrins and the like can be incorporated as required,

Dose is appropriately decided by its form of preparation, method of administration, purpose of use and age, body weight and symptom of the patient to be treated and it is not constant. But, usually, the amount of at least one of the compound selected from the compound of the present invention, an optically active substance thereof or a salt thereof contained in the preparation is from 0.1 microgram to 100 mg/kg per day (for adults). Thus, the total quantity of compound in a particular pharmaceutical composition may range from 1 to 1000 mg, at concentration levels ranging from about 0.5% to about 90% by weight of the total composition. In a preferred embodiment, the composition may contain 20 to 500 mg of the compound, at concentration levels ranging from about 10% to about 70% by weight of the total composition. Of course, the dose may vary depending upon various conditions and, there ore, the dose less than above may be sufficient in some cases while, in other cases, the dose more than above may be necessary. The dosage form can have a slow, delayed or controlled release of active ingredients in addition to immediate release dosage forms.

### PREPARATORY EXAMPLES

The novel compounds of the present invention were prepared according to the procedure of the schemes as described hereinabove, using appropriate materials and are further exemplified by the following specific examples. The examples illustrate the preparation of the compounds of formula (I) and their incorporation into pharmaceutical compositions and as such are not to be considered nor construed as limiting the scope of the invention set forth in the claims appended thereto.

### Example 1

### 3-(3-Hydroxy-quinoxalin-2-yl)-1-[4-(4-methyl-piperazine-1-carbonyl-phenyl]-propenone (Compound No. 37)

Step A: Preparation of 3-hydroxy-quinoxalin-2-carboxaldehyde

To 3-methyl-quinoxalin-2-ol (1 g, 6.2 mmol) in 1,4-dioxane (30 ml) was added selenium dioxide (2 g, 18.7 mmol) and refluxed for 4 hours. The reaction mixture was then cooled, filtered through celite and partitioned between water and ethylacetate. The combined organic layers were successively washed with water (50 ml×2) and brine (50 ml×2), dried over anhydrous sodium sulphate and evaporated under vacuo to give 0.58 g of the title compound as a brown solid. This was used without purification in the next step.

¹H NMR(400 MHz, DMSOd₆) δ 7.63-7.69 (2H, m), 7.84-8.38 (2H, m), 10.19 (1H, s), 12.84 (1H, bs).

Step B: Preparation of 4-[3-(3-hydroxy-quinoxalin-2-yl) acryloyl] benzoic acid

A solution of 0.58 g (3.3 mmol) of the product of example 1, Step A and 4-acetyl benzoic acid (0.5 g, 3 mmol) in methanol (40 ml) was cooled to 0° C. To it was added dropwise a solution of sodium hydroxide (0.24 g, 6 mmol) in water (2 ml). The mixture was stirred at room temperature for 16 hours. After completion of the reaction, the mixture was cooled to 0° C., diluted with water (20 ml) and aqueous hydrochloric acid was added to adjust the pH to 4. The precipitate was filtered, washed successively with water (20 mix 2) and brine (10 ml×2) and dried under vacuo at 60° C. for 4 hours to afford 0.5 g of the title compound as a yellow solid.

¹H NMR (400 MHz, DMSOd₆) δ 7.32-7.39 (2H, m), 7.59-7.63 (1H, t), 7.84-7.89 (1H, m), 7.91 (1H, s), 8.05 (1H, s), 8.12-8.17 (3H, t), 8.37-8.41 (1H, d), 12.77 (1H, s), 13.37 (1H, bs).

Step C: Preparation of 3-(3-Hydroxy-quinoxalin-2-yl)-1-[4-(4-methyl-piperazine-1-carbonyl-phenyl]-propenone

To 0.3 g (0.9 mmol) of the product of example 1, Step B in dry tetrahydrofuran (25 ml) was cooled to 0° C., followed by addition of N-ethyldiisopropyl amine (0.2 g, 1.8 mmol) and 1-hydroxybenzotriazole (0.15 g, 1.1 mmol) and the mixture was stirred for 30 minutes. To it was added N-methyl piperazine (0.18 g, 11.8 mmol) and 1-ethyl-3-(3-dimethylaminopropyl) carbodiimide (EDCI, 0.46 g, 2.4 mmol). The reaction mixture was allowed to attain room temperature, stirred overnight and partitioned between water and ethyl acetate. The combined organic layer were successively washed with water (20 ml×2) and brine (10 ml×2), dried over anhydrous sodium sulphate and evaporated under vacuo. The residue was purified by column chromatography on silica gel using 5% methanol in ethyl acetate as the eluent. Trituration of the residue in diethyl ether (20 ml×3) followed by collection of the solid by vacuum filtration provided the title compound (0.12 g) as yellow solid.

¹H NMR (400 MHz, DMSOd₆) δ 2.2 (3H, s), 2.28 (2H, bs), 2.38 (2H, bs), 3.28 (2H, bs), 3.65 (2H, bs), 7.35-7.39 (2H, t), 7.59-7.63 (3H, t), 7.86-7.91 (2H, m), 8.1-8.13 (2H, d), 8.39-8.43 (1H, d), 12.75 (1H, s);

MS, m/z 401

### Example 2

### 1-[4-(4-Methyl-piperazine-1-carbonyl)-phenyl]-3-(6-trifluoromethyl-quinolin-2-yl)-propenone (Compound No. 32)

Step A: Preparation of 2-methyl-6-trifluoromethyl quinoline

To a solution of 4-(trifluoromethyl) aniline (5 g, 31 mmol) in aqueous hydrochloric acid (6N, 90 ml) was added m-nitrobenzene sulphonic acid sodium salt (7 g, 31 mmol), ferrous sulphate (8.62 g, 31 mmol) and boric acid (7.7 g, 124 mmol). The reaction mixture was refluxed under vigorous stirring for 1 hour. To it, crotonaldehyde (3.25 g, 46 mmol) was then added dropwise and the mixture refluxed for 8 hours. After, cooling to 60° C., methanol (10 ml) was added and filtered through celite. The pH of the filtrate was adjusted to 7 with an aqueous solution of sodium hydroxide (1 N). The volatiles were evaporated under vacuo. The reaction mixture was partitioned between water and ethyl acetate. The combined organic layers were successively washed with water (100 ml×2) and brine (50 ml×2), dried over sodium sulphate and evaporated under vacuo. The residue was purified by column chromatography on silica gel using 30% ethyl acetate in hexane as the eluent to afford 4.2 g of the title compound as a brown solid.

¹H NMR (400 MHz, DMSOd₆) δ 2.3 (3H, s), 7.86-7.89 (1H, m), 7.95 (1H, m), 8.17 (2H, d), 8.21-8.26 (1H, d).

Step B: Preparation of 6-trifluoromethyl quinoline-2-carboxaldehyde

To 1 g (4.7 mmol) of the product of example 2, Step A in 1,4-dioxane (20 ml) was added selenium dioxide (0.8 g, 7 mmol) and heated to 60° C. for 8 hours. The reaction mixture was filtered through celite and the filtrate partitioned between water and ethyl acetate. The combined organic layers were successively washed with water (100 ml×2) and brine (50 ml×2), dried over anhydrous sodium sulphate and evaporated under vacuo. Trituration of the residue in hexane (20 ml×3) followed by collection of the solid by vacuum filtration provided 0.7 g of the title compound as a brown solid.

¹H NMR (400 MHz, DMSOd₆)δ 7.86-7.89 (1H, m), 7.95 (1H, m), 8.17 (2H, d), 8.21-8.26 (1H, d), 10.07 (1H, s).

Step C: Preparation of 4-[-3-(6-trifluoromethyl-quinolin-2-yl)-acryloyl]-benzoic acid

To 0.7 g (3 mmol) of the product of example 2, Step B and 4-acetyl benzoic acid (0.5 g, 3 mmol) in methanol (40 ml) was cooled to 0° C., and to it was added dropwise an aqueous solution of sodium hydroxide [0.25 g, 6 mmol in water (2 ml)]. The reaction mixture was stirred at room temperature for 8 hours. The mixture was then cooled to 0° C., diluted with water (20 ml) and aqueous hydrochloric acid was added to adjust the pH to 4. The precipitate was filtered, washed successively with water (20 ml×2) and brine (10 ml×2), and dried under vacuo at 60° C. for 4 hours to afford 0.7 g of the title compound as a yellow solid.

¹H NMR (400 MHz, DMSOd₆) δ 7.58-7.6 (2H, dd), 7.76-7.79 (1H, d), 7.94-7.99 (2H, m), 8.17-8.19 (3H, dd), 8.21-8.26 (1H, d), 8.27-8.29 (1H, d), 8.36 (1H, d), 1.1 (1H, bs).

Step D: Preparation of 1-[4-(4-Methyl-piperazine-1-carbonyl)-phenyl]-3-(6-trifluoromethyl-quinolin-2-yl)-propenone

A solution of 0.2 g (0.5 mmol) of the product of example 2, Step C in dry tetrahydrofuran (25 ml) was cooled to 0° C. N-ethyldiisopropyl amine (0.17 g, 1.25 mmol) and 1- hydroxybenzotriazole (0.15 g, 0.6 mmol) were added to the above solution. After stirring the reaction mixture for 30 minutes, N-methyl piperazine (0.07 g, 1 mmol) and 1-ethyl-3-(3-dimethylaminopropyl) carbodiimide (EDCI, 0.4 g, 2 mmol) were then added. The reaction mixture was allowed to attain room temperature and stirred for 8 hours. The mixture was partitioned between water and ethyl acetate. The combined organic layer were successively washed with water (20 ml×2) and brine (10 ml×2), dried over anhydrous sodium sulphate and evaporated under vacuo. The residue was purified by column chromatography on silica gel using 90% ethyl acetate in hexane as the eluent. Trituration of the residue in hexane followed by collection of the solid by vacuum filtration provided the title compound (0.12 g) as a yellow solid.

¹H NMR (400 MHz, DMSOd₆) δ 2.36 (3H, s), 2.37-2.39 (2H, m), 2.54 (2H, bs), 3.45 (2H, bs), 3.85 (2H, bs), 7.58-7.6 (2H, dd), 7.76-7.79 (1H, d), 7.94-7.99 (2H, m), 8.17-8.19 (3H, dd), 8.21-8.26 (1H, d), 8.27-8.29 (1H, d), 8.33-8.36 (1H, d);

MS, m/z 454

### Example 3

### 2-{3-[4-(3-Dimethylamino-pyrazole-1-carbonyl)-phenyl]-3-oxo-propenyl}-quinoline-6-sulfonic acid amide (Compound No. 46)

Step A: Preparation of 2-methyl-quinolin-6-sulphonamide

To a solution of sulphanilamide (1 g, 5.8 mmol) in aqueous hydrochloric acid (6N, 20 ml) was added m-nitrobenzene sulphonic acid sodium salt (1.3 g, 5.8 mmol), ferrous sulphate (1.6 g, 5.8 mmol) and boric acid (1.4 g, 23 mmol). The reaction mixture was refluxed under vigorous stirring for 1 hour. To it, crotonaldehyde (0.7 g, 8.7 mmol) was then added dropwise and the reaction mixture refluxed for 8 hours. After cooling to 60° C., methanol (2 ml) was added and filtered through celite. The pH of the filtrate was adjusted to 7 with aqueous 1N sodium hydroxide solution. The reaction mixture was partitioned between ethyl acetate and water. The combined organic layer were successively washed with water (10 ml×2) and brine (5 ml×2), dried over sodium sulphate and evaporated under vacuo. The residue was purified by column chromatography on silica gel using 30% ethyl acetate in hexane as the eluent to afford 0.9 g of the title compound as a brown solid.

¹NMR (400 MHz, DMSOd₆) δ 2.68-2.7 (3H, s), 7.51 (2H, s), 7.55-7.57 (1H, d), 8.04-8.09 (2H, t), 8.44-8.49 (2H, t).

Step B: Preparation of 2-formyl-quinolin-6-sulphonamide

To 0.8 g (3.6 mmol) of the product of example 3, Step A in 1, 4-dioxane (20 ml) was added selenium dioxide (0.5 g, 4.5 mmol) and heated to 60° C. for 8 hours. The reaction mixture was filtered through celite and the filtrate partitioned between water and ethyl acetate. The combined organic layers were successively washed with water (50 ml×2) and brine (25 ml×2), dried over anhydrous sodium sulphate and evaporated under vacuo. The residue was decolorised with activated charcoal, filtered through celite and washed with methanol (20 ml). The filtrate was evaporated under vacuo and trituration of the residue in hexane (20 ml×3) followed by collection of the solid by vacuum filtration provided 0.5 g of the title compound as a yellow solid.

¹H NMR (400 MHz, DMSOd₆)δ 2.68-2.7 (3H, s), 7.51 (2H, s), 7.55-7.57 (1H, d), 8.04-8.09 (2H, t), 8.44-8.49 (2H, t), 10.11 (1H, s).

Step C: Preparation of 4-[3-(6-sulphamoyl-quinolin-2-yl)-acryloyl]-benzoic acid

A solution of 4-acetyl benzoic acid (0.5 g, 3 mmol) and 0.8 g (3.3 mmol) of the product of example 3, Step B in methanol (40 ml) was cooled to 0° C. To it was added dropwise an aqueous solution of sodium hydroxide [0.25 g, 6 mmol in water (2 ml)]. The reaction mixture was stirred at room temperature for 8 hours. The mixture was then cooled to 0° C., diluted with water (20 ml) and aqueous hydrochloric acid was added to adjust the pH to 4. The precipitate was filtered, washed with water (20 ml×2) and brine (10 ml×2) and dried under vacuo at 60° C. for 4 hours to afford 0.6 g of the title compound as a yellow solid.

¹H NMR (400 MHz, DMSOd₆) δ 7.62 (2H, bs), 7.8 (1H, d), 8.02 (2H, d), 8.08 (2H, m), 8.14 (1H, m), 8.2 (1H, d), 8.36 (1H, d), 8.4 (1H, d), 8.5 (1H, m), 8.7 (1H, d), 11.12(1H, bs).

Step D: Preparation of N,N-dimethyl-(1H-pyrazol-3-yl) amine

A solution of 3-amino pyrazole (1 g, 14 mmol) in methanol (20 ml) was cooled to 0° C. and to it was added 1.3 g (4.2 mmol) of paraformaldehyde. The reaction mixture was stirred at ambient temperature for 2 hours. It was then cooled to 0° C. followed by addition of sodium cyanoborohydride (2.7 g, 4.2 mmol) and stirred at ambient temperature for another 3 hours. The reaction mixture was partitioned between water and ethyl acetate. The combined organic layers were successively washed with water (25 ml×2) and brine (10 ml×2), dried over anhydrous sodium sulphate and evaporated under vacuo. The residue was purified by column chromatography on silica gel using 50% ethylacetate in hexane as the eluent to afford the 1.1 g of the title compound as a brown oil.

¹H NMR (400 MHz, DMSOd₆) δ 2.89 (6H, s), 5.68-5.69 (1H, d), 6.42-6.43 (1H, bs), 7.39-7.4 (1H, d).

Step E: Preparation of 2-{3-[4-(3-Dimethylamino-pyrazole-1-carbonyl)-phenyl]-3-oxo-propenyl}-quinoline-6-sulfonic acid amide

To a solution of 0.3 g (0.7 mmol) of the product of example 3, Step C in dry tetrahydrofuran (25 ml), pre-cooled to 0° C., was added N-ethyldiisopropyl amine (0.17 g, 1.2 mmol), 1-hydroxybenzotriazole (0.12 g, 0.9 mmol) and stirred for 30 minutes. To it, 0.1 g (0.9 mmol) of the product of example 3, Step D and 1-ethyl-3-(3-dimethyl aminopropyl) carbodiimide (EDCI, 0.4 g, 2 mmol) were added. The reaction mixture was allowed to attain room temperature and stirred for 8 hours. The mixture was partitioned between water and ethyl acetate. The combined organic layers were washed with water (20 ml×2) and brine (10 ml×2), dried over anhydrous sodium sulphate and evaporated under vacuo. The residue was purified by column chromatography on silica gel using 60% ethyl acetate in hexane as the eluent. Trituration of the residue in hexane followed by collection of the solid under vacuum filtration provided the title compound (0.06 g) as yellow solid.

¹H NMR (400 MHz, DMSOd₆) δ 2.89 (6H, s), 6.47-6.49 (1H, d), 7.61 (2H, s), 7.88-7.92 (1H, d), 8.14-8.17 (2H, m), 8.24-8.32 (4H, m), 8.35-8.45 (3H, m), 8.54 (1H, d), 8.74-8.76 (1H, d);

MS, m/z 474

### Example 4

### 1-{4-(morpholine-4-carbonyl)phenyl}-3-quinolin-2-yl-propenone (Compound No. 1)

Step A: Preparation of 4-[3-quinolin-2-yl)-acryloyl]-benzoic acid

To a solution of quinoline-2-carboxaldehyde (1 g, 6.3 mol) and 4-acetyl benzoic acid (1 g, 6.3 mol) in methanol (60 ml) was added dropwise an aqueous solution of sodium hydroxide [0.5 g, 12.7 mol, in water (2 ml)]. The reaction mixture was refluxed for 8 hours. The mixture was then cooled to 0° C., diluted with water (20 ml) and aqueous hydrochloric acid was added to adjust the pH to 4. The precipitate was filtered, successively washed with water (20 ml×2) and brine (10 ml×2), and dried under vacuo at 60° C. for 4 hours to afford 0.6 g of the title compound as a colourless solid.

¹H NMR (400 MHz, DMSOd₆) δ 7.65-7.68 (1H, t), 7.8-7.85(1H, t), 7.89(1H, s), 8.02-8.04 (1H, d), 8.08-8.14 (3H, m), 8.31-8.35 (1H, d), 8.49-8.51 (1H, d), 11.84 (1H, s).

Step B: Preparation of 1-{4-(morpholine-4-carbonyl)phenyl}-3-quinolin-2-yl-propenone

To a solution of 0.15 g (0.5 mmol) of the product of example 4, Step A in dry tetrahydrofuran (20 ml), cooled to 0° C. was added N-ethyldiisopropyl amine (0.17 g, 1.25 mmol) and 1-hydroxybenzotriazole (0.15 g, 0.6 mmol), and stirred for 30 minutes. To it, N-methyl piperazine (0.085 g, 1 mmol) and 1-ethyl-3-(3-dimethylaminopropyl) carbodiimide (EDCI, 0.4 g, 2 mmol) were then added. The reaction mixture was left to attain room temperature and stirred for 8 hours. The mixture was partitioned between water and ethyl acetate. The combined organic layers were washed with water (20 ml×2) and brine (10 ml×2), dried over anhydrous sodium sulphate and evaporated under vacuo. The residue was purified by column chromatography on silica gel using 80% ethyl acetate in hexane as the eluent. Trituration of the residue in hexane followed by collection of the solid by vacuum filtration provided 0.12 g of the title compound as a brown solid.

¹H NMR (400 MHz, DMSOd₆) δ 3.57-3.68 (8H, d), 7.63-7.69 (3H, m), 7.81-7.89 (2H, m), 8.02-8.04 (1H, d), 8.08-8.11 (1H, d), 8.22-8.25 (3H, m), 8.3-8.35 (1H, d), 8.49-8.51 (1H, d);

MS, m/z 373

### Example 5

### Sodium salt of Compound No. 9, i.e., 1-[4-(3-Quinolin-2-yl-acryloyl)-benzoyl]-piperidine-4-carboxylic acid

Following the process described in Example 4, 1-[4-(3-Quinolin-2-yl-acryloyl)-benzoyl]-piperidine-4-carboxylic acid isopropyl ester was prepared.

To the solution of 1-[4-(3-Quinolin-2-yl-acryloyl)-benzoyl]-piperidine-4-carboxylic acid isopropyl ester (0.33 g, 0.7mmol) in methanol (10 ml) at 0° C. was added dropwise a solution of sodium hydroxide (0.2% in methanol). The mixture was stirred at 0° C. to ambient temperature for 5 hours. The solid was filtered, washed with diethyl ether and dried under vacuo to afford 0.3 g of the title compound as a brown solid.

¹H NMR (400 MHz, DMSOd₆) δ 1.54 (2H, bs), 1.77 (1H, bs), 1.92 (1H, bs), 2.59-2.67 (1H, m), 2.92-2.98 (1H, m), 3.11-3.16 (1H, m), 3.49-3.52 (1H, d), 7.6-7.62 (2H, d), 7.65-7.69 (1H, t), 7.81-7.88 (2H, m), 8.02-8.04 (1H, d), 8.08-8.1 (1H, d), 8.2-8.24 (3H, d), 8.31-8.35 (1H, d), 8.49-8.51 (1H, d)

MS, m/z 414

### Example 6

### 1-(4-Nitro-phenyl)-4-[4-(3-quinolin-2-yl-acryloyl)-benzoyl]-piperazin-2-one (Compound No. 8)

Step A: Preparation of {2-(4-Amino-phenylamino)-ethylamino}-acetic acid ethyl ester

To a solution of 1-fluoro-4-nitrobenzene (40 g, 283 mmol) and N-ethyl diisopropylamine (55 g, 425 mmol) in acetonitrile, cooled to 0° C., was added dropwise 42.5 g (710 mmol) of ethylene diamine. The reaction mixture was stirred at ambient temperature for 10 hours. The mixture was partitioned between water and ethyl acetate. The combined organic layers were washed with water (20 ml×2), dried over anhydrous sodium sulphate and evaporated under vacuo to give 27 g of the title compound as a yellow solid residue. The solid thus obtained (4 g, 22 mmol) and N-ethyl diisopropylamine (4.25 g, 33 mmol) were dissolved in dimethylformamide, cooled to 0° C. and ethyl bromoacetate (3.67 g, 22 mmol) diluted with dimethylformamide (10 ml) was added dropwise. The reaction was stirred at ambient temperature for 10 hours. The mixture was partitioned between water and ethyl acetate. The combined organic layers were washed with water (20 ml×2) and brine (10ml×2), dried over anhydrous sodium sulphate and evaporated under vacuo. The residue was purified by column chromatography on silica gel using 60% ethyl acetate in hexane as the eluent. Trituration of the residue in hexane followed by collection of the solid by vacuum filtration provided 2.2 g of the title compound as a yellow solid.

¹H NMR (400 MHz, DMSOd₆) δ 1.33-1.35 (3H, d), 2.72-2.81 (2H, t), 3.19-3.24 (2H, m), 3.89-3.91 (2H, d), 3.4-3.42 (2H, m), 6.6-6.69 (2H, dd), 7.25-7.38 (2H,. m), 7.98-8.03 (2H, m).

Step B: Preparation of {tert-butoxycarbonyl-[2-(4-nitrophenylamino)-ethyl]-amino}-acetic acid

2.5 g (9.4 mmol) of the ester from example 6, Step A was dissolved in tetrahydrofuran (25 ml) containing N-ethyl diisopropylamine (1.2 g, 9.3 mmol), cooled to 0° C. and di-tert-butyl-dicarbonate (t-Boc, 2.5 g, 11 mmol) was added. The reaction mixture was stirred at ambient temperature for 4 hours. The mixture was partitioned between water and ethyl acetate. The combined organic layers were washed with water (20 ml×2) and brine (10 ml×2), dried over anhydrous sodium sulphate and evaporated under vacuo to provide 3 g of a yellow solid. The solid was dissolved in methanol (15 ml) and to it, aqueous sodium hydroxide [1 g, 28 mmol in water (2 ml)] was added dropwise at room temperature. The reaction mixture was stirred for 10 hours. The mixture was then cooled to 0° C., diluted with water (20 ml) and aqueous hydrochloric acid was added to adjust the pH to 7. The mixture was partitioned between water and ethyl acetate. The combined organic layers were washed with water (20 ml×2), dried over anhydrous sodium sulphate and evaporated under vacuo to afford 1.7 g of the title compound as a yellow viscous liquid

¹H NMR (400 MHz, DMSOd₆) δ 1.3-1.35 (9H, d), 2.77-2.81 (2H, t), 3.19-3.24 (2H, m), 3.89-3.91 (2H, d), 3.4-3.42 (2H, m), 6.66-6.69 (2H, dd), 7.25-7.39 (1H, bs), 7.98-8.03 (2H, m), 12.64 (1H, bs).

Step C: Preparation of trifluoroacetic acid salt of 1-(4-nitrophenyl)-piperazine-2-one

To 1.7 g of the product from example 6, Step B in dry tetrahydrofuran (20 ml), cooled to 0° C. was added N-ethyl diisopropyl amine (0.17 g, 1.25 mmol) and 1-hydroxy benzotriazole (0.15 g, 0.6 mmol), and stirred for 30 minutes. To it, 1-ethyl-3-(3-dimethylaminopropyl) carbodiimide (EDCI, 0.4 g, 2 mmol) was then added. The reaction mixture was allowed to attain room temperature and stirred for 10 hours. The mixture was partitioned between water and ethyl acetate. The combined organic layers were washed successively with water (20 ml×2) and brine (10 ml×2), dried over anhydrous sodium sulphate and evaporated under vacuo to provide 4-(4-nitrophenyl)-3-oxo-piperazine-1-carboxylic acid tert-butyl ester (1 g) as a yellow solid. The solid (1 g, 3 mmol) was dissolved in dichloromethane (5 ml), cooled to 0° C. and to it, trifluoroacetic acid (1.36 g, 12 mmol) was added dropwise. The reaction mixture was stirred at 0° C. for an hour. The volatiles were eliminated by co-evaporating with dichloromethane (10 ml×2) to afford 0.8 g of the title compound as a yellow solid, which was used as such in the next step.

¹H NMR (400 MHz, DMSOd₆) δ 3.69-3.7 (2H, t), 3.84-3.87 (2H, t), 4.14 (2H, s), 5.8 (1H, s), 7.68-7.71 (2H, d), 8.26-8.28 (2H, d).

Step D: Preparation of 1-(4-Nitro-phenyl)-4-[4-(3-quinolin-2-yl-acryloyl)-benzoyl]-piperazin-2-one

To a solution of 4-(3-quinolin-2-yl-carolyn) benzoic acid (0.25 g, 0.8 mmol) in dry tetrahydrofuran (25 ml), cooled to 0° C., N-ethyl diisopropylamine (0.8 g, 6.4 mmol) and 1-hydroxy benzotriazole (0.14 g, 1 mmol) were added. The mixture was stirred for 30 minutes. The crude TFA salt from example 6, Step C (0.3 g, 0.9 mmol) dissolved in dry tetrahydrofuran (5 ml), 1-ethyl-3-(3-dimethylaminopropyl) carbodiimide (EDCI, 0.4 g, 2 mmol) was then added. The reaction mixture was allowed to attain room temperature and stirred for 10 hours. The mixture was partitioned between water and ethyl acetate. The combined organic layers were successively washed with water (20 ml×2) and brine (10 ml×2), dried over anhydrous sodium sulphate and evaporated under vacuo. The residue was recrystallized from methanol/diethyl ether to afford 0.21 g of the title compound as a brown solid.

¹H NMR (400 MHz, DMSOd₆) δ 3.76 (1H, bs), 3.85-3.95 (2H, m), 4.03 (1H, bs), 4.23 (1H, bs), 4.44 (1H, bs), 7.65-7.76 (5H, m), 7.81-7.9 (2H, m), 8.02-8.04 (1H, d), 8.08-8.11 (1H, d), 8.23-8.37 (6H, m), 8.49-8.52 (1H, d);

MS, m/z 507

### Example 7

### 1-{4-[3-(5,6,7-trimethoxy-quinolin-2-yl)-acryloyl}-benzoyl}-piperidin-4-one (Compound No. 35)

Step A: Preparation of 5, 6, 7-trimethoxy-2-methyl-quinoline

To a suspension of 3, 4, 5-trimethoxy aniline (5 g, 27 mmol) in aqueous hydrochloric acid (6N, 20 ml) was added m-nitrobenzene sulphonic acid sodium salt (6.15 g, 27 mmol), ferrous sulphate (7.6 g, 27 mmol) and boric acid (6.6 g, 108 mmol), and refluxed under vigorous stirring. After 1 hour, crotonaldehyde (3.8 g, 48 mmol) was added dropwise under reflux. After cooling to room temperature, methanol (2 ml) was added and filtered. The pH of the filtrate was adjusted to 7 with an aqueous solution of sodium hydroxide (1N). The mixture was partitioned between ethyl acetate and water. The combined organic layers were successively washed with water (20 ml×2) and brine (20 ml×2), dried over sodium sulphate and evaporated under vacuo. The solid residue was purified by column chromatography on silica gel using the 40% ethyl acetate in hexane as the eluent to obtain 2.3 g of the title compound as a brown solid.

¹H NMR (400 MHz, DMSOd₆) δ 2.59 (3H, s), 3.84 (3H, s), 3.93-3.95 (6H, s), 7.17 (1H, s), 7.24-7.26 (1H, d), 8.18-8.2 (1H, d).

Step B: Preparation of 5, 6, 7-trimethoxy-quinoline-2-carboxaldehyde

To 1.4 g (6 mmol) of the product from example 7, Step A in 1,4-dioxane (50 ml) was added selenium dioxide (1 g, 9 mmol) and heated at 60° C. for 8 hours. The reaction mixture was filtered through celite and the filtrate partitioned between water and ethyl acetate. The combined organic layers were successively washed with, water (50 ml×2) and brine (25 ml×2), dried over anhydrous sodium sulphate and evaporated under vacuo. The solid residue was triturated with hexane (50 ml×2) to obtain 1.1 g of the title compound as brown solid.

¹ H NMR (400 MHz, DMSOd₆) δ 3.84 (3H, s), 3.93-3.95 (6H, s), 7.17 (1H, s), 7.24-7.26 (1H, d), 8.18-8.20 (1H, d), 10.01 (1H, s).

Step C: Preparation of 4-[3-(5, 6, 7-trimethoxy-quinolin-2-yl)-acryloyl]-benzoic acid

To a solution of 4-acetyl benzoic acid (0.66 g, 4 mmol) and the product from example 7, Step B (1 g, 4 mmol) in methanol (25 ml), cooled to 0° C., was added dropwise an aqueous solution of sodium hydroxide [0.32 g, 8 mmol in water (2 ml)]. The reaction mixture was stirred at room temperature for 16 hours. The mixture was then cooled to 0° C., diluted with water (20 ml) and aqueous hydrochloric acid was added to adjust the pH to 7. The precipitate was isolated by filtration with a Buchner funnel and successively washed with water (20 ml×2) and brine (10 ml×2) and dried under vacuo at 60° C. to afford 2.5 g of the title compound as a brown solid.

¹H NMR (400 MHz, DMSOd₆) δ 4 (9H, s), 7.82-7.86 (1H, m), 8.01-8.05 (3H, m), 8.12-8.14 (2H, m), 8.23-8.29 (3H, m), 11.2 (1H, bs).

Step D: Preparation of 1-{4-[3-(5, 6, 7-trimethoxy-quinolin-2-yl)-acryloyl}-benzoyl}-piperidin-4-one

To 0.2 g (0.5 mmol) of the product from example 7, Step C in dry tetrahydrofuran (25 ml), cooled to 0° C., was added N-ethyldiisopropyl amine (3.9 g, 3 mmol) and 1-hydroxy benzotriazole (0.1 g, 0.7 mmol). The reaction mixture was stirred for 30 minutes. To it, piperidine hydrochloride monohydrate salt (0.8 g, 5.2 mmol), 1-ethyl-3-(3-dimethylaminopropyl) carbodiimide (EDCI, 0.4 g, 2 mmol) were then added. The reaction mixture was allowed to attain room temperature and stirred for 12 hours. The mixture was partitioned between water and ethyl acetate. The combined organic layers were successively washed with water (20 ml×2) and brine (10 ml×2), dried over anhydrous sodium sulphate and evaporated under vacuo. The residue was purified by column chromatography on silica gel using 70% ethyl acetate in hexane as the eluent. Trituration of the residue in hexane followed by collection of the solid by vacuum filtration provided 0.09 g of the title compound as a brown solid.

¹H NMR (400 MHz, DMSOd₆) δ 2.41 (2H, m), 2.54 (2H, m), 3.56-3.61 (2H, m), 3.86-3.93 (5H, m), 3.98-4.02 (6H, d), 7.33 (1H, s), 7.7-7.72 (2H, d), 7.81-7.86 (1H, d), 8.03-8.05 (1H, d), 8.23-8.25 (2H, d), 8.28-8.32 (1H, d), 8.40-8.42 (1H, d);

MS, m/z 475

### Example 8

### 4-Morpholin-4-yl-2-{3-oxo-3-[4-(pyrrolidine-1-carbonyl)-phenyl]-propenyl}-quinoline-6-carboxylic acid methyl ester (Compound No. 13)

Step A: Preparation of 2-methyl-4-oxo-1,4-dihydro-quinoline-6-carboxylic acid

To 20 g (0.15 mol) of 4-amino benzoic acid in toluene (100 ml) containing acetic acid (2 ml) was added ethyl acetoacetate (28 g, 0.2 mol) and refluxed using Dean Stark apparatus to remove the reaction water for 8 hours. The reaction mixture was cooled to room temperature and the precipitate filtered, washed successively with water (100 ml×2) and diethyl ether (100 ml×2), and dried under vacuo overnight. The crude product (16.2 g) was suspended in diphenyl ether or Dowtherm and refluxed for a period of 3 hours with an air condensor. The precipitate was filtered, washed successively with water (100 ml×2) and diethyl ether (100 ml×2), and dried under vacuo at 60° C. for 6 hours to obtain 10.3 g of the title compound as a brown solid.

¹H NMR (400 MHz, DMSOd₆) δ 2.01 (3H, s), 7.49 (2H, s), 7.58 (2H, m), 10.1(1H, bs), 12.1 (1H, bs).

Step B: Preparation of 4-chloro-2-methyl-quinoline-6-carboxylic acid methyl ester

To 10 g (47 mmol) of the product from Example 8, step A in methanol (100 ml), cooled to 0° C., was added dropwise sulphuric acid (10 ml). The reaction mixture was stirred at ambient temperature for 6 hours. The reaction was then cooled to 0° C. and the pH of the solution was adjusted to 7 with aqueous ammonia. The precipitate was filtered, successively washed with water (100 ml×2), diethyl ether (100 ml×2) and dried under vacuo at 60° C. for 6 hours to obtain 9 g of 2-methyl-4-oxo-l,4-dihydro-quinoline-6-carboxylic acid methyl ester as a colorless solid. This 8 g (34 mmol) was dissolved in dry tetrahydrofuran (100 ml) containing a catalytic amount of dimethylformamide (0.5 ml) was cooled to 0° C. To it was added phosphorus oxychloride (3.8 g, 24.6 mmol) dropwise. The reaction mixture was allowed to attain room temperature and then heated to 60° C. for 5 hours. After cooling the reaction mixture to 0° C., it was diluted with water (60 ml), and the pH adjusted to 7 using saturated sodium bicarbonate solution. The mixture was partitioned between water and ethyl acetate. The combined organic layers were successively washed with water (100 ml×2) and brine (100 ml×2), dried over anhydrous sodium sulphate and evaporated under vacuo. Trituration of the residue in diethyl ether (50 ml×2) followed by collection of the solid by vacuum filtration provided 6.1 g of the title compound as a yellow solid.

¹H NMR (400 MHz, DMSOd₆) δ 2.2 (3H, s), 3.9 (3H, s), 6.9 (1H, s), 7.49 (1H, m), 7.61 (2H, m).

Step C: Preparation of 2-methyl-4-morpholin-4-yl-quinoline-6-carboxylic acid methyl ester

To 5 g (17.5 mmol) of the product from example 8, Step B in acetonitrile (100 ml) was added morpholine (3.4 g, 39 mmol) and refluxed for 8 hours. The mixture was partitioned between water and ethyl acetate. The combined organic layers were succesively washed with water (100 ml×2) and brine (25 ml×2), dried over anhydrous sodium sulphate and evaporated under vacuo. The solid residue was triturated with diethyl ether (50 ml×2) followed by collection of the solid by vacuum filtration to afford 5.6 g of the title compound as a brown solid.

¹H NMR (400 MHz, DMSOd₆) δ 2.25 (3H, s), 3.3 (4H, t), 3.78 (3H, s), 3.9 (4H, t), 7(1H, s), 7.49 (1H, m), 7.62 (2H, m).

Step D: Preparation of 2-formyl-4-morpholin-4-yl-quinoline-6-carboxylic acid methyl ester

A solution of 5 g (17.5 mmol) of the product from example 8, Step C and selenium dioxide (2.4 g, 21 mmol) in 1,4-dioxane (100 ml) was stirred at 60° C. for 8 hours. After cooling the reaction mixture, it was filtered through celite and the filtrate was partitioned between water and ethyl acetate. The combined organic layers were successively washed with water (50 ml×2) and brine (50 ml×2), dried over anhydrous sodium sulphate and evaporated under vacuo. The solid residue was triturated with hexane (50 ml×2) followed by collection of the solid by vacuum filtration to afford 3.1 g of the title compound as a yellow solid.

¹H NMR (400 MHz, DMSOd₆) δ 3.3 (4H, t), 3.81 (3H, s), 3.9 (4H, t), 7.1 (1H, s), 7.49 (1H, m), 7.65 (2H, m), 9.89 (1H, s).

Step E: Preparation of 4-Morpholin-4-yl-2-{3-oxo-3-[4-(pyrrolidine-1-carbonyl)-phenyl]-propenyl}-quinoline-6-carboxylic acid methyl ester

To 4-Acetyl-benzoic acid (1 g, 6.1 mmol) in carbon tetrachloride:methanol (1:1 v/v, 40 ml), cooled to 0° C., was added dropwise a solution of hydrogen bromide (65 mg, 0.8 mmol, in acetic acid, 45% w/v). The reaction mixture was stirred for 30 minutes and to it was added dropwise bromine (0.278 g, 1.7 mmol). The mixture was stirred for further 2 hours at ambient temperature. The pH of reaction mixture was adjusted to 7 using an aqueous solution of sodium hydroxide (2N) and partitioned between water and ethyl acetate. The combined organic layers were successively washed with water (20 ml×2) and brine (10 ml×2), dried over anhydrous sodium sulphate and evaporated under vacuo to provide 4-(2-bromo-acetyl)-benzoic acid (0.2 g) as a brown solid. A solution of 4-(2-bromo-acetyl)-benzoic acid (0.2 g, 0.8 mmol) and triphenyl phosphine (0.215, 0.8 mmol) in toluene (20 ml) was refluxed for 8 hours. After cooling the reaction mixture, it was partitioned between water and ethyl acetate. The combined organic layers were successively washed with water (50 ml×2) and brine (50 ml×2), dried over anhydrous sodium sulphate and evaporated under vacuum. Trituration of the solid residue in hexane (20 ml×3) followed by collection of the solid by vacuum filtration gave the ylide (0.3 g) as colourless solid. This ylide (0.3 g, 0.7 mmol) and 0.2 g (0.6 mmol) of the product from example 8, Step D in pyridine (30 ml) was refluxed for 4 hours. After cooling the reaction mixture, it was partitioned between water and ethyl acetate. The combined organic layers were successively washed with water (50 ml×2) and brine (50 ml×2), dried over anhydrous sodium sulphate and evaporated under vacuo. Trituration of the solid residue in hexane (20 ml×3) followed by collection of the solid by vacuum filtration provided 0.18 g of the title compound as a yellow solid.

¹H NMR (400 MHz, DMSOd₆) δ 2.56 (4H, bs), 3.3 (4H, bs), 3.9 (8H, bs), 3.94 (3H, s), 7.62 (1H, s), 7.68-7.7 (1H, d), 7.7-7.8 (1H, m), 8-8.02 (1H, m), 8.08-8.1 (2H, d), 8.18-8.2 (2H, t), 8.26-8.32 (1H, m), 8.63 (1H, s);

MS, m/z 500

### Example 9

### 1-[4-(Pyrazole-1-carbonyl)-phenyl]-3-(3,4,5,6-tetrahydro-2H-[1,2]bipyridinyl-5′-yl-but-2-en-1-one (Compound No. 161)

Step A: Preparation of 1-(3, 4, 5, 6-Tetrahydro-2H-[1,2′]bipyridinyl-5′-yl)-ethanone

To a suspension of anhydrous magnesium dichloride (2 g, 21 mmol) in toluene (20 ml) containing triethylamine (0.4 g, 42 mmol) and pre-cooled to 0° C., was added diethyl malonate (4 g, 25 mmol) and stirred at ambient temperature for 1 hour. To it, 6-chloro-3-nicotinyl chloride (4.4 g, 27 mmol) was then added and stirred for 3 hours. The mixture was partitioned between water and ethyl acetate. The combined organic layers were successively washed with water (100 ml×2) and brine (25 ml×2), dried over anhydrous sodium sulphate and evaporated under vacuum to afford 4.2 g of the title compound as a colourless oil.

This ester (4 g, 17 mmol) in dimethyl sulphoxide (50 ml) was heated to 160° C. for 6 hours. The mixture was partitioned between water and ethyl acetate. The combined organic layers were successively washed with water (100 ml×2) and brine (25 ml×2), dried over anhydrous sodium sulphate and evaporated under vacuo to provide 1-(6-Chloro-pyridin-3-yl)-ethanone (2.3 g) as a colourless oil.

A solution of 1-(6-Chloro-pyridin-3-yl)-ethanone (2 g, 12.8 mmol) arid piperidine (1.47 g, 17.3 mmol) in acetonitrile (30 ml) was refluxed for 8 hours. The mixture was partitioned between water and ethyl acetate. The combined organic layers were successively washed with water (100 ml×2) and brine (25 ml×2), dried over anhydrous sodium sulphate and evaporated under vacuum to provide 2.1 g of the title compound as a brown oil.

¹H NMR (400 MHz, DMSOd₆) δ 1.7 (6H, s), 2.54 (3H, s), 3.6 (4H, s), 6.72-6.79 (1H, d), 7.5-7.53 (1H, d), 7.7 (1H, s).

Step B: Preparation of {4-(3-3,4,5,6-Tetrahydro-2H-[1,2′]bipyridinyl-5′-yl-but-2-enoyl)-benzoic acid

To a solution of 4-acetyl benzoic acid (1 g, 6.1 mmol) in dichloromethane (30 ml) cooled to 0° C., was added trimethylsilyl trifluromethane sulphonate and stirred for 4 hours. The volatiles were evaporated under vacuo to provide the 4-(1-tert-butylsilanyloxy-vinyl)-benzoic acid (0.7 g) as colourless solid.

To the solution of 4-(1-tert-Butylsilanyloxy-vinyl)-benzoic acid (0.7 g, 2.8 mmol) in dichloromethane containing triethylamine (0.57 g, 5.6 mmol) at 0° C. was added dropwise 1-(3,4,5,6-Tetrahydro-2H-[1,2′]bipyridinyl-5′-yl)-ethanone (0.573 g, 2.8 mmol) followed by trifluoroacetic anhydride (1.185 g, 5.6 mmol) and titanium tetrachloride (1,071 g, 5.6 mmol). The reaction mixture was allowed to attain room temperature and stirred for 6 hours. The mixture was partitioned between water and ethyl acetate. The combined organic layers were successively washed with water (20 ml×2) and brine (10 ml×2), dried over anhydrous sodium sulphate and evaporated under vacuo to provide 0.5 g of the title compound as a yellow solid.

¹H NMR (400 MHz, DMSOd₆) δ 1.5 (3H, s) 1.69-1.83 (6H, d), 3.3 (4H, bs), 6.96-6.98 (1H, d), 7.15-7.18 (1H, d), 7.56-7.61 (1H, d), 7.65-7.69 (1H, t), 7.71-7.75 (2H, d), 7.82-7.84 (2H, dd), 8.1 (1H, d), 11.84 (1H, bs)

Step C: Preparation of 1-[4-(Pyrazole-1-carbonyl)-phenyl]-3-(3,4,5,6-tetrahydro-2H-[1,2]bipyridinyl-5′-yl-but-2-en-1-one

To the solution of 4-(3-3,4,5,6-Tetrahydro-2H-[1,2′]bipyridinyl-5′-yl-but-2-enoyl)-benzoic acid (0.5 g, 1.4 mmol) in dry tetrahydrofuran (25 ml), cooled to 0° C. was added N-ethyldiisopropyl amine (0.183 g, 1.4 mmol), 1-hydroxy benzotriazole (0.193 g, 1.42 mmol) and stirred for 30 minutes. The pyrazole (97 mg, 1.43 mmol), and 1-ethyl-3-(3-dimethylaminopropyl) carbodiimide (EDCI, 0.407 g, 2.1 mmol) was then added. The reaction mixture was left to warm to room temperature and stirred for 12 hours. The mixture was partitioned between water and ethyl acetate. The combined organic layers were washed with water (20 ml×2) and brine (10 ml×2), dried over anhydrous sodium sulphate and evaporated under vacuo. The residue was purified by column chromatography on silica gel using 70% ethyl acetate in hexane as the eluent. Trituration of the residue in hexane followed by collection of the solid by vacuum filtration provided the titled compound (0.09 g) as brown solid.

¹H NMR (400 MHz, DMSOd₆) δ 1.69 (3H, s), 1.7 (6H, s), 3.6(4H, s), 6.58-6.59 (1H, s), 6.72-6.79 (1H, d), 7.5-7.53 (1H, s), 7.7 (1H, s), 8.11-8.27 (6H, m), 8.49 (1H, s).

MS, m/z: 400.

### Example 10

### 1-{4-[3-(4-piperidin-1-yl-6-trifluoromethyl-quinolin-2yl)-acryloyl]-phenyl}-3-(3,4,5-trimethoxy-phenyl)-urea (Compound No. 53)

Step A: Preparation of 2-methyl-6-trifluoromethyl-3H-quinolin-4-one

To the solution of 4-trifluoromethyl aniline (5 g, 32 mmol) in toluene (100 ml) containing acetic acid (1 ml) was added ethyl acetoacetate (11.3 g, 87 mmol) and refluxed for 20 hours using Dean Stark apparatus to remove water. The reaction mixture was cooled to room temperature, washed with saturated sodium bicarbonate solution (50 ml×2) and partitioned between water and ethyl acetate. The combined organic layers were dried over anhydrous sodium sulphate and evaporated under vacuo. The crude ester oil was refluxed in a solvent diphenyl ether or Dowtherm® for a period of 12 hours using an air condensor. The precipitate was filtered, washed successively with water (50 ml×2) and diethyl ether (100 ml×2), and dried under vacuo at 60° C. for 6 hours to afford 1.65 g of the title compound as a brown solid.

¹H NMR (400 MHz, DMSOd₆) δ 2.37 (3H, s), 6.04 (1H, s), 7.67-7.69 (1H, d), 7.9-7.93 (1H, dd), 8.3 (1H, s), 11.94 (1H, bs).

Step B: Preparation of 4-chloro-2-methyl-6-trifluoromethyl-quinoline

To 1.65 g (7.2 mmol) of the product from example 10, Step A in dry tetrahydrofuran (100 ml) containing a catalytic amount of dimethylformamide (0.5 ml) and pre-cooled to 0° C., was added dropwise 0.8 g (8.7 mmol) of phosphoryl chloride. The reaction mixture was allowed to attain room temperature and stirred for 4 hours. It was diluted with water (20 ml) and the pH adjusted to 7 using saturated sodium bicarbonate solution. The reaction mixture was partitioned between water and ethyl acetate. The combined organic layers were successively washed with water (50 ml×2) and brine (50 ml×2), dried over anhydrous sodium sulphate and evaporated under vacuo to give 1.3 g of the title compound as red oil.

Step C: Preparation of 2-Methyl-4-piperidine-1-yl-6-trifluoromethyl-quinoline

1 g (4 mmol) of the product from example 10, Step B was refluxed in piperidine (20 ml) for 28 hours. The reaction mixture was diluted with water (20 ml) and the pH adjusted to 7 using dilute hydrochloric acid. The mixture was partitioned between water and ethyl acetate. The combined organic layers were successively washed with water (50 ml×2) and brine (50 ml×2), dried over anhydrous sodium sulphate and evaporated under vacuo. The residue was purified by column chromatography on silica gel using 60% ethyl acetate in hexane as the eluent to afford 1 g of the title compound as brown oil.
¹H NMR (400 MHz, DMSOd₆) δ 1.68-1.69 (2H, m), 1.8-1.89 (4H, m), 2.3 (3H, s), 3.37-3.48 (4H, m), 6.8 (1H, s), 7.67-7.69 (1H, d), 7.9-7.93 (1H, dd), 8.3 (1H, d).
   Step D: Preparation of 4-piperidin-1-yl-6-trifluoromethyl-quinoline-2-carboxaldehyde

To 1 g (3.4 mmol) of the oil obtained from example 10, Step C in 1,4-dioxane (20 ml) was added selenium dioxide (0.6 g, 5.6 mmol) and heated to 60° C. for 4 hours. After cooling to room temperature, the mixture was filtered through celite. The filtrate was partitioned between water and ethyl acetate. The combined organic layers were successively washed with water (50 ml×2) and brine (50 ml×2), dried over anhydrous sodium sulphate and evaporated under vacuo to afford 1 g of the title compound as a brown solid.

¹H NMR (400 MHz, DMSOd₆) δ 1.68-1.69 (2H, m), 1.8-1.89 (4H, m), 3.37-3.48 (4H, m), 6.8 (1H, s), 7.67-7.69 (1H, d), 7.9-7.93 (1H, dd), 8.3 (1H, d), 10.06 (1H, s).

Step E: Preparation of 1-(4-Acetyl-phenyl)-3-(3, 4, 5-trimethoxy-phenyl)-urea

3, 4, 5-trimethoxyaniline (0.6 g, 3.2 mmol) and 4-acetyl phenyl isocyanate (0.51 g, 3.2 mmol) in toluene (30 ml) was refluxed for 12 hours. The precipitate was filtered, washed with water (10 ml×2) and dried under vacuo to afford 0.8 g of the title compound as a colourless solid.

¹H NMR (400 MHz, DMSOd₆) δ 2.53 (3H, s), 4.01 (9H, s), 7.42-7.45 (2H, d), 7.59-7.64 (2H, t), 7.84-7.86 (2H, dd), 8.74(1H, bs), 11.04 (1H, bs).

Step F: Preparation of 1-{4-[3-(4-piperidin-1-yl-6-trifluoromethyl-quinolin-2yl)-acryloyl]-phenyl}-3-(3, 4, 5-trimethoxy-phenyl)-urea

To 0.1 g (0.3 mmol) of the product from example 10, Step D and 0.11 g (0.3 mmol) of the product from example 10, Step E in methanol (20 ml), pre-cooled to 0° C., was added dropwise an aqueous solution of sodium hydroxide (0.05 g, 1.2 mmol). The reaction mixture was stirred at room temperature for 24 hours. After completion of reaction, the mixture was cooled to 0° C., diluted with water (20 ml) and the the pH adjusted to 7 using aqueous hydrochloric acid. The volatiles was evaporated under vacuo. The mixture was partitioned between water and ethyl acetate. The combined organic layers were successively washed with water (50 ml×2) and brine (50 ml×2), dried over anhydrous sodium sulphate and evaporated under vacuo. The residue was purified by column chromatography on silica gel using 75% ethyl acetate in hexane as the eluent to provide 0.04 g of the title compound.

¹H NMR (400 MHz, DMSOd₆) δ 1.69 (2H, m), 1.83 (4H, m), 3.28-3.3 (4H, m), 3.61-3.62 (3H, d), 3.75-3.77 (6H, d), 6.81-6.83 (2H, d), 7.67-7.69 (3H, t), 7.76-7.8(1H, d), 7.97-7.99 (1H, dd), 8.15-8.2 (4H, m), 8.32-8.36 (1H, d), 8.84 (1H, s), 9.21(1H, s);

MS, m/z 633.

### Example 11

### 1-{4-[3-(6-Morpholin-4-yl-pyridin-2-yl)-acryloyl]-phenyl}-3-[2-(pyridin-2-yl-sulfanyl)-ethyl]-urea (Compound No. 132)

Step A: Preparation of 6-morpholin-4-yl-pyridine-2-carboxaldehyde

A solution of 6-bromo-pyridine-2-carboxaldehyde (1.9 g, 10 mmol), morpholine (1.75 g, 20 mmol) and potassium carbonate (3 g, 22 mmol) in acetonitrile (20 ml) was refluxed for 20 hours. The reaction mixture was cooled to room temperature, diluted with water (50 ml) and the pH adjusted to 7. The mixture was partitioned between water and ethyl acetate. The combined organic layers were successively washed with water (25 ml×2) and brine (25 ml×2), dried over anhydrous sodium sulphate and evaporated under vacuo. The gummy residue was purified by column chromatography over silica gel using 40% ethyl acetate in hexane as the eluent to afford 1.5 g of the title compound as a brown liquid.

¹H NMR (400 MHz, DMSOd₆) δ 3.55-3.58 (4H, t), 3.91-3.94 (4H, t), 7.15-7.18 (1H, d), 7.56-7.61 (1H, d), 7.65-7.69 (1H, t), 9.98 (1H, s).

Step B: Preparation of 4-[3-(6-morpholin-4-yl-pyridin-2-yl)-acryloyl]-benzoic acid

To a solution of 4-acetyl benzoic acid (1 g, 6 mmol) and the aldehyde (1.1 g, 6 mmol) from example 11, Step A in methanol (25 ml), pre-cooled to 0° C., was added dropwise an aqueous solution of sodium hydroxide [0.5 g, 16 mmol in water (2 ml)]. The reaction mixture was stirred at room temperature for 16 hours. The mixture was then cooled to 0° C., diluted with water (20 ml) and the pH adjusted to 7 using aqueous hydrochloric acid. The precipitate was isolated by filtration with a Buchner funnel and successively washed with water (20 ml×2) and brine (10 ml×2), and dried under vacuo at 60° C. to afford 1.35 g of the title compound as a yellow solid.

¹NMR (400 MHz, DMSOd₆) δ 3.55-3.58 (4H, t), 3.91-3.94 (4H, t), 6.96-6.98 (1H, d), 7.15-7.18 (1H, d), 7.56-7.61 (1H, d), 7.65-7.69 (1H, t), 7.71-7.75 (2H, d), 7.82-7.84 (2H, dd), 8.1 (1H, d), 11.84 (1H, bs).

Step C: Preparation of 4-[3-(6-morpholin-4-yl-pyridin-2-yl)-acryloyl]-benzoyl azide

The product from example 11, Step B (1.35 g, 4 mmol) was dissolved in dry . dimethylformamide (20 ml) containing N-ethyl diisopropylamine (1 g, 8 mmol), cooled to 0° C. followed by dropwise addition of ethyl chloroformate (0.65 g, 6 mmol). The reaction mixture was stirred for 1 hour. To it, an aqueous solution of sodium azide [0.8 g, 12 mmol, in water (2 ml)] was then added and stirred for another 1 hour. The mixture was diluted with water (50 ml) and the resulting precipitate was filtered, successively washed with water (25 ml×2) and hexane (25 ml×2), and dried under vacuo to afford 0.8 g of the title compound as a yellow solid.

Step D: Preparation of 2-(pyridin-2-ylsulfanyl)-ethylamine

A solution of pyridin-2-thiol (0.2 g, 1.8 mmol), 2-bromo-ethylamine (0.55 g, 2.7 mmol) and potassium carbonate (1 g, 7.2 mmol) in acetonitrile (30 ml) was refluxed for 20 hours. The mixture was partitioned between water and ethyl acetate. The combined organic layers were washed successively with water (25 ml×2) and brine (25 ml×2), dried over anhydrous sodium sulphate and evaporated under vacuo to provide 0.16 g of the title compound as a gummy residue, which was used without further purification in the next step.

¹H NMR (400 MHz, DMSOd₆) δ 2.76 (2H, t), 3.13 (2H, t), 3.3(2H, bs), 7.07-7.11 (1H, m), 7.2-7.3 (1H, m), 7.6-7.64 (1H, m), 8.4-8.42 (1H, m).

Step E: Preparation of 1-{4-[3-(6-Morpholin-4-yl-pyridin-2-yl)-acryloyl]-phenyl}-3-[2-(pyridin-2-yl-sulfanyl)-ethyl]-urea

A solution of the product from example 11, Step C (0.2 g, 0.55 mmol) and the product from example 9, Step D (0.16 g, 1 mmol) in toluene (20 ml) was refluxed for 16 hours. The reaction mixture was partitioned between water and ethyl acetate. The combined organic layers were successively washed with water (25 ml×2) and brine (25 ml×2), dried over anhydrous sodium sulphate and evaporated under vacuo. The solid residue was purified by column chromatography over silica gel using 3% methanol in dichloromethane as the eluent to afford 0.16 g of the title compound as a yellow solid.

¹H NMR (400 MHz, DMSOd₆) δ 3.25-3.29 (2H, m), 3.39-3.41 (2H, t), 3.54-3.56 (4H, t), 3.72-3.74 (4H, t), 6.61-6.67 (1H, m), 6.92-6.95 (1H, d), 7.11-7.14 (2H, t), 7.35-7.37 (1H, d), 7.51-7.55 (1H, d), 7.58-7.6 (2h, d), 7.62-7.68 (3H, m), 7.99-8.03 (2H, m), 8.43-8.44 (1H, d), 9.12 (1H, s);

MS, m/z 488

### Example 12

### 1-{4-[3-(6-Morpholin-4-yl-pyridin-2-yl)-acryloyl]-phenyl}-3-[2-pyridin-2-ylsulfonyl)-ethyl]-urea (Compound No. 131)

To a solution of 1-{4-[3-(6-morpholin-4-yl-pyridin-2-yl)-acryloyl]-phenyl}-3-[2-pyridin-2-ylsulfanyl)-ethyl]-urea (0.13 g, 0.26 mmol) in methanol (2 ml), pre-cooled to 0° C. was added dropwise a solution of oxone [0.08 g, 0.13 mmol in water (2 ml)]. The reaction mixture was stirred at 0° C. for 30 minutes. The mixture was diluted with water (20 ml) and the volatiles were evaporated under vacuo. The precipitate was filtered, successively washed with water (10 ml×2) and hexane (10 ml×2), and dried under vacuo at 60° C. for 4 hours to afford 0.1 g of the title compound as a yellow solid.

¹H NMR (400 MHz, DMSOd₆) δ 3.25-3.29 (2H, t), 3.36 (2H, t), 3.55-3.56 (4H, t), 3.72-3.75 (4H, t), 6.92-6.95 (1H, d), 7.11-7.14 (1H, d), 7.35-7.37 (1H, d), 7.51-7.59 (3H, m), 7.62-7.67 (2H, m), 7.99-8.03 (3H, m), 8.08-8.14 (1H, m), 8.43-8.45 (1H, d), 8.67-8.68 (1H, d), 9.08-9.13 (1H, d);

MS, m/z 528

### Example 13

### 1-[2-(4-Methyl-piperazin-1-yl)-ethyl]-3-{4-[3-(6-morpholin-4-yl-pyridin-2-yl)-acryloyl}-phenyl}-urea (Compound No. 133)

Step A: Preparation of 2-(4-methyl-piperazin-1-yl)-ethylamine

A solution of N-methyl piperazine (0.2 g, 2 mmol), 2-bromo-ethylamine (0.6 g, 3 mmol) and potassium carbonate (1.1 g, 8 mmol) in acetonitrile (30 ml) was refluxed for 20 hours.

The mixture was partitioned between water and ethyl acetate. The combined organic layers were successively washed with water (25 ml×2) and brine (25 ml×2), dried over anhydrous sodium sulphate and evaporated under vacuo to afford 0.1 g of the title compound as a gummy residue, which was used without purification in the next step.

¹H NMR (400 MHz, DMSOd₆) δ 2.2 (3H, s), 2.28 (2H, bs), 2.4 (2H, bs), 2.7 (2H, t), 3.12 (2H, t), 3.3 (2H, bs), 3.32 (2H, bs), 3.6 (2H, bs).

Step B: Preparation of 1-[2-(4-Methyl-piperazin-1-yl)-ethyl]-3-{4-[3-(6-morpholin-4-yl-pyridin-2-yl)-acryloyl}-phenyl}-urea

A solution of 0.2 g (0.55 mmol) of the product from example 11, Step C and 0.1 g (0.7 mmol) the product from example 13, Step A in toluene (20 ml) was refluxed for 16 hours. The reaction mixture was partitioned between water and ethyl acetate. The combined organic layers were successively washed with water (25 ml×2) and brine (25 ml×2), dried over anhydrous sodium sulphate and evaporated under vacuo. The solid residue was purified by column chromatography over silica gel using 10% methanol in dichloromethane as the eluent to afford 0.02 g of the title compound as a yellow solid.

¹H NMR (400 MHz, DMSOd₆) δ 2.15 (3H, s), 2.29-2.33 (3H, bs), 2.37-2.39 (5H, t), 3.50-3.55 (8H, bs), 3.74 (4H, bs), 6.92-6.94 (1H, d), 7.11-7.17 (1H, d), 7.51-7.52 (1H, d), 7.55-7.62 (3H, m), 7.99-8.03 (3H, m), 8.11-8.14 (1H, m), 8.34 (1H, s);

MS, m/z 479

### Example 14

### 1-{4-[3-(6-Morpholin-4-yl-pyridin-2-yl)-acryloyl]-phenyl}-3-(2-oxo-2-piperidin-1-yl-ethyl)-urea (Compound No. 126)

Step A: Preparation of (2-oxo-2-piperidin-1-yl-ethyl)-carbamic acid tert-butyl ester

To a solution of tert-butoxycarbonylamino-acetic acid (0.7 g, 4 mmol) in dry tetrahydrofuran (25 ml), pre-cooled to 0° C., was added N-ethyldiisopropyl amine (1 g, 8 mmol) and 1-hydroxy benzotriazole (0.65 g, 4.8 mmol). The mixture, was stirred for 30 minutes, followed by addition of piperidine (0.5 g, 6 mmol) and 1-ethyl-3-(3-dimethylaminopropyl) carbodiimide (EDCI, 1.6 g, 8.4 mmol). The reaction mixture was allowed to attain room temperature and stirred for 12 hours. The mixture was partitioned between water and ethyl acetate. The combined organic layers were successively washed with water (20 ml×2) and brine (10 ml×2), dried over anhydrous sodium sulphate and evaporated under vacuo. The residue was triturated with hexane followed by collection of the solid by vacuum filtration to afford 0.5 g of the title compound as a colourless solid.

Step B: Preparation of trifluoroacetate salt of (2-oxo-2-piperidin-1-yl-ethyl)-carbamic acid tert-butyl ester

The product from example 14, Step A (0.5 g, 2 mmol) was dissolved in dry dichloromethane (10 ml), cooled to 0° C. and to it was added dropwise a solution of trifluoroacetic acid [1 g, 8 mmol, in dichloromethane (2 ml)]. The reaction mixture was stirred for 3 hours at room temperature. The volatiles were then evaporated under vacuo to obtain the title compound, which was used as such in the next step.

¹H NMR (400 MHz, DMSOd₆) δ 1.57-1.59 (6H, m), 3.12-3.21 (2H, s), 3.59-3.61 (4H, t), 3.61-3.67 (2H, bs).

Step C: Preparation of 1-{4-[3-(6-Morpholin-4-yl-pyridin-2-yl)-acryloyl]-phenyl}-3-(2-oxo-2-piperidin-1-yl-ethyl)-urea

A solution of 0.2 g (0.55 mmol) of the product from example 11, Step C and 0.22 g of the product from example 14, Step B in toluene (20 ml) was refluxed for 16 hours. The reaction mixture was partitioned between water and ethyl acetate. The combined organic layers were successively washed with water (25 ml×2) and brine (25 ml×2), dried over anhydrous sodium sulphate and evaporated under vacuo. The solid residue was purified by column chromatography over silica gel using 1.5% methanol in dichloromethane as the eluent to afford 0.04 g of the title compound as a yellow, solid.

¹H NMR (400 MHz, DMSOd₆) δ 2.68-2.72 (1H, d), 2.93 (1H, bs), 3.3-3.33 (1H, m), 3.38 (1H, bs), 3.41-3.43 (1H, m), 3.53 (4H, bs), 3.69-3.74 (6H, bs), 3.74-3.76 (2H, m), 3.78-3.97 (4H, m), 6.91-6.93 (1H, d), 7.1-7.12 (1H, dd), 7.49 (1H, t), 7.53-7.57 (1H, t), 7.62-7.67 (2H, m), 7.99-8.01 (1H, d), 8.03-8.08 (2H, t), 9.38 (1H, s);

MS, m/z 478

### Example 15

### N-(2-{3-[4-(3-Quinoxalin-2-yl-acryloyl)-phenyl]-ureido}-ethyl)-benzene sulfonamide (Compound No. 136)

Step A: Preparation of N-(2-amino-ethyl)-benzenesulfonamide

To a solution of ethylene diamine (0.1 g, 1.2 mmol) in dry dichloromethane (20 ml) containing triethylamine, pre-cooled to 0° C., was added benzenesulphonyl chloride (0.15 g, 0.84 mmol) dropwise. The reaction mixture was stirred at 0° C. for 1 hour and at room temperature for another two hours. The mixture was partitioned between water and ethyl acetate. The combined organic layers were successively washed with water (25 ml×2) and brine (25 ml×2), dried over anhydrous sodium sulphate and evaporated under vacuo to afford 0.13 g of the title compound as yellow oil.

¹H NMR (400 MHz, DMSOd₆) δ 2.4-2.52 (2H, t), 2.7-2.74 (2H, t), 7.56-7.66 (5H, m), 7.72-7.74 (1H, dd), 7.78-7.8(2H, dd).

Step B: Preparation of quinoxalin-2-carboxaldehyde

To 2-methyl-quinoxaline (1 g, 7 mmol) in 1, 4-dioxane (30 ml) was added selenium dioxide (2.3 g, 21 mmol) and the mixture heated to 60° C. for 2 hours. After cooling the reaction mixture, it was filtered over celite and partitioned between water and ethylacetate. The combined organic phases were successively washed with water (50 ml×2) and brine (50 ml×2), dried over anhydrous sodium sulphate and evaporated under vacuo, to afford 0.6 g of the crude title compound as a brown solid, which was used without purification in the next step.

¹H NMR (400 MHz, DMSOd₆) δ 7.98-8.09 (3H, m), 8.21-8.24 (1H, m), 8.28-8.31 (1H, m), 10.19 (1H, s).

Step C: Preparation of 4-(3-quinoxalin-2-yl-acryloyl)-benzoic acid

To 0.6 g (3.8 mmol) of the product from example 15, step B and 4-acetyl benzoic acid (0.55 g, 3.4 mmol) in methanol (40 ml), pre-cooled to 0° C., was added dropwise a solution of sodium hydroxide [(0.27 g, 6.8 mmol) in water (2 ml)]. The mixture was stirred at room temperature for 16 hours. After completion of reaction, the mixture was cooled to 0° C., diluted with water (20 ml) and the pH adjusted to 7 using aqueous hydrochloric acid. The precipitate was isolated by filtration with a Buchner funnel and successively washed with water (20 ml×2) and brine (10 ml×2), dried under vacuo at 60° C. to afford 0.5 g of the title compound as a yellow solid.

¹H NMR (400 MHz, DMSOd₆) δ 6.6 (2H, d), 7.5 (1H, d), 7.56 (1H, m), 7.69 (2H, m), 7.8-7.82 (2H, m), 7.83-7.84 (2H, q), 7.91-7.92 (1H, d) 12.14 (1H, s).

Step D: Preparation of 4-(3-quinoxalin-2-yl-acryloyl)-benzoyl azide

The product from example 15, step C (0.5 g, 1.6 mmol) was dissolved in dry dimethylformamide (20 ml) containing N-ethyl diisopropylamine (0.4 g, 3.2 mmol), cooled to 0° C., and to it, ethyl chloroformate (0.26 g, 2.4 mmol) was added dropwise. The reaction mixture was stirred for 1 hour. An aqueous solution of sodium azide [0.31 g, 4.8 mmol, in water (1 ml)] was then added to the reaction mixture and stirred for another 1 hour. The mixture was diluted with water (20 ml) and the resulting precipitate was filtered, successively washed with water (25 ml×2) and hexane (25 ml×2), and dried under vacuo to afford 0.4 g of the title compound as a yellow solid.

Step E: Preparation of N-(2-{3-[4-(3-Quinoxalin-2-yl-acryloyl)-phenyl]-ureido}-ethyl)-benzene sulfonamide

A solution of the product from example 15, step D (0.2 g, 0.6 mmol) and N-(2-amino-ethyl)-benzenesulfonamide (0.13 g, 0.65 mmol) in toluene was refluxed for 16 hours. The reaction mixture was partitioned between water and ethyl acetate. The combined organic layers were successively washed with water (25 ml×2) and brine (25 ml×2), dried over anhydrous sodium sulphate and evaporated under vacuo. The solid residue was purified by column chromatography over silica gel using 3% methanol in dichloromethane as the eluent to afford 0.15 g of the title compound as a brown solid.

¹H NMR (400 MHz, DMSO d₆) δ 2.81-2.86 (2H, m), 3.15-3.2 (2H, m), 7.58-7.64 (4H, m), 7.65-7.84 (4H, m), 7.87-7.93 (3H, m), 8.13-8.18 (4H, m), 8.28-8.31(1H, d), 8.46-8.5 (1H, d), 9.18 (1H, s), 9.58-9.59 (1H, d);

MS, m/z 502

### Example 16

### 1-Benzenesulfonyl-hydrazino-3-[4-(3-quinoxalin-2-yl-acryloyl)-phenyl]-urea (Compound No. 139)

A solution of the 4-(3-quinoxalin-2-yl-acryloyl)-benzoyl azide (0.2 g, 0.6 mmol) and benzene sulphonyl hydrazide (155 mg, 0.9 mmol) in toluene was refluxed for 16 hours. The reaction mixture was partitioned between water and ethyl acetate. The combined organic layers were successively washed with water (25 ml×2) and brine (25 ml×2), dried over anhydrous sodium sulphate and evaporated under vacuo to provide 0.17 g of the title compound as a yellow solid.

¹H NMR (400 MHz, DMSOd₆) δ 7.57-7.61 (2H, t), 7.63-7.66 (3H, t), 7.83-7.89 (3H, m), 7.9-7.97 (2H, m), 8.05-8.8 (1H, d), 8.11-8.14 (2H, d), 8.15-8.19 (2H, m), 8.45-8.49 (1H, d), 8.97-9.01(1H, d), 9.58-9.60 (1H, d), 9.79(1H, bs);

MS, m/z 473

### Example 17

### 1-(Morpholine-4-sulfonyl)-3-[4-(3-quinolin-2-yl-acryloyl)-phenyl]-urea (Compound No. 142)

Step A: Preparation of 1-(4-amino-phenyl)-3-quinolin-2-yl-propenone

To a solution of quinoline-2-carboxaldehyde (1 g, 6.3 mol) and 4-amino acetophenone (0.85 g, 6.3 mol) in methanol (60 ml) was added dropwise an aqueous solution of sodium hydroxide [0.5 g, 12.7 mol, in water (2 ml)]. The reaction mixture was stirred at room temperature for 18 hours. The mixture was then cooled to 0° C., diluted with water (20 ml) and the pH adjusted to 7 using aqueous hydrochloric acid. The precipitate was isolated by filtration with a Buchner funnel and successively washed with water (20 ml×2) and brine (10 ml×2), and dried under vacuo at 60° C. to afford 0.8 g of the title compound as a yellow solid.

¹H NMR (400 MHz, DMSOd₆)δ 6.03 (1H, bs), 6.27(1H, s), 6.56-6.66 (2H, dd), 7.64 (2H, bs), 7.72-7.8 (2H, t), 7.95-8.05 (3H, m), 8.12-8.17 (1H, d), 8.18-8.29 (1H, d), 8.43-8.45 (1H, d).

Step B: Preparation of 11-(Morpholine-4-sulfonyl)-3-[4-(3-quinolin-2-yl-acryloyl)-phenyl]-urea

To a suspension of 0.25 g (0.67 mmol) of the product from example 17, step A in dry toluene (20 ml) was added chlorosulphonyl isocyanate (0.14 g, 1 mmol) and refluxed for 2 hours. Morpholine (0.5 g, 5.7 mmol) was then added to the reaction mixture and refluxed for another 4 hours. The reaction mixture was partitioned between water and ethyl acetate. The combined organic layers were successively washed with water (25 ml×2) and brine (25 ml×2), dried over anhydrous sodium sulphate and evaporated under vacuo. The solid residue was purified by column chromatography over silica gel using 60% ethyl acetate in hexane as the eluent to afford 0.05 g of the title compound as a yellow solid.

¹H NMR (400 MHz, DMSOd₆) δ 3.71-3.88 (8H, m), 7.4-7.5 (1H, m), 7.54-7.6 (3H, m), 7.66-7.7 (1H, m), 7.77-7.83 (2H, m), 7.83-7.85 (1H, d), 7.92-7.96 (1H, m), 8-8.04 (1H, m), 8.07-8.10 (1H, m), 8.11-8.17 (1H, m), 8.2-8.22 (1H, d), 9.22(1H, s);

MS, m/z 466

### Example 18

### Hydrochloride salt of Compound No. 160, i.e., 1-{2-[N-(6-Methyl-pyridin-2-yl)-hydrazino]-ethyl}-3-[4-(3-quinoxalin-2-yl-acryloyl)-phenyl] urea

Step A: Preparation of N′-(6-methyl-pyridin-2-yl)-hydrazinecarboxylic acid tert-butyl ester

A solution of 2-chloro-6-methyl-pyridine (1 g, 7.8 mmol), tert-butyl carbazate (1.55 g, 11.4 mmol) and potassium carbonate (4.3 g, 31 mmol) in acetonitrile (50 ml) was refluxed for 20 hours. The reaction mixture was partitioned between water and ethyl acetate. The combined organic layers were successively washed with water (25 ml×2) and brine (25 ml×2), dried over anhydrous sodium sulphate and evaporated under vacuo to provide 1.1 g of the title compound as yellow oil, which was used without purification in the next step.

¹H NMR (400 MHz, DMSOd₆) δ 1.38 (9H, s), 2.44 (3H, s), 3.91 (1H, s), 7.26-7.31 (2H, m), 7.72-7.76 (1H, t), 7.85 (1H, bs).

Step B: Preparation of N′-(2-aminoethyl)-N′-(6-methyl-pyridin-2yl)-hydrazinecarboxylic acid tert-butyl ester

A solution of bromoethylamine hydrobromide (1.5 g, 7.4 mmol), potassium carbonate (2.7 g, 19.5 mmol) and 1.1 g (4.9 mmol) of the product from example 18, step A in acetonitrile (50 ml) was refluxed for 20 hours. The reaction mixture was partitioned between water and ethyl acetate. The combined organic layers were successively washed with water (25 ml×2) and brine (25 ml×2), dried over anhydrous sodium sulphate and evaporated under vacuo to afford 1 g of the title compound as yellow oil, which was used without purification in the next step.

Step C: Preparation of hydrochloride salt of 1-{2-[N-(6-Methyl-pyridin-2-yl)-hydrazino]-ethyl}-3-[4-(3-quinoxalin-2-yl-acryloyl)-phenyl]urea

A solution of 4-(3-quinoxalin-2-yl-acryloyl)-benzoyl azide (0.2 g, 0.6 mmol) and the product from example 18, step B (0.18 g, 0.7 mmol) in toluene (20 ml) was refluxed for 16 hours. The reaction mixture was partitioned between water and ethyl acetate. The combined organic layers were successively washed with water (25 ml×2) and brine (25 ml×2), dried over anhydrous sodium sulphate and evaporated under vacuo. The solid residue was purified by column chromatography over silica gel using 80% ethyl acetate in hexane as the eluent. The solid residue thus obtained was dissolved in acetonitrile—hydrochloric acid (10%, 2 ml) and stirred for 2 hours. The precipitate was filtered and washed with diethyl ether (10 ml×2), dried under vacuo at 60° C. for 4 hours to afford 0.07 g of the title compound as a yellow solid.

¹H NMR (400 MHz, DMSOd₆) δ 3.4-3.44 (2H, t), 3.45-3.5(2H, t) 7.13-7.14 (1H, m), 7.35-7.36 (1H, d) 7.62-7.67 (2H, t), 7.83-7.95 (4H, m), 8.13-8.19 (5H, m), 8.27-8.29 (2H, d), 8.46-8.50 (2H, d), 9.79 (2H, s);

MS, m/z 504

### Example 19

### {4-[3-(4-Morpholin-4-yl-quinolin-2-yl)-acryloyl]-phenyl}-carbamic acid thiophen-2-yl-methyl ester (Compound No. 68)

Step A: Preparation of 2-methyl-4-morpholin-4-yl-quinoline

To a solution of 4-chloro-2-methyl-quinoline (6 g, 34 mmol) in dry acetonitrile (100 ml) was added morpholine (8.7 g, 100 mmol), and the reaction was heated to 60° C. for 7 hours. The reaction mixture was then cooled, filtered through celite and the filtrate partitioned between water and ethyl acetate. The combined organic layers were successively washed with water (50 ml×2) and brine (20 ml×2), dried over anhydrous sodium sulphate and evaporated under vacuo. The residue was purified by column chromatography on silica gel using 40% ethyl acetate in hexane as the eluent to afford 7 g of the title compound as a brown solid.

¹H NMR (400 MHz, DMSOd₆) δ 2.63 (3H, s), 3.25-3.27 (4H, t), 3.88-3.9 (4H, t), 7.39 (1H, s), 7.7-7.74 (1H, t), 7.83-7.87 (1H, t), 8.13-8.18 (2H, t).

Step B: Preparation of 4-morpholine-4-yl-quinoline-2-carboxaldehyde

To 5 g (22 mmol) of the product from example 19, step A in 1,4-dioxane (50 ml) was added selenium dioxide (3 g, 133 mmol) and heated to 60° C. for 7 hours. The mixture was cooled to room temperature and partitioned between water and ethyl acetate. The combined organic layers were successively washed with water (20 ml×2) and brine (10 ml×2), dried over anhydrous sodium sulphate and evaporated under vacuo to provide 3.2 g of the title compound as a brown solid, which was used without purification in the next step.

¹H NMR (400 MHz, DMSOd₆) δ 3.25-3.27 (4H, t), 3.88-3.9 (4H, t), 7.39 (1H, s), 7.7-7.74 (1H, t), 7.83-7.87 (1H, t), 8.13-8.18 (2H, t), 10.06 (1H, s).

Step C: Preparation of (4-acetyl-phenyl)-carbamic acid-thiophene-2yl-methyl ester

To a solution of 4-acetyl phenyl isocyanate (0.5 g, 3 mmol) in toluene (20 ml) was added thiophen-2-yl-methanol (0.43 g, 3 mmol), and the mixture refluxed for 7 hours. The mixture was then cooled to room temperature; the precipitate was filtered, washed successively with water (25 ml×2) and hexane (25 ml×2), and dried under vacuo at 60° C. for 4 hours to provide 0.75 g of the title compound as a colourless solid.

Step D: Preparation of {4-[3-(4-Morpholin-4-yl-quinolin-2-yl)-acryloyl]-phenyl}-carbamic acid thiophen-2-yl-methyl ester

To 0.2 g (0.82 mmol) of the product from example 19, step B and 0.38 g (1.4 mmol) of the product from example 17, step C in methanol (20 ml), pre-cooled to 0° C., was added dropwise an aqueous solution of sodium hydroxide [0.5 g, 1.2 mmol, in water (1 ml)]. The mixture was allowed to attain room temperature and stirred for 8 hours. The precipitate was filtered, washed successively with water (10 ml×2) and diethyl ether (10 ml×2), and dried under vacuo at 60° C. for 4 hours to afford 0.4 g of the title compound as a colourless solid.

¹H NMR (400 MHz, DMSOd₆)δ 3.27-3.28 (4H, t), 3.9-3.93 (4H, t), 5.37 (2H, s), 7.05-7.07 (1H, m), 7.24-7.26 (1H, d), 7.57-7.62 (3H, m), 7.69-7.79 (4H, m), 8.01-8.08 (2H, dd), 8.16-8.18 (2H, d), 8.25-8.29 (1H, d), 10.29 (1H, s);

### Example 20

### {4-[3-(6-[1, 2, 3] Thiadiazol-4-yl-quinolin-2-yl)-acryloyl]-phenyl}-carbamic acid ethyl ester (Compound No. 64)

Step A: Preparation of N′-[1-(2-methyl-quinolin-6-yl)-ethyl]-hydrazine carboxylic acid methyl ester

A solution of 1-(2-methyl-quinolin-6-yl)-ethanone (2 g, 10.8 mmol), p-toluene sulphonic acid (2 g, 10.5 mmol) and methyl carbazate (1 g, 11.8 mmol) in toluene was refluxed using Dean Stark apparatus for 12 hours. The reaction mixture was then cooled, diluted with water (20 ml) and the pH adjusted to 7 using an aqueous solution of sodium bicarbonate. The mixture was partitioned between water and ethyl acetate. The combined organic layers were successively washed with water (50 ml×2) and brine (50 ml×2), dried over anhydrous sodium sulphate and evaporated under vacuo, to obtain 2 g of the title compound as a solid residue, which was used without purification in the next step.

¹H NMR (400 MHz, DMSOd₆) δ 2.3 (3H, s), 2.65 (3H, s), 3.74 (3H, s), 7.42 (1H, d), 7.9 (1H, d), 8.22 (2H, m), 8.3 (1H, d), 10.3 (1H, s).

Step B: Preparation of 2-methyl-6-[1, 2, 3] thiadiazol-4-yl-quinoline

The product from example 20, step A was suspended in thionyl chloride (20 ml) and heated to 60° C. for 2 hours. The reaction mixture was then cooled to 10° C. and partitioned between water and ethyl acetate. The combined organic layers were successively washed with saturated bicarbonate solution (50 ml×2), water (50 ml×2) and brine (50 ml×2), dried over anhydrous sodium sulphate and evaporated under vacuo. The solid residue was triturated with hexane (50 ml×2) to afford 1.7 g of the title compound as a brown solid.

¹HNMR (400 MHz, DMSOd₆) δ 2.69 (3H, s), 7.5 (1H, d), 8.08 (1H, d), 8.39 (1H, d), 8.45 (1H, m), 8.77 (1H, d), 9.77 (1H, s);

Step C: Preparation of 6-[1, 2, 3] thiadiazol-4-yl-quinoline-2-carboxaldehyde

1.7 g (8 mmol) of the product from Example 20, step B and selenium dioxide (1.8 g, 16 mmol) in 1,4-dioxane (25 ml) was stirred at 60° C. for 4 hours. After cooling the reaction mixture, it was filtered through celite and the filtrate was partitioned between water and ethyl acetate. The combined organic layers were successively washed with water (50 ml×2) and brine (50 ml×2), dried over anhydrous sodium sulphate and evaporated under vacuo. The solid residue was triturated with hexane (50 ml×2) followed by collection of the solid by vacuum filtration to afford 0.5 g of the title compound as a brown solid.

¹H NMR (400 MHz, DMSOd₆) δ 8.12 (1H, d), 8.45 (3H, m), 8.75 (1H, d), 8.9 (1H, s), 10.28 (1H, s).

Step D: Preparation of 1-(4-amino-phenyl)-3-(6-[1, 2, 3] thiadiazol-4-yl-quinolin-2-yl)-propenone

To the solution of 0.5 g (2 mmol) of the product from example 20, step C and 4-amino acetophenone (0.3 g, 2.2 mmol) in methanol (20 ml), pre-cooled to 0° C., was added anaqueous solution of sodium hydroxide [0.16 g, 4 mmol in water (1 ml)]. The mixture was stirred at room temperature for 18 hours. After completion of the reaction, the mixture was cooled to 0° C., diluted with water (20 ml) and the pH adjusted to 7 using aqueous hydrochloric acid. The volatiles were evaporated under vacuo. The mixture was partitioned between water and ethyl acetate. The combined organic layers were successively washed with water (50 ml×2) and brine (50 ml×2), dried over anhydrous sodium sulphate and evaporated under vacuo. The solid residue was triturated with hexane (50 ml×2) followed by collection of the solid by vacuum filtration to afford 0.5 g of the title compound as a brown solid.

¹H NMR (400 MHz, DMSOd₆) δ 6.21 (2H, bs), 6.67 (1H, d), 7.78 (1H, d), 7.98 (2H, d), 8.23 (2H, t), 8.3 (1H, d), 8.53 (1H, d), 8.54 (1H, m), 8.55 (1H, d), 8.8 (1H, d), 9.83 (1H, s).

Step E: Preparation of {4-[3-(6-[1,2,3]thiadiazol-4-yl-quinolin-2-yl)-acryloyl]-phenyl}carbamic acid ethyl ester

To the solution of 0.2 g (0.5 mmol) of the product from example 20, step D in dry tetrahydrofuran (20 ml) containing N-ethyl diisopropyl amine (0.2 g, 1.6 mmol), pre-cooled to 0° C., was added dropwise ethyl chloroformate (0.09 g, 0.8 mmol). The reaction mixture was stirred at room temperature for 3 hours. The mixture was partitioned between water and ethyl acetate. The combined organic layers were successively washed with water (50 ml×2) and brine (50 ml×2), dried over anhydrous sodium sulphate and evaporated under vacuo. The solid residue was triturated with hexane (50 ml×2) followed by collection of the solid by vacuum filtration to afford 0.4 g of the title compound as a solid.

¹H NMR (400 MHz, DMSOd₆) δ 1.26-1.29 (3H, t), 4.16-421 (2H, m), 7.69-7.71 (2H, d), 7.82-7.86 (1H, d), 8.16-8.19 (2H, d), 8.23-8.29 (2H, m), 8.34-8.38 (1H, d), 8.54-8.57 (1H, dd), 8.61-8.63 (1H, d), 8.87 (1H, s), 9.84 (1H, s), 10.15 (1H, s);

MS, m/z 431

### Example 21

### {4-[3-(6-Morpholin-4-yl-pyridin-2-yl)-acryloyl]-phenyl}-carbamic acid 2-morpholin-4-yl-ethyl ester (Compound No. 67)

A solution of 0.2 g (0.55 mmol) of the product from example 11, Step C and 2-Morpholin-4-yl-ethanol (0.12 g, 0.9 mmol) in toluene (20 ml) was refluxed for 16 hours. The reaction mixture was partitioned between water and ethyl acetate. The combined organic layers were successively washed with water (25 ml×2) and brine (25 ml×2), dried over anhydrous sodium sulphate and evaporated under vacuo. The solid residue was purified by column chromatography over silica gel using 40% ethyl acetate in hexane as the eluent to afford 0.06 g of the title compound as a yellow solid.

¹H NMR (400 MHz, DMSOd₆) δ 2.55-2.57 (4H, t), 2.7-2.73(2H, t), 3.62-3.64 (4H, t), 3.75-3.77 (4H, t), 3.88-3.9 (4H, t), 4.33-4.37 (2H, t), 6.7-6.73 (1H, d), 6.86-6.88 (1H, d), 6.97 (1H, s), 7.53-7.58 (3H, m), 7.63-7.67 (1H, d), 7.98-8.02 (1H, d), 8.06-8.08 (2H, d);

MS, m/z 467

### Example 22

### Hydrochloride salt of Compound No. 143, i.e., {4-[3-(6-Morpholin-4-yl-pyridin-2-yl)-acryloyl]-phenyl}-carbamic acid piperidin-4-yl ester

Step A: Preparation of {4-[3-(6-Morpholin-4-yl-pyridin-2-yl)-acryloyl]-phenyl}-carbamic acid piperidin-4-yl ester

A solution of 0.25 g (0.68 mmol) of the product from example 11, Step C and 4-hydroxy-piperidine-1-carboxylic acid tert-butyl ester (0.15 g, 0.66 mmol) in dry toluene (20 ml) was refluxed for 16 hours. The reaction mixture was partitioned between water and ethyl acetate. The combined organic layers were successively washed with water (25 ml×2) and brine (25 ml×2), dried over anhydrous sodium sulphate and evaporated under vacuo. The solid residue was purified by column chromatography over silica gel using ethyl acetate as the eluent to provide 0.13 g of the title compound as a yellow solid.

Step B: Preparation of hydrochloride salt of {4-[3-(6-Morpholin-4-yl-pyridin-2-yl)-acryloyl]-phenyl}-carbamic acid piperidin-4-yl ester

The solid from Example 22, step A was added to a cooled solution (0° C.) of acetonitrile-hydrochloric acid (10%, 2 ml) and was stirred for 2 hours. The precipitate was filtered, washed with diethyl ether (50 ml×2) and dried under vacuo at 60° C. for 4 hours to afford 0.08 g of the title compound as a yellow solid.

¹H NMR (400 MHz, DMSOd₆) δ 1.6-1.62 (2H, m), 1.9-1.93 (2H, m), 2.84-2.91 (4H, m), 3.1-3.19 (4H, m), 3.59-3.62 (1H, t), 3.7-3.75 (4H, m), 4.4 (1H, bs), 7.04-7.52 (2H, m), 7.63-7.73 (1H, m), 8.05-8.09 (1H, t), 8.96-9.09 (4H, d), 9.3-9.45 (1H, d), 10.3 (1H, s);

MS, m/z 472

### Example 23

### {4-[3-(6-Morpholin-4-yl-pyridin-2-yl)-acryloyl]-phenyl}-carbamic acid 2-(pyridine-2-sulfonyl)-ethyl ester (Compound No. 156)

Step A: Preparation of 2-(pyridin-2-ylsulfanyl)-ethanol

A solution of pyridin-2-thiol (0.5 g, 4.5 mmol), 2-bromo-ethanol (0.84 g, 6.7 mmol) and potassium carbonate (2.5 g, 18 mmol) in acetonitrile (50 ml) was refluxed for 20 hours. The mixture was partitioned between water and ethyl acetate. The combined organic layers were successively washed with water (25 ml×2) and brine (25 ml×2), dried over anhydrous sodium sulphate and evaporated under vacuo to afford 0.15 g of the title compound as yellow oil, which was used without further purification in the next step.

¹H NMR (400 MHz, DMSOd₆) δ 2.76 (2H, t), 3.13 (2H, t), 7.07-7.11 (1H, m), 7.2-7.3 (1H, m), 7.6-7.64 (1H, m), 8.4-8.42 (1H, m), 6.2 (1H, bs).

Step B: Preparation of {4-[3-(6-Morpholin-4-yl-pyridin-2-yl)-acryloyl]-phenyl}-carbamic acid 2-(pyridine-2-sulfanyl)-ethyl ester

A solution of 0.2 g (0.55 mmol) of the product from example 11, Step C and 0.1 g (0.66 mmol) of the product from Example 23, step A in toluene (20 ml) was refluxed for 16 hours. The reaction mixture was partitioned between water and ethyl acetate. The combined organic layers were successively washed with water (25 ml×2) and brine (25 ml×2), dried over anhydrous sodium sulphate and evaporated under vacuo. The solid residue was purified by column chromatography over silica gel using 60% ethyl acetate in hexane as the eluent to afford 0.1 g of the title compound as a yellow solid.

¹H NMR (400 MHz, DMSOd₆)δ 3-3.11 (2H, t), 3.28 (2H, t), 3.55-3.56 (4H, t), 3.72-3.75 (4H, t), 6.92-6.95 (1H, d), 7.11-7.14 (1H, d), 7.35-7.37 (1H, d), 7.51-7.59 (3H, m), 7.62-7.67 (2H, m), 7.99-8.03 (3H, m), 8.08-8.14 (1H, m), 8.43-8.45 (1H, d), 9.08-9.13 (1H, d).

Step C: Preparation of {4-[3-(6-Morpholin-4-yl-pyridin-2-yl)-acryloyl]-phenyl}-carbamic acid 2-(pyridine-2-sulfonyl)-ethyl ester

To 0.07 g (0.14 mmol) of the product from example 23, step C in methanol (2 ml), pre-cooled to 0° C., was added dropwise a solution of oxone [0.04 g, 0.07 mmol in water (1 ml)]. The reaction mixture was stirred at 0° C. for 30 minutes. The mixture was diluted with water (10 ml) and the volatile were evaporated under vacuo. The precipitate was filtered, washed successively with water (10 ml×2) and hexane (10 ml×2), and dried under vacuo at 60° C. for 4 hours to afford 0.05 g of the title compound as a yellow solid.

¹H NMR (400 MHz, DMSOd₆) δ 3.25-3.29 (2H, t), 3.36 (2H, t), 3.55-3.56 (4H, t), 3.72-3.75 (4H, t), 6.92-6.95 (1H, d), 7.11-7.14 (1H, d), 7.35-7.37 (1H, d), 7.51-7.59 (3H, m), 7.62-7.67 (2H, m), 7.99-8.03 (3H, m), 8.08-8.14 (1H, m), 8.43-8.45 (1H, d), 9.08-9.13(1H, d);

MS, m/z 522

### Example 24

### N-{4-[3-(2-Morpholin-4-yl-quinolin-3-yl)-acryloyl]-phenyl}-oxalamide (Compound No. 77)

Step A: Preparation of 2-morpholin-4-yl-quinoline-3-carboxaldehyde

A suspension of 2-chloro-quinoline-3-carboxaldehyde (3 g, 15.6 mmol) in morpholine (25 ml) was refluxed for 30 hours. After cooling the reaction mixture, the precipitate was filtered, successively washed with water (50 ml×2) and diethyl ether (50 ml×2), and dried it under vacuo for 4 hours to obtain 2 g of the title compound as a yellow solid.

¹H NMR (400 MHz, DMSOd₆) δ 3.32 (4H, t), 4.03 (4H, t), 7.5 (1H, d), 7.65 (1H, m), 7.79 (1H, m), 8.19 (1H, d), 8.22 (1H, s), 10.19 (1H, s).

Step B: Preparation of 1-(4-amino-phenyl)-3-(2-morpholin-4-yl-quinolin-3-yl)-propenone

To the solution of 2 g (8.2 mmol) of the product from example 24, step A and 4-amino acetophenone (1.2 g, 8.2 mmol) in methanol (20 ml), pre-cooled to 0° C., was added an aqueous solution of sodium hydroxide [0.6 g, 16.4 mmol in water (2 ml)]. The mixture was stirred at room temperature for 16 hours. After completion of reaction, the mixture was cooled to 0° C., diluted with water (20 ml) and the pH adjusted to 7 using aqueous hydrochloric acid. The volatiles were evaporated under vacuo. The mixture was partitioned between water and ethyl acetate. The combined organic layers were successively washed with water (50 ml×2) and brine (50 ml×2), dried over anhydrous sodium sulphate and evaporated under vacuo. The solid residue was purified by column chromatography on silica gel using the 30% ethyl acetate in hexane as the eluent to afford 1.2 g of the title compound as a yellow solid.

¹H NMR (400 MHz, DMSOd₆) δ 3.8 (4H, t), 3.9 (4H, t), 6.64-6.67 (1H, s), 7.6 (1H, m), 7.8 (1H, d), 7.82 (1H, d), 7.9 (1H, m), 8.03 (2H, d), 8.18 (2H, m), 8.3 (2H, d), 8.6 (1H, d).

Step C: Preparation of N-{4-[3-(2-morpholin-4-yl-quinolin-3-yl)-acryloyl]-phenyl}-oxalamic acid ethyl ester

To 1.2 g (3.3 mmol) of the product from example 24, step B in dry dichloromethane (30 ml) containing triethylamine (0.5 g, 5 mmol), pre-cooled to 0° C., was added ethyl oxalyl chloride (0.5 g, 4 mmol). The reaction mixture was stirred at room temperature for 4 hours. The mixture was partitioned between water and ethyl acetate. The combined organic layers were successively washed with water (50 ml×2) and brine (50 ml×2), dried over anhydrous sodium sulphate and evaporated under vacuo. The solid residue was triturated with hexane (25 ml×2) to afford 1 g of the title compound as a brown solid.

¹H NMR (400 MHz, DMSOd₆) δ 1.32 (3H, t), 3.8 (4H, t), 3.9 (4H, t), 4.3 (2H, m), 7.6 (1H, m), 7.8 (1H, d), 7.82 (1H, d), 7.9 (1H, m), 8.03 (2H, d), 8.18 (2H, m), 8.3 (2H, d), 8.6(1H, dd), 11.2(1H, s).

Step D: Preparation of N-{4-[3-(2-Morpholin-4-yl-quinolin-3-yl)-acryloyl]-phenyl}-oxalamide

To 0.15 g (0.32 mmol) of the product from example 24, step C was added ammonia solution (20 ml) and stirred at room temperature for 6 hours. The precipitate was filtered, successively washed with water (20 ml×2) and diethyl ether (20 ml×2), dried under vacuo at 60° C. for 4 hours to afford 25 mg of the title compound as a brown solid.

¹H NMR (400 MHz, DMSOd₆) δ 3.29 (4H, bs), 3.82 (4H, bs), 7.46-7.49 (1H, t), 7.7-7.72 (1H, t), 7.78-7.83 (2H, t), 7.87-7.91 (2H, t), 8.07-8.11 (3H, t), 8.2-8.23 (2H, d), 8.4 (1H, s), 8.87 (1H, s), 10.99 (1H, s);

MS, m/z 431

### Example 25

### 2-Morpholin-4-yl-N-{4-[3-(4-morpholin-4-yl-quinolin-2yl-acryloyl)-phenyl}-2-oxo-acetamide (Compound No. 78)

Step A: Preparation of 1-(4-amino-phenyl)-3-(4-morpholin-4-yl-quinolin-3-yl)-propenone

To 4-morpholine-4-yl-quinoline-2-carboxaldehyde (1 g, 4.1 mmol), prepared as shown in example 19, step B and 4-amino acetophenone (0.56 g, 4.1 mmol) in methanol (20 ml), cooled to 0° C., was added an aqueous solution of sodium hydroxide [0.33 g, 8.2 mmol in water (2 ml)]. The mixture was stirred at room temperature for 16 hours. After completion of reaction, the mixture was cooled to 0° C., diluted with water (20 ml) and aqueous hydrochloric acid was added to adjust the pH to 7. The volatiles were evaporated under vacuum. The mixture was partitioned between water and ethyl acetate. The combined organic layers were successively washed with water (50 ml×2) and brine (50 ml×2), dried over anhydrous sodium sulphate and evaporated under vacuum. The residue was purified by column chromatography on silica gel using the 30% ethyl acetate in hexane as the eluent to afford 0.6 g of the title compound as a yellow solid.

¹H NMR (400 MHz, DMSOd₆) δ 3.8 (4H, t), 3.9 (4H, t), 6.2 (2H, bs), 6.6 (2H, d), 7.5 (2H, m), 7.6 (1H, s), 7.7 (1H, m), 7.9 (2H, d), 8.02 (1H, d), 8.06 (1H, d), 8.25 (1H, d).

Step B: Preparation of N-{4-[3-(4-morpholin-4-yl-quinolin-3-yl)-acryloyl]-phenyl}-oxalamic acid ethyl ester

To 0.6 g (1.7 mmol) of the product of example 25, step A in dry dichloromethane (30 ml) containing triethylamine (0.5 g, 5 mmol), cooled to 0° C. and to it was added drop wise ethyl oxalyl chloride (0.28 g, 2.1 mmol). The reaction mixture was stirred at room temperature for 4 hours. The mixture was partitioned between water and ethyl acetate. The combined organic layers were successively washed with water (50 ml×2) and brine (50 ml×2), dried over anhydrous sodium sulphate and evaporated under vacuo. Trituration of the residue in hexane (20 ml×3) followed by the collection of the solid by vacuum filtration provided 0.55 g of the title compound as a yellow solid.

¹H NMR (400 MHz, DMSOd₆) δ 1.32 (3H, t), 3.8 (4H, t), 3.9 (4H, t), 4.3 (2H, m), 7.6 (1H, m), 7.8 (1H, d), 7.82 (1H, d), 7.9 (1H, m), 8.03 (2H, d), 8.18 (2H, m), 8.3 (2H, d) 8.6 (1H, dd), 11.2 (1H, s).

Step C: Preparation of 2-Morpholin-4-yl-N-{4-[3-(4-morpholin-4-yl-quinolin-2yl-acryloyl)-phenyl}-2-oxo-acetamide

To 0.2 g (0.4 mmol) of the product from example 25, step B in xylene (20 ml), was added morpholine (1 g, 11.5 mmol) and refluxed for 12 hours. The mixture was partitioned between water and ethyl acetate. The combined organic layers were successively washed with water (50 ml×2) and brine (50 ml×2), dried over anhydrous sodium sulphate and evaporated under vacuo. The solid residue was purified by column chromatography on silica gel using 50% ethyl acetate in hexane to obtain 6.8 g of the title compound as a yellowish brown solid.

¹H NMR (400 MHz, DMSOd₆) δ 3.48-3.56 (4H, m), 3.6-3.67 (4H, m), 3.85-3.91 (8H, d), 7.58-7.65 (1H, m), 7.77-7.82 (3H, m), 7.89-7.92 (2H, ,d), 7.97-8.13 (3H, m), 8.22-8.24 (2H, d), 11.25 (1H, s);

MS, m/z 501

### Example 26

### N-(2-Morpholin-4-yl-ethyl)-N′-[4-(3-quinoxalin-2-yl-acryloyl)-phenyl]-oxalamide (Compound No. 130)

Step A: Preparation of quinoxalin-2-carboxaldehyde

To 2-methyl-quinoxalin-2-ol (2 g, 12.4 mmol) in 1,4-dioxane (50 ml) was added selenium dioxide (4 g, 37 mmol) and the mixture was heated to 60° C. for 4 hours. After cooling the reaction mixture, it was filtered over celite and partitioned between water and ethylacetate. The combined organic phases were successively washed with water (50 ml×2) and brine (50 ml×2), dried over anhydrous sodium sulphate and evaporated under vacuo to afford 1.3 g of the crude title compound as a brown solid, which was used without purification in the next step.

¹H NMR (400 MHz, DMSOd₆) δ 7.98-8.09 (3H, m), 8.21-8.24 (1H, m), 8.28-8.31 (1H, m), 10.19 (1H, s).

Step B: Preparation of 1-(4-amino-phenyl)-3-quinoxalin-2-yl-propenone

To the solution of 1.3 g (8.2 mmol) of the product from example 26, step A in methanol (40 ml), pre-cooled to 0° C., was added dropwise an aqueous solution of sodium hydroxide [0.65 g, 16.4 mmol in water (2 ml)]. The mixture was stirred at room temperature for 18 hours. After completion of reaction, the mixture was cooled to 0° C., diluted with water (20 ml) and the pH adjusted to 7 using aqueous hydrochloric acid. The volatiles were evaporated under vacuo. The precipitate was isolated by filtration with a Buchner funnel and successively washed with water (20 ml×2) and brine (10 ml×2), and dried under vacuo at 60° C. to afford 1.5 g of the title compound as a yellow solid.

¹H NMR (400 MHz, DMSOd₆) δ 6.27 (2H, bs), 6.6 (2H, d), 7.5 (1H, d), 7.56 (1H, m), 7.69 (2H, m), 7.8-7.82 (2H, m), 7.83-7.84 (2H, q), 7.91-7.92 (1H, d).

Step C: Preparation of N-{4-(3-quinoxalin-2-yl-acryloyl)-phenyl}-oxalamic acid ethyl ester

To 1.5 g (5.5 mmol) of the product from example 26, step B in dry dichloromethane (40 ml), pre-cooled to 0° C., was added ethyl oxalyl chloride (1.1 g, 8 mmol). The reaction mixture was stirred at room temperature for 12 hours. The mixture was partitioned between water and ethyl acetate. The combined organic layers were successively washed with water (50 ml×2) and brine (50 ml×2), dried over anhydrous sodium sulphate and evaporated under vacuo. The solid residue was triturated with hexane (25 ml×2) to afford 1.4 g of the title compound as a brown solid.

¹H NMR (400 MHz, DMSOd₆) δ 1.32 (3H, t), 4.32 (2H, q), 6.61 (2H, d), 7.5-7.52 (1H, d), 7156 (1H, m), 7.69-7.71 (2H, m), 7.81-7.83 (2H, m), 7.83-7.84 (2H, d), 8.09-8.11 (1H, d), 11.2 (1H, bs).

Step D: Preparation of N-(2-Morpholin-4-yl-ethyl)-N,-[4-(3-quinoxalin-2-yl-acryloyl)-phenyl]-oxalamide

The ester from example 26, step C (0.2 g, 0.53 mmol) was suspended in xylene (20 ml). To it, 2-morpholine-4-yl-ethylamine (5.2 g, 4 mmol) was added and refluxed for 12 hours. The reaction was partitioned between water and ethyl acetate. The combined organic layers were successively washed with water (50 ml×2) and brine (50 ml×2) and dried under vacuo. The solid residue was purified by column chromatography on silica gel using 60% ethyl acetate in hexane as the eluent to provide 0.025 g of the title compound as a solid.

¹H NMR (400 MHz, DMSOd₆) δ 2.41 (4H, bs), 2.54 (2H, bs), 3.57 (4H, bs), 3.64-3.66 (2H, t), 7.77-7.8 (2H, m), 7.92-8.04 (8H, m), 8.91 (1H, bs), 8.98 (1H, s), 10.98 (1H, s);

MS, m/z 460

### Example 27

### 2-Morpholin-4-yl-N-{4-[3-(6-morpholin-4-yl-pyridin-2-yl)-acryloyl]phenyl}-2-oxo-acetamide (Compound No. 83)

Step A: Preparation of 6-morpholin-4-yl-pyridine-2-carboxaldehyde

A solution of 6-bromo-pyridine-2-carboxaldehyde (1.9 g, 10 mmol), morpholine (1.75 g, 20 mmol) and potassium carbonate (3 g, 22 mmol) in acetonitrile (20 ml) was refluxed for 20 hours. The reaction mixture was cooled to room temperature, diluted with water (50 ml) and the pH adjusted to 7. The mixture was partitioned between water and ethyl acetate. The combined organic layers were successively washed with water (25 ml×2) and brine (25 ml×2), dried over anhydrous sodium sulphate and evaporated under vacuo. The gummy residue was purified by column chromatography over silica gel using 40% ethyl acetate in hexane as the eluent to afford 1.5 g of the title compound as brown liquid.

¹H NMR (400 MHz, DMSOd₆) δ 3.55-3.58 (4H, t), 3.91-3.94 (4H, t), 7.15-7.18 (1H, d), 7.56-7.61 (1H, d), 7.65-7.68 (1H, t), 9.98 (1H, s).

Step B: Preparation of 1-(4-amino-phenyl)-3-(6-morpholin-4-yl-pyridin-2-yl)-propenone

To 1.5 g (7.8 mmol) of the product from example 27, step A and 4-amino acetophenone (1 g, 7.8 mmol) in methanol, pre-cooled to 0° C., was added dropwise an aqueous solution of sodium hydroxide [{0.6 g, 15.5 mmol, in water (2 ml)]. The reaction mixture was stirred for 16 hours. The mixture was then diluted with water (20 ml) and the pH adjusted to 7 using aqueous solution of hydrochloric acid. The volatiles were evaporated under vacuo. The mixture was partitioned between water and ethyl acetate. The combined organic layers were successively washed with water (25 ml×2) and brine (25 ml×2), dried over anhydrous sodium sulphate and evaporated under vacuo. The residue was purified by column chromatography over silica gel using 60% ethyl acetate in hexane as the eluent to afford 0.6 g of the title as a yellow solid.

¹H NMR (400 MHz, DMSOd₆) δ 3.52-3.54 (4H, m), 3.76-3.8(4H, t), 6.09 (1H, s), 6.54-6.56 (1H, d), 6.59-6.61 (2H, d), 6.97(1H, s), 7.52-7.6 (1H, d), 7.68-7.72 (1H, d), 7.89-7.91 (2H, d), 8.13-8.16 (1H, m), 8.41(1H, s).

Step C: Preparation of N-{4-[3-(6-morpholin-4-yl-pyridin-2-yl)-acryloyl]-phenyl}-oxalamic acid ethyl ester

To 0.6 g (2 mmol) of the product from example 27, step B in dichloromethane, pre-cooled to 0° C., was added ethyl oxalyl chloride (1.2 g, 9 mmol) dropwise. The reaction mixture was stirred at room temperature for 30 minutes. The precipitate was filtered, successively washed with water (25 ml×2) and diethyl ether (25 ml×2), and evaporated under vacuo to afford 0.5 g of the title compound as a solid.

Step D: Preparation of 2-Morpholin-4-yl-N-{4-[3-(6-morpholin-4-yl-pyridin-2-yl)-acryloyl]phenyl}-2-oxo-acetamide

To 0.2 g (0.5 mmol) of the product from example 27, step C and morpholine (1 g, 12 mmol) in xylene (20 ml) was refluxed for 8 hours. The mixture was partitioned between water and ethyl acetate. The combined organic layers were successively washed with water (25 ml×2) and brine (25 ml×2), dried over anhydrous sodium sulphate and evaporated under vacuo. The residue was purified by column chromatography over silica gel using 80% ethyl acetate in hexane as the eluent to afford 0.6 g of the title compound as brown solid.

¹H NMR (400 MHz, DMSOd₆) δ 3.51-3.57 (8H, m), 3.61-3,67 (4H, m), 3.72-3.75 (4H, t), 6.94-6.96 (1H, d), 7.12-7.14 (1H, d), 7.55-7.59 (1H, d), 7.63-7.67 (1H, t), 7.85-7.87 (2H, d), 7.99-8.03 (1H, d), 8.09-8.12 (2H, d), 11.2 (1H, s);

MS, m/z 449

The following representative compounds as shown in Table 1 may be prepared by following the synthetic routes as described above:

TABLE 1

| Ex | Comp | | |
|---|---|---|---|
| No. | No. | ¹H NMR(400MHz, δ, DMSOd₆) | Mass, m/z |
| 28 | 2 | 2.43(2H, bs), 2.54(2H, bs), 3.61(2H, bs), 3.93(2H, bs), 7.65-7.73(3H, | 385 |
| | | m), 7.81-7.89(2H, m), 8.02-8.05(1H, d), 8.09-8.11(1H, d), | |
| | | 8.23-8.28(3H, t), 8.32-8.37(1H, d), 8.49-8.52(1H, d) | |
| 29 | 3 | 2.21(3H, s), 2.28(2H, bs), 2.39(2H, bs), 3.33(2H, bs), 3.66(2H, bs), | 386 |
| | | 7.59-7.62(2H, d), 7.65-7.71(1H, t), 7.81-7.89(2H, m), 8.02-8.05(1H, d), | |
| | | 8.08-8.11(1H, d), 8.22-8.25(3H, m), 8.3-8.35(1H, d), 8.49-8.51(1H, | |
| | | d) | |
| 30 | 4 | 1.77-1.87(4H, m), 3.55-3.63(2H, m), 4.37-4.4(1H, m), 7.56-7.58(1H, | 400 |
| | | d), 7.65-7.69(1H, t), 7.77-7.89(4H, m), 8.02-8.05(1H, d), | |
| | | 8.08-8.1(1H, d), 8.18-8.21(1H, d), 8.23-8.26(3H, d), 8.31-8.37(1H, dd), | |
| | | 8.49-8.51(1H, d) | |
| 31 | 5 | 1.18-1.19(6H, d), 1.54(2H, bs), 1.77(1H, bs), 1.92(1H, bs), | 457 |
| | | 2.59-2.67(1H, m), 2.92-2.98(1H, m), 3.11-3.16(1H, m), 3.49-3.52(1H, d), | |
| | | 4.34-4.37(1H, d), 4.88-4.96(1H, m), 7.6-7.62(2H, d), 7.65-7.69(1H, t), | |
| | | 7.81-7.88(2H, m), 8.02-8.04(1H, d), 8.08-8.1(1H, d), 8.2-8.24(3H, d), | |
| | | 8.31-8.35(1H, d), 8.49-8.51(1H, d) | |
| 32 | 6 | 2.64-2.67(2H, m), 2.76(2H, bs), 2.98(1H, bs), 3.28(2H, bs), 3.57(2H, | 372 |
| | | bs), 7.55-7.61(2H, d), 7.65-7.69(1H, t), 7.81-7.89(2H, m), | |
| | | 7.99-8.04(1H, d), 8.08-8.10(1H, d), 8.20-8.25(3H, m), 8.29-8.37(1H, m), | |
| | | 8.47-8.51(1H, m) | |
| 33 | 7 | 1.98-2.03(3H, d), 3.29-3.35(2H, d), 3.43(2H, bs), 3.56-3.69(4H, t), | 414 |
| | | 7.59-7.67(3H, m), 7.8-7.84(2H, d), 7.97-7.99(1H, d), 8.04-8.1(2H, t), | |
| | | 8.16-8.24(3H, m), 8.43-8.45(1H, d) | |
| 34 | 10 | 2.62(2H, bs), 2.72(2H, bs), 3.53-3.57(2H, m), 3.9(2H, bs), | 389 |
| | | 7.62-7.71(3H, m), 7.81-7.89(2H, m), 8.00-8.05(1H, d), 8.08-8.11(1H, d), | |
| | | 8.23-8.26(3H, d), 8.32-8.36(1H, d), 8.49-8.52(1H, d) | |
| 35 | 11 | 1.82-1.91(4H, m), 3.37-3.4(2H, t), 3.48-3.52(2H, t), 7.65-7.73(3H, | 357 |
| | | m), 7.8-7.88(2H, m), 8.02-8.04(1H, d), 8.08-8.11(1H, d), | |
| | | 8.22-8.26(3H, d), 8.32-8.36(1H, t), 8.49-8.52(1H, d) | |
| 36 | 12 | 1.47(2H, bs), 1.62(4H, bs), 3.27-3.34(2H, m), 3.62(2H, bs), | 371 |
| | | 7.58-7.6(2H, d), 7.65-7.69(1H, t), 7.8-7.89(2H, d), 8.02-8.04(1H, d), | |
| | | 8.08-8.1(1H, d), 8.22-8.26(3H, m), 8.32-8.36(1H, d), 8.49-8.52(1H, d) | |
| 37 | 14 | 0.86-0.88(6H, d), 1.29-1.35(2H, m), 1.54-1.61(1H, m), 2.29-2.33(4H, | 442 |
| | | t), 2.44-2.5(2H, m), 3.25-3.4(2H, m), 3.64(2H, s), 7.59-7.61(2H, d), | |
| | | 7.65-7.69(1H, t), 7.8-7.89(2H, m), 8.02-8.04(1H, d), 8.08-8.1(1H, d), | |
| | | 8.22-8.25(3H, d) 8.31-8.35(1H, d), 8.49-8.51(1H, d) | |
| 38 | 15 | 3.2-3.34(4H, m), 3.46(2H, s), 3.79(2H, s), 6.81-6.99(3H, m), | 482 |
| | | 7.21-7.26(1H, t), 7.66-7.68(3H, t), 7.82-7.9(2H, m), 8.02-8.1(2H, d), | |
| | | 8.23-8.27(3H, t), 8.32-8.37(1H, d), 8.49-8.52(1H, d) | |
| 39 | 16 | 2.98-3.09(4H, d), 3.5(2H, s), 3.84(2H, s), 7.18(1H, t), 7.35(2H, d), | 517 |
| | | 7.67-7.69(4H, d), 7.8-7.89(2H, m), 8.02-8.1(2H, m), 8.25(3H, t), 8.32-8.36(1H, | |
| | | d) | |
| 40 | 17 | 2.04(3H, s), 2.38(1H, s), 3.72-3.78(3H, m), 4.02-4.15(1H, d), 4.36(1H, | 519 |
| | | s), 7.27-7.3(2H, d), 7.58-7.63(2H, t), 7.65-7.69(1H, t), 7.73-7.75(2H, | |
| | | d), 7.8-7.89(2H, m), 7.98-8.15(2H, m), 8.22-8.37(4H, m), | |
| | | 8.49-8.52(1H, d), 10.02(1H, s) | |
| 41 | 18 | 3.37-3.39(3H, t), 7.28-7.3(3H, d), 7.59-7.62(1H, d), 7.64-7.66(3H, d), | 560 |
| | | 7.83-7.86(1H, d), 7.91-7.94(3H, d), 7.98-8.01(2H, d), 9.87(1H, s) | |
| 42 | 19 | 1.85-1.87(4H, m), 3.27-3.34(4H, m), 3.4-3.42(4H, m), 3.92-3.93(4H, | 457 |
| | | m), 7.58-7.66(2H, m), 7.73-7.82(4H, m), 8.01-8.08(2H, m), | |
| | | 8.11-8.13(2H, d), 8.27-8.32(1H, d), 8.62(1H, s) | |
| 43 | 20 | 1.82-1.85(2H, t), 2.03(2H, bm), 3.47-3.77(10H, m), 4.65-4.71(1H, d), | 470 |
| | | 7.64-7.69(3H, m), 7.81-7.89(2H, m), 8.02-8.11(2H, dd), | |
| | | 8.24-8.26(3H, t), 8.32-8.36(1H, d), 8.5-8.52(1H, d) | |
| 44 | 21 | 3.43(2H, bs), 3.74-3.8(6H, bd), 6.64-6.65(1H, d), 7.04-7.05(1H, d), | 466 |
| | | 7.66-7.69(3H, d), 7.8-7.92(3H, m), 8.02-8.11(2H, dd), 8.23-8.27(3H, | |
| | | m), 8.32-8.36(1H, d), 8.49-8.52(1H, d) | |
| 45 | 22 | 3.35-3.45(6H, d), 3.77(2H, s), 6.83-6.84(2H, d), 7.65-7.69(3H, t), | 449 |
| | | 7.8-7.87(2H, m), 8.02-8.1(2H, dd), 8.18-8.19(2H, d), 8.23-8.27(3H, t), | |
| | | 8.32-8.36(1H, d), 8.49-8.51(1H, d) | |
| 46 | 23 | 1.33-1.37(3H, t), 3.23-3.3(4H, t), 3.91-3.93(4H, t), 4.15-4.21(2H, | 432 |
| | | m), 7.57-7.61(2H, m), 7.68-7.79(4H, m), 8.01-8.08(2H, m), | |
| | | 8.15-8.17(2H, d), 8.25-8.29(1H, d), 10.15(1H, s) | |
| 47 | 24 | 1(3H, s), 1.02(3H, s), 2-2.04(1H, m), 3.3-3.32(4H, t), 4-4.05(6H, m), | 460 |
| | | 6.88(1H, s), 7.12(1H, s), 7.5-7.59(3H, m), 7.7-7.75(1H, m), | |
| | | 7.88-7.92(1H, d), 8.02-8.04(1H, d), 8.12-8.2(4H, m) | |
| 48 | 25 | 3.23-3.27(4H, m), 3.87-3.96(4H, m), 7.32-7.35(1H, m), 7.57-7.59(1H, m), | 547 |
| | | 7.63(1H, s), 7.65-7.73(4H, m), 7.75-7.77(1H, m), 7.8(1H, s), | |
| | | 7.92-7.95(1H, d), 8.01-8.08(2H, m), 8.18-8.2(2H, d), 8.29-8.32(2H, m), 9.9(1H, | |
| 49 | 26 | 3.26-3.28(4H, m), 3.9(4H, m), 7.48-7.51(3H, m), 7.56-7.63(4H, m), | 544 |
| | | 7.7-7.76(2H, m), 7.8-7.86(3H, m), 8-8.07(4H, m), 8.22-8.27(1H, d), 9.12(1H, | |
| | | m) | |
| 50 | 27 | 1.29-1.34(3H, m), 3.2-3.35(4H, m), 3.91-3.93(4H, m), 4.25-4.32(2H, m), | 551 |
| | | 7.57-7.64(4H, m), 7.69-7.8(4H, m), 7.89-7.97(2H, m), 8.02-8.08(2H, m) | |
| | | 8.17-8.22(2H, d), 8.28-8.32(1H, d), 9.28-9.35(2H, t) | |
| 51 | 28 | 0.96-1(3H, t), 2.33-2.37(4H, m), 2.42-2.5(2H, m), 3.31(2H, bs), | 400 |
| | | 3.64(2H, bs), 7.57-7.59(2H, d), 7.64-7.68(1H, t), 7.8-7.86(2H, m), | |
| | | 8-8.02(1H, d), 8.07-8.09(1H, d), 8.14-8.16(1H, d), 8.19-8.21(2H, d), | |
| | | 8.25-8.29(1H, d), 8.46-8.48(1H, d) | |
| 52 | 29 | 1.27-1.33(3H, t), 1.61-1.67(2H, bs), 1.82(4H, bs), 3.23(4H, bs), | 430 |
| | | 4.15-4.2(2H, m), 7.55-7.59(2H, t), 7.68-7.77(4H, m), 7.98-8(2H, d), | |
| | | 8.14-8.17(2H, d), 8.23-8.27(1H, d), 10.15(1H, s) | |
| 53 | 30 | 0.94-0.96(6H, d), 1.67(2H, bs), 1.82(4H, bs), 1.92-1.97(1H, m), | 458 |
| | | 3.23(4H, bs), 3.91-3.93(2H, d), 7.56-7.59(2H, t), 7.69-7.78(4H, m), | |
| | | 7.98-8(2H, d), 8.15-8.17(2H, d), 8.23-8.27(1H, d), 10.15(1H, s) | |
| 54 | 31 | 1.25-1.28(3H, t), 1.9(4H, bs), 3.62(4H, bs), 4.11-4.2(2H, m), | 416 |
| | | 7.25-7.28(1H, m), 7.55-7.59(2H, m), 7.65-7.68(2H, d), 7.76-7.78(1H, d), | |
| | | 7.82-7.86(1H, d), 7.99-8.04(1H, d), 8.14-8.17(2H, d), 8.57(1H, s), 10.12(1H, s) | |
| 55 | 33 | 1.91(4H, bs), 3.63(4H, bs), 7.04-7.06(1H, m), 7.27(1H, m), | 464 |
| | | 7.52-7.59(3H, m), 7.65-7.66(2H, d), 7.73-7.79(4H, m), 7.92-7.95(2H, d), | |
| | | 8.18-8.2(1H, d), 8.31(1H, m), 9.57-9.59(1H, d), 10.81-10.85(1H, m) | |
| 56 | 34 | 2.2(3H, s), 2.27(2H, bs), 2.38(2H, bs), 2.63(3H, s), 2.67(2H, m), | 432 |
| | | 3.65(2H, bs), 7.59-7.61(2H, d), 7.67-7.69(1H, dd), 7.78-7.86(2H, t), | |
| | | 7.96-7.99(1H, d), 8.19-8.24(3H, t), 8.26-8.31(1H, d), 8.36-8.39(1H, d) | |
| 57 | 36 | 2.62-2.73(4H, d), 3.3(2H, m), 3.53(2H, bs), 3.9-3.91(3H, d), | 479 |
| | | 3.99-4.01(6H, t), 7.33(1H, s), 7.61-7.64(2H, d), 7.81-7.85(1H, d), 8.03-8.06(1H, | |
| | | d), 8.21-8.23(2H, d), 8.28-8.31(1H, d), 8.4-8.42(1H, d) | |
| 58 | 38 | 2.42(2H, bs), 2.54(2H, bs), 3.6(2H, bs), 3.92(2H, bs), 7.35-7.39(2H, t), | 400 |
| | | 7.59-7.64(1H, t), 7.7-7.72(2H, d), 7.87-7.92(2H, m), 8.13-8.15(2H, | |
| | | d), 8.39-8.44(1H, d), 12.75(1H, s) | |
| 59 | 39 | 2.36(3H, s), 2.38(2H, bs), 2.54(2H, bs), 3.45(2H, bs), 3.85(2H, bs), | 387 |
| | | 7.59-7.61(2H, d), 7.82-7.88(2H, m), 8.01-8.05(1H, d), 8.14-8.20(4H, m), | |
| | | 8.29-8.33(1H, d), 9.08(1H, s) | |
| 60 | 40 | 1.82-1.91(4H, m), 3.36-3.4(2H, t), 3.48-3.52(2H, t), 7.72-7.74(2H, | 358 |
| | | d), 7.89-7.94(3H, m), 8.14-8.19(2H, m), 8.24-8.26(2H, d), | |
| | | 8.48-8.52(1H, d), 9.6(1H, s) | |
| 61 | 41 | 2.42(2H, bs), 2.54(2H, bs), 3.6(2H, bs), 3.92(2H, bs), 7.7-7.4(2H, d), | 384 |
| | | 7.9-7.95(3H, m), 8.14-8.19(2H, m), 8.27-8.29(2H, d), 8.48-8.52(1H, | |
| | | d), 9.61(1H, s) | |
| 62 | 42 | 6.76-6.77(1H, m), 7.35-7.39(2H, t), 7.59-7.64(1H, t), 7.87-7.94(2H, t), | 369 |
| | | 8.01(1H, s), 8.19(4H, s), 8.38-8.42(1H, d), 8.64-8.65(1H, d), 12.76(1H, s) | |
| 63 | 43 | 3.07-3.11(2H, t), 3.96-3.98(2H, m), 6.5-6.54(1H, t), 6.68-6.7(1H, d), | 420 |
| | | 6.93-6.99(2H, m), 7.23-7.31(4H, m), 7.49-7.53(1H, t), 7.65-7.73(1H, | |
| | | d), 7.71-7.73(2H, d), 8.11-8.13(2H, d), 12.48(1H, s) | |
| 64 | 44 | 2.99(6H, s), 6.12-6.13(1H, d), 7.82-7.88(2H, m), 8.02-8.07(1H, d), | 398 |
| | | 8.24-8.26(5H, m), 8.33-8.38(3H, m), 9.09(1H, s) | |
| 65 | 45 | 2.38-2.4(2H, bs), 2.61-2.63(2H, d), 3.37-3.38(2H, m), 3.71(2H, m), | 401 |
| | | 7.66-7.69(2H, d), 7.9-7.94(3H, m), 8.14-8.19(2H, m), 8.26-8.31(2H, | |
| | | t), 8.48-8.52(1H, d), 9.58-9.60(1H, s), 10.56(1H, s) | |
| 66 | 47 | 2.17(3H, s), 2.61(3H, s), 6,.34(1H, s), 7.61(2H, s), 7.88-7.92(1H, d), | 459 |
| | | 8.01-8.04(2H, d), 8.15-8.17(1H, dd), 8.26-8.29(3H, m), | |
| | | 8.35-8.43(2H, m), 8.54(1H, d), 8.74-8.76(1H, d) | |
| 67 | 48 | 3.90-3.91(3H, d), 3.99-4.01(6H, t), 6.76(1H, s), 7.34(1H, s), | 444 |
| | | 7.84-7.88(1H, d), 8.01-8.05(2H, t), 8.17-8.19(2H, d), 8.27-8.32(3H, m), | |
| | | 8.41-8.42(1H, d), 8.64-8.65(1H, d) | |
| 68 | 49 | 2.17(3H, s), 2.6(3H, s), 6.34(1H, s), 7.66-7.7(1H, t), 7.82-7.86(1H, t), | 404 |
| | | 7.87-7.91(1H, d), 8.01-8.05(3H, t), 8.09-8.12(1H, d), 8.25-8.29(3H, | |
| | | t), 8.34-8.38(1H, d), 8.52-8.54(1H, d) | |
| 69 | 50 | 1.85-1.9(4H, bs), 2.98-3.09(4H, m), 3.47(2H, m), 3.61-3.63(4H, t), | 586 |
| | | 3.84-3.88(2H, m), 7.17-7.19(1H, m), 7.27-7.29(1H, m), | |
| | | 7.30-7.35(3H, m), 7.58-7.6(2H, m), 7.6-7.68(2H, m), 7.74-7.81(2H, m), | |
| | | 8.23-8.27(2H, m), 8.62(1H, s) | |
| 70 | 51 | 2.21(3H, s), 2.27-2.28(2H, m), 2.33-2.38(2H, m), 3.31(2H, m), | 476 |
| | | 3.65(2H, m), 3.88(3H, s), 3.99-4(6H, d), 7.33(1H, s), 7.59-7.61(1H, d), | |
| | | 7.66-7.68(1H, m), 7.81-7.85(1H, d), 8.02-8.04(1H, d), 8.20-8.3(3H, | |
| | | m), 8.4-8.42(1H, d) | |
| 71 | 52 | 2.17(3H, s), 2.6(3H, s), 3.91(3H, s), 3.99-4.01(6H, d), 6.34(1H, s), | 472 |
| | | 7.34(1H, s), 7.83-7.88(1H, d), 8-8.06(3H, m), 8.24-8.27(2H, d), | |
| | | 8.29.8.33(1H, d), 8.4-8.43(1H, d) | |
| 72 | 54 | 1.63(4H, m), 1.78-1.84(4H, m), 3.52(4H, m), 3.55-3.6(5H, m), | 440 |
| | | 7.54-7.57(1H, d), 7.65-7.69(1H, m), 7.81-7.9(2H, m), 8.02-8.11(2H, m), | |
| | | 8.2-8.25(3H, m), 8.3-8.36(2H, m), 8.49-8.51(1H, m) | |
| 73 | 55 | 7.39-7.43(1H, t), 7.52-7.56(1H, t), 7.65-7.69(1H, t), 7.71-7.73(1H, | 399 |
| | | d), 7.84-7.89(1H, d), 7.97-7.99(1H, d), 8.02-8.04(1H, t), | |
| | | 8.08-8.11(1H, s), 8.13-8.15(2H, d), 8.22-8.24(1H, d), 8.26-8.29(2H, d), | |
| | | 8.31-8.35(1H, d), 8.49-8.52(1H, d) | |
| 74 | 56 | 1.82-1.91(4H, m), 3.34-3.41(2H, t), 3.46-3.52(2H, t), 4.06(3H, s), | 503 |
| | | 7.13(1H, s), 7.72(2H, d), 7.86-7.9(1H, d), 8.03-8.06(1H, dd), | |
| | | 8.2-8.25(3H, t), 8.32-8.34(2H, t), 8.36-8.4(1H, d), 8.56-8.59(1H, d) | |
| 75 | 57 | 1.15-1.23(4H, m), 1.27-1.34(2H, m), 1.53(1H, m), 1.65-1.69(1H, m), | 485 |
| | | 1.8-1.83(1H, m), 2.52(4H, s), 3.27(2H, m), 3.91(4H, s), 7.58-7.64(4H, | |
| | | m), 7.73-7.77(1H, d), 7.91-7.93(2H, d), 8.01-8.03(1H, d), | |
| | | 8.07-8.13(3H, m), 8.89(1H, s), 9.28(1H, s) | |
| 76 | 58 | 1.28(9H, s), 6.76-6.78(1H, m), 7.85-7.89(1H, d), 7.98-8.04(3H, m), | 453 |
| | | 8.16-8.2(3H, t), 8.27-8.31(3H, t), 8.38-8.4(1H, d), 8.46(1H, s), | |
| | | 8.65-8.66(1H, d), 9.62(1H, s) | |
| 77 | 59 | 1.76-1.81(6H, d), 3.83(4H, s), 7.58-7.6(2H, d), 7.63-7.67(2H, t), | 609 |
| | | 7.71-7.79(2H, m), 7.95-8(2H, m), 8.18-8.2(4H, d), 8.32-8.36(2H, m), | |
| | | 8.64-8.68(1H, d), 9.78(1H, s), 11.24-11.29(1H, m) | |
| 78 | 60 | 1.25-1.27(6H, d), 1.83-1.86(2H, m), 2.02-2.11(2H, m), | 547 |
| | | 2.56-2.59(2H, m), 3.08(2H, m), 4.03(3H, s), 4.04(3H, s), 4.09(3H, s), | |
| | | 4.54-4.56(1H, m), 5.02-5.08(1H, m), 7.3(1H, d), 7.55-7.57(3H, d), | |
| | | 7.91-7.95(1H, d), 8.09-8.17(3H, m), 8.42-8.44(1H, d) | |
| 79 | 61 | 1.51-1.59(6H, m), 1.9(4H, t), 3.44-3.47(4H, t), 3.6-3.62(4H, t), | 455 |
| | | 7.57-7.62(2H, m), 7.68-7.71(4H, dd), 7.92-7.99(2H, m), 8.09-8.13(3H, | |
| | | m), 8.97(1H, s) | |
| 80 | 62 | 1.82-1.88(4H, m), 2.63(3H, s), 3.24-3.25(4H, t), 3.38-3.4(2H, d), | 510 |
| | | 3.48-3.52(2H, t), 3.9-3.91(4H, t), 7.63-7.66(3H, m), 7.12-7.73(2H, d), | |
| | | 7.77-7.81(1H, d), 7.92-7.95(1H, d), 8.2-8.22(2H, d), 8.24-8.28(1H, d) | |
| 81 | 63 | 1.94-2.02(4H, m), 3.17(3H, s), 3.35-3.37(4H, t), 3.43-3.46(2H, t), | 520 |
| | | 3.68-3.72(2H, t), 4.05-4.07(4H, t), 7.18(1H, s), 7.69-7.71(2H, d), | |
| | | 7.86-7.9(1H, d), 8.11-8.14(1H, dd), 8.16-8.18(2H, d), 8.25-8.29(2H, m), | |
| | | 8.67-8.68(1H, d) | |
| 82 | 64 | 1.26-1.29(3H, t), 4.16-4.21(2H, m), 7.69-7.71(2H, d), 7.82-7.86(1H, | 431 |
| | | d), 8.16-8.19(2H, d), 8.23-8.29(2H, m), 8.34-8.38(1H, d), | |
| | | 8.54-8.57(1H, dd), 8.61-8.63(1H, d), 8.87(1H, s), 9.84(1H, s), 10.15(1H, s) | |
| 83 | 65 | 7.24-7.26(1H, m), 7.43-7.53(5H, m), 7.82-7.91(3H, m), | 506 |
| | | 8.04-8.14(2H, t), 8.18-8.2(1H, d), 8.26-8.29(1H, d), 8.31-8.36(1H, m), | |
| | | 8.36-8.49(2H, m), 8.5-8.64(1H, m), 8.77(1H, s), 9.79(1H, s) | |
| 84 | 66 | 7.27-7.32(3H, m), 7.44-7.48(2H, t), 7.7-7.76(2H, d), 7.86-7.9(1H, d), | 479 |
| | | 8.22-8.25(2H, d), 8.27-8.3(1H, d), 8.33-8.36(1H, d), 8.4-8.45(1H, d), | |
| | | 8.57-8.6(1H, dd), 8.68-8.71(1H, d), 8.9-8.91(1H, d), 9.86(1H, s), | |
| | | 10.77(1H, s) | |
| 85 | 69 | 3.26(4H, bs), 3.82(3H, s), 3.85(3H, s), 3.91(4H, bs), 7.12-7.14(2H, d), | 448 |
| | | 7.25-7.28(1H, d), 7.5-7.53(1H, t), 7.59-7.63(1H, t), 7.85-7.87(2H, d), | |
| | | 8.01-8.04(1H, d), 8.18-8.20(2H, d), 9.49(1H, s) | |
| 86 | 70 | 0.82-0.86(3H, t), 1.15-1.19(3H, t), 1.47-1.52(2H, m), 1.74(4H, bs), | 458 |
| | | 3.41(4H, s), 3.69-3.73(2H, t), 4.08-4.14(2H, m), 7.51-7.54(3H, d), | |
| | | 7.75-7.79(2H, m), 7.91-7.93(2H, m), 8.05-8.09(1H, d), | |
| | | 8.17-8.19(2H, d), 8.94(1H, s) | |
| 87 | 71 | 3.88(8H, s), 7.26-7.32(3H, m), 7.44-7.48(2H, t), 7.67-7.71(1H, t), | 480 |
| | | 7.75-7.84(4H, m), 7.97-8.01(1H, t), 8.16-8.19(1H, d), 8.3-8.33(2H, | |
| | | d), 8.35-8.37(1H, d), 8.82-8.86(1H, d), 10.82(1H, s) | |
| 88 | 72 | 1.08(9H, s), 1.37-1.47(2H, m), 1.64(1H, bs), 1.78(1H, bs), | 471 |
| | | 2.87-2.9(1H, t), 3.12-3.19(1H, t), 3.49-3.53(1H, m), 3.85-3.87(1H, m), | |
| | | 4.44(1H, bs), 7.24-7.26(1H, d), 7.58-7.6(2H, d), 7.9-7.94(3H, m), | |
| | | 8.15-8.19(2H, m), 8.26-8.29(2H, d), 8.47-8.51(1H, d), 9.59(1H, s) | |
| 89 | 73 | 1.5-1.57(3H, m), 1.75(1H, bs), 2.82(3H, s), 2.91-2.98(2H, m), 3.04(3H, | 443 |
| | | s), 3.13-3.27(1H, m), 3.53-3.57(1H, m), 4.48-4.52(1H, m), | |
| | | 7.60-7.63(2H, d), 7.9-7.94(3H, m), 8.14-8.19(2H, m), 8.25-8.27(2H, d), | |
| | | 8.47-8.51(1H, d), 9.6(1H, s) | |
| 90 | 74 | 3.72(3H, s), 7.69-7.71(2H, d), 7.83-7.87(1H, d), 8.17-8.2(2H, d), | 415 |
| | | 8.24-8.3(2H, d), 8.34-8.38(1H, d), 8.54-8.63(2H, m), 8.86-8.88(1H, m), | |
| | | 9.84-9.85(1H, d), 10.19(1H, s) | |
| 91 | 75 | 1.63-1.81(6H, d), 2.34-2.47(5H, m), 3.64(2H, s), 6.72-6.78(1H, m), | 504 |
| | | 7.18-7.20(1H, d), 7.33-7.37(3H, t), 7.5-7.61(3H, m), 7.82-7.89(3H, | |
| | | m), 7.96-8.12(3H, m), 8.8-8.85(1H, t) | |
| 92 | 76 | 1.32-1.37(3H, t), 1.62-1.70(6H, d), 2.59-2.62(2H, s), 3.64-3.66(2H, | 380 |
| | | bs), 4.23-4.31(2H, m), 6.71-6.77(1H, m), 6.85-6.9(1H, m), | |
| | | 7.47-7.69(4H, m), 7.93-8.03(2H, m), 8.08-8.1(1H, d) 9.9(1H, s) | |
| 93 | 77 | 3.29(4H, bs), 3.82(4H, bs), 7.46-7.49(1H, t), 7.7-7.72(1H, t), | 431 |
| | | 7.78-7.83(2H, t), 7.87-7.91(2H, t), 8.07-8.11(3H, t), 8.2-8.23(2H, d), | |
| | | 8.4(1H, s), 8.87(1H, s), 10.99(1H, s) | |
| 94 | 79 | 3.46-3.62(12H, m), 3.75-3.81(4H, m), 7.47-7.49(1H, m), | 501 |
| | | 7.67-7.89(6H, m), 7.94-7.95(1H, m), 8-8.1(2H, m), 8.85(1H, s), 11.2(1H, s) | |
| 95 | 80 | 1.63-1.73(6H, m), 3.26(4H, bs), 7.43-7.46(1H, m), 7.65-7.69(1H, m), | 429 |
| | | 7.73-7.76(1H, d), 7.82-7.88(2H, t), 8.07-8.11(4H, t), 8.2-8.23(2H, d), | |
| | | 8.41(1H, bs), 8.84(1H, s), 10.98(1H, s) | |
| 96 | 81 | 1.58-1.73(6H, d), 3.22(4H, bs), 3.54-3.57(4H, m), 3.62-3.66(4H, m), | 499 |
| | | 7.42-7.46(1H, m), 7.66-7.7(1H, m), 7.74-7.8(1H, m), 7.87-7.89(3H, | |
| | | d), 7.97-7.99(1H, d), 8.06-8.1(1H, d), 8.21-8.24(2H, d), 8.83(1H, s), | |
| | | 11.21(1H, s) | |
| 97 | 82 | 0.85-0.89(3H, t), 1.5-1.56(2H, m), 3.15-3.2(2H, m), 3.34(4H, bs), | 471 |
| | | 3.9-3.92(4H, m), 7.58-7.66(2H, m), 7.74-7.8(2H, m), 8.01-8.11(4H, m), | |
| | | 8.21-8.24(2H, d), 8.29-8.33(1H, d), 9.08-9.11(1H, t), 11.04(1H, s) | |
| 98 | 84 | 1.16-1.18(6H, d), 3.35-3.39(2H, m), 3.57-3.61(2H, m), 4.3-4.34(2H, d), | 456 |
| | | 6.95-6.98(1H, d), 7.25-7.31(3H, m), 7.4-7.47(3H, m), 7.61-7.69(3H, | |
| | | m), 7.76-7.8(1H, d), 8.14-8.17(2H, d), 8.45-8.46(1H, d), 10.67(1H, s) | |
| 99 | 85 | 1.56-1.64(6H, m), 3.27(4H, bs), 3.4-3.43(2H, m), 3.49-3.52(2H, m), | 499 |
| | | 3.92(4H, s), 7.57-7.62(2H, m), 7.73-7.81(2H, m), 7.87-7.9(2H, d), | |
| | | 8.01-8.1(2H, m), 8.2-8.23(2H, d), 8.26-8.3(1H, d), 11.17(1H, s) | |
| 100 | 86 | 1.25-1.28(2H, t), 1.56-1.58(2H, m), 1.89-1.92(2H, m), 3.09-3.19(4H, | 470 |
| | | m), 4.13-4.16(2H, m), 4.3-4.4(1H, m), 6.7(1H, s), 7.21-7.28(4H, m), | |
| | | 7.37-7.45(5H, m), 7.65-7.77(2H, m), 7.86-7.97(2H, d), 9.73(1H, s) | |
| 101 | 87 | 1.49-1.63(6H, m), 3.47-3.63(4H, m), 6.41-6.43(2H, t), 7.27-7.28(2H, | 477 |
| | | t), 7.56-7.62(3H, m), 7.75-7.8(3H, m), 7.87-7.97(3H, t), | |
| | | 8.03-8.06(2H, d), 11.09(1H, s) | |
| 102 | 88 | 3.52-3.66(8H, m), 6.46-6.48(2H, t), 7.36-7.37(2H, t), 7.7-7.74(1H, | 479 |
| | | t), 7.8-7.84(1H, t), 7.87-7.93(3H, m), 7.95-7.99(1H, m), | |
| | | 8.18-8.21(1H, d), 8.25-8.31(3H, t), 8.42-8.46(1H, d), 11.23(1H, s) | |
| 103 | 89 | 1.44-1.47(2H, m), 1.93-1.95(2H, m), 3-3.06(1H, m), 3.06-3.18(1H, | 517 |
| | | m), 3.5-3.65(2H, m), 4.37(1H, bs), 7.59-7.6(2H, d), 7.9-7.96(4H, m), | |
| | | 8.14-8.19(2H, m), 8.26-8.28(2H, d), 8.47-8.51(1H, d), 9.59(1H, s) | |
| 104 | 90 | 2.04-2.07(4H, m), 3.54-3.56(4H, t), 5.37(2H, s), 7.05-7.08(1H, d), | 451 |
| | | 7.3(1H, s), 7.41-7.5(3H, m), 7.54-7.59(2H, t), 7.61-7.63(1H, d), | |
| | | 7.66-7.7(1H, d), 7.73-7.78(1H, m), 8.04-8.15(3H, m), 8.65-8.66(1H, d) | |
| 105 | 91 | 2.04-2.07(4H, m), 3.52-3.57(4H, m), 5.21(2H, s), 6.44-6.46(1H, d), | 446 |
| | | 6.72-6.74(1H, d), 6.87(1H, s), 7.07-7.12(2H, t), 7.41-7.48(3H, m), | |
| | | 7.52-7.54(2H, d), 7.62-7.66(1H, d), 8.03-8.1(3H, t) | |
| 106 | 92 | 1.25-1.27(2H, m), 1.71(8H, s), 2.29(3H, s), 2.64(3H, s), 3.65(4H, bs), | 474 |
| | | 6.02(1H, s), 6.72-6.79(2H, m), 7.48-7.52(1H, m), 7.62-7.66(1H, d), | |
| | | 7.76-7.78(2H, d), 8.01-8.05(1H, d), 8.1-8.13(2H, d), 9.52(1H, s) | |
| 107 | 93 | 1.59-1.7(6H, d), 3.64(4H, s), 5.21(2H, s), 6.41-6.51(2H, dd), | 454 |
| | | 6.71-6.77(2H, dd), 6.89(1H, s) 7.47-7.55(4H, m), 7.61-7.65(1H, d), | |
| | | 7.9-8.02(1H, d), 8.05-8.08(2H, d) | |
| 108 | 94 | 1.7(6H, bs), 3.65(4H, bs), 4.87-4.89(2H, d), 6.32-6.4(1H, m), | 468 |
| | | 6.72-6.78(3H, m), 6.89(1H, bs), 7.29-7.31(1H, m), 7.34-7.38(2H, t), | |
| | | 7.42-7.45(2H, d), 7.47-7.49(1H, m), 7.51-7.57(2H, d), 7.61-7.65(1H, d), | |
| | | 7.99-8.03(1H, d), 8.06-8.11(2H, d) | |
| 109 | 95 | 1.7-2.14(12H, m), 2.66(2H, bs), 2.83(1H, bs), 3.2-3.27(1H, m), | 463 |
| | | 3.6-3.69(5H, m), 3.81(1H, bs), 4.37-4.39(1H, t), 4.82(1H, bs), | |
| | | 6.71-6.77(1H, m), 7.43-7.49(1H, t), 7.54-7.69(4H, m), 7.98-8.15(4H, m) | |
| 110 | 96 | 3.26-3.28(4H, t), 3.72(3H, s), 3.9-3.93(4H, t), 7.57-7.62(2H, m), | 416 |
| | | 7.68-7.79(4H, m), 8.01-8.08(2H, m), 8.16-8.18(2H, d), 8.25-8.29(1H, d), | |
| | | 10.18(1H, s) | |
| 111 | 97 | 3.55-3.66(8H, m), 7.35-7.39(2H, m), 7.56-7.65(3H, m), 7.87-7.88(1H, | 388 |
| | | t), 7.9-7.92(1H, t), 8.11-8.13(2H, d), 8.38-8.43(1H, d), 12.75(1H, s) | |
| 112 | 98 | 2.61(2H, bs), 2.71(2H, bs), 3.53(2H, bs), 3.89(2H, bs), 7.35-7.39(2H, t), | 404 |
| | | 7.59-7.64(3H, t), 7.87-7.91(2H, t), 8.11-8.13(2H, d), 8.39-8.43(1H, | |
| | | d), 12.75(1H, s) | |
| 113 | 99 | 2.17(3H, s), 2.6(3H, s), 6.34(1H, s), 7.35-7.39(2H, t), 7.59-7.64(1H, t), | 397 |
| | | 7.87-7.94(2H, m), 8.01-8.2(2H, d), 8.15-8.2(2H, d), 8.39-8.43(1H, d), | |
| | | 12.76(1H, s) | |
| 114 | 100 | 2.98(2H, bs), 3.09(2H, bs), 3.5(2H, bs), 3.84(2H, bs), 7.17-7.19(1H, m), | 531 |
| | | 7.31-7.39(4H, m), 7.59-7.63(1H, t), 7.67-7.69(2H, d), 7.87-7.92(2H, | |
| | | m), 8.12-8.15(2H, d), 8.39-8.44(1H, d), 12.75(1H, s) | |
| 115 | 101 | 3.2(2H, bs), 3.27(2H, bs), 3.45(2H, bs), 3.78(2H, bs), 6.81-6.84(1H, dd), | 497 |
| | | 6.91-6.94(1H, dd), 6.98-6.99(1H, t), 7.21-7.26(1H, t), 7.35-7.39(2H, | |
| | | t), 7.52-7.6(1H, t), 7.66-7.68(2H, d), 7.87-7.92(2H, m), | |
| | | 8.12-8.15(2H, d), 8.38-8.44(1H, d), 12.76(1H, s) | |
| 116 | 102 | 1.47-1.51(3H, m), 1.68-1.75(1H, m), 2.81(3H, s), 2.89-2.91(2H, m), | 459 |
| | | 3.04(3H, s), 3.15-3.25(1H, m), 3.53(1H, s), 4.46-4.51(1H, m), | |
| | | 7.35-7.42(2H, m), 7.5-7.53(1H, m), 7.59-7.64(2H, m), 7.89(1H, t), | |
| | | 7.91-7.98(1H, m), 8.11-8.15(2H, m), 8.39-8.44(1H, d), 12.74(1H, s) | |
| 117 | 103 | 1.08(9H, s), 1.22-1.49(2H, m), 1.63-1.77(2H, d), 2.89-2.92(1H, m), | 485 |
| | | 3.14(1H, t), 3.41-3.52(1H, m), 3.85-3.89(1H, m), 4.44(1H, bs), | |
| | | 7.17-7.25(1H, m), 7.32-7.41(2H, m), 7.48-7.63(4H, m), 7.84-7.92(2H, m), | |
| | | 8.12-8.15(1H, d). 8.39-8.44(1H, t), 12.74(1H, s) | |
| 118 | 104 | 1.18-1.19(6H, d), 1.54(2H, bs), 1.78-1.99(2H, d), 2.62-2.69(1H, bs), | |
| | | 2.98(1H, bs), 3.16(1H, bs), 3.4-3.51(1H, bs), 4.29-4.37(1H, bs), | |
| | | 4.87-4.94(1H, m), 7.18-7.2(2H, t), 7.35-7.39(3H, t), 7.59-7.63(2H, m), | |
| | | 8.1-8.12(2H, d), 8.39-8.43(1H, d), 12.74(1H, s) | |
| 119 | 105 | 1.77-1.88(4H, m), 3.39-3.42(1H, m), 3.55-3.62(1H, m), | 415 |
| | | 4.37-4.41(1H, m), 7(1H, s), 7.35-7.39(2H, t), 7.45(1H, s), 7.56-7.63(2H, m), | |
| | | 7.77-7.79(1H, d), 7.87-7.92(2H, m), 8.05-8.14(2H, m), 8.39-8.43(1H, d), | |
| | | 12.74(1H, s) | |
| 120 | 106 | 3.33-3.48(6H, m), 3.76-3.77(2H, m), 6.83-6.84(2H, d), 7.36-7.39(2H, | 464 |
| | | t), 7.59-7.63(1H, m), 7.67-7.69(2H, d), 7.87-7.92(2H, m), | |
| | | 8.13-8.15(1H, d), 8.32-8.34(3H, d), 8.4-8.44(1H, d), 12.75(1H, s) | |
| 121 | 107 | 3.56-3.65(8H, d), 7.33-7.45(2H, m), 7.55-7.57(2H, d), 7.59-7.69(2H, m), | 388 |
| | | 7.71-7.77(1H, m), 8-8.13(2H, d), 8.16-8.21(1H, m), 12.24(1H, s) | |
| 122 | 108 | 6.77-6.85(1H, m), 7.37-7.41(1H, d), 7.59-7.62(1H, t), 7.78-7.8(1H, | 369 |
| | | m), 7.86-7.88(1H, m), 8.01(1H, s), 8.16-8.2(3H, m), 8.23-8.28(2H, d), | |
| | | 8.35-8.42(1H, m), 8.65-8.68(1H, m), 12.69(1H, s) | |
| 123 | 109 | 2.2(3H, s), 2.27(2H, bs), 2.38(2H, bs), 3.29(2H, bs), 3.59-3.64(2H, bs), | 401 |
| | | 7.33-7.37(1H, d), 7.51-7.53(1H, d), 7.57-7.63(2H, m), 7.77-7.79(1H, | |
| | | d), 7.85-7.89(1H, m), 8.07-8.09(1H, d), 8.15-8.21(2H, m), | |
| | | 8.37-8.41(1H, d), 12.18(1H, s) | |
| 124 | 110 | 1.59-1.66(2H, m), 1.91-1.95(2H, bs), 3.11-3.13(2H, bs), | 494 |
| | | 3.61-3.74(3H, m), 7.24-7.31(3H, m), 7.44-7.48(3H, m), 7.69-7.75(3H, m), | |
| | | 7.78-7.83(2H, m), 7.88-7.91(1H, d), 7.95-8(1H, m), 8.07-8.11(1H, d), | |
| | | 8.21-8.23(2H, d), 8.87(1H, s), 10.74(1H, s) | |
| 125 | 111 | 1.57-1.66(2H, m), 1.9-1.92(2H, m), 2.99-3.05(2H, t), 3.56-3.59(2H, | 432 |
| | | d), 3.69-3.7(1H, m), 3.72(3H, s), 7.41-7.45(1H, t), 7.56-7.59(1H, m), | |
| | | 7.64-7.75(3H, t), 7.73-7.75(1H, d), 7.88-7.98(2H, m), 8.04-8.08(1H, | |
| | | d), 8.16-8.19(2H, d), 8.88(1H, s), 10.16(1H, s) | |
| 126 | 112 | 1.27-1.36(3H, t), 1.51-1.65(2H, m), 1.83-1.92(2H, m), 2.98-3.05(2H, | 446 |
| | | m), 3.56-3.62(2H, m), 3.65-3.72(1H, m), 4.12-4.21(2H, m), 7.08(1H, | |
| | | s), 7.41-7.45(1H, t), 7.56-7.59(2H, m), 7.64-7.69(3H, t), 7.73-7.8(1H, | |
| | | t), 7.84-7.97(3H, m), 8.15-8.18(1H, d), 10.13(1H, s) | |
| 127 | 113 | 1.14-1.23(6H, m), 3.48-3.6(4H, m), 4.29-4.32(2H, d), 7.45-7.52(2H, m), | 519 |
| | | 7.56-7.59(1H, t), 7.62-7.66(3H, t), 7.71-7.75(2H, m), 7.86-7.88(2H, | |
| | | d), 7.97-7.99(3H, d), 8.06-8.09(1H, d), 9.40-9.42(1H, d), 11.1(1H, bs) | |
| 128 | 114 | 1.54-1.63(6H, d), 3.3-3.44(2H, m), 3.51-3.57(6H, m), 3.72-3.74(4H, t), | 449 |
| | | 6.94-6.96(1H, d), 7.12-7.14(1H, d), 7.55-7.59(1H, d), 7.63-7.67(1H, t), | |
| | | 7.83-7.86(2H, d), 7.99-8(1H, t), 8.09-8.12(2H, d), 11.16(1H, s) | |
| 129 | 115 | 3.55(4H, bs), 3.73(4H, bs), 6.93-6.95(1H, d), 7.13-7.14(1H, d), | 379 |
| | | 7.54-7.65(2H, m), 8-8.17(6H, m), 8.4(1H, bs), 10.97(1H, s) | |
| 130 | 116 | 1.55(4H, bs), 1.63(4H, bs), 1.72(4H, bs), 3.26(4H, bs), 3.39-3.44(2H, | 497 |
| | | m), 3.51-3.52(2H, t), 7.42-7.46(1H, t), 7.65-7.69(1H, t), | |
| | | 7.74-7.76(1H, d), 7.83-7.88(3H, m), 8.06-8.1(1H, d), 8.21-8.23(2H, d), | |
| | | 8.31-8.32(1H, d), 8.82(1H, s), 11.16(1H, s) | |
| 131 | 117 | 1.55(4H, bs), 1.63-1.64(2H, bs), 3.39-3.42(2H, t), 3.52-3.54(2H, t), | 413 |
| | | 7.87-7.95(5H, m), 8.14-8.18(2H, m), 8.23-8.25(2H, d), 8.45-8.5(1H, | |
| | | d), 9.59(1H, s), 11.2(1H, s) | |
| 132 | 118 | 3.53-3.58(4H, m), 3.6-3.68(4H, m), 7.87-7.98(5H, m), 8.14-8.18(2H, | 415 |
| | | m), 8.18-8.26(2H, d), 8.47-8.51(1H, d), 9.58(1H, s), 11.25(1H, s) | |
| 133 | 119 | 0.85-0.89(3H, t), 1.51-1.56(2H, m), 3.15-3.19(2H, m), 7.9-7.94(3H, | 387 |
| | | m), 8.02-8.09(2H, d), 8.1-8.18(2H, m), 8.23-8.26(2H, d), | |
| | | 8.48-8.52(1H, d), 9.07-9.1(1H, t), 9.6(1H, s), 11.03(1H, s) | |
| 134 | 120 | 7.9-7.96(3H, m), 8-8.06(1H, m), 8.08-8.11(2H, d), 8.14-8.18(3H, | 345 |
| | | m), 8.23-8.25(2H, m), 8.48-8.53(1H, dd), 9.6(1H, s), 11.02(1H, s) | |
| 135 | 121 | 3.26(3H, s), 3.38-3.41(2H, t), 3.45-3.48(2H, t), 7.9-7.95(3H, m), | 403 |
| | | 8.08-8.11(2H, d), 8.14-8.18(2H, m), 8.23-8.26(2H, d), 8.48-8.52(1H, d), | |
| | | 9.6(1H, s), 11.07(1H, s) | |
| 136 | 122 | 3.18-3.21(2H, bs), 3.33(2H, bs), 3.39-3.46(2H, bs), 3.79(2H, bs), | 483 |
| | | 6.82-6.84(1H, dd), 6.92-6.95(1H, dd), 6.98-6.99(1H, t), 7.22-7.26(1H, t), | |
| | | 7.67-7.69(2H, d), 7.90-7.95(3H, m), 8.15-8.19(2H, m), 8.27-8.29(2H, | |
| | | d), 8.48-8.52(1H, d), 9.6(1H, s) | |
| 137 | 123 | 2.62(2H, bs), 2.69-2.72(2H, bs), 3.53(2H, bs), 3.9(2H, bs), | 390 |
| | | 7.63-7.65(2H, d), 7.89-7.92(1H, d), 7.92-7.94(2H, m), 8.14-8.19(2H, m), | |
| | | 8.25-8.28(2H, d), 8.48-8.52(1H, d), 9.6(1H, s) | |
| 138 | 124 | 1.18-1.19(6H, d), 1.54(2H, bs), 1.78-1.82(1H, bs), 1.93-1.96(1H, bs), | 458 |
| | | 2.6(1H, bs), 2.98(1H, bs), 3.12(1H, bs), 3.39-3.49(1H, bs), 4.34(1H, bs), | |
| | | 4.87-4.94(1H, m), 7.61-7.63(2H, d), 7.9-7.91(1H, d), 7.92-7.94(2H, t), | |
| | | 8.14-8.19(2H, m), 8.24-8.27(2H, d), 8.47-8.51(1H, d), 9.6(1H, s) | |
| 139 | 125 | 3.43-3.44(4H, bs), 3.5-3.55(2H, bs), 3.78(2H, bs), 6.88-6.9(2H, d), | 450 |
| | | 7.68-7.71(2H, d), 7.91-7.92(1H, d), 7.93-7.95(2H, m), 8.15-8.17(2H, m), | |
| | | 8.2-8.22(2H, m), 8.28-8.3(2H, d), 8.48-8.52(1H, d), 9.61(1H, s) | |
| 140 | 127 | 2.38-2.44(4H, bs), 3.38-3.42(2H, t), 3.52-3.56(4H, m), 3.57(6H, m), | 464 |
| | | 3.72-3.75(4H, t), 6.94-6.98(1H, d), 7.15-7.13(1H, d), 7.55-7.59(2H, m), | |
| | | 7.64-7.68(1H, t), 7.98-8.02(3H, m), 8.12-8.14(2H, d), 9.16(1H, s) | |
| 141 | 128 | 1.07-1.08(9H, d), 1.37-1.47(2H, m), 1.64-1.67(1H, m), 1.78(1H, bs), | 471 |
| | | 2.89-2.93(1H, m), 3.15-3.18(1H, m), 3.44-3.52(1H, m), 3.85-3.87(1H, m), | |
| | | 4.47(1H, bs), 7.24-7.26(1H, d), 7.55-7.58(2H, d), 7.9-7.94(3H, m), | |
| | | 8.14-8.19(2H, m), 8.26-8.29(2H, d), 8.47-8.51(1H, d), 9.59(1H, s) | |
| 142 | 129 | 2.69-2.73(1H, d), 2.89(1H, s), 3.13(4H, bs), 3.57(4H, bs), 3.74(8H, bs), | 466 |
| | | 3.79(2H, bs), 6.97-6.99(1H, d), 7.19-7.21(1H, d), 7.56-7.6(1H, d), | |
| | | 7.67-7.72(3H, m), 8.02-8.06(3H, t), 9.31(1H, bs), 9.36(1H, s) | |
| 143 | 134 | 1.86-1.94(3H, m), 2.15-2.2(4H, t), 2.69(4H, s), 3.28-3.36(6H, t), | 494 |
| | | 3.56-3.57(3H, m), 6.93-6.96(1H, d), 7.13-7.15(1H, d), 7.55-7.59(1H, d), | |
| | | 7.63-7.67(1H, t), 7.86-7.89(1H, d), 7.94-7.97(2H, m), 8-8.06(1H, d), | |
| | | 8.09-8.12(1H, d), 8.9(1H, m), 10.97-11.03(1H, d) | |
| 144 | 135 | 2.41(4H, t), 3.24-3.28(2H, m), 3.42-3.44(2H, m), 3.58-3.59(4H, m), | 432 |
| | | 7.65-7.68(1H, d), 7.85(1H, d), 7.9-7.93(3H, m), 8.02-8.04(2H, d), | |
| | | 8.12-8.22(4H, m), 8.46-8.52(1H, m), 9.6(1H, s) | |
| 145 | 137 | 7.64-7.68(1H, t), 7.8-7.85(2H, m), 8.0-8.04(2H, d), 8.07-8.09(3H, d), | 346 |
| | | 8.21-8.26(3H, t), 8.32-8.41(2H, t), 8.48-8.51(1H, d), 10.99(1H, s) | |
| 146 | 138 | 3.51-3.59(4H, m), 3.62-3.68(4H, m), 7.64-7.69(1H, t), 7.8-7.84(1H, | 416 |
| | | t), 7.86(1H, s), 7.88-7.9(2H, d), 8.02-8.04(1H, d), 8.08-8.1(1H, d), | |
| | | 8.21-8.24(3H, d), 8.31-8.35(1H, d), 8.48-8.51(1H, d), 11.23(1H, s) | |
| 147 | 140 | 3.42-3.47(2H, s), 3.65-3.72(8H, m), 7.41-7.45(2H, t), 7.49-7.56(2H, | 443 |
| | | m), 7.56-7.34(2H, m), 7.74-7.83(2H, m), 8-8.02(1H, d), 8.04(3H, s), | |
| | | 8.09-8.11(1H, d), 9.21(1H, s) | |
| 148 | 141 | 3.59-3.63(2H, t), 4.4-4.6(2H, t), 6.83(1H, d), 7.65-7.69(2H, t), | 446 |
| | | 7.81-7.9(2H, m), 7.94-7.99(5H, m), 8.02-8.07(1H, d), 8.11-8.14(1H, d), | |
| | | 8.22-8.25(3H, dd), 8.27-8.31(1H, d), 8.43-8.45(1H, d), 9.12(1H, s) | |
| 149 | 144 | 3.27(4H, t), 3.92(4H, t), 7.57-7.63(4H, m), 7.69-7.73(2H, d), | 523 |
| | | 7.87-7.93(4H, m), 8.02-8.09(2H, m), 8.17-8.19(2H, d), 8.28-8.32(1H, d), | |
| | | 9.25(1H, s), 9.36(1H, s), 12.66(1H, s) | |
| 150 | 145 | 1.67-1.83(6H, m), 3.24(4H, bs), 7.57-7.61(4H, t), 7.69-7.79(3H, m), | 521 |
| | | 7.87-7.93(5H, m), 7.96-8(2H, m), 8.17-8.19(1H, d), 9.22-9.26(2H, d), | |
| | | 12.63(1H, s) | |
| 151 | 146 | 1.69-1.83(6H, m), 2.53(3H, s), 3.29(4H, bs), 6.7(1H, s), 7.59-7.63(3H, | 566 |
| | | d), 7.68-7.81(2H, m), 7.92-7.99(3H, m), 8.17-8.24(2H, t), 9.55(1H, s), | |
| | | 10.49(1H, s) | |
| 152 | 147 | 1.28(9H, s), 2.9(6H, s), 6.47-6.48(1H, d), 7.85-7.89(1H, d), | 496 |
| | | 8-8.04(2H, m), 8.16-8.18(1H, d), 8.24-8.3(5H, m), 8.33-8.39(2H, m), | |
| | | 8.46(1H, bs), 9.62(1H, s) | |
| 153 | 148 | 3.31(2H, bs), 3.54(2H, bs), 3.64(4H, bs), 3.88(3H, s), 3.97-3.99(6H, d), | 463 |
| | | 7.26(1H, s), 7.59-7.61(2H, d), 7.76-7.81(1H, d), 8.07-8.09(1H, d), | |
| | | 8.16-8.19(2H, d), 8.25-8.29(1H, d), 8.59-8.61(1H, d) | |
| 154 | 149 | 1.69-1.83(6H, d), 3.3(4H, bs), 7.03-7.08(1H, m), 7.52-7.59(2H, m), | 546 |
| | | 7.65-7.69(2H, t), 7.75-7.81(3H, m), 7.92-7.99(2H, m), 8.18-8.24(2H, | |
| | | t), 8.3-8.38(2H, m), 9.57-9.59(1H, d), 10.81-10.84(1H, d) | |
| 155 | 150 | 1.1-1.32(7H, m), 1.52-1.55(2H, m), 1.64-1.67(4H, m), 1.82(4H, bs), | 551 |
| | | 3.24-3.29(2H, t), 3.44-3.5(2H, m), 6.24-6.26(1H, d), 7.49(2H, d), | |
| | | 7.58-7.6(1H, d), 7.68-7.77(1H, m), 7.83-7.9(2H, d), 7.96-7.99(1H, d), | |
| | | 8.1-8.13(1H, d), 8.17-8.35(1H, m), 8.74(1H, s), 8.88(1H, s) | |
| 156 | 151 | 1.72-1.79(2H, m), 1.97-2.01(1H, m), 3.23(1H, bs), 3.39(3H, s), | 423 |
| | | 3.52-3.67(4H, d), 4.04(1H, bs), 7.56-7.6(3H, m), 7.68-7.7(1H, d), 7.78(1H, bs), | |
| | | 7.85-7.87(1H, d), 7.95-8.03(1H, d), 8.15-8.17(4H, d), 8.23-8.25(1H, d) | |
| 157 | 152 | 1.7(6H, s), 3.65(4H, s), 6.58-6.59(1H, m), 6.72-6.79(1H, m), 7.5-7.53(1H, | 387 |
| | | t), 7.61-7.7(1H, d), 7.97-8.01(1H, d), 8.12-8.28(6H, m), 8.49(1H, d) | |
| 158 | 153 | 1.27(6H, bs), 2.28(3H, s), 2.67(3H, s), 3.65(4H, s), 6.11(1H, s), | 415 |
| | | 6.73-6.79(2H, m), 7.48-7.53(1H, m), 7.62-7.67(1H, d), 7.95-7.99(1H, d), 8.1(4H, s) | |
| 159 | 154 | 1.25-1.27(6H, d), 1.69(9H, s), 1.7-2.06(3H, m), 3.09-3.11(2H, bs), | 512 |
| | | 3.64-3.7(4H, s), 4.54-4.56(1H, bs), 5.02-5.08(1H, m), 6.73-6.78(2H, t), | |
| | | 7.48-7.55(3H, m), 7.6-7.64(1H, d), 7.93-7.97(1H, d), 8.06-8.08(1H, d) | |
| 160 | 155 | 3.4-3.44(2H, t), 3.57-3.62(2H, t), 7.13-7.14(1H, m), 7.35-7.36(1H, | 454 |
| | | d), 7.62-7.67(2H, t), 7.83-7.95(4H, m), 8.13-8.19(5H, m), | |
| | | 8.27-8.29(2H, d), 8.46-8.5(1H, d), 9.59(1H, s) | |
| 161 | 157 | 3.25-3.29(2H, m), 3.39-3.41(2H, t), 3.54-3.56(4H, t), 3.72-3.74(4H, | 490 |
| | | t), 6.61-6.67(1H, m), 6.92-6.95(1H, d), 7.11-7.14(2H, t), | |
| | | 7.35-7.37(1H, d), 7.51-7.55(1H, d), 7.58-7.6(2H, d), 7.62-7.68(3H, m), | |
| | | 7.99-8.03(2H, m), 9.12(1H, s) | |
| 162 | 158 | 3.05-3.08(2H, t), 4.11-4.15(2H, t), 7.6-7.65(3H, m), 7.68-7.7(2H, d), | 502 |
| | | 7.89-7.93(3H, m), 7.81-7.85(3H, m), 8.14-8.2(4H, m), 8.46-8.5(1H, | |
| | | d), 9.58(1H, s), 10.21(1H, s) | |
| 163 | 159 | 3.25-3.29(2H, t), 3.36(2H, t), 3.55-3.56(4H, t), 3.72-3.75(4H, t), | 488 |
| | | 6.92-6.95(1H, d), 7.11-7.14(1H, d), 7.35-7.37(1H, d), 7.51-7.59(3H, m), | |
| | | 7.62-7.67(2H, m), 7.99-8.03(3H, m), 8.08-8.14(1H, m), | |
| | | 8.43-8.45(1H, d), 9.08-9.13(1H, d), 9.8(2H, bs) | |
| 164 | 162 | 3.57-3.68(8H, d), 7.62-7.69(3H, m), 7.81-7.89(2H, m), 8.02-8.04(1H, | 440 |
| | | s), 8.08-8.11(1H, d), 8.22-8.25(3H, m), 8.49-8.51(1H, s) | |
| 165 | 163 | 1.54-1.63(6H, d), 1.68(3H, s), 3.39-3.44(2H, m), 3.51-3.57(6H, m), | 462 |
| | | 3.72-3.74(4H, t), 6.92-6.93(1H, d), 7.12-7.14(1H, d), | |
| | | 7.55-7.59(1H, s), 7.63-7.67(1H, t), 7.83-7.86(2H, d), 8-8.18(2H, d), 10.1(1H, s) | |
| 166 | 164 | 3.51-3.6(4H, m), 3.62-3.69(4H, m), 7.64-7.69(1H, t), 7.84-7.86(2H, | 483 |
| | | m), 7.88-7.9(2H, d), 8.02-8.04(1H, s), 8.08-8.1(1H, m), 8.21-8.24(3H, | |
| | | m), 11.23(1H, s) | |
| 167 | 165 | 3.43-3.6(12H, bs), 3.75-3.81(4H, bs), 7.58-7.67(2H, m), | 540 |
| | | 7.73-7.83(4H, m), 8.01-8.08(2H, m), 8.11-8.13(2H, d), 8.49(1H, s) | |
| 168 | 166 | 1.53-1.64(6H, bs), 1.69(3H, s), 3.39-3.48(2H, bs), 3.51-3.57(6H, s), | 436 |
| | | 3.72-3.74(4H, t), 6.92-6.94(1H, d), 7.12-7.14(1H, d), 7.55-7.59(1H, | |
| | | s), 7.63-7.67(1H, t), 7.83-7.86(2H, d), 8-8.18(2H, d), 8.34(1H, s) | |
| 169 | 167 | 3.25-3.29(2H, t), 3.36(2H, t), 3.55-3.56(4H, t), 3.72-3.75(4H, t), | 500 |
| | | 6.92-6.95(1H, d), 7.11-7.14(1H, d), 7.35-7.37(1H, d), 7.51-7.59(2H, m), | |
| | | 7.62-7.67(2H, m), 7.99-8.03(3H, m), 8.08-8.14(2H, m), | |
| | | 8.43-8.45(1H, d), 9.08-9.13(1H, d), 9.8(2H, bs) | |
| 170 | 168 | 1.72-1.79(2H, m), 1.97-2.03(2H, m), 2.84-2.91(4H, m), | 444 |
| | | 3.6-3.63(3H, m), 4.4(1H, m), 4.5-4.8(2H, t), 7.43-7.45(2H, t), 7.49-7.56(2H, | |
| | | m), 7.58-7.63(2H, m), 7.76-7.83(2H, m), 8-8.02(1H, d), 8.06(3H, s), | |
| | | 8.09-8.13(1H, d), 9.28(1H, s) | |

Biological Evaluation

### Example (I)

### Effect of Compounds of the Instant Invention on Cellular Expression of HSP

1.1 Background:

Experiments set forth in this section were conducted to determine whether the compounds of the present invention are able to elevate the expression of HSP-70 gene in cells.

1.2 In vitro Screening for HSP Induction

1.21 Materials and Methods:

1.21 (a) Cell Culture Conditions

HeLa cells were obtained from American Type Culture Collection (ATCC) (CCL-2). The cells were seeded in a 96 well flat bottom plate (Corning), at a density of 20,000 cells/well in 200 μl culture medium consisting of Minimum Essential Medium (MEM) and 10% Fetal Bovine Serum (Hyclone, USA), and allowed to grow for 24 hours at 37° C. in a CO₂ incubator to reach a confluency of 75-80%.

1.21 (b) Compound Treatment

A 200× stock of the test compounds was prepared in the appropriate solvent and 1 μl of said stock was added to each well, so that the final DMSO (Dimethyl sulfoxide) concentration per well was 0.5%. Each test compound was tested in triplicate. The plate was incubated at 37° C. in a CO₂ incubator for 4 hours. At the end of the incubation period, the total RNA was isolated from the cells as described hereinbelow.

1.21 (c) RNA Isolation & Quantification

RNA was isolated using either Tri Reagent (Sigma) or Trizol (Invitrogen). RNase AWAY (Molecular Bioproducts) was applied to working surfaces and pipettes in order to inactivate RNases. The RNA pellet obtained after isopropyl alcohol precipitation was finally reconstituted in Diethyl pyrocarbonate (DEPC) treated water (0.01% v/v) and quantitated by taking OD (Optical density) values at 260 and 280 nm on a Beckman spectrophotometer. OD value of 1 at 260 nm corresponds to an RNA concentration of 40 μg/ml.

1.21 (d) DNase Treatment

RNA was treated with DNase (MBI Fermetas) to remove the genomic DNA contamination. RNA was treated with 1U of DNase/μg of RNA in 10× DNase buffer in a 15 μl reaction and the reaction mix was incubated at 37° C. for 30 minutes. After the reaction was over, 1.5 μl of 25 mM of EDTA (MBI Fermentas) was added per sample and the reaction mix incubated further at 65° C. for 10 minutes.

1.21 (e) cDNA Synthesis:

In this step, RNA is converted into a single stranded cDNA employing High capacity cDNA archive kit (Part No. 4322171 Applied Biosystems USA) with the reverse transcriptase enzyme using random hexamers and dNTPs (Deoxyribonucleotide Triphosphate). For each DNase treated 1 μg of RNA sample, the following components were added for cDNA synthesis: 2.5 μl 10× reverse transcriptase buffer, 1 μl 25× dNTPs, 2.5 μl 10× Random primer, 1.25 μl Multiscribe reverse transcriptase and Diethyl pyrocarbonate (DEPC) treated water (0.01% v/v) to make a final 25 μl reaction mix. The cDNA reaction mix was incubated further at 25° C. for 10 minutes followed by incubation at 37° C. for 120 minutes.

1.21 (f) Real Time PCR Reaction Setup

Real time PCR reaction was performed using ABI 7000 SDS under universal cycling conditions. A multiplex real time PCR reaction was set up as follows. The final reaction mix contained 2.5 μl cDNA reaction mix, 0.625 μl 20× human HSP-70 Taqman probe and primer mix (Part No 4331182 Applied Biosystems USA), 0.625 μl 20×18S rRNA Taqman probe and primer mix (Part No 4319413 Applied Biosystems USA) (as internal control) and 6.25 μl 2× Taqman universal master mix (Part No; 4304437 Applied Biosystems USA) in a final volume of 12.5 μl. In each plate, a reaction without cDNA was also run to serve as NTC (No Template Control). Each condition was run in duplicate.

1.21 (g) Data Analysis

Data analysis was performed by comparative CT (Cycle Threshold) method. HSP-70b mRNA expression was normalized relative to the expression of 18S ribosomal RNA for that sample. The results for test compounds were expressed as fold induction of HSP-70b mRNA relative to vehicle treated control and are as shown in Table 2

In the Table 2, 0 indicates < 4 fold, while +, ++, +++ and ++++ indicate 4-24 fold, 25-192 fold, 193-1536 fold, and >1536 fold induction of HSP-70b mRNA, respectively, relative to the vehicle treated control.

TABLE 2

| | | HSP-70b mRNA |
|---|---|---|
| | | induction |
| Comp. | | (at a concentration |
| No. | Compound | of 25 μM) |
| 2 | 1-[4-(3-Quinolin-2-yl-acryloyl)-benzoyl]-piperidin-4-one | +++ |
| 3 | 1-[4-(4-Methyl-piperazine-1-carbonyl)-phenyl]-3-quinolin-2-yl- | +++ |
| | propenone | |
| 6 | 1-[4-(Piperazine-1-carbonyl)-phenyl]-3-quinolin-2-yl-propenone | +++ |
| 10 | 3-Quinolin-2-yl-1-[4-(thiomorpholine-4-carbonyl)-phenyl]-propenone | ++++ |
| 12 | 1-[4-(Piperidine-1-carbonyl)-phenyl]-3-quinolin-2-yl-propenone | +++ |
| 15 | 1-{4-[4-(3-Chloro-phenyl)-piperazine-1-carbonyl]-phenyl}-3-quinolin-2- | +++ |
| | yl-propenone | |
| 16 | 1-{4-[4-(2,3-Dichloro-phenyl)-piperazine-1-carbonyl]-phenyl}-3- | ++++ |
| | quinolin-2-yl-propenone | |
| 17 | N-(4-{2-Oxo-4-[4-(3-quinolin-2-yl-acryloyl)-benzoyl]-piperazin-1-yl}- | ++++ |
| | phenyl)-acetamide | |
| 18 | 4-Imidazol-1-yl-2-[3-oxo-3-(4-trichloromethoxycarbonylamino-phenyl)- | 0 |
| | propenyl]-quinoline-6-carboxylic acid methyl ester | |
| 20 | 3-Quinolin-2-yl-1-{4-[4-(tetrahydro-furan-2-carbonyl)-piperazine-1- | + |
| | carbonyl]-phenyl}-propenone | |
| 21 | 1-{4-[4-(Furan-2-carbonyl)-piperazine-1-carbonyl]-phenyl}-3-quinolin-2- | ++ |
| | yl-propenone | |
| 23 | {4-[3-(4-Morpholin-4-yl-quinolin-2-yl)-acryloyl]-phenyl}-carbamic acid | ++++ |
| | ethyl ester | |
| 24 | {4-[3-(4-Morpholin-4-yl-quinolin-2-yl)-acryloyl]-phenyl}-carbamic acid | ++++ |
| | 2,2-dimethyl-propyl ester | |
| 25 | 1-{4-[3-(4-Morpholin-4-yl-quinolin-2-yl)-acryloyl]-phenyl}-3-(2- | ++++ |
| | trifluoromethyl-phenyl)-urea | |
| 26 | 1-Benzenesulfonyl-3-{4-[3-(4-morpholin-4-yl-quinolin-2-yl)-acryloyl]- | ++++ |
| | phenyl}-urea | |
| 29 | {4-[3-(4-Piperidin-1-yl-quinolin-2-yl)-acryloyl]-phenyl}-carbamic acid | +++ |
| | ethyl ester | |
| 30 | {4-[3-(4-Piperidin-1-yl-quinolin-2-yl)-acryloyl]-phenyl}-carbamic acid | ++ |
| | isobutyl ester | |
| 31 | {4-[3-(2-Pyrrolidin-1-yl-quinolin-3-yl)-acryloyl]-phenyl}-carbamic acid | + |
| | ethyl ester | |
| 43 | 1-[4-(2,3-Dihydro-indole-1-carbonyl)-phenyl]-3-(3-hydroxy-quinoxalin-2- | ++++ |
| | yl)-propenone | |
| 45 | 1-[4-(3-Quinoxalin-2-yl-acryloyl)-benzoyl]-piperidin-4-one oxime | +++ |
| 59 | 1-Benzenesulfonyl-3-{4-[3-(6-methyl-4-piperidin-1-yl-quinolin-2-yl)- | ++++ |
| | acryloyl]-phenyl}-urea | |
| 66 | {4-[3-(6-[1,2,3]Thiadiazol-4-yl-quinolin-2-yl)-acryloyl]-phenyl}- | + |
| | carbamic acid phenyl ester | |
| 67 | {4-[3-(6-Morpholin-4-yl-pyridin-2-yl)-acryloyl]-phenyl}-carbamic acid 2- | +++ |
| | morpholin-4-yl-ethyl ester | |
| 69 | {5-Methoxy-2-[3-(4-morpholin-4-yl-quinolin-2-yl)-acryloyl]-phenyl}- | 0 |
| | carbamic acid methyl ester | |
| 70 | Propyl-{4-[3-(2-pyrrolidin-1-yl-quinolin-3-yl)-acryloyl]-phenyl}- | 0 |
| | carbamic acid ethyl ester | |
| 71 | {4-[3-(4-Morpholin-4-yl-quinolin-2-yl)-acryloyl]-phenyl}-carbamic acid | +++ |
| | phenyl ester | |
| 74 | {4-[3-(6-[1,2,3]Thiadiazol-4-yl-quinolin-2-yl)-acryloyl]-phenyl}- | +++ |
| | carbamic acid methyl ester | |
| 75 | 1-(4-Methyl-benzenesulfonyl)-3-{4-[3-(3,4,5,6-tetrahydro-2H- | + |
| | [1,2′]bipyridinyl-6′-yl)-acroyloyl]-phenyl}-urea | |
| 76 | {4-[3-(3,4,5,6-Tetrahydro-2H-[1,2′]bipyridinyl-6′-yl)-acryloyl]-phenyl}- | +++ |
| | carbamic acid ethyl ester | |
| 84 | (4-{3-[6-(3,5-Dimethyl-morpholin-4-yl)-pyridin-3-yl]-acryloyl}-phenyl)- | 0 |
| | carbamic acid phenyl ester | |
| 86 | 5′-[3-Oxo-3-(4-phenoxycarbonylamino-phenyl)-propenyl]-3,4,5,6- | 0 |
| | tetrahydro-2H-[1,2′]bipyridinyl-4-carboxylic acid | |
| 90 | {4-[3-(6-Pyrrolidin-1-yl-pyridin-2-yl)-acryloyl]-phenyl}-carbamic acid | ++++ |
| | pyridin-2-ylmethyl ester | |
| 91 | {4-[3-(6-Pyrrolidin-1-yl-pyridin-2-yl)-acryloyl]-phenyl}-carbamic acid 4- | ++++ |
| | fluoro-benzyl ester | |
| 92 | {4-[3-(3,4,5,6-Tetrahydro-2H-[1,2′]bipyridinyl-6′-yl)-acryloyl]-phenyl}- | ++ |
| | carbamic acid 2-(3,5-dimethyl-pyrazol-1-yl)-ethyl ester | |
| 93 | {4-[3-(3,4,5,6-Tetrahydro-2H-[1,2′]bipyridinyl-6′-yl)-acryloyl]-phenyl}- | +++ |
| | carbamic acid furan-2-ylmethyl ester | |
| 94 | {4-[3-(3,4,5,6-Tetrahydro-2H-[1,2′]bipyridinyl-6′-yl)-acryloyl]-phenyl}- | 0 |
| | carbamic acid 3-phenyl-allyl ester | |
| 95 | {4-[3-(3,4,5,6-Tetrahydro-2H-[1,2′]bipyridinyl-6′-yl)-acryloyl]-phenyl}- | ++++ |
| | carbamic acid 2-piperidin-1-yl-ethyl ester | |
| 96 | {4-[3-(4-Morpholin-4-yl-quinolin-2-yl)-acryloyl]-phenyl}-carbamic acid | ++ |
| | methyl ester | |
| 110 | (4-{3-[2-(4-Hydroxy-piperidin-1-yl)-quinolin-3-yl]-acryloyl}-phenyl)- | ++ |
| | carbamic acid phenyl ester | |
| 111 | (4-{3-[2-(4-Hydroxy-piperidin-1-yl)-quinolin-3-yl]-acryloyl}-phenyl)- | +++ |
| | carbamic acid methyl ester | |
| 112 | (4-{3-[2-(4-Hydroxy-piperidin-1-yl)-quinolin-3-yl]-acryloyl}-phenyl)- | +++ |
| | carbamic acid ethyl ester | |
| 113 | 1-Benzenesulfonyl-3-(4-{3-[6-(3,5-dimethyl-morpholin-4-yl)-pyridin-3- | 0 |
| | yl]-acryloyl}-phenyl)-urea | |
| 121 | N-(2-Methoxy-ethyl)-N′-[4-(3-quinoxalin-2-yl-acryloyl)-phenyl]- | +++ |
| | oxalamide | |
| 126 | 1-{4-[3-(6-Morpholin-4-yl-pyridin-2-yl)-acryloyl]-phenyl}-3-(2-oxo-2- | ++++ |
| | piperidin-1-yl-ethyl)-urea | |
| 127 | 1-(2-Morpholin-4-yl-ethyl)-3-{4-[3-(6-morpholin-4-yl-pyridin-2-yl)- | ++++ |
| | acryloyl]-phenyl}-urea | |
| 129 | {4-[3-(6-Morpholin-4-yl-pyridin-2-yl)-acryloyl]-phenyl}-carbamic acid 2- | ++++ |
| | piperazin-1-yl-ethyl ester | |
| 130 | N-(2-Morpholin-4-yl-ethyl)-N′-[4-(3-quinoxalin-2-yl-acryloyl)-phenyl]- | 0 |
| | oxalamide | |
| 131 | 1-{4-[3-(6-Morpholin-4-yl-pyridin-2-yl)-acryloyl]-phenyl}-3-[2- | ++ |
| | (pyridine-2-sulfonyl)-ethyl]-urea | |
| 132 | 1-{4-[3-(6-Morpholin-4-yl-pyridin-2-yl)-acryloyl]-phenyl}-3-[2-(pyridin- | ++++ |
| | 2-ylsulfanyl)-ethyl]-urea | |
| 133 | 1-[2-(4-Methyl-piperazin-1-yl)-ethyl]-3-{4-[3-(6-morpholin-4-yl-pyridin- | ++ |
| | 2-yl)-acryloyl]-phenyl}-urea | |

Discussion

As seen in Table 2, HSP-70b mRNA levels were increased over control after treatment with compounds of the invention. The compounds of the instant invention have the ability to induce HSP-70.

### Example (II)

In vitro Screening for TNF-α Expression

1.1 Background

The purpose of the present study was to determine the inhibition of lipopolysaccharide (LPS)-induced TNF-α expression in phorbol merstyl ester (PMA) differentiated THP-1 cells.

1.2 Materials and Methods:

1.21 (a) Cell Culture Conditions

Human monocytic leukaemia cell line, THP-1, were obtained from American Type Culture Collection (ATCC) (TIB-202). Human monocytic leukaemia cell line, THP-1 differentiated into macrophage-like cells by Phorbol merystyl ester (PMA) (Sigma) treatment was employed for the experiments. The cells were seeded in a 24 well flat bottom plate (Corning), at a density of 300,000 cells/well in a 2 ml culture medium comprising RPMI 1640 Medium and 10% Fetal Bovine Serum (Hyclone, USA) containing PMA (25 ng/ml) and allowed to differentiate for 44 hours at 37° C. in a CO₂ incubator.

1.22 (b) Compound Treatment

The differentiated cells were then treated with either LPS (Sigma) (1 ug/ml) alone or with LPS (1 ug/ml) and the test compound for 4 hours. At the end of the incubation period, the total RNA was isolated from the cells as described hereinbelow.

1.22 (c) RNA Isolation & Quantification

RNA was isolated using either Tri Reagent (Sigma) or Trizol (Invitrogen). RNase AWAY (Molecular Bioproducts) was applied to working surfaces and pipettes in order to inactivate RNases. The RNA pellet obtained after isopropyl alcohol precipitation was finally reconstituted in Diethyl pyrocarbonate (DEPC) treated water (0.01% v/v) and quantitated by taking OD (Optical density) values at 260 and 280 nm on a Beckman spectrophotometer. OD value of 1 at 260 nm corresponds to an RNA concentration of 40 μ/ml.

1.22 (d) DNase Treatment

RNA was treated with DNase (MBI Fermetas) to remove the genomic DNA contamination. RNA was treated with 1 U of DNase/μg of RNA in 10× DNase buffer in a 15 μl reaction and the reaction mix was incubated at 37° C. for 30 minutes. After the reaction was over, 1.5 μl of 25 mM of EDTA (MBI Fermentas) was added per sample and the reaction mix incubated further at 65° C. for 10 minutes.

1.22 (e) cDNA Synthesis:

In this step, RNA is converted into a single stranded cDNA employing High capacity cDNA archive kit (Part No. 4322171 Applied Biosystems USA) with the reverse transcriptase enzyme using random hexamers and dNTPs (Deoxyribonucleotide Triphosphate). For each DNase treated 1 μg of RNA sample, the following components were added for cDNA synthesis: 2.5 μl 10× reverse transcriptase buffer, 1 μl 25× dNTPs, 2.5 μl 10× Random primer, 1.25 μl Multiscribe reverse transcriptase and Diethyl pyrocarbonate (DEPC) treated water (0.01% v/v) to make a final 25 μl reaction mix. The cDNA reaction mix was incubated further at 25° C. for 10 minutes followed by incubation at 37° C. for 120 minutes.

1.22 (f) Real Time PCR Reaction Setup

Real time PCR reaction was performed using ABI 7000 SDS under universal cycling conditions. A multiplex real time PCR reaction was set up as follows. The final reaction mix contained 2.5 μl cDNA reaction mix, 0.625 μl 20× Human TNF-α Taqman probe and primer mix (Part No 4327055F Applied Biosystems USA), 0.625 μl 20× 18s rRNA Taqman probe and primer mix (Part No 4319413E Applied Biosystems USA) (as internal control) and 6.25 μl 2× Taqman universal master mix (Part No. 4304437 Applied Biosystems USA) in a final volume of 12.5 μl. In each plate, a reaction without cDNA was also run to serve as NTC (No Template Control). Each condition was run in duplicate.

1.22 (g) Data Analysis

Data analysis was performed by comparative CT method. TNF-α mRNA expression was normalized relative to the expression of 18S ribosomal RNA for that sample. Considering TNF-α expression for cells treated with LPS alone as 100%; the results for test compounds were expressed as % inhibition of TNF-α expression and are as shown in Table 3

In the Table 3, 0 indicates <20% while +, ++, +++, ++++ indicate 21-40%, 41-60%, 61-80% and >80% inhibition of TNF-α expression, respectively.

TABLE 3

| | | TNF alpha mRNA |
|---|---|---|
| Comp. | | inhibition (at a |
| No. | Compound | concentration of 25 μM) |
| 2 | 1-[4-(3-Quinolin-2-yl-acryloyl)-benzoyl]-piperidin-4-one | ++++ |
| 3 | 1-[4-(4-Methyl-piperazine-1-carbonyl)-phenyl]-3-quinolin-2-yl- | ++++ |
| | propenone | |
| 6 | 1-[4-(Piperazine-1-carbonyl)-phenyl]-3-quinolin-2-yl-propenone | ++ |
| 10 | 3-Quinolin-2-yl-1-[4-(thiomorpholine-4-carbonyl)-phenyl]-propenone | +++ |
| 12 | 1-[4-(Piperidine-1-carbonyl)-phenyl]-3-quinolin-2-yl-propenone | ++++ |
| 15 | 1-{4-[4-(3-Chloro-phenyl)-piperazine-1-carbonyl]-phenyl}-3- | ++ |
| | quinolin-2-yl-propenone | |
| 16 | 1-{4-[4-(2,3-Dichloro-phenyl)-piperazine-1-carbonyl]-phenyl}-3- | + |
| | quinolin-2-yl-propenone | |
| 25 | 1-{4-[3-(4-Morpholin-4-yl-quinolin-2-yl)-acryloyl]-phenyl}-3-(2- | ++++ |
| | trifluoromethyl-phenyl)-urea | |
| 26 | 1-Benzenesulfonyl-3-{4-[3-(4-morpholin-4-yl-quinolin-2-yl)- | ++++ |
| | acryloyl]-phenyl}-urea | |
| 67 | {4-[3-(6-Morpholin-4-yl-pyridin-2-yl)-acryloyl]-phenyl}-carbamic | ++++ |
| | acid 2-morpholin-4-yl-ethyl ester | |
| 92 | {4-[3-(3,4,5,6-Tetrahydro-2H-[1,2′]bipyridinyl-6′-yl)-acryloyl]- | ++++ |
| | phenyl}-carbamic acid 2-(3,5-dimethyl-pyrazol-1-yl)-ethyl ester | |
| 93 | {4-[3-(3,4,5,6-Tetrahydro-2H-[1,2′]bipyridinyl-6′-yl)-acryloyl]- | ++++ |
| | phenyl}-carbamic acid furan-2-ylmethyl ester | |
| 95 | {4-[3-(3,4,5,6-Tetrahydro-2H-[1,2′]bipyridinyl-6′-yl)-acryloyl]- | ++++ |
| | phenyl}-carbamic acid 2-piperidin-1-yl-ethyl ester | |
| 121 | N-(2-Methoxy-ethyl)-N′-[4-(3-quinoxalin-2-yl-acryloyl)-phenyl]- | +++ |
| | oxalamide | |
| 126 | 1-{4-[3-(6-Morpholin-4-yl-pyridin-2-yl)-acryloyl]-phenyl}-3-(2-oxo- | ++++ |
| | 2-piperidin-1-yl-ethyl)-urea | |
| 127 | 1-(2-Morpholin-4-yl-ethyl)-3-{4-[3-(6-morpholin-4-yl-pyridin-2-yl)- | ++++ |
| | acryloyl]-phenyl}-urea | |

Discussion

As seen in Table 3, LPS-induced TNF-α expression was inhibited by the treatment with compounds of the invention.

In Vivo Activity

### Example (III)

### Assessment of Neuroprotective Efficacy

1.1 Materials and Methods:

Male Sprague-Dawley rats weighing 240-270 g were used. During the operation, the body temperature of the animals was maintained constant at 37° C., using a homoeothermic blanket. Anaesthesia was induced with chloral hydrate. Permanent cerebral ischemia was induced in these animals by intraluminal suture occlusion technique. A 25 mm length of Poly-L-Lysine coated Ethilon suture (3-0) was inserted from the proximal external carotid artery into the lumen of the internal carotid artery until mild resistance was felt [Longa E. Z. et al., Stroke, 1989, 20, 84-91]. At the end of 24 hrs, all the animals were sacrificed and the infarct characterized after staining with triphenyl tetrazolium chloride (TTC). The stained slices were captured using an ordinary document scanner and was analysed for infarct size and edema using the Scion® image analysis software. Neurological scores were obtained at two time points, one after the recovery of the animals from anaesthesia and the other, at the end of 24 hours just before the animals were sacrificed.

TABLE 4
Neurological Scores

| Score | Parameters |
|---|---|
| 0 | No Deficit |
| 1 | Failure to extend Right forepaw fully (Mild focal |
| | neurological deficit) |
| 2 | Circling to the contra lateral side (Moderate focal |
| | neurological deficit) |
| 3 | Falling to the contra lateral side |
| | (Moderate to severe focal neurological deficit) |
| 4 | Depressed level of Consciousness (severe focal |
| | neurological deficit) |

Statistical analysis on the data obtained was performed using student's t-test, and the level of significance was p<0.05. The results are shown in Table 5.

TABLE 5

| | | | | % Improvement |
|---|---|---|---|---|
| | i.v. | % Reduc- | % Reduc- | Mean |
| | Dose | tion | tion | Neurological |
| Treatment | (mg/kg) | Infarct | Edema | Score |
| HCl salt of | 4.6 | 13.1 | 53.4 | 75.0 |
| Compound No. 3 | | | | |
| Compound No. 16 | 3.89 | 26.4 | 77.4 | Nil |
| Compound No. 16 | 3.89 | 24.5 | 65.6 | 100.0 |
| (Multiple Dose) | | | | |
| HCl salt of | 0.81 | 6.5 | 3.7 | Nil |
| Compound No. 23 | | | | |

1.2 Discussion of Test Results:

The ability of a neuronal population to survive an ischemic trauma (like cerebral ischemia) is correlated with increased expression of HSP 70. Test compounds presented in Table 2 shows the ability to induce HSP-70 in vitro. Further it is also observed (Table 3) that test compounds of the present invention also inhibit TNF-α in cultured cells incubated with the above said drugs. HSP-70 mRNA was induced in neurons at the periphery of ischemia (penumbra). It is proposed that the peripheral zone of ischemia (penumbra) can be rescued from getting infarcted by pharmacological agents. [Dienel G. A. et al., J. Cereb. Blood Flow Metab., 1986, Vol. 6, pp. 505-510; Kinouchi H. et al., Brain Research, 1993, Vol. 619, pp. 334-338].The in vivo experiments carried out with the representative test compounds, the hydrochloride salt of Compound No. 3, the hydrochloride salt of Compound No. 23 and Compound No. 16 to assess their neuroprotective effect show neuronal protection, i.e. reduced infarct size and reduced brain edema following cerebral ischemic insult. These results very well correlate with results of our in vitro data. Hence, it can be concluded that compounds of the instant invention would be useful as neuroprotective agents by virtue of their HSP-70 inducing activity.

### Example (IV)

### Assessment of Anti-Inflammatory Activity

1.1 Materials and Methods:

Anti-inflammatory activity of the test compound was determined using standard procedures. [Enna S J, Williams M, Ferkany J W, Eds., “Current Protocols in Pharmacology”, John Wiley & Sons Inc., 1998, pp. 5.4.1 to 5.4.3]. Male Sprague-Dawley rats of 200-250 g body weight were used for the study. The animals were divided randomly into two groups—Vehicle (saline control) and Treatment group. For induction of acute inflammation, 50 μl of 0.5% Carrageenan solution was injected into the right hind paw of all rats. The representative test compound, the hydrochloride salt of Compound No. 3, was administered by intraperitoneal route twice to all the animals in the treatment group i.e. 4.6 mg/kg, 2 hrs prior and 2.3 mg/kg, 2 hrs later to the Carrageenan injection, while all the animals in the vehicle group received vehicle (saline) by i.p. route at similar time points. The volume of the right (injected) paw was measured at 0, 1, 3 and 6 hours using a plethysmometer. The volume of the right paw for each animal was measured and the mean difference in volume for the drug and vehicle group was calculated. Statistical analysis on the data obtained was performed using student's t-test, and the level of significance was p< 0.05. The results are shown in Table 6.

TABLE 6

| | | % Reduction in | | |
|---|---|---|---|---|
| | | Inflammation at | | |
| | i.p. Dose | different time intervals | | |
| Treatment | (mg/kg) | 0 hr | 1 hr | 6 hr |
| HCl salt of Compound | 4.6 | 0.0 | 52.7 | 36.2 |
| No. 3 | | | | |

1.2 Discussion of Test Results:

It can be seen from the Tables 2 and 3 that the compounds of the present invention induce HSP-70 as well as inhibit TNF-α in cultured cells incubated with the above said drugs.

Many investigators have shown anti-inflammatory properties exhibited by constitutive and inducible heat shock proteins. It has been demonstrated that HSP-72 expression occurs in inflamed tissue and this effect is associated with the remission of the inflammatory reaction. [Ianaro A et. al., Mol Pharmacol., 2003 Jul; Vol 64(1), pp. 85-93]. Hence, therapy with HSP inducing compounds would be useful in treating acute and chronic inflammatory conditions including arthritis. The representative test compound, the hydrochloride salt of Compound No. 3 when investigated for its anti-inflammatory activity in a rat model of inflammation, was shown to possess anti-inflammatory activity. This activity well correlates with our in vitro finding of increased HSP-70 mRNA levels with the hydrochloride salt of Compound No. 3, and further it was also observed that there was an inhibition of TNF-α in cultured cells incubated with the above said drugs suggesting the role of HSPs in anti-inflammatory activity and hence it can be concluded that the compounds of the instant invention would be useful for the treatment of inflammatory disorders.

### Example (V)

### Assessment of the Efficacy of Compounds of the Instant Invention in Myocardial Infarction (MI)

1.1 Materials and Methods:

Male Sprague-Dawley rats weighing 250-300 g were anaesthetized using Urethane. The body temperature of the anaesthetized animal was maintained at 37° C., using homoeothermic blanket. The carotid artery and the jugular vein were cannulated for recording the blood pressure and for the intravenous administration of test compounds, respectively. ECG (lead II) was recorded with electrode fixed on to the limbs. Tracheotomy was performed to allow artificial ventilation using an animal ventilator.

The left anterior descending coronary artery (LADCA) was ligated by the standard method of Thiemermann et al. 1989 [Thiemermann, C. et al., Brit. J. Pharmacol., 1989, Vol. 97, pp. 401-408], and a successful occlusion, was confirmed by a fall in blood pressure and elevation of the ST segment region of the ECG. The test compound/vehicle was administered intravenously immediately on occlusion and 2 hrs post occlusion. The occlusion was continued for an hour and was then released to enable reperfusion for the next two hours. At the end of reperfusion period the animals were sacrificed and heart was isolated and Evan's blue dye is passed through the coronaries to differentiate the area at risk from the normally perfused tissue and infarct characterized using TTC staining technique. The infarct as a percentage of left ventricle and area at risk was calculated. Statistical analysis on the data obtained was performed using student's t-test, and the level of significance was p<0.05. The results are shown in Table 7.

TABLE 7

Percentage
Mean

| Treatment | INF/LV | INF/AAR |
|---|---|---|
| Vehicle* | 26.8 | 45.9 |
| HCl salt of | 9.3 | 15.6 |
| Compound No. 3 | | |
| (3.43 mg/kg)** | | |

| | |
|---|---|
| % Reduction by Test | 65.4 |
| Compound | |

*Vehicle (0.9% saline) was administered to Control Group
**Compound was administered intravenously to Test Group.
INF = Infarct;
LV = Left ventricle;
AAR = Area at risk;
INF/LV = Infarct as a percent of left ventricle;
INF/AAR = Infarct as a percent of area at risk

1.2 Discussion of Test Results

There is abundant evidence, which supports the role of HSP-70 in protection against ischemic myocardial injury. Induction of HSP-70 has been shown to confer protection against subsequent ischemia as is evidenced by a direct correlation to post-ischemic myocardial preservation, reduction in infarct size and improved metabolic and functional recovery. [Liu X. et. al., Circulation, 1992, Vol. 86, pp. II358-II363; Martin J. L., Circulation, 1997, Vol. 96, pp. 4343-4348]. In this context, we found (as indicated in Table 7) that there was a greater reduction in myocardial infarct size in a rat model of Myocardial infarction, following treatment with a representative compound, the hydrochloride salt of Compound No. 3. The correlation between the in vivo and in vitro results showing increased HSP-70 mRNA levels subsequent to incubation with the hydrochloride salt of Compound No. 3 in cultured cells substantiates the earlier evidence about the potential role of HSP-70 in protection of ischemic myocardial injury. Hence, it can be concluded that compounds of the instant invention would be useful in the treatment of myocardial infarction by virtue of their HSP inducing activity.

It is to be understood that this invention is not limited to the particular methodology, protocols, cell lines, constructs, and reagents described herein and as such may vary. It is also to be understood that the terminology used herein is for the purpose of describing particular embodiments only, and is not intended to limit the scope of the present invention, which will be limited only by the appended claims.

### FORMULATION EXAMPLES

The following pharmaceutical formulations are suggested by way of examples alone and in no way restrict the forms in which they can be used.

### Example 1

A parenteral formulation of the following formula can be prepared as follows:

| Ingredient | Quantity | |
|---|---|---|
| Compound of formula (I) | 2.0 | mg/ml |
| N-Methyl-2-pyrrolidone | 10% | w/v |
| Buffer pH 9.2 | q.s. to 1 | ml |

Compound is dissolved in N-Methyl-2-pyrrolidone with sonication. The volume is made up with pH 9.2 Buffer, and the resulting solution is filtered through a 0.22 micron filter.

### Example 2

A typical parenteral formulation of the following formula can be prepared as follows:

| Ingredient | Quantity |
|---|---|
| Compound of formula (I) | 1 mg/ml |
| Dichloromethane | 1 ml |
| Tween-80 | 0.5% w/v |
| Water for injection | q.s. |

Compound is dissolved in dichloromethane. Tween-80 containing solution is mixed with the drug solution and homogenized to get a nanosuspension.

### Example 3

A typical parenteral emulsion formulation of the following formula can be prepared as follows:

| Ingredient | Quantity | |
|---|---|---|
| Compound of general formula I | 10 | mg/ml |
| Oleic acid | 10% | w/v |
| Tween-80 | 0.5% | w/v |
| Purified water | q.s. to 1 | ml |

Compound is mixed with Oleic acid & Tween-80 and the mixture warmed to a temperature of 40-50° C. Purified water pre-warmed to 40-50° C. is mixed with above mixture.

### Example 4

A typical solid pharmaceutical formulation can be prepared with the following materials together in the proportions by weight specified below:

| Ingredient | Quantity |
|---|---|
| Compound of formula (I) | 25 |
| Microcrystalline Cellulose pH 102 | 69 |
| Colloidal Silicon Dioxide | 0.5 |
| Sodium starch glycolate | 5.0 |
| Magnesium Stearate | 0.5 |

Compound of formula (I) is mixed with Microcrystalline Cellulose pH 102, Aerosil and blended with Sodium Starch Glycolate and Magnesium Stearate. Blend was compressed into tablets using 7 mm punches to contain 25 mg of compound of formula (I). Other tablets may be compressed to contain 50, 75, 100, 150 and 200 mg of compound of formula (I).

Other Formulations

Using an active ingredient of formula (I), various other formulations such as gels, creams, lotions, pastes, oral rinse, transdermal, ophthalmic solutions etc. may be prepared.

The above examples are provided by way of illustration alone and in no way restrict the scope of the invention.

1. A compound of general formula (I), 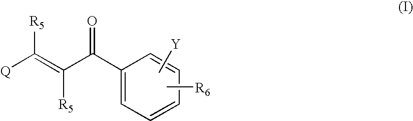
or its tautomeric forms, steroisomers, polymorphs, pharmaceutically acceptable salts, pharmaceutically acceptable solvates or prodrug, wherein Q represents a heteroaryl ring, said heteroaryl ring containing up to 2 nitrogen atoms and is selected from: 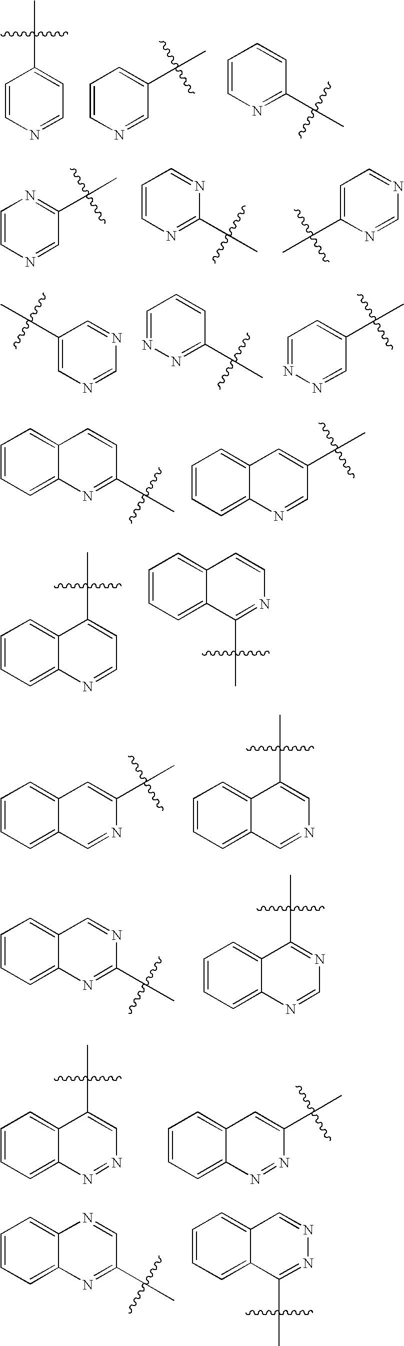 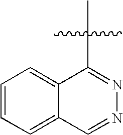
wherein Q is optionally substituted by R₁ and/or R₂, and the number of substitutents are selected from one to six;
R₁ in independently selected at each occurrence from —SO₂OR₇, —SO₂O(C₁₋₈alkyl), —NHNH₂, —NHNHSO₂R₇, —NH(CH₂)ₙR₄, —NHCO₂R₇, —NHCO₂(C₁₋₈alkyl), —NHSO₂O(C₁₋₈alkyl), —NHSO₂OR₇, —NHSO₂NH₂, —NH(CH₂)ₙCOR₄, —NH(CH₂)ₙOR₄, —NH(CH₂)ₙSR₇, —NH(CH₂)ₙSO₂R₇, —NH(CH₂ )ₙNHCOR₄, —NH(CH₂)ₙN(C₁₋₈alkyl)COR₄, —N(C₁₋₈alkyl)(CH₂)ₙNHCOR₄, —NH(CH₂)ₙNHNHSO₂R₇, —NH(CH₂)ₙNHSO₂R₄, —NH(CH₂)ₙN(C₁₋₈alkyl)SO₂R₄, —NH(CH₂)ₙN(NH₂)R₇, —NH(CH₂)ₙN[N(C₁₋₈alkyl)₂]R₇, —N(C₁₋₈alkyl)CO₂R₇, —N(C₁₋₈alkyl)CO₂(C₁₋₈alkyl), —N(C₁₋₈alkyl)SO₂O(C₁₋₈alkyl), —N(C₁₋₈alkyl)SO₂OR₇, —N(C₁₋₈alkyl)SO₂NH₂, —N(C₁₋₈alkyl)N(C₁₋₈alkyl)₂, —N(C₁₋₈alkyl)NH₂ , —NHNHCO(C₁₋₈alkyl), —N(C₁₋₈alkyl)NHCO(C₁₋₈alkyl), —NHNHCOR₇, —N(C₁₋₈alkyl)NHCOR₇, —N(C₁₋₈alkyl)—(CH₂)ₙR₄, —N(C₁₋₈alkyl)(CH₂)ₙCOR₄, —(CH₂)ₙSO₂R₇, —(CH₂)ₙCOR₄, —(CH₂)ₙR₄, —(CH₂)ₙNHSO₂ R₄, —(CH₂ )ₙN(C₁₋₈alkyl)SO₂R₄, —(CH₂)ₙNHCOR₇, —(CH₂ )ₙN(C₁₋₈alkyl)COR₇, —(CH₂ )ₙOR₄, —(CH₂)ₙSR₄, —(CH₂)ₙSR₃, —(CH₂)ₙSO₂R₇, —(CH₂)ₙNHNHSO₂R₇, —(CH₂)ₙN(NH₂)R₇, or —(CH₂)ₙN[N(C₁₋₈alkyl)₂]R₇;
R₂ is independently selected at each occurrence from hydrogen, hydroxy, halo, amino, C₁₋₈alkyl, —O(C₁₋₈alkyl), —S(C₁₋₈alkyl), —SO₂(C₁₋₈alkyl), oxo, thioxo, mono(C₁₋₈alkyl)amino, di(C₁₋₈alkyl)amino, —NHCO(C₁₋₈alkyl), —N(C₁₋₈alkyl)CO(C₁₋₈alkyl), —NHSO₂(C₁₋₈alkyl), —NHSO₂CF₃, —N(C₁₋₈alkyl)SO₂CF₃, —NHSO₂O(C₁₋₈alkyl), —N(C₁₋₈alkyl)SO₂(C₁₋₈alkyl), —N(C₁₋₈alkyl)SO₂O(C₁₋₈alkyl), —COOH, —CO₂(C₁₋₈alkyl), —NHCO₂(C₁₋₈alkyl), —N(C₁₋₈alkyl)CO₂(C₁₋₈alkyl), —CONH(C₁₋₈alkyl), —CON(C₁₋₈alkyl)₂, formyl, CF₃, CN, (CH₂)ₙOH, —(CH₂)ₙNH₂, —(CH₂)ₙNH(C₁₋₈alkyl), —(CH₂)ₙN(C₁₋₈alkyl)₂, —(CH₂)ₙO(C₁₋₈alkyl), —SO₃H, —SO₂O(C₁₋₈alkyl), —SO₂NH₂, —SO₂N(C₁₋₈alkyl)₂, —SO₂NH(C₁₋₈alkyl), —OSO₂(C₁₋₈alkyl), —N(C₁₋₈alkyl)SO₂NH₂, —NHSO₂NH(C₁₋₈alkyl), —NHSO₂N(C₁₋₈alkyl)₂, —N(C₁₋₈alkyl)SO₂N(C₁₋₈alkyl)₂, —NHSO₂NH₂, —NHC(NH)NH₂, —NHCONH₂, —NHC(O)NH(C₁₋₈alkyl), —NHC(O)N(C₁₋₈alkyl)₂, —N(C₁₋₈alkyl)C(O)N(C₁₋₈alkyl)₂, —NHNH₂, —N(C₁₋₈alkyl)N(C₁₋₈alkyl)₂, —N(C₁₋₈alkyl)NH₂, tetrazolyl, or three- to seven- membered heterocyclyl or heteroaryl ring having upto three heteroatoms independently selected from N, O, or S, wherein said three- to seven- membered heterocyclyl or heteroaryl ring is optionally substituted with 1, 2 or 3 substituents independently selected from the group consisting of halo, hydroxy, C₁₋₈alkyl, —O(C₁₋₈alkyl), nitro, amino, mono(C₁₋₈alkyl)amino, di(C₁₋₈alkyl)amino, —NHCO(C₁₋₈alkyl), —N(C₁₋₈alkyl)CO(C₁₋₈alkyl), —NHSO₂(C₁₋₈alkyl), —N(C₁₋₈alkyl)SO₂(C₁₋₈alkyl), —NHSO₂CF₃, —N(C₁₋₈alkyl)SO₂CF₃, —COOH, —CONH₂, —CONH(C₁₋₈alkyl), —CON(C₁₋₈alkyl)₂, —CO₂(C₁₋₈alkyl), —NHCO₂(C₁₋₈alkyl), —N(C₁₋₈alkyl)CO₂(C₁₋₈alkyl), CF₃, CN, —(CH₂)ₙOH, —(CH₂)ₙNH₂, —(CH₂)ₙNH(C₁₋₈alkyl), —(CH₂)ₙN(C₁₋₈alkyl)₂, —CH₂O(C₁₋₈alkyl), —NHSO₂NH₂, —N(C₁₋₈alkyl)SO₂NH₂, —NHSO₂NH(C₁₋₈alkyl), —NHSO₂N(C₁₋₈alkyl)₂, —N(C₁₋₈alkyl)SO₂N(C₁₋₈alkyl)₂, —NHCONH₂, —NHCONH(C₁₋₈alkyl), —NHCON(C₁₋₈alkyl)₂, —N(C₁₋₈alkyl)CON(C₁₋₈alkyl)₂, —S(C₁₋₈alkyl), —SO₂(C₁₋₈alkyl), —SO₃H, —SO₂O(C₁₋₈alkyl), —SO₂NH₂, —SO₂N(C₁₋₈alkyl)₂, —SO₂NH(C₁₋₈alkyl), or —NHC(NH)NH₂;
‘Y’ is selected from the group consisting of:
(a) —C(O)NRₐR_{b},
(b) —NR_{c}C(X)NRₐR_{b},
(C) —NR_{c}C(X)NR_{d}Rₑ,
(d) —NR_{c}C(O)OR_{f},
(e) —NR_{c}C(O)C(O)R_{g};
X is selected from O or S;
Rₐ and R_{b} together with the atoms with which they are attached form a three- to ten-membered monocyclic or bicyclic heterocyclyl or heteroaryl ring selected from the group consisting of aziridinyl, azepanyl, azetindinyl, azocanyl, azepinyl, diazepanyl, diazocanyl, hexahydropyridazinyl, hexahydropyrimidinyl, isothiazolidinyl, isoxazolidonyl, imidazolyl, imidazolidinyl, morpholinyl, oxazolidonyl, oxazolanyl, oxazetanyl, piperazinyl, piperazinonyl, piperidinyl, piperidonyl, pyrrolidinyl, pyrrolinyl, pyrroyl, pyrrolonyl, pyrrolidonyl, pyrazolyl, pyrazolonyl, thiomorpholinyl, thiomorpholin-1,1-dioxide, thiazolidinyl, thiazepanyl, thiazinyl, thiazocanyl, thiazetanyl, triazolyl, indolyl, indolinyl, indazolyl, tetrahydroquinolyl, tetrahydroisoquinolyl, or benzimidazolyl, wherein, said three- to ten-membered monocyclic or bicyclic heterocyclyl or heteroaryl ring is optionally substituted with 1, 2 or 3 substituents independently selected from the group consisting of
(1) halo, (2) hydroxy, (3) optionally substituted C₁₋₈alkyl, where the substituents are amino, C₁₋₃ alkoxy, mono(C₁₋₃alkyl)amino, di(C₁₋₃alkyl)amino, and hydroxy, (4) —O(C₁₋₈alkyl), (5) nitro, (6) amino, (7) mono(C₁₋₈alkyl)amino, (8) di(C₁₋₈alkyl)amino, (9) —COOH, (10) —CO(C₁₋₈alkyl), (11) —CONH₂, (12) —CONH(C₁₋₈alkyl), (13) —CON(C₁₋₈alkyl)₂, (14) —CO₂(C₁₋₈alkyl), (15) formyl, (16) =NOH, (17) CF₃, (18) CN, (19) —NHSO₂NH₂, (20) —NHCO(C₁₋₈alkyl), (21) —N(C₁₋₈alkyl)CO(C₁₋₈alkyl), (22) —NHSO₂(C₁₋₈alkyl), (23) —N(C₁₋₈alkyl)SO₂(C₁₋₈alkyl), (24) —NHSO₂CF₃, (25) —N(C₁₋₈alkyl)CO₂(C₁₋₈alkyl), (26) —N(C₁₋₈alkyl)SO₂CF₃, (27) —N(C₁₋₈alkyl)SO₂NH₂, (28) —NHSO₂NH(C₁₋₈alkyl), (29) —NHSO₂N(C₁₋₈alkyl)₂, (30) —N(C₁₋₈alkyl)SO₂N(C₁₋₈alkyl)₂, (31) —NHCONH₂, (32) —NHCONH(C₁₋₈alkyl), (33) —NHCON(C₁₋₈alkyl)₂, (34) —N(C₁₋₈alkyl)CO(C₁₋₈alkyl), (35) —N(C₁₋₈alkyl)CO₂(C₁₋₈alkyl), (36) —N(C₁₋₈alkyl)CON(C₁₋₈alkyl)₂, (37) —S(C₁₋₈alkyl), (38) —SO₂(C₁₋₈alkyl), (39) —SO₃H, (40) —SO₂O(C₁₋₈alkyl), (41) —SO₂NH₂, (42) —SO₂N(C₁₋₈alkyl)₂, (43) —SO₂NH(C₁₋₈alkyl), (44) —NHC(NH)NH₂, (45) phenyl, unsubstituted or substituted with one to two substituents selected from halo, nitro, C₁₋₃alkyl, C₁₋₃alkoxy, hydroxy, amino, mono(C₁₋₈alkyl)amino, di(C₁₋₈alkyl)amino, —NHCO(C₁₋₈alkyl), —N(C₁₋₈alkyl)CO(C₁₋₈alkyl), —NHCO₂(C₁₋₈alkyl), —N(C₁₋₈alkyl)CO₂(C₁₋₈alkyl), —NHNH₂, —N(C₁₋₈alkyl)N(C₁₋₈alkyl)₂, and —N(C₁₋₈alkyl)NH₂, (46) pyridyl, unsubstituted or substituted with one to two substituents selected from halo, C₁₋₃alkyl and C₁₋₃alkoxy, (47) —CO—(optionally substituted heteroaryl), (48) —CO—(optionally substituted heterocyclyl), (49) —O— (optionally substituted heteroaryl), (50) —O—(optionally substituted heterocyclyl), (51) optionally substituted heterocyclyl, (52) —NH—(optionally substituted heterocyclyl),
wherein the substituents on the optionally substituted heteroaryl and heterocyclyl are one to two groups independently selected from hydroxy, C₁₋₈alkyl, —O(C₁₋₈alkyl), oxo, thioxo, amino, mono(C₁₋₈alkyl)amino, di(C₁₋₈alkyl)amino, —NHCO(C₁₋₈alkyl), —N(C₁₋₈alkyl)CO(C₁₋₈alkyl), —NHCO₂(C₁₋₈alkyl), —N(C₁₋₈alkyl)CO₂(C₁₋₈alkyl), —NHNH₂, —N(C₁₋₈alkyl)N(C₁₋₈alkyl)₂, —NHSO₂(C₁₋₈alkyl), —NHSO₂NH₂ or —N(C₁₋₈alkyl)NH₂;
R_{c} and R_{d} are independently selected from hydrogen or C₁₋₆alkyl;
Rₑ is selected from R₇, —SO₂R₇, —SO₂R₃, —SO₂R₄, —COR₇, —(CH₂)ₙR₇, —(CH₂)ₙR₇, —CH₂)ₙCOR₇, —(CH₂)ₙSR₇, —(CH₂)ₙSO₂R₇, —(CH₂)ₙNHCOR₇, —(CH₂)ₙNHSO₂R₇, —CH₂)ₙN(C₁₋₈alkyl)COR₇, —(CH₂)ₙNHNHSO₂R₇, —(CH₂)ₙNHSO₂R₄, —(CH₂)ₙN(C₁₋₈alkyl)SO₂R₄, —(CH₂)ₙN(NH₂)R₇, —(CH₂)ₙN[N(C₁₋₈alkyl)₂]R₇, —NHSO₂R₇, optionally substituted C₁₋₈alkyl, wherein the substitutents are C₁₋₃ alkoxy, amino, mono(C₁₋₃alkyl)amino, di(C₁₋₃alkyl)amino, or hydroxy;
R_{f} is selected from the group consisting of (1) optionally substituted C₁₋₈alkyl, wherein the substituents are selected from C₁₋₃alkoxy, amino, mono(C₁₋₃alkyl)amino, di(C₁₋₃alkyl)amino, C₁₋₃alkyl, phenyl, or hydroxy, (2) —R₃, (3) —R₄, (4) phenyl, unsubstituted or substituted with R₂, (5) —(CH₂)ₙR₇, (6) —(CH₂)ₙCOR₇, (7) —(CH₂)ₙNR_{c}R₇, (8) —(CH₂)ₙNHSO₂R₇, (9) —(CH₂)ₙN(C₁₋₈alkyl)SO₂R₇, (10) —(CH₂)ₙNHCOR₇, (11) —(CH₂)ₙN(C₁₋₈alkyl)COR₇, (12) —(CH₂)ₙOR₇, (13) —(CH₂)ₙSR₇, (14) —(CH₂)ₙSO₂R₇, (15) —(CH₂)ₙNHNHSO₂R₇, (16) —(CH₂)ₙN(NH₂)R₇, (17) —(CH₂)ₙN{N(C₁₋₈alkyl)₂}R₇ or, (18) CCl₃;
R_{g} is selected from the group consisting of (1) mono(C₁₋₈alkyl)amino (2) di(C₁₋₈alkyl)amino, (3) NH₂, (4) —NHR₇, (5) —NR_{c}(CH₂)ₙR₇, (6) —NR_{c}(CH₂)ₙCOR₇, (7) —NH(CH₂)ₙO(C₁₋₈alkyl), (8) —NR_{c}(CH₂)ₙOR₇, (9) —NR_{c}(CH₂)ₙNHSO₂R₇, (10) —NR_{c}(CH₂)ₙN(C₁₋₈alkyl)SO₂R₇, (11) —NR_{c}(CH₂)ₙSO₂R₇, (12) —NR_{c}SO₂R₇, (13) —NR_{c}(CH₂)ₙSR₇, (14) —N(NH₂)R₇, (15) —N[N(C₁₋₈alkyl)₂]R₇, (16) —NR_{c}(CH₂)ₙNHNHSO₂R₇, (17) —NR_{c}(CH₂)ₙN(NH₂)R₇, (18) —NR_{c}(CH₂)ₙN[N(C₁₋₈alkyl)₂]R₇, (19) —NR_{c}(CH₂)ₙNHCOR₇, (20) —NHNHSO₂R₇, (21) optionally substituted three- to ten-membered monocyclic or bicyclic heterocyclyl or heteroaryl ring attached through the ring nitrogen atom and selected from the group consisting of aziridinyl, azepanyl, azetindinyl, azocanyl, azepinyl, diazepanyl, diazocanyl, hexahydropyridazinyl, hexahydropyrimidinyl, isothiazolidinyl, isoxazolidonyl, imidazolyl, imidazolidinyl, morpholinyl, oxazolidonyl, oxazolanyl, oxazetanyl, piperazinyl, piperazinonyl, piperidinyl, piperidonyl, pyrrolidinyl, pyrrolinyl, pyrroyl, pyrrolonyl, pyrrolidonyl, pyrazolyl, pyrazolonyl, thiomorpholinyl, thiomorpholin-1,1-dioxide, thiazolidinyl, thiazepanyl, thiazinyl, thiazocanyl, thiazetanyl, triazolyl, indolyl, indolinyl, indazolyl, tetrahydroquinolyl, tetrahydroisoquinolyl, or benzimidazolyl,
wherein, the substituents on said optionally substituted three- to ten-membered monocyclic or bicyclic heterocyclyl or heteroaryl ring are 1, 2 or 3 groups independently selected from (1) halo, (2) hydroxy, (3) C₁₋₈alkyl, unsubstituted or substituted with C₁₋₃alkoxy, amino, mono(C₁₋₃alkyl)amino, di(C₁₋₃alkyl)amino, C₁₋₃alkyl, and hydroxy, (4) —O(C₁₋₈alkyl), (5) nitro, (6) amino, (7) mono(C₁₋₈alkyl)amino, (8) di(C₁₋₈alkyl)amino, (9) —COOH, (10) —CO(C₁₋₈alkyl), (11) —CONH₂, (12) —CONH(C₁₋₈alkyl), (13) —CON(C₁₋₈alkyl)₂, (14) —CO₂(C₁₋₈alkyl), (15) formyl, (16) =NOH, (17) CF₃, (18) CN, (19) —NHSO₂NH₂, (20) —NHCO(C₁₋₈alkyl), (21) —N(C₁₋₈alkyl)CO(C₁₋₈alkyl), (22) —NHSO₂(C₁₋₈alkyl), (23) —N(C₁₋₈alkyl)SO₂(C₁₋₈alkyl), (24) —NHSO₂CF₃, (25) —N(C₁₋₈alkyl)CO₂(C₁₋₈alkyl), (26) —N(C₁₋₈alkyl)SO₂CF₃, (27) —N(C₁₋₈alkyl)SO₂NH₂, (28) —NHSO₂NH(C₁₋₈alkyl), (29) —NHSO₂N(C₁₋₈alkyl)₂, (30) —N(C₁₋₈alkyl)SO₂N(C₁₋₈alkyl)₂, (31) —NHCONH₂, (32) —NHCONH(C₁₋₈alkyl), (33) —NHCON(C₁₋₈alkyl)₂, (34) —N(C₁₋₈alkyl)CO (C₁₋₈alkyl), (35) —N(C₁₋₈alkyl)CO₂(C₁₋₈alkyl), (36) —N(C₁₋₈alkyl)CON(C₁₋₈alkyl)₂, (37) —S(C₁₋₈alkyl), (38) —SO₂(C₁₋₈alkyl), (39) —SO₃H, (40) —SO₂O(C₁₋₈alkyl), (41) —SO₂NH₂, (42) —SO₂N(C₁₋₈alkyl)₂, (43) —SO₂NH(C₁₋₈alkyl), (44) —NHC(NH)NH₂,
n is independently selected at each occurrence, from 1, 2 or 3;
R₃ at each occurrence is optionally substituted monocyclic three- to seven-membered heteroaryl ring having one to three heteroatoms independently selected from N, O, or S, wherein the substitution is by 1,2 or 3 substituents represented by R₂;
R₄ at each occurrence is optionally substituted monocyclic three- to seven-membered heterocyclyl ring having one to three heteroatoms independently selected from N, O or S, wherein the substitution is by 1,2 or 3 substituents represented by R₂;
R₅ at each occurrence is independently selected from hydrogen, C₁₋₆alkyl or CF₃;
R₆ at each occurrence are 1 or 2 groups independently selected from hydrogen, —O(C₁₋₈alkyl), halo, C₁₋₆alkyl, mono(C₁₋₆alkyl)amino or di(C₁₋₆alkyl)amino;
R₇ at each occurrence is
1. optionally substituted monocyclic three- to seven-membered aryl;
2. optionally substituted monocyclic three- to seven-membered heteroaryl or heterocyclyl having one to three heteroatoms independently selected from N, O or S,
wherein the substitution on R₇ is by 1, 2 or 3 substituents represented by R₂.
with the proviso that when Y is NR_{c}C(X)NR_{d}Rₑ and Rₑ=R₇, R₇ is not furan, thiophene, isooxazole, isothiazole and phenyl.

2. The compound as claimed in claim 1, wherein Q represents a heteroaryl ring as defined in claim 1, said Q may be unsubstituted by 1 to 6 substituents represented by R₂,
R₂ is independently selected at each occurrence from hydrogen, hydroxy, halo, amino, C₁₋₈alkyl, —O(C₁₋₈alkyl), —S(C₁₋₈alkyl), —SO₂(C₁₋₈alkyl), oxo, thioxo, mono(C₁₋₈alkyl)amino, di(C₁₋₈alkyl)amino, —NHCO(C₁₋₈alkyl), —N(C₁₋₈alkyl)CO(C₁₋₈alkyl), —NHSO₂(C₁₋₈alkyl), —NHSO₂CF₃, —N(C₁₋₈alkyl)SO₂CF₃, —NHSO₂O(C₁₋₈alkyl), —N(C₁₋₈alkyl)SO₂(C₁₋₈alkyl), —N(C₁₋₈alkyl)SO₂O(C₁₋₈alkyl), —COOH, —CO₂(C₁₋₈alkyl), —NHCO₂(C₁₋₈alkyl), —N(C₁₋₈alkyl)CO_{2(C}₁₋₈alkyl), —CONH₂, —CONH(C₁₋₈alkyl), —CON(C₁₋₈alkyl)₂, formyl, CF₃, CN, —(CH₂)ₙOH, —(CH₂)ₙNH₂, —(CH₂)ₙNH(C₁₋₈alkyl), —(CH₂)ₙN(C₁₋₈alkyl)₂, —(CH₂)ₙO(C₁₋₈alkyl), —SO₃H, —SO₂O(C₁₋₈alkyl), —SO₂NH₂, —SO₂N(C₁₋₈alkyl)₂, —SO₂NH(C₁₋₈alkyl), —OSO₂(C₁₋₈alkyl), —N(C₁₋₈alkyl)SO₂NH₂, —NHSO₂NH(C₁₋₈alkyl), —NHSO₂N(C₁₋₈alkyl)₂, —N(C₁₋₈alkyl)SO₂N(C₁₋₈alkyl)₂, —NHSO₂NH₂, —NHC(NH)NH₂, —NHCONH₂, —NHC(O)NH(C₁₋₈alkyl), —NHC(O)N(C₁₋₈alkyl)₂, —N(C₁₋₈alkyl)C(O)N(C₁₋₈alkyl)₂, —NHNH₂, —N(C₁₋₈alkyl)N(C₁₋₈alkyl)₂, —N(C₁₋₈alkyl)NH₂, tetrazolyl or three- to seven-membered heterocyclyl or heteroaryl ring having upto three heteroatoms independently selected from N, O, or S, wherein said three- to seven-membered heterocyclyl or heteroaryl ring is optionally substituted with 1, 2 or 3 substituents independently selected from the group consisting of halo, hydroxy, C₁₋₈alkyl, —O(C₁₋₈alkyl), nitro, amino, mono(C₁₋₈alkyl)amino, di(C₁₋₈alkyl)amino, —NHCO(C₁₋₈alkyl), —N(C₁₋₈alkyl)CO(C₁₋₈alkyl), —NHSO₂(C₁₋₈alkyl), —N(C₁₋₈alkyl)SO₂(C₁₋₈alkyl), —NHSO₂CF₃, —N(C₁₋₈alkyl)SO₂CF₃, —COOH, —CONH₂, —CONH(C₁₋₈alkyl), —CON(C₁₋₈alkyl)₂, —CO₂(C₁₋₈alkyl), —NHCO₂(C₁₋₈alkyl), —N(C₁₋₈alkyl)CO₂(C₁₋₈alkyl), CF₃, CN, —(CH₂)ₙOH, —(CH₂)ₙNH₂, —(CH₂)ₙNH(C₁₋₈alkyl), —(CH₂)ₙN(C₁₋₈alkyl)₂, —CH₂O(C₁₋₈alkyl), —NHSO₂NH₂, —N(C₁₋₈alkyl)SO₂NH₂, —NHSO₂NH(C₁₋₈alkyl), —NHSO₂N(C₁₋₈alkyl)₂, —N(C₁₋₈alkyl)SO₂N(C₁₋₈alkyl)₂, —NHCONH₂, —NHCONH(C₁₋₈alkyl), —NHCON(C₁₋₈alkyl)₂, —N(C₁₋₈alkyl)CON(C₁₋₈alkyl)₂, —S(C₁₋₈alkyl), —SO₂(C₁₋₈alkyl), —SO₃H, —SO₂O(C₁₋₈alkyl), —SO₂NH₂, —SO₂N(C₁₋₈alkyl)₂, —SO₂NH(C₁₋₈alkyl), or —NHC(NH)NH₂;
‘Y’ is selected from the group consisting of:
(a) —C(O)NRₐR_{b},
(b) —NR_{c}C(X)NRₐR_{b},
(c) —NR_{c}C(X)NR_{d}Rₑ,
(d) —NR_{c}C(O)OR_{f},
(e) —NR_{c}C(O)C(O)R_{g};
X is selected from O or S;
Rₐ and R_{b} together with the atoms with which they are attached form a three- to ten-membered monocyclic or bicyclic heterocyclyl or heteroaryl ring selected from the group consisting of aziridinyl, azepanyl, azetindinyl, azocanyl, azepinyl, diazepanyl, diazocanyl, hexahydropyridazinyl, hexahydropyrimidinyl, isothiazolidinyl, isoxazolidonyl, imidazolyl, imidazolidinyl, morpholinyl, oxazolidonyl, oxazolanyl, oxazetanyl, piperazinyl, piperazinonyl, piperidinyl, piperidonyl, pyrrolidinyl, pyrrolinyl, pyrroyl, pyrrolonyl, pyrrolidonyl, pyrazolyl, pyrazolonyl, thiomorpholinyl, thiomorpholin-1,1-dioxide, thiazolidinyl, thiazepanyl, thiazinyl, thiazocanyl, thiazetanyl, triazolyl, indolyl, indolinyl, indazolyl, tetrahydroquinolyl, tetrahydroisoquinolyl, or benzimidazolyl, wherein, said three- to ten- membered monocyclic or bicyclic heterocyclyl or heteroaryl ring is optionally substituted with 1, 2, or 3 substituents independently selected from the group consisting of
(1) phenyl, unsubstituted or substituted with a single substituent selected from hydroxy, mono(C₁₋₈alkyl)amino, di(C₁₋₈alkyl)amino, —NHCO(C₁₋₈alkyl), —N(C₁₋₈alkyl)CO(C₁₋₈alkyl), —NHCO₂(C₁₋₈alkyl), —N(C₁₋₈alkyl)CO₂(C₁₋₈alkyl), —NHNH₂, —N(C₁₋₈alkyl)N(C₁₋₈alkyl)₂, and —N(C₁₋₈alkyl)NH₂,
(2) =NOH, (3) optionally substituted heterocyclyl, (4) —O-optionally substituted heteroaryl, (5) —O-optionally substituted heterocyclyl, (6) —CO-optionally substituted heteroaryl, or (7) —CO-optionally substituted heterocyclyl,
wherein the substituent on the optionally substituted heteroaryl and heterocyclyl is a single group selected from hydroxy, C₁₋₈alkyl, —O(C₁₋₈alkyl), oxo, thioxo, amino, mono(C₁₋₈alkyl)amino, di(C₁₋₈alkyl)amino, —NHCO(C₁₋₈alkyl), —N(C₁₋₈alkyl)CO(C₁₋₈alkyl), NHCO₂(C₁₋₈alkyl), —N(C₁₋₈alkyl)CO₂(C₁₋₈alkyl), —NHNH₂, —N(C₁₋₈alkyl)N(C₁₋₈alkyl)₂, or —N(C₁₋₈alkyl)NH₂;
R_{c} and R_{d} are independently selected from hydrogen or C₁₋₆alkyl;
Rₑ is independently selected from —SO₂R₃, —SO₂R₄, —(CH₂)ₙR₄, —(CH₂)ₙR₄, —(CH₂)ₙCOR₄, —(CH₂)ₙOR₄, —(CH₂)ₙSR₇, —(CH₂)ₙSO₂R₇, —(CH₂)ₙNHCOR₇, —(CH₂)ₙN(C₁₋₈alkyl)COR₇, —(CH₂)ₙNHNHSO₂R₇, —(CH₂)ₙNHSO₂R₄, —(CH₂)ₙN(C₁₋₈alkyl)SO₂R₄, —(CH₂)ₙN(NH₂ )R₇, —(CH₂)ₙN[N(C₁₋₈alkyl)₂]R₇, or —NHSO₂R₇;
R_{f} is selected from the group consisting of (1) optionally substituted C₁₋₈alkyl, wherein the substituents are selected from oxo, thioxo, amino, C₁₋₃alkoxy, mono(C₁₋₃)alkylamino, di(C₁₋₃alkyl)amino, or hydroxy, (2) —R₃, (3) —R₄, (4) phenyl, unsubstituted or substituted with R₂, (5) —(CH₂)ₙR₇, (6) —(CH₂)ₙCOR₇, (7) —(CH₂)ₙNR_{c}R₇, (8) —(CH₂)ₙNHSO₂R₇, (9) —(CH₂)ₙN(C₁₋₈alkyl)SO₂R₇, (10) —(CH₂)ₙNHCOR₇, (11) —(CH₂)ₙN(C₁₋₈alkyl)COR₇, (12) —(CH₂)ₙOR₇, (13) —(CH₂)ₙSR₇, (14) —(CH₂)ₙSO₂R₇, (15) —CH₂)ₙNHNHSO₂R₇, (16) —(CH₂)ₙN(NH₂)R₇, or (17) —(CH₂)ₙN{N(C₁₋₈alkyl)₂}R₇;
R_{g} is selected from the group consisting of —NR_{c}(CH₂)ₙR₄, —NR_{c}(CH₂)ₙCOR₄, —NR_{c}(CH₂)ₙOR₄, —NR_{c}(CH₂)ₙNHSO₂R₄, —NR_{c}(CH₂)ₙN(C₁₋₈alkyl)SO₂R₄, —NR_{c}(CH₂)ₙSO₂R₇, —NR_{c}SO₂R₇, —NR_{c}(CH₂)ₙSR₇, —N(NH₂)R₇, —N[N(C₁₋₈alkyl)₂]R₇, —NR_{c}(CH₂)ₙNHNHSO₂R₇, —NR_{c}(CH₂ )ₙN(NH₂)R₇, —NR_{c}(CH₂)ₙN[N(C₁₋₈alkyl)₂]R₇, or —NR_{c}(CH₂)ₙNHCOR₇;
n is independently selected at each occurrence, from 1, 2 or 3;
R₃ at each occurrence is optionally substituted monocyclic three to seven membered heteroaryl ring having one to three heteroatoms independently selected from N, O, or S, wherein the substitution is by 1, 2 or 3 substituents represented by R₂;
R₄ at each occurrence is optionally substituted monocyclic three to seven membered heterocyclyl ring having one to three heteroatoms independently selected from N, O or S, wherein the substitution is by 1, 2 or 3 substituents represented by R₂;
R₅ at each occurrence is independently selected from hydrogen, C₁₋₆alkyl or CF₃;
R₆ at each occurrence are 1 or 2 groups independently selected from hydrogen, —O(C₁₋₈alkyl), halo, C₁₋₆alkyl, mono(C₁₋₆alkyl)amino or di(C₁₋₆alkyl)amino;
R₇ at each occurrence is
1. optionally substituted monocyclic three- to seven-membered aryl;
2. optionally substituted monocyclic three- to seven-membered heteroaryl or heterocyclyl having one to three heteroatoms independently selected from N, O or S,
wherein the substitution on R₇ is by 1, 2 or 3 substituents represented by R₂;
with the proviso that,
when R_{f} is C₁₋₈alkyl, aryl, or R₃, then R₂ is an optionally substituted three- to seven-membered heterocyclyl or heteroaryl ring having upto three heteroatoms independently selected from N, O, or S.

3. The compound as claimed in claim 1, wherein Q represents a heteroaryl ring as defined in claim 1, said Q is substituted by either R₁ or both R₁ and R₂, said substituents are selected from one to six;
R₁ is independently selected at each occurrence from, —SO₂OR₇, —SO₂O(C₁₋₈alkyl), —NHNH₂, —NHNHSO₂R₇, —NH(CH₂)ₙR₄, —NHCO₂R₇, —NHCO₂(C₁₋₈alkyl), —NHSO₂O(C₁₋₈alkyl), —NHSO₂OR₇, —NHSO₂NH₂, —NH(CH₂)ₙCOR₄, —NH(CH₂)ₙOR₄, —NH(CH₂)ₙSR₇, —NH(CH₂)ₙSO₂R₇, —NH(CH₂ )ₙNHCOR₄, —NH(CH₂)ₙN(C₁₋₈alkyl)COR₄, —N(C₁₋₈alkyl)(CH₂)ₙNHCOR₄, —NH(CH₂)ₙNHNHSO₂R₇, —NH(CH₂)ₙNHSO₂R₄, —NH(CH₂)ₙN(C₁₋₈alkyl)SO₂R₄, —NH(CH₂)ₙN(NH₂)R₇, —NH(CH₂)ₙN[N(C₁₋₈alkyl)₂]R₇, —N(C₁₋₈alkyl)CO₂R₇, —N(C₁₋₈alkyl)CO₂(C₁₋₈alkyl), —N(C₁₋₈alkyl)SO₂O(C₁₋₈alkyl), —N(C₁₋₈alkyl)SO₂OR₇, —N(C₁₋₈alkyl)SO₂NH₂, —N(C₁₋₈alkyl)N(C₁₋₈alkyl)₂, —N(C₁₋₈alkyl)NH₂, —NHNHCO(C₁₋₈alkyl), —N(C₁₋₈alkyl)NHCO(C₁₋₈alkyl), —NHNHCOR₇, —N(C₁₋₈alkyl)NHCOR₇, —N(C₁₋₈alkyl)—CH₂)ₙR₄, —N(C₁₋₈alkyl)(CH₂)ₙCOR₄, —(CH₂)ₙSO₂R₇, —(CH₂)ₙCOR₄, —(CH₂)ₙR₄, —(CH₂)ₙNHSO₂R₄, —(CH₂)ₙN(C₁₋₈alkyl)SO₂R₄, —(CH₂)ₙNHCOR₇, —(CH₂)ₙN(C₁₋₈alkyl)COR₇, —(CH₂)ₙOR₄, —(CH₂)ₙSR₄, —(CH₂)ₙSR₃, —(CH₂)ₙSO₂R₇, —(CH₂)ₙNHNHSO₂R₇, —(CH₂)ₙN(NH₂)R₇, or —(CH₂)ₙN[N(C₁₋₈alkyl)₂]R₇;
R₂ is as defined in claim 1;
‘Y’ is selected from the group consisting of:
(a) —C(O)NRₐR_{b},
(b) —NR_{c}C(X)NRₐR_{b},
(c) —NR_{c}C(X)NR_{d}Rₐ,
(d) —NR_{c}C(O)OR_{f},
(e) —NR_{c}C(O)C(O)R_{g};
X is selected from O or S;
Rₐ and R_{b} together with the atoms with which they are attached form a three- to ten-membered monocyclic or bicyclic heterocyclyl or heteroaryl ring selected from the group consisting of aziridinyl, azepanyl, azetindinyl, azocanyl, azepinyl, diazepanyl, diazocanyl, hexahydropyridazinyl, hexahydropyrimidinyl, isothiazolidinyl, isoxazolidonyl, imidazolyl, imidazolidinyl, morpholinyl, oxazolidonyl, oxazolanyl, oxazetanyl, piperazinyl, piperazinonyl, piperidinyl, piperidonyl, pyrrolidinyl, pyrrolinyl, pyrroyl, pyrrolonyl, pyrrolidonyl, pyrazolyl, pyrazolonyl, thiomorpholinyl, thiomorpholin-1,1-dioxide, thiazolidinyl, thiazepanyl, thiazinyl, thiazocanyl, thiazetanyl, triazolyl, indolyl, indolinyl, indazolyl, tetrahydroquinolyl, tetrahydroisoquinolyl, or benzimidazolyl, wherein, said three- to ten-membered monocyclic or bicyclic heterocyclyl or heteroaryl ring is optionally substituted with 1, 2, or 3 substituents independently selected from the group consisting of
(1) halo, (2) hydroxy, (3) optionally substituted C₁₋₈alkyl, wherein the substituents are amino, C₁₋₃alkoxy, mono(C₁₋₃alkyl)amino, di(C₁₋₃alkyl)amino, and hydroxy, (4) —O(C₁₋₈alkyl), (5) nitro, (6) amino, (7) mono(C₁₋₈alkyl)amino, (8) di(C₁₋₈alkyl)amino, (9) —COOH, (10) —CO(C₁₋₈alkyl), (11) —CONH₂, (12) —CONH(C₁₋₈alkyl), (13) —CON(C₁₋₈alkyl)₂, (14) —CO₂(C₁₋₈alkyl), (15) formyl, (16) =NOH, (17) CF₃, (18) CN, (19) —NHSO₂NH₂ (20) —NHCO(C₁₋₈alkyl), (21) —N(C₁₋₈alkyl)CO(C₁₋₈alkyl), (22) —NHSO₂(C₁₋₈alkyl), (23) —N(C₁₋₈alkyl)SO₂(C₁₋₈alkyl), (24) —NHSO₂CF₃, (25) —N(C₁₋₈alkyl)CO₂(C₁₋₈alkyl), (26) —N(C₁₋₈alkyl)SO₂CF₃, (27) —N(C₁₋₈alkyl)SO₂NH₂, (28) —NHSO₂NH(C₁₋₈alkyl), (29) —NHSO₂N(C₁₋₈alkyl)₂, (30) —N(C₁₋₈alkyl)SO₂N(C₁₋₈alkyl)₂, (31) —NHCONH₂, (32) —NHCONH(C₁₋₈alkyl), (33) —NHCON(C₁₋₈alkyl)₂, (34) —N(C₁₋₈alkyl)CO(C₁₋₈alkyl), (35) —N(C₁₋₈alkyl)CO₂(C₁₋₈alkyl), (36) —N(C₁₋₈alkyl)CON(C₁₋₈alkyl)₂, (37) —S(C₁₋₈alkyl), (38) —SO₂(C₁₋₈alkyl), (39) —SO₃H, (40) —SO₂O(C₁₋₈alkyl), (41) —SO₂NH₂ , (42) —SO₂N(C₁₋₈alkyl)₂, (43) —SO₂NH(C₁₋₈alkyl), (44) —NHC(NH)NH₂, (45) phenyl, unsubstituted or substituted with one to two substitutents selected from halo, nitro, C₁₋₃alkyl, C₁₋₃alkoxy, hydroxy, amino, mono(C₁₋₈alkyl)amino, di(C₁₋₈alkyl)amino, —NHCO(C₁₋₈alkyl), —N(C₁₋₈alkyl)CO(C₁₋₈alkyl), —NHCO₂(C₁₋₈alkyl), —NHCO₂(C₁₋₈alkyl), —N(C₁₋₈alkyl)CO₂(C₁₋₈alkyl), —NHNH₂, —N(C₁₋₈alkyl)N(C₁₋₈alkyl)₂, and —N(C₁₋₈alkyl)NH₂, (46) pyridyl, unsubstituted or substituted with one to two substitutents selected from halo, C₁₋₃alkyl and C₁₋₃alkoxy, (47) —CO—(optionally substituted heteroaryl), (48) —CO—(optionally substituted heterocyclyl), (49) —O— (optionally substituted heteroaryl), (50) —O—(optionally substituted heterocyclyl), (51) optionally substituted heterocyclyl, (52) —NH—(optionally substituted heterocyclyl),
wherein the substituents on the optionally substituted heteroaryl and heterocyclyl are one to two groups independently selected from hydroxy, C₁₋₈alkyl, —O(C₁₋₈alkyl), oxo, thioxo, amino, mono(C₁₋₈alkyl)amino, di(C₁₋₈alkyl)amino, —NHCO(C₁₋₈alkyl), —N(C₁₋₈alkyl)CO(C₁₋₈alkyl), —NHCO₂(C₁₋₈alkyl), —N(C₁₋₈alkyl)CO₂(C₁₋₈alkyl), —NHNH₂, —N(C₁₋₈alkyl)N(C₁₋₈alkyl)₂, NHSO₂(C₁₋₈alkyl), —NHSO₂NH₂ or —N(C₁₋₈alkyl)NH₂;
R_{c} and R_{d} are independently selected from hydrogen or C₁₋₆alkyl;
Rₑ is selected from R₇, —SO₂R₇, —SO₂R₃, —SO₂R₄, —COR₇, —(CH₂)ₙR₇, —(CH₂)ₙCOR₇, —(CH₂)ₙOR₇, —(CH₂)ₙSR₇, —(CH₂)ₙSO₂R₇, —(CH₂)ₙNHCOR₇, —(CH₂)ₙNHSO₂R₇, —(CH₂)ₙN(C₁₋₈alkyl)COR₇, —(CH₂)ₙNHNHSO₂R₇, —(CH₂)ₙNHSO₂R₄, —(CH₂)ₙN(C₁₋₈alkyl)SO₂R₄, —(CH₂)ₙN(NH₂)R₇, —(CH₂)ₙN[N(C₁₋₈alkyl)₂]R₇, —NHSO₂R₇, optionally substituted C₁₋₈alkyl, wherein the substituents are C₁₋₃ alkoxy, amino, mono(C₁₋₃alkyl)amino, di(C₁₋₃alkyl)amino, or hydroxy;
R_{f} is selected from the group consisting of (1) optionally substituted C₁₋₈alkyl, wherein the substituents are selected from C₁₋₃alkoxy, amino, mono(C₁₋₃alkyl)amino, di(C₁₋₃alkyl)amino, C₁₋₃alkyl, phenyl, or hydroxy, (2) —R₃, (3) —R₄, (4) phenyl, unsubstituted or substituted with R₂, (5) —(CH₂)ₙR₇, (6) —(CH₂)ₙCOR₇, (7) —(CH₂)ₙNR_{c}R₇, (8) —(CH₂)ₙNHSO₂R₇, (9) —(CH₂)ₙN(C₁₋₈alkyl)SO₂R₇, (10) —(CH₂ )ₙNHCOR₇, (11) —CH₂)ₙN(C₁₋₈alkyl)COR₇, (12) —(CH₂)ₙOR₇, (13) —(CH₂)ₙSR₇, (14) —(CH₂)ₙSO₂R₇, (15) —(CH₂)ₙNHNHSO₂R₇, (16) —(CH₂)ₙN(NH₂)R₇, (17) —(CH₂)ₙN{N(C₁₋₈alkyl)₂}R₇ or (18) CCl₃;
R_{g} is selected from the group consisting of (1) mono(C₁₋₈alkyl)amino (2) di(C₁₋₈-alkyl)amino, (3) NH₂, (4) —NHR₇, (5) —NR_{c}(CH₂)ₙR₇, (6) —NR_{c}(CH₂)ₙCOR₇, (7) —NH(CH₂)ₙO(C₁₋₈alkyl), (8) —NR_{c}(CH₂)ₙOR₇, (9) —NR_{c}(CH₂)ₙNHSO₂R₇, (10) —NR_{c}(CH₂)ₙN(C₁₋₈alkyl)SO₂R₇, (11) —NR_{c}(CH₂)ₙSO₂R₇, (12) —NR_{c}SO₂R₇, (13) —NR_{c}(CH₂)ₙSR₇, (14) —N(NH₂)R₇, (15) —N[N(C₁₋₈alkyl)₂]R₇, (16) —NR_{c}(CH₂)ₙNHNHSO₂R₇, (17) —NR_{c}(CH₂)ₙN(NH₂)R₇, (18) —NR_{c}(CH₂)ₙN[N(C₁₋₈alkyl)₂]R₇, (19) —NR_{c}(CH₂)ₙNHCOR₇, (20) —NHNHSO₂R₇, (21) optionally substituted three- to ten-membered monocyclic or bicyclic heterocyclyl or heteroaryl ring attached through the ring nitrogen atom and selected from the group consisting of aziridinyl, azepanyl, azetindinyl, azocanyl, azepinyl, diazepanyl, diazocanyl, hexahydropyridazinyl, hexahydropyrimidinyl, isothiazolidinyl, isoxazolidonyl, imidazolyl, imidazolidinyl, morpholinyl, oxazolidonyl, oxazolanyl, oxazetanyl, piperazinyl, piperazinonyl, piperidinyl, piperidonyl, pyrrolidinyl, pyrrolinyl, pyrroyl, pyrrolonyl, pyrrolidonyl, pyrazolyl, pyrazolonyl, thiomorpholinyl, thiomorpholin-1,1-dioxide, thiazolidinyl, thiazepanyl, thiazinyl, thiazocanyl, thiazetanyl, triazolyl, indolyl, indolinyl, indazolyl, tetrahydroquinolyl, tetrahydroisoquinolyl, or benzimidazolyl,
wherein, the substituents on said optionally substituted three- to ten-membered monocyclic or bicyclic heterocyclyl or heteroaryl ring are 1, 2 or 3 groups independently selected from (1) halo, (2) hydroxy, (3) C₁₋₈alkyl, unsubstituted or substituted with C₁₋₃alkoxy, amino, mono(C₁₋₃alkyl)amino, di(C₁₋₃alkyl)amino, C₁₋₃alkyl, and hydroxy, (4) —O(C₁₋₈alkyl), (5) nitro, (6) amino, (7) mono(C₁₋₈alkyl)amino, (8) di(C₁₋₈alkyl)amino, (9) —COOH, (10) —CO(C₁₋₈alkyl), (11) —CONH₂, (12) —CONH(C₁₋₈alkyl), (13) —CON(C₁₋₈alkyl)₂, (14) —CO₂(C₁₋₈alkyl), (15) formyl, (16) =NOH, (17) CF₃, (18) CN, (19) —NHSO₂NH₂, (20) —NHCO(C₁₋₈alkyl), (21) —N(C₁₋₈alkyl)CO(C₁₋₈alkyl), (22) —NHSO₂(C₁₋₈alkyl), (23) —N(C₁₋₈alkyl)SO₂(C₁₋₈alkyl), (24) —NHSO₂CF₃, (25) —N(C₁₋₈alkyl)CO₂(C₁₋₈alkyl), (26) —N(C₁₋₈alkyl)SO₂CF₃, (27) —N(C₁₋₈alkyl)SO₂NH₂, (28) —NHSO₂NH(C₁₋₈alkyl), (29) —NHSO₂N(C₁₋₈alkyl)₂, (30) —N(C₁₋₈alkyl)SO₂N(C₁₋₈alkyl)₂, (31) —NHCONH₂, (32) —NHCONH(C₁₋₈alkyl), (33) —NHCON(C₁₋₈alkyl)₂, (34) —N(C₁₋₈alkyl)CO (C₁₋₈alkyl), (35) —N(C₁₋₈alkyl)CO₂(C₁₋₈alkyl), (36) —N(C₁₋₈alkyl)CON(C₁₋₈alkyl)₂, (37) —S(C₁₋₈alkyl), (38) —SO₂(C₁₋₈alkyl), (39) —SO₃H, (40) —SO₂O(C₁₋₈alkyl), (41) —SO₂NH₂, (42) —SO₂N(C₁₋₈alkyl)₂, (43) —SO₂NH(C₁₋₈alkyl), or (44) —NHC(NH)NH₂,
n is independently selected at each occurrence, from 1, 2 or 3;
R₃ at each occurrence is optionally substituted monocyclic three to seven membered heteroaryl ring having one to three heteroatoms independently selected from N, O, or S, wherein the substitution is by 1, 2 or 3 substituents represented by R₂;
R₄ at each occurrence is optionally substituted monocyclic three to seven membered heterocyclyl ring having one to three heteroatoms independently selected from N, O or S, wherein the substitution is by 1, 2 or 3 substituents represented by R₂;
R₅ at each occurrence is independently selected from hydrogen, C₁₋₆alkyl or CF₃;
R₆ at each occurrence are 1 or 2 groups independently selected from hydrogen, —O(C₁₋₈alkyl), halo, C₁₋₆alkyl, mono(C₁₋₆alkyl)amino or di(C₁₋₆alkyl)amino;
R₇ at each occurrence is
1. optionally substituted monocyclic three- to seven-membered aryl;
2. optionally substituted monocyclic three- to seven-membered heteroaryl or heterocyclyl having one to three heteroatoms independently selected from N, O or S,
wherein the substituent on R₇ is by 1, 2 or 3 substituents represented by R,
with the proviso that when Y is NR_{c}C(X)NR_{d}Rₑ and R′ₑ=R₇, R₇ is not furan, thiophene, isooxazole, isothiazole and phenyl.

4. The compound as claimed in claim 1, where R₂ is optionally substituted three- to seven-membered heterocyclyl or heteroaryl ring having up to three heteroatoms independently selected from N, O, or S.

5. The compound as claimed in claim 1, wherein said optionally substituted heterocyclyl or heteroaryl ring is selected from piperazinyl, piperidinyl, piperidonyl, morpholinyl, thiomorpholinyl, thiomorpholin-1,1-dioxide, pyrrolidinyl pyrrolyl, pyrazolyl, oxazolyl, isoxazolyl, imidazolyl, oxadiazolyl, thiadiazolyl, triazolyl, or thiazolidinyl.

6. The compound as claimed in claim 1, wherein Rₐ and R_{b} are selected from optionally substituted piperazinyl, piperidinyl, piperidonyl, morpholinyl, thiomorpholinyl, thiomorpholin-1,1-dioxide, pyrrolidinyl pyrrolyl, pyrazolyl, triazolyl or imidazolyl.

7. The compound as claimed in claim 1, wherein X is O.

8. The compound as claimed in claim 1, wherein R₅ is independently selected from hydrogen or methyl.

9. The compound as claimed in claim 1, wherein n is independently selected from 1 or 2.

10. The compound of claim 1, wherein said compound is selected from the group consisting of:
1-[4-(Morpholine-4-carbonyl)-phenyl]-3-quinolin-2-yl-propenone (Compound No. 1);
1-[4-(3-Quinolin-2-yl-acryloyl)-benzoyl]-piperidin-4-one (Compound No. 2);
1-[4-(4-Methyl-piperazine-1-carbonyl)-phenyl]-3-quinolin-2-yl-propenone (Compound No. 3);
1-[4-(3-Quinolin-2-yl-acryloyl)-benzoyl]-pyrrolidine-2-carboxylic acid amide (Compound No. 4);
1-[4-(3-Quinolin-2-yl-acryloyl)-benzoyl]-piperidine-4-carboxylic acid isopropyl ester (Compound No. 5);
1-[4-(Piperazine-1-carbonyl)-phenyl]-3-quinolin-2-yl-propenone (Compound No. 6);
1-[4-(4-Acetyl-piperazine-1-carbonyl)-phenyl]-3-quinolin-2-yl-propenone (Compound No. 7);
1-(4-Nitro-phenyl)-4-[4-(3-quinolin-2-yl-acryloyl)-benzoyl]-piperazin-2-one (Compound No. 8);
1-[4-(3-Quinolin-2-yl-acryloyl)-benzoyl]-piperidine-4-carboxylic acid (Compound No. 9);
3-Quinolin-2-yl-1-[4-(thiomorpholine-4-carbonyl)-phenyl]-propenone (Compound No. 10);
1-[4-(Pyrrolidine-1-carbonyl)-phenyl]-3-quinolin-2-yl-propenone (Compound No. 11);
1-[4-(Piperidine-1-carbonyl)-phenyl]-3-quinolin-2-yl-propenone (Compound No. 12);
4-Morpholin-4-yl-2-{3-oxo-3-[4-(pyrrolidine-1-carbonyl)-phenyl]-propenyl}-quinoline-6-carboxylic acid methyl ester (Compound No. 13);
1-{4-[4-(3-Methyl-butyl)-piperazine-1-carbonyl]-phenyl}-3-quinolin-2-yl-propenone (Compound No. 14);
1-{4-[4-(3-Chloro-phenyl)-piperazine-1-carbonyl]-phenyl}-3-quinolin-2-yl-propenone (Compound No. 15);
1-{4-[4-(2,3-Dichloro-phenyl)-piperazine-1-carbonyl]-phenyl}-3-quinolin-2-yl-propenone (Compound No. 16);
N-(4-{2-Oxo-4-[4-(3-quinolin-2-yl-acryloyl)-benzoyl]-piperazin-1-yl}-phenyl)-acetamide (Compound No. 17);
4-Imidazol-1-yl-2-[3-oxo-3-(4-trichloromethoxycarbonylamino-phenyl)-propenyl]-quinoline-6-carboxylic acid methyl ester (Compound No. 18);
Pyrrolidine-1-carboxylic acid {4-[3-(4-morpholin-4-yl-quinolin-2-yl)-acryloyl] -phenyl}-amide (Compound No. 19);
3-Quinolin-2-yl-1-{4-[4-(tetrahydro-furan-2-carbonyl)-piperazine-1-carbonyl]-phenyl}-propenone (Compound No. 20);
1-{4-[4-(Furan-2-carbonyl)-piperazine-1-carbonyl]-phenyl}-3-quinolin-2-yl-propenone (Compound No. 21);
1-[4-(4-Pyridin-4-yl-piperazine-1-carbonyl)-phenyl]-3-quinolin-2-yl-propenone (Compound No. 22);
{4-[3-(4-Morpholin-4-yl-quinolin-2-yl)-acryloyl]-phenyl}-carbamic acid ethyl ester (Compound No: 23);
{4-[3-(4-Morpholin-4-yl-quinolin-2-yl)-acryloyl]-phenyl}-carbamic acid 2,2-dimethyl-propyl ester (Compound No. 24);
1-{4-[3-(4-Morpholin-4-yl-quinolin-2-yl)-acryloyl]-phenyl}-3-(2-trifluoromethyl-phenyl)urea (Compound No. 25);
1-Benzenesulfonyl-3-{4-[3-(4-morpholin-4-yl-quinolin-2-yl)-acryloyl]-phenyl}-urea (Compound No. 26);
4-(3-{4-[3-(4-Morpholin-4-yl-quinolin-2-yl)-acryloyl]-phenyl}-ureido)-benzoic acid ethyl ester (Compound No. 27);
1-[4-(4-Ethyl-piperazine-1-carbonyl)-phenyl]-3-quinolin-2-yl-propenone (Compound No. 28);
{4-[3-(4-Piperidin-1-yl-quinolin-2-yl)-acryloyl]-phenyl}-carbamic acid ethyl ester (Compound No. 29);
{4-[3-(4-Piperidin-1-yl-quinolin-2-yl)-acryloyl]-phenyl}-carbamic acid isobutyl ester (Compound No. 30);
{4-[3-(2-Pyrrolidin-1-yl-quinolin-3-yl)-acryloyl]-phenyl}-carbamic acid ethyl ester (Compound No. 31);
1-[4-(4-Methyl-piperazine-1-carbonyl)-phenyl]-3-(6-trifluoromethyl-quinolin-2-yl)-propenone (Compound No. 32);
1-Pyridin-2-yl-3-{4-[3-(2-pyrrolidin-1-yl-quinolin-3-yl)-acryloyl]-phenyl}-urea (Compound No. 33);
1-[4-(4-Methyl-piperazine-1-carbonyl)-phenyl]-3-(6-methylsulfanyl-quinolin-2-yl)-propenone (Compound No. 34);
1-{4-[3-(5,6,7-Trimethoxy-6,7-dihydro-quinolin-2-yl)-acryloyl]-benzoyl}-piperidin-4-one (Compound No. 35);
1-[4-(Thiomorpholine-4-carbonyl)-phenyl]-3-(5,6,7-trimethoxy-quinolin-2-yl)-propenone (Compound No. 36);
3-(3-Hydroxy-quinoxalin-2-yl)-1-[4-(4-methyl-piperazine-1-carbonyl)-phenyl]-propenone (Compound No. 37);
1-{4-[3-(3-Hydroxy-quinoxalin-2-yl)-acryloyl]-benzoyl}-piperidin-4-one (Compound No. 38);
1-[4-(4-Methyl-piperazine-1-carbonyl)-phenyl]-3-quinoxalin-2-yl-propenone (Compound No. 39);
1-[4-(Pyrrolidine-1-carbonyl)-phenyl]-3-quinoxalin-2-yl-propenone (Compound No. 40);
1-[4-(3-Quinoxalin-2-yl-acryloyl)-benzoyl]-piperidin-4-one (Compound No. 41);
3-(3-Hydroxy-quinoxalin-2-yl)-1-[4-(pyrazole-1-carbonyl)-phenyl]-propenone (Compound No. 42);
1-[4-(2,3-Dihydro-indole-1-carbonyl)-phenyl]-3-(3-hydroxy-quinoxalin-2-yl)-propenone (Compound No. 43);
1-[4-(3-Dimethylamino-pyrazole-1-carbonyl)-phenyl]-3-quinoxalin-2-yl-propenone (Compound No. 44);
1-[4-(3-Quinoxalin-2-yl-acryloyl)-benzoyl]-piperidin-4-one oxime (Compound No. 45);
2-{3-[4-(3-Dimethylamino-pyrazole-1-carbonyl)-phenyl]-3-oxo-propenyl}-quinoline-6-sulfonic acid amide (Compound No. 46);
2-{3-[4-(3,5-Dimethyl-pyrazole-1-carbonyl)-phenyl]-3-oxo-propenyl}-quinoline-6-sulfonic acid amide (Compound No. 47);
1-[4-(Pyrazole-1-carbonyl)-phenyl]-3-(5,6,7-trimethoxy-quinolin-2-yl)-propenone (Compound No. 48);
1-[4-(3,5-Dimethyl-pyrazole-1-carbonyl)-phenyl]-3-quinolin-2-yl-propenone (Compound No. 49);
1-{4-[4-(2,3-Dichloro-phenyl)-piperazine-1-carbonyl]-phenyl}-3-(2-pyrrolidin-1-yl-quinolin-3-yl)-propenone (Compound No. 50);
1-[4-(4-Methyl-piperazine-1-carbonyl)-phenyl]-3-(5,6,7-trimethoxy-quinolin-2-yl)-propenone (Compound No. 51);
1-[4-(3,5-Dimethyl-pyrazole-1-carbonyl)-phenyl]-3-(5,6,7-trimethoxy-quinolin-2-yl)-propenone (Compound No. 52);
1-{4-[3-(4-Piperidin-1-yl-6-trifluoromethyl-quinolin-2-yl)-acryloyl]-phenyl}-3-(2,3,4-trimethoxy-phenyl)-urea (Compound No. 53);
1-[4-(4-Pyrrolidin-1-yl-piperidine-1-carbonyl)-phenyl]-3-quinolin-2-yl-propenone (Compound No. 54);
1-[4-(Pyrazole-1-carbonyl)-phenyl]-3-quinolin-2-yl-propenone (Compound No. 55);
3-[6-(1-Methyl-5-trifluoromethyl-1H-pyrazol-3-yl)-quinolin-2-yl]-1-[4-(pyrrolidine-1-carbonyl)-phenyl]-propenone (Compound No. 56);
1-Cyclohexyl-3-{4-[3-(4-morpholin-4-yl-quinolin-2-yl)-acryloyl]-phenyl}-urea (Compound No. 57);
2,2-Dimethyl-N-(2-{3-oxo-3-[4-(pyrazole-1-carbonyl)-phenyl]-propenyl}-quinolin-6-yl)propionamide (Compound No. 58);
1-Benzenesulfonyl-3-{4-[3-(6-methyl-4-piperidin-1-yl-quinolin-2-yl)-acryloyl]-phenyl}-urea (Compound No. 59);
1-{4-[3-(5,6,7-Trimethoxy-quinolin-2-yl)-acryloyl]-benzoyl}-piperidine-4-carboxylic acid isopropyl ester (Compound No. 60);
Piperidine-1-carboxylic acid {4-[3-(2-pyrrolidin-1-yl-quinolin-3-yl)-acryloyl]-phenyl}-amide (Compound No. 61);
3-(6-Methylsulfanyl-4-morpholin-4-yl-quinolin-2-yl)-1-[4-(pyrrolidine-1-carbonyl)phenyl]-propenone (Compound No. 62);
3-(6-Methanesulfonyl-4-morpholin-4-yl-quinolin-2-yl)-1-[4-(pyrrolidine-1-carbonyl)phenyl]-propenone (Compound No. 63);
{4-[3-(6-[1, 2, 3]Thiadiazol-4-yl-quinolin-2-yl)-acryloyl]-phenyl}-carbamic acid ethyl ester (Compound No. 64);
1-Benzoyl-3-{4-[3-(6-[1,2,3]thiadiazol-4-yl-quinolin-2-yl)-acryloyl]-phenyl}-urea (Compound No: 65);
{4-[3-(6-[1, 2, 3]Thiadiazol-4-yl-quinolin-2-yl)-acryloyl]-phenyl}-carbamic acid phenyl ester (Compound No. 66);
{4-[3-(6-Morpholin-4-yl-pyridin-2-yl)-acryloyl]-phenyl}-carbamic acid 2-morpholin-4-yl-ethyl ester (Compound No. 67);
{4-[3-(4-Morpholin-4-yl-quinolin-2-yl)-acryloyl]-phenyl}-carbamic acid thiophen-2-ylmethyl ester (Compound No. 68);
{5-Methoxy-2-[3-(4-morpholin-4-yl-quinolin-2-yl)-acryloyl]-phenyl}-carbamic acid methyl ester (Compound No. 69);
Propyl-{4-[3-(2-pyrrolidin-1-yl-quinolin-3-yl)-acryloyl]-phenyl}-carbamic acid ethyl ester (Compound No. 70);
{4-[3-(4-Morpholin-4-yl-quinolin-2-yl)-acryloyl]-phenyl}-carbamic acid phenyl ester (Compound No. 71);
2,2-Dimethyl-N-{1-[4-(3-quinoxalin-2-yl-acryloyl)-benzoyl]-piperidin-4-yl}-propionamide (Compound No. 72);
1-[4-(3-Quinoxalin-2-yl-acryloyl)-benzoyl]-piperidine-4-carboxylic acid dimethylamide (Compound No. 73);
{4-[3-(6-[1, 2, 3]Thiadiazol-4-yl-quinolin-2-yl)-acryloyl]-phenyl}-carbamic acid methyl ester (Compound No. 74);
1-(4-Methyl-benzenesulfonyl)-3-{4-[3-(3,4,5,6-tetrahydro-2H-[1,2′]bipyridinyl-6′-yl)-acryloyl]-phenyl}-urea (Compound No. 75);
{4-[3-(3,4,5,6-Tetrahydro-2H-[1,2′]bipyridinyl-6′-yl)-acryloyl]-phenyl}-carbamic acid ethyl ester (Compound No. 76);
N-{4-[3-(2-Morpholin-4-yl-quinolin-3-yl)-acryloyl]-phenyl}-oxalamide (Compound No. 77);
2-Morpholin-4-yl-N-{4-[3-(4-morpholin-4-yl-quinolin-2-yl)-acryloyl]-phenyl}-2-oxo-acetamide (Compound No. 78);
2-Morpholin-4-yl-N-{4-[3-(2-morpholin-4-yl-quinolin-3-yl)-acryloyl]-phenyl}-2-oxo-acetamide (Compound No. 79);
N-{4-[3-(2-Piperidin-1-yl-quinolin-3-yl)-acryloyl]-phenyl}-oxalamide (Compound No. 80);
2-Morpholin-4-yl-2-oxo-N-{4-[3-(2-piperidin-1-yl-quinolin-3-yl)-acryloyl]-phenyl}-acetamide (Compound No. 81);
N-{4-[3-(4-Morpholin-4-yl-quinolin-2-yl)-acryloyl]-phenyl}-N′-propyl-oxalamide (Compound No. 82);
2-Morpholin-4-yl-N-{4-[3-(6-morpholin-4-yl-pyridin-2-yl)-acryloyl]-phenyl.}-2-oxo-acetamide (Compound No. 83);
(4-{3-[6-(3,5-Dimethyl-morpholin-4-yl)-pyridin-3-yl]-acryloyl}-phenyl)-carbamic acid phenyl ester (Compound No. 84);
N-{4-[3-(4-Morpholin-4-yl-quinolin-2-yl)-acryloyl]-phenyl}-2-oxo-2-piperidin-1-yl-acetamide (Compound No. 85);
5′-[3-Oxo-3-(4-phenoxycarbonylamino-phenyl)-propenyl]-3,4,5,6-tetrahydro-2H-[1,2′]bipyridinyl-4-carboxylic acid (Compound No. 86);
2-Oxo-2-piperidin-1-yl-N-{4-[3-(4-pyrrol-1-yl-quinolin-2-yl)-acryloyl]-phenyl}-acetamide (Compound No. 87);
2-Morpholin-4-yl-2-oxo-N-{4-[3-(4-pyrrol-1-yl-quinolin-2-yl)-acryloyl]-phenyl}-acetamide (Compound No. 88);
C,C,C-Trifluoro-N-{1-[4-(3-quinoxalin-2-yl-acryloyl)-benzoyl]-piperidin-4-yl}-methanesulfonamide (Compound No. 89);
{4-[3-(6-Pyrrolidin-1-yl-pyridin-2-yl)-acryloyl]-phenyl}-carbamic acid pyridin-2-ylmethyl ester (Compound No. 90);
{4-[3-(6-Pyrrolidin-1-yl-pyridin-2-yl)-acryloyl]-phenyl}-carbamic acid 4-fluoro-benzyl ester (Compound No. 91);
{4-[3-(3,4,5,6-Tetrahydro-2H-[1,2′]bipyridinyl-6′-yl)-acryloyl]-phenyl}-carbamic acid 2-(3,5-dimethyl-pyrazol-1-yl)-ethyl ester (Compound No. 92);
{4-[3-(3,4,5,6-Tetrahydro-2H-[1,2′]bipyridinyl-6′-yl)-acryloyl]-phenyl}-carbamic acid furan-2-ylmethyl ester (Compound No. 93);
{4-[3-(3,4,5,6-Tetrahydro-2H-[1,2′]bipyridinyl-6′-yl)-acryloyl]-phenyl}-carbamic acid 3-phenyl-allyl ester (Compound No. 94);
{4-[3-(3,4,5,6-Tetrahydro-2H-[1,2′]bipyridinyl-6′-yl)-acryloyl]-phenyl}-carbamic acid 2-piperidin-1-yl-ethyl ester (Compound No. 95);
{4-[3-(4-Morpholin-4-yl-quinolin-2-yl)-acryloyl]-phenyl}-carbamic acid methyl ester (Compound No. 96);
3-(3-Hydroxy-quinoxalin-2-yl)-1-[4-(morpholine-4-carbonyl)-phenyl]-propenone (Compound No. 97);
3-(3-Hydroxy-quinoxalin-2-yl)-1-[4-(thiomorpholine-4-carbonyl)-phenyl]-propenone (Compound No. 98);
1-[4-(3,5-Dimethyl-pyrazole-1-carbonyl)-phenyl]-3-(3-hydroxy-quinoxalin-2-yl)-propenone (Compound No. 99);
1-{4-[4-(2,3-Dichloro-phenyl)-piperazine-1-carbonyl]-phenyl}-3-(3-hydroxy-quinoxalin-2-yl)-propenone (Compound No. 100);
1-{4-[4-(3-Chloro-phenyl)-piperazine-1-carbonyl]-phenyl}-3-(3-hydroxy-quinoxalin-2-yl)-propenone (Compound No. 101);
1-{4-[3-(3-Hydroxy-quinoxalin-2-yl)-acryloyl]-benzoyl}-piperidine-4-carboxylic acid dimethylamide (Compound No. 102);
N-(1-{4-[3-(3-Hydroxy-quinoxalin-2-yl)-acryloyl]-benzoyl}-piperidin-4-y l)-2,2-dimethyl-propionamide (Compound No. 103);
1-{4-[3-(3-Hydroxy-quinoxalin-2-yl)-acryloyl]-benzoyl}-piperidine-4-carboxylic acid isopropyl ester (Compound No. 104);
1-{4-[3-(3-Hydroxy-quinoxalin-2-yl)-acryloyl]-benzoyl}-pyrrolidine-2-carboxylic acid amide (Compound No. 105);
3-(3-Hydroxy-quinoxalin-2-yl)-1-[4-(4-pyridin-4-yl-piperazine-1-carbonyl)-phenyl]-propenone (Compound No. 106);
2-{3-[4-(Morpholine-4-carbonyl)-phenyl]-3-oxo-propenyl}-3H-quinazolin-4-one (Compound No. 107);
2-{3-Oxo-3-[4-(pyrazole-1-carbonyl)-phenyl]-propenyl}-3H-quinazolin-4-one (Compound No. 108);
2-{3-[4-(4-Methyl-piperazine-1-carbonyl)-phenyl]-3-oxo-propenyl}-3H-quinazolin-4-one (Compound No. 109);
(4-{3-[2-(4-Hydroxy-piperidin-1-yl)-quinolin-3-yl]-acryloyl}-phenyl)-carbamic acid phenyl ester (Compound No. 110);
(4-{3-[2-(4-Hydroxy-piperidin-1-yl)-quinolin-3-yl]-acryloyl}-phenyl)-carbamic acid methyl ester (Compound No. 111);
(4-{3-[2-(4-Hydroxy-piperidin-1-yl)-quinolin-3-yl]-acryloyl}-phenyl)-carbamic acid ethyl ester (Compound No. 112);
1-Benzenesulfonyl-3-(4-{3-[6-(3,5-dimethyl-morpholin-4-yl)-pyridin-3-yl]-acryloyl}-phenyl)-urea (Compound No. 113);
N-{4-[3-(6-Morpholin-4-yl-pyridin-2-yl)-acryloyl]-phenyl}-2-oxo-2-piperidin-1-yl-acetamide ( Compound No. 114);
N-{4-[3-(6-Morpholin-4-yl-pyridin-2-yl)-acryloyl]-phenyl}-oxalamide (Compound No. 115);
2-Oxo-2-piperidin-1-yl-N-{4-[3-(2-piperidin-1-yl-quinolin-3-yl)-acryloyl]-phenyl}-acetamide (Compound No. 116);
2-Oxo-2-piperidin-1-yl-N-[4-(3-quinoxalin-2-yl-acryloyl)-phenyl]-acetamide (Compound No. 117);
2-Morpholin-4-yl-2-oxo-N-[4-(3-quinoxalin-2-yl-acryloyl)-phenyl]-acetamide (Compound No. 118);
N-Propyl-N′-[4-(3-quinoxalin-2-yl-acryloyl)-phenyl]-oxalamide (Compound No. 119);
N-[4-(3-Quinoxalin-2-yl-acryloyl)-phenyl]-oxalamide (Compound No. 120);
N-(2-Methoxy-ethyl)-N′-[4-(3-quinoxalin-2-yl-acryloyl)-phenyl]-oxalamide (Compound No. 121);
1-{4-[4-(3-Chloro-phenyl)-piperazine-1-carbonyl]-phenyl}-3-quinoxalin-2-yl-propenone (Compound No. 122);
3-Quinoxalin-2-yl-1-[4-(thiomorpholine-4-carbonyl)-phenyl]-propenone (Compound No. 123);
1-[4-(3-Quinoxalin-2-yl-acryloyl)-benzoyl]-piperidine-4-carboxylic acid isopropyl ester (Compound No. 124);
1-[4-(4-Pyridin-4-yl-piperazine-1-carbonyl)-phenyl]-3-quinoxalin-2-yl-propenone (Compound No. 125);
1-{4-[3-(6-Morpholin-4-yl-pyridin-2-yl)-acryloyl]-phenyl}-3-(2-oxo-2-piperidin-1-yl-ethyl)-urea (Compound No. 126);
1-(2-Morpholin-4-yl-ethyl)-3-{4-[3-(6-morpholin-4-yl-pyridin-2-yl)-acryloyl]-phenyl}-urea (Compound No. 127);
2,2-Dimethyl-N-{1-[4-(3-quinoxalin-2-yl-acryloyl)-benzoyl]-piperidin-4-yl}-propionamide (Compound No. 128);
{4-[3-(6-Morpholin-4-yl-pyridin-2-yl)-acryloyl]-phenyl}-carbamic acid 2-piperazin-1-yl-ethyl ester (Compound No. 129);
N-(2-Morpholin-4-yl-ethyl)-N′-[4-(3-quinoxalin-2-yl-acryloyl)-phenyl]-oxalamide (Compound No. 130);
1-{4-[3-(6-Morpholin-4-yl-pyridin-2-yl)-acryloyl]-phenyl}-3-[2-(pyridine-2-sulfonyl)-ethyl]-urea (Compound No. 131);
1-{4-[3-(6-Morpholin-4-yl-pyridin-2-yl)-acryloyl]-phenyl}-3-[2-(pyridin-2-ylsulfanyl)-ethyl]-urea (Compound No. 132);
1-[2-(4-Methyl-piperazin-1-yl)-ethyl]-3-{4-[3-(6-morpholin-4-yl-pyridin-2-yl)-acryloyl]-phenyl}-urea (Compound No. 133);
N-(2-Morpholin-4-yl-ethyl)-N′-{4-[3-(6-morpholin-4-yl-pyridin-2-yl)-acryloyl]-phenyl}-oxalamide (Compound No. 134);
1-(2-Morpholin-4-yl-ethyl)-3-[4-(3-quinoxalin-2-yl-acryloyl)-phenyl]-urea (Compound No. 135);
N-(2-{3-[4-(3-Quinoxalin-2-yl-acryloyl)-phenyl]-ureido}-ethyl)-benzenesulfonamide (Compound No. 136);
N-[4-(3-Quinolin-2-yl-acryloyl)-phenyl]-oxalamide (Compound No. 137);
2-Morpholin-4-yl-2-oxo-N-[4-(3-quinolin-2-yl-acryloyl)-phenyl]-acetamide (Compound No. 138);
1-Benzenesulfonyi-hydrazino-3-[4-(3-quinoxalin-2-yl-acryloyl)-phenyl]-urea (Compound No. 139);
1-(2-Morpholin-4-yl-2-oxo-ethyl)-3-[4-(3-quinolin-2-yl-acryloyl)-phenyl]-urea (Compound No. 140);
1-[2-(Pyridin-4-yloxy)-ethyl]-3-[4-(3-quinolin-2-yl-acryloyl)-phenyl]-urea (Compound No. 141);
1-(Morpholine-4-sulfonyl)-3-[4-(3-quinolin-2-yl-acryloyl)-phenyl]-urea (Compound No. 142);
{4-[3-(6-Morpholin-4-yl-pyridin-2-yl)-acryloyl]-phenyl}-carbamic acid piperidin-4-yl ester (Compound No. 143);
4-(3-{4-[3-(4-Morpholin-4-yl-quinolin-2-yl)-acryloyl]-phenyl}-ureido)-benzoic acid (Compound No. 144);
4-(3-{4-[3-(4-Piperidin-1-yl-quinolin-2-yl)-acryloyl]-phenyl}-ureido)-benzoic acid (Compound No. 145);
1-(4-Methyl-thiophen-2-yl)-3-{4-[3-(4-piperidin-1-yl-6-trifluoromethyl-quinolin-2-yl)-acryloyl]-phenyl}-urea (Compound No. 146);
N-(2-{3-[4-(3-Dimethylamino-pyrazole-1-carbonyl)-phenyl]-3-oxo-propenyl}-quinolin-6-yl)-2,2-dimethyl-propionamide (Compound No. 147);
1-[4-(Morpholine-4-carbonyl)-phenyl]-3-(5,6,7-trimethoxy-quinolin-2-yl)-propenone (Compound No. 148);
1-{4-[3-(4-Piperidin-1-yl-6-trifluoromethyl-quinolin-2-yl)-acryloyl]-phenyl}-3-pyridin-2-yl-urea (Compound No. 149);
1-Cyclohexyl-3-{4-[3-(4-piperidin-1-yl-6-trifluoromethyl-quinolin-2-yl)-acryloyl]-phenyl}-urea (Compound No. 150);
1-[4-(4-Methoxy-piperidine-1-carbonyl)-phenyl]-3-quinolin-2-yl-propenone (Compound No. 151);
1-[4-(Pyrazole-1-carbonyl)-phenyl]-3-(3,4,5,6-tetrahydro-2H-[1,2′]bipyridinyl-6′-yl)-propenone ( Compound No. 152);
1-[4-(3,5-Dimethyl-pyrazole-1-carbonyl)-phenyl]-3-(3,4,5,6-tetrahydro-2H-[1,2′]bipyridinyl-6′-yl)-propenone (Compound No. 153);
1-{4-[3-(3,4,5,6-Tetrahydro-2H-[1,2′]bipyridinyl-6′-yl)-acryloyl]-benzoyl}-piperidine-4-carboxylic acid isopropyl ester (Compound No. 154);
1-[2-(Pyridin-2-ylsulfanyl)-ethyl]-3-[4-(3-quinoxalin-2-yl-acryloyl)-phenyl]-urea (Compound No. 155);
{4-[3-(6-Morpholin-4-yl-pyridin-2-yl)-acryloyl]-phenyl}-carbamic acid 2-(pyridine-2-sulfonyl)-ethyl ester (Compound No. 156);
{4-[3-(6-Morpholin-4-yl-pyridin-2-yl)-acryloyl]-phenyl}-carbamic acid 2-(pyridin-2-ylsulfanyl)-ethyl ester (Compound No. 157);
[4-(3-Quinoxalin-2-yl-acryloyl)-phenyl]-carbamic acid 2-benzenesulfonylamino-ethyl ester (Compound No. 158);
{4-[3-(6-Morpholin-4-yl-pyridin-2-yl)-acryloyl]-phenyl}-carbamic acid 2-(N-pyridin-2-yl-hydrazino)-ethyl ester (Compound No. 159);
1-{2-[N-(6-Methyl-pyridin-2-yl)-hydrazino]-ethyl}-3-[4-(3-quinoxalin-2-yl-acryloyl)-phenyl]-urea (Compound No. 160);
1-[4-(Pyrazole-1-carbonyl)-phenyl]-3-(3,4,5,6-tetrahydro-2H-[1,2′]bipyridinyl-5′-yl)-but-2-en-1-one (Compound No. 161);
4,4,4-Trifluoro-1-[4-(morpholine-4-carbonyl)-phenyl]-3-quinolin-3-yl-but-2-en-1-one (Compound No. 62);
N-{4-[3-(6-Morpholin-4-yl-pyridin-3-yl)-but-2-enoyl]-phenyl}-2-oxo-2-piperidin-1-yl-acetamide (Compound No. 163);
2-Morpholin-4-yl-2-oxo-N-[4-(4,4,4-trifluoro-3-quinolin-3-yl-but-2-enoyl)-phenyl]-acetamide (Compound No. 164);
Morpholine-4-carboxylic acid {4-[4,4,4-trifluoro-3-(4-morpholin-4-yl-quinolin-3-yl)-but-2-enoyl]-phenyl}-amide (Compound No. 165);
Morpholine-4-carboxylic acid {4-[3-(6-morpholin-4-yl-pyridin-3-yl)-but-2-enoyl]-phenyl}-amide (Compound No. 166);
N-[2-(3-{4-[3-(6-Morpholin-4-yl-pyridin-2-yl)-acryloyl]-phenyl}-ureido)-ethyl]-nicotinamide (Compound No. 167);
1-[2-(Piperidin-4-yloxy)-ethyl]-3-[4-(3-quinolin-2-yl-acryloyl)-phenyl]-urea (Compound No. 168);
(4-{3-[6-(4-Methyl-piperazin-1-yl)-pyridin-2-yl]-acryloyl}-phenyl)-carbamic acid furan-2-ylmethyl ester (Compound No. 169);
(4-{3-[6-(4-Methyl-piperazin-1-yl)-pyridin-2-yl]-acryloyl}-phenyl)-carbamic acid pyridin-2-ylmethyl ester (Compound No. 170);
(4-{3-[6-(4-Methyl-piperazin-1-yl)-pyridin-2-yl]-acryloyl}-phenyl)-carbamic acid 2-(3,5-dimethyl-pyrazol-1-yl)-ethyl ester (Compound No. 171);
{4-[3-(6-Morpholin-4-yl-pyridin-2-yl)-acryloyl]-phenyl}-carbamic acid 2-[1,2,4]triazol-1-yl-ethyl ester (Compound No. 172);
{4-[3-(6-Piperazin-1-yl-pyridin-2-yl)-acryloyl]-phenyl}-carbamic acid 2-[1,2,4]-triazol-1-yl-ethyl ester (Compound No. 173);
(4-{3-[6-(4-Methyl-piperazin-1-yl)-pyridin-2-yl]-acryloyl}-phenyl)-carbamic acid thiophen-2-ylmethyl ester (Compound No. 174);
2-{3-Oxo-3-[4-(thiophen-2-ylmethoxycarbonylamino)-phenyl]-propenyl}-quinoline-6-carboxylic acid (Compound no. 175);
{4-[3-(6-[1,2,4]Triazol-1-yl-pyridin-2-yl)-acryloyl]-phenyl}-carbamic acid thiophen-2-ylmethyl ester (Compound no. 176);
{4-[3-(6-Tetrazol-1-yl-pyridin-2-yl)-acryloyl]-phenyl}-carbamic acid thiophen-2-yl methyl ester (Compound No. 177);
{4-[3-(6-Morpholin-4-yl-pyridin-2-yl)-acryloyl]-phenyl}-carbamic acid pyridin-2yl methyl ester (Compound No. 178);
4-(3-Quinolin-2-yl-acryloyl)-phenyl]-carbamic acid pyridin-2-ylmethyl ester (Compound No. 179);
(4-{3-[6-(4-Methyl-piperazin-1-yl)-pyridin-2-yl]-acryloyl}-phenyl)-carbamic acid piperidin-4-yl ester (Compound No. 180);
{4-[3-(6-Morpholin-4-yl-pyridin-2-yl)-acryloyl]-phenyl}-carbamic acid 1-methyl- 1H-pyrrol-3-yl ester (Compound No. 181);
(4-{3-[6-(4-Methyl-piperazin-1-yl)-pyridin-2-yl]-acryloyl}-phenyl)-carbamic acid isoxazol-3-yl ester (Compound No. 182);
[4-(3-Quinolin-2-yl-acryloyl)-phenyl]-carbamic acid 1-methyl-1H-imidazol-4-ylmethyl ester (Compound No. 183);
[4-(3-Pyridin-2-yl-acryloyl)-phenyl]-carbamic acid 2-(4-methyl-piperazine-1-sulfonylamino)-ethyl ester (Compound No. 184);
[4-(3-Quinolin-2-yl-acryloyl)-phenyl]-carbamic acid 2-(4-methyl-piperazine-1-sulfonylamino)-ethyl ester (Compound No. 185);
(4-{3-[6-(4-Methyl-piperazin-1-yl)-pyridin-2-yl]-acryloyl}-phenyl)-carbamic acid 2-benzenesulfonylamino-ethyl ester (Compound No. 186);
{4-[3-(6-Morpholin-4-yl-pyridin-2-yl)-acryloyl]-phenyl}-carbamic acid 2-[(4-methyl-piperazine-1-carbonyl)-amino]-ethyl ester (Compound No. 187);
[4-(3-Quinolin-2-yl-acryloyl)-phenyl]-carbamic acid 2-[(4-methyl-piperazine-1-carbonyl)-amino]-ethyl ester (Compound No. 188);
[4-(3-Pyridin-2-yl-acryloyl)-phenyl]-carbamic acid 2-(N-pyridin-2-yl-hydrazino)-ethyl ester (Compound No. 189);
[4-(3-Pyridin-2-yl-acryloyl)-phenyl]-carbamic acid 2-[N-(5-methyl-isoxazol-3-yl)-hydrazino]-ethyl ester (Compound No. 190);
[4-(3-Pyridin-2-yl-acryloyl)-phenyl]-carbamic acid 2-(N′-benzenesulfonyl-hydrazino)-ethyl ester (Compound No. 191);
1-{4-[3-(6-Morpholin-4-yl-pyridin-2-yl)-acryloyl]-phenyl}-3-(2-oxo-2-piperazin-1-yl-ethyl)-urea (Compound No. 192);
1-[2-(4-Methyl-piperazin-1-yl)-2-oxo-ethyl]-3-[4-(3-quinolin-2-yl-acryloyl)-phenyl]-urea (Compound No. 193);
1-(2-Morpholin-4-yl-2-oxo-ethyl)-3-{4-[3-(6-piperazin-1-yl-pyridin-2-yl)-acryloyl]-phenyl}-urea (Compound No. 194);
1-[2-(Pyridine-2-sulfonyl)-ethyl]-3-[4-(3-pyridin-2-yl-acryloyl)-phenyl]-urea (Compound No. 195);
1-{4-[3-(6-Piperazin-1-yl-pyridin-2-yl)-acryloyl]-phenyl}-3-[2-(piperidine-4-sulfonyl)-ethyl]-urea (Compound No. 196);
4-Methyl-piperazine-1-sulfonic acid(2-{3-[4-(3-quinolin-2-yl-acryloyl)-phenyl]-ureido}-ethyl)-amide (Compound No. 197);
1-{4-[3-(6-Morpholin-4-yl-pyridin-2-yl)-acryloyl]-phenyl}-3-[2-(piperidin-4-ylsulfanyl)-ethyl]-urea (Compound No. 198);
1-{2-[N-(1-Methyl-piperidin-4-yl)-hydrazino]-ethyl}-3-[4-(3-pyridin-2-yl-acryloyl)-phenyl]-urea (Compound No. 199);
1-{2-[N-(1-Methyl-piperidin-4-yl)-hydrazino]-ethyl}-3-[4-(3-quinoxalin-2-yl-acryloyl)-phenyl]-urea (Compound No. 200);
1-{2-[N-(6-Methyl-pyridin-2-yl)-hydrazino]-ethyl}-3-{4-[3-(6-morpholin-4-yl-pyridin-2-yl)-acryloyl]-phenyl}-urea (Compound No. 201);
1-(2-Piperazin-1-yl-ethyl)-3-[4-(3-quinoxalin-2-yl-acryloyl)-phenyl]-urea (Compound No. 202);
1-(2-Morpholin-4-yl-ethyl)-3-[4-(3-quinolin-2-yl-acryloyl)-phenyl]urea (Compound No. 203);
1-(2-Piperazin-1-yl-ethyl)-3-[4-(3-quinolin-2-yl-acryloyl)-phenyl]-urea (Compound No. 204);
1-{4-[3-(6-Piperazin-1-yl-pyridin-2-yl)-acryloyl]-phenyl}-3-(2-piperidin-1-yl-ethyl)-urea (Compound No. 205);
1-{4-[3-(6-Piperazin-1-yl-pyridin-2-yl)-acryloyl]-phenyl}-3-(2-thiomorpholin 1,1-dioxide-4-yl-ethyl)-urea (Compound No. 206);
1-(2-Piperazin-1-yl-ethyl)-3-{4-[3-(3,4,5,6-tetrahydro-2H-[1,2′]bipyridinyl-6′-yl)-acryloyl]-phenyl}-urea (Compound No. 207);
Piperidine-4-carboxylic acid [2-(3-{4-[3-(6-morpholin-4-yl-pyridin-2-yl)-acryloyl]-phenyl}-ureido)-ethyl]-amide (Compound No. 208);
N-(2-{3-[4-(3-Pyridin-2-yl-acryloyl)-phenyl]-ureido}-ethyl)-nicotinamide (Compound No. 209);
1-[2-(N′-Benzenesulfonyl-hydrazino)-ethyl]-3-[4-(3-pyridin-2-yl-acryloyl)-phenyl]-urea (Compound No. 210);
1-[2-(N′-Benzenesulfonyl-hydrazino)-ethyl]-3-[4-(3-quinolin-2-yl-acryloyl)-phenyl]-urea (Compound No. 211);
1-(Piperazine-1-sulfonyl)-3-[4-(3-pyridin-2-yl-acryloyl)phenyl]-urea (Compound No. 212);
1-{4-[3-(6-Morpholin-4-yl-pyridin-2-yl)-acryloyl]-phenyl}-3-(piperazine-1-sulfonyl)-urea (Compound No. 213);
1-(Piperazine-1-sulfonyl)-3-[4-(3-quinoxalin-2-yl-acryloyl)-phenyl]-urea (Compound No. 214);
1-(Piperazine-1-sulfonyl)-3-[4-(3-quinolin-2-yl-acryloyl) phenyl]-urea (Compound No. 215);
N-(2-Piperazin-1-yl-ethyl)-N′-[4-(3-pyridin-2-yl-acryloyl)-phenyl]-oxalamide (Compound No. 216);
N-{4-[3-(6-Morpholin-4-yl-pyridin-2-yl)-acryloyl]-phenyl}-N′-(2-piperazin-1-yl-ethyl)-oxalamide (Compound No. 217);
1-[2-({4-[3-(6-Morpholin-4-yl-pyridin-2-yl)-acryloyl]-phenylaminooxalyl}-amino)-acetyl]-piperidine-4-carboxylic acid (Compound No. 218);
N-(2-Oxo-2-piperazin-1-yl-ethyl)-N′-[4-(3-pyridin-2-yl-acryloyl)-phenyl]-oxalamide (Compound No. 219);
N-[4-(3-Pyridin-2-yl-acryloyl)-phenyl]-N′-[2-(pyridine-2-yl-sulfonyl)-ethyl]-oxalamide (Compound No. 220);
N-[2-(Piperidin-4-ylsulfanyl)-ethyl]-N′-[4-(3-pyridin-2-yl-acryloyl)-phenyl]-oxalamide (Compound No. 221);
N-[2-(Pyridine-2-sulfonyl)-ethyl]-N′-[4-(3-quinolin-2-yl-acryloyl)-phenyl]-oxalamide (Compound No. 222);
2-[(6-Methyl-pyridin-2-yl)-hydrazino]-2-oxo-N-[4-(3-quinolin-2-yl-acryloyl)-phenyl]-acetamide (Compound No. 223);
2-Oxo-2-(N-phenyl-hydrazino)-N-[4-(3-pyridin-2-yl-acryloyl)-phenyl]-acetamide (Compound No. 224);
2-Oxo-2-(piperazine-1-sulfonylamino)-N-[4-(3-pyridin-2-yl-acryloyl)-phenyl]-acetamide (Compound No. 225);
2-Benzenesulfonylamino-2-oxo-N-[4-(3-pyridin-2-yl-acryloyl)-phenyl]-acetamide (Compound No. 226);
N-[2-(Piperazine-1-sulfonylamino)-ethyl]-N′-[4-(3-pyridin-2-yl-acryloyl)-phenyl]-oxalamide (Compound No. 227);
2-(N′-Benzenesulfonyl-hydrazino)-2-oxo-N-[4-(3-pyridin-2-yl-acryloyl)-phenyl]-acetamide (Compound No. 228);
N-{2-[N-(6-Methyl-pyridin-2-yl)-hydrazino]-ethyl}-N′-[4-(3-pyridin-2-yl-acryloyl)-phenyl]-oxalamide (Compound No. 229);
N-{2-[(Piperazine-1-carbonyl)-amino]-ethyl}-N′-[4-(3-pyridin-2-yl-acryloyl)-phenyl]-oxalamide (Compound No. 230);
N-{2-[(Pyridine-3-carbonyl)-amino]-ethyl}-N′-[4-(3-pyridin-2-yl-acryloyl)-phenyl]-oxalamide (Compound No. 231);
N-{2-[(Piperidine-4-carbonyl)-amino]-ethyl}-N′-[4-(3-pyridin-2-yl-acryloyl)-phenyl]-oxalamide (Compound No. 232);
1-[4-(4-Methyl-piperazine-1-carbonyl)-phenyl]-3-{6-[2-(4-methyl-piperazin-1-yl)-ethylamino]-pyridin-2-yl}-propenone (Compound No. 233);
(2-{3-[4-(4-Methyl-piperazine-1-carbonyl)-phenyl]-3-oxo-propenyl}-quinolin-6-yl)-carbamic acid ethyl ester (Compound No. 234);
(6-{3-[4-(4-Methyl-piperazine-1-carbonyl)-phenyl]-3-oxo-propenyl}-pyridin-2-yl)-carbamic acid phenyl ester (Compound No. 235);
1-[4-(Morpholine-4-carbonyl)-phenyl]-3-{6-[(piperidin-1-ylmethyl)-amino]-pyridin-2-yl}-propenone (Compound No. 236);
1-[4-(3-Dimethylamino-pyrazole-1-carbonyl)-phenyl]-3-[6-(2-oxo-2-piperidin-1-yl-ethylamino)-pyridin-2-yl]-propenone (Compound No. 237);
1-[4-(Piperazine-1-carbonyl)-phenyl]-3-{6-[2-(pyridine-2-sulfonyl)-ethylamino]-pyridin-2-yl}-propenone (Compound No. 238);
Benzenesulfonic acid N′-(6-{3-oxo-3-[4-(piperidine-1-carbonyl)-phenyl]-propenyl}-pyridin-2-yl)-hydrazide (Compound No. 239);
(6-{3-[4-(3-Benzenesulfonyl-ureido)-phenyl]-3-oxo-propenyl}-pyridin-2-yl)-carbamic acid ethyl ester (Compound No. 240);
Morpholine-4-carboxylic acid (4-{3-[6-(2-piperidin-1-yl-ethylamino)-pyridin-2-yl]-acryloyl}-phenyl)-amide (Compound No. 241);
{2-[3-(4-Ethoxycarbonylamino-phenyl)-3-oxo-propenyl]-quinolin-6-yl}-carbamic acid ethyl ester (Compound No. 242);
{2-[3-Oxo-3-(4-phenoxycarbonylamino-phenyl)-propenyl]-quinolin-4-yl}-carbamic acid ethyl ester (Compound No. 243);
[2-(3-{4-[2-(4-Methyl-piperazin-1-yl)-2-oxo-acetylamino]-phenyl}-3-oxo-propenyl)-quinolin-4-yl]-carbamic acid methyl ester (Compound No. 244);
1-(2-Morpholin-4-yl-ethyl)-3-(4-{3-[6-(2-piperidin-1-yl-ethylamino)-pyridin-2-yl]-acryloyl}-phenyl)-urea (Compound No. 245);
1-[4-(3-Dimethylamino-pyrazole-1-carbonyl)-phenyl]-3-{6-[2-(4-methyl-piperazin-1-yl)-2-oxo-ethylamino]-pyridin-2-yl}-propenone (Compound No. 246);
N-(1-{4-[4-(3-Pyridin-2-yl-acryloyl)-benzoyl]-piperazin-1-yl}-piperidin-4-yl)-methanesulfonamide (Compound No. 247);
(1-{4-[4-(3-Pyridin-2-yl-acryloyl)-benzoyl]-piperazin-1-yl}-piperidin-4-yl)-sulfonylurea (Compound No. 248);
1-{4-[4-(4-Cyclohexylamino-piperidin-1-yl)-piperazine-1-carbonyl]-phen yl}-3-pyridin-2-yl-propenone (Compound No. 249);
[6-(3-Oxo-3-{4-[4-(pyrrolidine-1-carbonyl)-piperidine-1-carbonyl]-phenyl}-propenyl)-pyridin-2-yl]-carbamic acid methyl ester (Compound No. 250);
[6-(3-Oxo-3-{4-[4-(pyridin-2-yloxy)-piperidine-1-carbonyl]-phenyl}-propenyl)-pyridin-2-yl]-carbamic acid ethyl ester (Compound No. 251).

11. The compound as claimed in claim 2, wherein said compound is selected from the group consisting of:
N-(4-{2-Oxo-4-[4-(3-quinolin-2-yl-acryloyl)-benzoyl]-piperazin-1-yl}-phenyl)-acetamide (Compound No. 17);
3-Quinolin-2-yl-1-{4-[4-(tetrahydro-furan-2-carbonyl)-piperazine-1-carbonyl]-phenyl}-propenone (Compound No. 20);
1-{4-[4-(Furan-2-carbonyl)-piperazine-1-carbonyl]-phenyl}-3-quinolin-2-yl-propenone (Compound No. 21);
{4-[3-(4-Morpholin-4-yl-quinolin-2-yl)-acryloyl]-phenyl}-carbamic acid ethyl ester (Compound No. 23);
{4-[3-(4-Morpholin-4-yl-quinolin-2-yl)-acryloyl]-phenyl}-carbamic acid 2,2-dimethyl-propyl ester (Compound No. 24);
{4-[3-(4-Piperidin-1-yl-quinolin-2-yl)-acryloyl]-phenyl}-carbamic acid ethyl ester (Compound No. 29);
{4-[3-(4-Piperidin-1-yl-quinolin-2-yl)-acryloyl]-phenyl}-carbamic acid isobutyl ester (Compound No. 30);
{4-[3-(2-Pyrrolidin-1-yl-quinolin-3-yl)-acryloyl]-phenyl}-carbamic acid ethyl ester (Compound No. 31);
1-[4-(2,3-Dihydro-indole-1-carbonyl)-phenyl]-3-(3-hydroxy-quinoxalin-2-yl)-propenone (Compound No. 43);
1-[4-(3-Quinoxalin-2-yl-acryloyl)-benzoyl]-piperidin-4-one oxime (Compound No. 45);
1-Benzenesulfonyl-3-{4-[3-(6-methyl-4-piperidin-1-yl-quinolin-2-yl)-acryloyl]-phenyl}-urea (Compound No. 59);
{4-[3-(6-[1,2,3]Thiadiazol-4-yl-quinolin-2-yl)-acryloyl]-phenyl}-carbamic acid ethyl ester (Compound No. 64);
{4-[3-(6-[1,2,3]Thiadiazol-4-yl-quinolin-2-yl)-acryloyl]-phenyl}-carbamic acid phenyl ester (Compound No. 66);
{4-[3-(6-Morpholin-4-yl-pyridin-2-yl)-acryloyl]-phenyl}-carbamic acid 2-morpholin-4-yl-ethyl ester (Compound No. 67);
{5-Methoxy-2-[3-(4-morpholin-4-yl-quinolin-2-yl)-acryloyl]-phenyl}-carbamic acid methyl ester (Compound No. 69);
Propyl-{4-[3-(2-pyrrolidin-1-yl-quinolin-3-yl)-acryloyl]-phenyl}-carbamic acid ethyl ester (Compound No. 70);
{4-[3-(4-Morpholin-4-yl-quinolin-2-yl)-acryloyl]-phenyl}-carbamic acid phenyl ester (Compound No. 71);
{4-[3-(6-[1, 2, 3]Thiadiazol-4-yl-quinolin-2-yl)-acryloyl]-phenyl}-carbamic acid methyl ester (Compound No. 74);
1-(4-Methyl-benzenesulfonyl)-3-{4-[3-(3,4,5,6-tetrahydro-2H-[1,2′]bipyridinyl-6′-yl)-acryloyl]-phenyl}-urea (Compound No. 75);
{4-[3-(3,4,5,6-Tetrahydro-2H-[1,2′]bipyridinyl-6′-yl)-acryloyl]-phenyl}-carbamic acid ethyl ester (Compound No. 76);
(4-{3-[6-(3,5-Dimethyl-morpholin-4-yl)-pyridin-3-yl]-acryloyl}-phenyl)-carbamic acid phenyl ester (Compound No. 84);
5′-[3-Oxo-3-(4-phenoxycarbonylamino-phenyl)-propenyl]-3,4,5,6-tetrahydro-2H-[1,2′]bipyridinyl-4-carboxylic acid (Compound No. 86);
{4-[3-(6-Pyrrolidin-1-yl-pyridin-2-yl)-acryloyl]-phenyl}-carbamic acid pyridin-2-ylmethyl ester (Compound No. 90);
{4-[3-(6-Pyrrolidin-1-yl-pyridin-2-yl)-acryloyl]-phenyl}-carbamic acid 4-fluoro-benzyl ester (Compound No. 91);
{4-[3-(3,4,5,6-Tetrahydro-2H-[1,2′]bipyridinyl-6′-yl)-acryloyl]-phenyl}-carbamic acid 2-(3,5-dimethyl-pyrazol-1-yl)-ethyl ester (Compound No. 92);
{4-[3-(3,4,5,6-Tetrahydro-2H-[1,2′]bipyridinyl-6′-yl)-acryloyl]-phenyl}-carbamic acid furan-2-ylmethyl ester (Compound No. 93);
{4-[3-(3,4,5,6-Tetrahydro-2H-[1,2′]bipyridinyl-6′-yl)-acryloyl]-phenyl}-carbamic acid 3-phenyl-allyl ester (Compound No. 94);
{4-[3-(3,4,5,6-Tetrahydro-2H-[1,2′]bipyridinyl-6′-yl)-acryloyl]-phenyl}-carbamic acid 2-piperidin-1-yl-ethyl ester (Compound No. 95);
{4-[3-(4-Morpholin-4-yl-quinolin-2-yl)-acryloyl]-phenyl}-carbamic acid methyl ester (Compound No. 96);
(4-{3-[2-(4-Hydroxy-piperidin-1-yl)-quinolin-3-yl]-acryloyl}-phenyl)-carbamic acid phenyl ester (Compound No. 110);
(4-{3-[2-(4-Hydroxy-piperidin-1-yl)-quinolin-3-yl]-acryloyl}-phenyl)-carbamic acid methyl ester (Compound No. 111);
(4-{3-[2-(4-Hydroxy-piperidin-1-yl)-quinolin-3-yl]-acryloyl}-phenyl)-carbamic acid ethyl ester (Compound No. 112);
1-Benzenesulfonyl-3-(4-{3-[6-(3,5-dimethyl-morpholin-4-yl) -pyridin-3-yl]-acryloyl}-phenyl)-urea (Compound No. 113);
1-{4-[3-(6-Morpholin-4-yl-pyridin-2-yl)-acryloyl]-phenyl}-3-(2-oxo-2-piperidin-1-yl-ethyl)-urea (Compound No. 126);
1-(2-Morpholin-4-yl-ethyl)-3-{4-[3-(6-morpholin-4-yl-pyridin-2-yl)-acryloyl]-phenyl}-urea (Compound No. 127);
{4-[3-(6-Morpholin-4-yl-pyridin-2-yl)-acryloyl]-phenyl}-carbamic acid 2-piperazin-1-yl-ethyl ester (Compound No. 129);
N-(2-Morpholin-4-yl-ethyl)-N′-[4-(3-quinoxalin-2-yl-acryloyl)-phenyl]-oxalamide (Compound No. 130);
1-{4-[3-(6-Morpholin-4-yl-pyridin-2-yl)-acryloyl]-phenyl}-3-[2-(pyridine-2-sulfonyl)-ethyl]-urea (Compound No. 131);
1-{4-[3-(6-Morpholin-4-yl-pyridin-2-yl)-acryloyl]-phenyl}-3-[2-(pyridin-2-ylsulfanyl)-ethyl]-urea (Compound No. 132);
1-[2-(4-Methyl-piperazin-1-yl)-ethyl]-3-{4-[3-(6-morpholin-4-yl-pyridin-2-yl)-acryloyl]-phenyl}-urea (Compound No. 133);
N-(2-Morpholin-4-yl-ethyl)-N′-{4-[3-(6-morpholin-4-yl-pyridin-2-yl)-acryloyl]-phenyl}-oxalamide (Compound No. 134);
1-(2-Morpholin-4-yl-ethyl)-3-[4-(3-quinoxalin-2-yl-acryloyl)-phenyl]-urea (Compound No. 135);
1-Benzenesulfonyl-hydrazino-3-[4-(3-quinoxalin-2-yl-acryloyl)-phenyl]-urea (Compound No. 139);
1-(2-Morpholin-4-yl-2-oxo-ethyl)-3-[4-(3-quinolin-2-yl-acryloyl)-phenyl]-urea (Compound No. 140);
1-(Morpholine-4-sulfonyl)-3-[4-(3-quinolin-2-yl-acryloyl)-phenyl]-urea (Compound No. 142);
{4-[3-(6′-Morpholin-4-yl-pyridin-2-yl)-acryloyl]-phenyl}-carbamic acid piperidin-4-yl ester (Compound No. 143);
1-[2-(Pyridin-2-ylsulfanyl)-ethyl]-3-[4-(3-quinoxalin-2-yl-acryloyl)-phenyl]-urea (Compound No. 155);
{4-[3-(6-Morpholin-4-yl-pyridin-2-yl)-acryloyl]-phenyl}-carbamic acid 2-(pyridine-2-sulfonyl)-ethyl ester (Compound No. 156);
{4-[3-(6-Morpholin-4-yl-pyridin-2-yl)-acryloyl]-phenyl}-carbamic acid 2-(pyridin-2-ylsulfanyl)-ethyl ester (Compound No. 157);
[4-(3-Quinoxalin-2-yl-acryloyl)-phenyl]-carbamic acid 2-benzenesulfonylamino-ethyl ester (Compound No. 158);
{4-[3-(6-Morpholin-4-yl-pyridin-2-yl)-acryloyl]-phenyl}-carbamic acid 2-(N-pyridin-2-yl-hydrazino)-ethyl ester (Compound No. 159);
1-{2-[N-(6-Methyl-pyridin-2-yl-hydrazino]-ethyl}-3-[4-(3-quinoxalin-2-yl-acryloyl)-phenyl]-urea (Compound No. 160);
1-[4-(Pyrazole-1-carbonyl)-phenyl]-3-(3,4,5,6-tetrahydro-2H-[l,2′]bipyridinyl-5′-yl)-but-2-en-1-one (Compound No. 161);
4,4,4-Trifluoro-1-[4-(morpholine-4-carbonyl)-phenyl]-3-quinolin-3-yl-but-2-en-1-one (Compound No. 162);
N-{4-[3-(6-Morpholin-4-yl-pyridin-3-yl)-but-2-enoyl]-phenyl}-2-oxo-2-piperidin-1-yl-acetamide (Compound No. 163);
2-Morpholin-4-yl-2-oxo-N-[4-(4,4,4-trifluoro-3-quinolin-3-yl-but-2-enoyl)-phenyl]-acetamide (Compound No. 164);
Morpholine-4-carboxylic acid {4-[4,4,4-trifluoro-3-(4-morpholin-4-yl-quinolin-3-yl)-but-2-enoyl]-phenyl}-amide (Compound No. 165);
Morpholine-4-carboxylic acid {4-[3-(6-morpholin-4-yl-pyridin-3-yl)-but-2-enoyl]-phenyl}-amide (Compound No. 166);
N-[2-(3-{4-[3-(6-Morpholin-4-yl-pyridin-2-yl)-acryloyl]-phenyl}-ureido)-ethyl]-nicotinamide (Compound No. 167);
1-[2-(Piperidin-4-yloxy)-ethyl]-3-[4-(3-quinolin-2-yl-acryloyl)-phenyl]-urea (Compound No. 168);
(4-{3-[6-(4-Methyl-piperazin-1-yl)-pyridin-2-yl]-acryloyl}-phenyl)-carbamic acid furan-2-ylmethyl ester (Compound No. 169);
(4-{3-[6-(4-Methyl-piperazin-1-yl)-pyridin-2-yl]-acryloyl}-phenyl)-carbamic acid pyridin-2-ylmethyl ester (Compound No. 170);
(4-{3-[6-(4-Methyl-piperazin-1-yl)-pyridin-2-yl]-acryloyl}-phenyl)-carbamic acid 2-(3,5-dimethyl-pyrazol-1-yl)-ethyl ester (Compound No. 171);
{4-[3-(6-Morpholin-4-yl-pyridin-2-yl)-acryloyl]-phenyl}-carbamic acid 2-[1,2,4]triazol-1-yl-ethyl ester (Compound No. 172);
{4-[3-(6-Piperazin-1-yl-pyridin-2-yl)-acryloyl]-phenyl}-carbamic acid 2-[1,2,4]-triazol-1-yl-ethyl ester (Compound No. 173);
(4-{3-[6-(4-Methyl-piperazin-1-yl)-pyridin-2-yl]-acryloyl}-phenyl)-carbamic acid thiophen-2-ylmethyl ester (Compound No. 174);
2-{3-Oxo-3-[4-(thiophen-2-ylmethoxycarbonylamino)-phenyl]-propenyl}-quinoline-6-carboxylic acid (Compound no. 175);
{4-[3-(6-[1,2,4]Triazol-1-yl-pyridin-2-yl)-acryloyl]-phenyl}-carbamic acid thiophen-2-ylmethyl ester (Compound no. 176);
{4-[3-(6-Tetrazol-1-yl-pyridin-2-yl)-acryloyl]-phenyl}-carbamic acid thiophen-2-yl methyl ester (Compound No. 177);
{4-[3-(6-Morpholin-4-yl-pyridin-2-yl)-acryloyl]-phenyl}-carbamic acid pyridin-2yl methyl ester (Compound No. 178);
4-(3-Quinolin-2-yl-acryloyl)-phenyl]-carbamic acid pyridin-2-ylmethyl ester (Compound No. 179);
(4-{3-[6-(4-Methyl-piperazin-1-yl)-pyridin-2-yl]-acryloyl}-phenyl)-carbamic acid piperidin-4-yl ester (Compound No. 180);
{4-[3-(6-Morpholin-4-yl-pyridin-2-yl)-acryloyl]-phenyl}-carbamic acid 1-methyl-1H-pyrrol-3-yl ester! (Compound No. 181);
(4-{3-[6-(4-Methyl-piperazin-1-yl)-pyridin-2-yl]-acryloyl}-phenyl)-carbamic acid isoxazol-3-yl ester (Compound No. 182);
[4-(3-Quinolin-2-yl-acryloyl)-phenyl]-carbamic acid 1-methyl-1H-imidazol-4-ylmethyl ester (Compound No. 183);
[4-(3-Pyridin-2-yl-acryloyl)-phenyl]-carbamic acid 2-(4-methyl-piperazine-1-sulfonylamino)-ethyl ester (Compound No. 184);
[4-(3-Quinolin-2-yl-acryloyl)-phenyl]-carbamic acid 2-(4-methyl-piperazine-1-sulfonylamino)-ethyl ester (Compound No. 185);
(4-{3-[6-(4-Methyl-piperazin-1-yl)-pyridin-2-yl]-acryloyl}-phenyl)-carbamic acid 2-benzenesulfonylamino-ethyl ester (Compound No. 186);
{4-[3-(6-Morpholin-4-yl-pyridin-2-yl)-acryloyl]-phenyl}-carbamic acid 2-[(4-methyl-piperazine-1-carbonyl)-amino]-ethyl ester (Compound No. 187);
[4-(3-Quinolin-2-yl-acryloyl)-phenyl]-carbamic acid 2-[(4-methyl-piperazine-1-carbonyl)-amino]-ethyl ester (Compound No. 188);
[4-(3-Pyridin-2-yl-acryloyl)-phenyl]-carbamic acid 2-(N-pyridin-2-yl-hydrazino)-ethyl ester (Compound No. 189);
[4-(3-Pyridin-2-yl-acryloyl)-phenyl]-carbamic acid 2-[N-(5-methyl-isoxazol-3-yl)-hydrazino]-ethyl ester (Compound No. 190);
[4-(3-Pyridin-2-yl-acryloyl)-phenyl]-carbamic acid 2-(N′-benzenesulfonyl- hydrazino)-ethyl ester (Compound No. 191);
1-{4-[3-(6-Morpholin-4-yl-pyridin-2-yl)-acryloyl]-phenyl}-3-(2-oxo-2-piperazin-1-yl-ethyl)-urea (Compound No. 192);
1-[2-(4-Methyl-piperazin-1-yl)-2-oxo-ethyl]-3-[4-(3-quinolin-2-yl-acryloyl)-phenyl]-urea (Compound No. 193);
1-(2-Morpholin-4-yl-2-oxo-ethyl)-3-{4-[3-(6-piperazin-1-yl-pyridin-2-yl)-acryloyl]-phenyl}-urea (Compound No. 194);
1-[2-(Pyridine-2-sulfonyl)-ethyl]-3-[4-(3-pyridin-2-yl-acryloyl)-phenyl]-urea (Compound No. 195);
1-{4-[3-(6-Piperazin-1-yl-pyridin-2-yl)-acryloyl]-phenyl}-3-[2-(piperidine-4-sulfonyl)-ethyl]-urea (Compound No. 196);
4-Methyl-piperazine-1-sulfonic acid(2-{3-[4-(3-quinolin-2-yl-acryloyl)-phenyl]-ureido}-ethyl)-amide (Compound No. 197);
1-{4-[3-(6-Morpholin-4-yl-pyridin-2-yl)-acryloyl]-phenyl}-3-[2-(piperidin-4-ylsulfanyl)-ethyl] -urea (Compound No. 198);
1-{2-[N-(1-Methyl-piperidin-4-yl)-hydrazino]-ethyl}-3-[4-(3-pyridin-2-yl-acryloyl)-phenyl]-urea (Compound No. 199);
1-{2-[N-(1-Methyl-piperidin-4-yl)-hydrazino]-ethyl}-3-[4-(3-quinoxalin-2-yl-acryloyl)-phenyl]-urea (Compound No. 200);
1-{2-[N-(6-Methyl-pyridin-2-yl)-hydrazino]-ethyl}-3-{4-[3-(6-morpholin-4-yl-pyridin-2-yl)-acryloyl]-phenyl}-urea (Compound No. 201);
1-(2-Piperazin-1-yl-ethyl)-3-[4-(3-quinoxalin-2-yl-acryloyl)-phenyl]-urea (Compound No. 202);
1-(2-Morpholin-4-yl-ethyl)-3-[4-(3-quinolin-2-yl-acryloyl)-phenyl]urea (Compound No. 203);
1-(2-Piperazin-1-yl-ethyl)-3-[4-(3-quinolin-2-yl-acryloyl)-phenyl]-urea (Compound No. 204);
1-{4-[3-(6-Piperazin-1-yl-pyridin-2-yl)-acryloyl]-phenyl}-3-(2-piperidin-1-yl-ethyl)-urea (Compound No. 205);
1-{4-[3-(6-Piperazin-1-yl-pyridin-2-yl)-acryloyl]-phenyl}-3-(2-thiomorpholin 1,1-dioxide-4-yl-ethyl)-urea (Compound No. 206);
1-(2-Piperazin-1-yl-ethyl)-3-{4-[3-(3)3,4,5,6-tetrahydro-2H-[1,2′]bipyridinyl-6′-yl)-acryloyl]-phenyl}-urea (Compound No. 207) ;
Piperidine-4-carboxylic acid [2-(3-{4-[3-(6-morpholin-4-yl-pyridin-2-yl)-acryloyl]-phenyl}-ureido)-ethyl]-amide (Compound No. 208);
N-(2-{3-[4-(3-Pyridin-2-yl-acryloyl)-phenyl]-ureido}-ethyl)-nicotinamide (Compound No. 209);
1-[2-(N′-Benzenesulfonyl-hydrazino)-ethyl]-3-[4-(3-pyridin-2-yl-acryloyl)-phenyl]-urea (Compound No. 210);
1-[2-(N′-Benzenesulfonyl-hydrazino)-ethyl]-3-[4-(3-quinolin-2-yl-acryloyl)-phenyl]-urea (Compound No. 211);
1-(Piperazine-1-sulfonyl)-3-[4-(3-pyridin-2-yl-acryloyl)phenyl]-urea (Compound No. 212);
1-{4-[3-(6-Morpholin-4-yl-pyridin-2-yl)-acryloyl]-phenyl}-3-(piperazine-1-sulfonyl)-urea (Compound No. 213);
1-(Piperazine-1-sulfonyl)-3-[4-(3′-quinoxalin-2-yl-acryloyl)-phenyl]-urea (Compound No. 214);
1-(Piperazine-1-sulfonyl)-3-[4-(3-quinolin-2-yl-acryloyl) phenyl]-urea (Compound No. 215);
N-(2-Piperazin-1-yl-ethyl)-N′-[4-(3-pyridin-2-yl-acryloyl)-phenyl]-oxalamide (Compound No. 216);
N-{4-[3-(6-Morpholin-4-yl-pyridin-2-yl)-acryloyl]-phenyl}-N′-(2-piperazin-1-yl-ethyl)-oxalamide (Compound No. 217);
1-[2-({4-[3-(6-Morpholin-4-yl-pyridin-2-yl)-acryloyl]-phenylaminooxalyl}-amino)-acetyl]-piperidine-4-carboxylic acid (Compound No. 218);
N-(2-Oxo-2-piperazin-1-yl-ethyl)-N′-[4-(3-pyridin-2-yl-acryloyl)-phenyl]-oxalamide (Compound No. 219);
N-[4-(3-Pyridin-2-yl-acryloyl)-phenyl]-N′-[2-(pyridine-2-yl-sulfonyl)-ethyl]-oxalamide (Compound No 220);
N-[2-(Piperidin-4-ylsulfanyl)-ethyl]-N′-[4-(3-pyridin-2-yl-acryloyl)-phenyl]-oxalamidea (Compound No. 221);
N-[2-(Pyridine-2-sulfonyl)-ethyl]-N′-[4-(3-quinolin-2-yl-acryloyl)-phenyl]-oxalamide (Compound No. 222);
2-[N-(6-Methyl-pyridin-2-yl)-hydrazino]-2-oxo-N-[4-(3-quinolin-2-yl-acryloyl)-phenyl]-acetamide (Compound No. 223);
2-Oxo-2-(N-phenyl-hydrazino)-N-[4-(3-pyridin-2-yl-acryloyl)-phenyl]-acetamide (Compound No. 224);
2-Oxo-2-(piperazine-1-sulfonylamino)-N-[4-(3-pyridin-2-yl-acryloyl)-phenyl]-acetamide (Compound No. 225);
2-Benzenesulfonylamino-2-oxo-N-[4-(3-pyridin-2-yl-acryloyl)-phenyl]-acetamide (Compound No. 226);
N-[2-(Piperazine-1-sulfonylamino)-ethyl]-N′-[4-(3-pyridin-2-yl-acryloyl)-phenyl]-oxalamide (Compound No. 227);
2-(N′-Benzenesulfonyl-hydrazino)-2-oxo-N-[4-(3-pyridin-2-yl-acryloyl)-phenyl]-acetamide (Compound No. 228);
N-{2-[N-(6-Methyl-pyridin-2-yl)-hydrazino]-ethyl}-N′-[4-(3-pyridin-2-yl-acryloyl)-phenyl]-oxalamide (Compound No. 229);
N-{2-[(Piperazine-1-carbonyl)-amino]-ethyl}-N′-[4-(3-pyridin-2-yl-acryloyl)-phenyl]-oxalamide (Compound No. 230);
N-{2-[(Pyridine-3-carbonyl)-amino]-ethyl}-N′-[4-(3-pyridin-2-yl-acryloyl)-phenyl]-oxalamide (Compound No. 231);
N-{2-[(Piperidine-4-carbonyl)-amino]-ethyl}-N′-[4-(3-pyridin-2-yl-acryloyl)-phenyl]-oxalamide (Compound No. 232);
N-(1-{4-[4-(3-Pyridin-2-yl-acryloyl)-benzoyl]-piperazin-1-yl}-piperidin-4-yl)-methanesulfonamide (Compound No. 247);
(1-{4-[4-(3-Pyridin-2-yl-acryloyl)-benzoyl]-piperazin-1-yl}-piperidin-4-yl)-sulfonylurea (Compound No. 248);
1-{4-[4-(4-Cyclohexylamino-piperidin-1-yl)-piperazine-1-carbonyl]-phenyl}-3-pyridin-2-yl-propenone (Compound No. 249).

12. The compound as claimed in claim 3, wherein said compound is selected from the group consisting of:
1-[4-(4-Methyl-piperazine-1-carbonyl)-phenyl]-3-{6-[2-(4-methyl-piperazin-1-yl)-ethylamino]-pyridin-2-yl}-propenone (Compound No. 233);
(2-{3-[4-(4-Methyl-piperazine-1-carbonyl)-phenyl]-3-oxo-propenyl}-quinolin-6-yl)-carbamic acid ethyl ester (Compound No. 234);
(6-{3-[4-(4-Methyl-piperazine-1-carbonyl)-phenyl]-3-oxo-propenyl}-pyridin-2-yl)-carbamic acid phenyl ester (Compound No. 235);
1-[4-(Morpholine-4-carbonyl)-phenyl]-3-{6-[(piperidin-1-ylmethyl)-amino]-pyridin-2-yl}-propenone (Compound No. 236);
1-[4-(3-Dimethylamino-pyrazole-1-carbonyl)-phenyl]-3-[6-(2-oxo-2-piperidin-1-yl-ethylamino)-pyridin-2-yl]-propenone (Compound No. 237);
1-[4-(Piperazine-1-carbonyl)-phenyl]-3-{6-[2-(pyridine-2-sulfonyl)-ethylamino]-pyridin-2-yl}-propenone (Compound No. 238);
Benzenesulfonic acid N-[2-(6-{3-oxo-3-[4-(piperidine-1-carbonyl)-phenyl]-propenyl}-pyridin-2-ylamino)-ethyl]-hydrazide (Compound No. 239);
(6-{3-[4-(3-Benzenesulfonyl-ureido)-phenyl]-3-oxo-propenyl}-pyridin-2-yl)-carbamic acid ethyl ester (Compound No. 240);
Morpholine-4-carboxylic acid (4-{3-[6-(2-piperidin-1-yl-ethylamino)-pyridin-2-yl]-acryloyl}-phenyl)-amide (Compound No. 241);
{2-[3-(4-Ethoxycarbonylamino-phenyl)-3-oxo-propenyl]-quinolin-6-yl}-carbamic acid ethyl ester (Compound No. 242);
{2-[3-Oxo-3-(4-phenoxycarbonylamino-phenyl)-propenyl]-quinolin-4-yl}-carbamic acid ethyl ester (Compound No. 243);
[2-(3-{4-[2-(4-Methyl-piperazin-1-yl)-2-oxo-acetylamino]-phenyl}-3-oxo-propenyl)-quinolin-4-yl]-carbamic acid methyl ester (Compound No. 244);
1-(2-Morpholin-4-yl-ethyl)-3-(4-{3-[6-(2-piperidin-1-yl-ethylamino)-pyridin-2-yl]-acryloyl}-phenyl)-urea (Compound No. 245);
1-[4-(3-Dimethylamino-pyrazole-1-carbonyl)-phenyl]-3-{6-[2-(4-methyl-piperazin-1-yl)-2-oxo-ethylamino]-pyridin-2-yl}-propenone (Compound No. 246);
[6-(3-Oxo-3-{4-[4-(pyrrolidine-1-carbonyl)-piperidine-1-carbonyl]-phenyl}-propenyl)-pyridin-2-yl]-carbamic acid methyl ester (Compound No. 250);
[6-(3-Oxo-3-{4-[4-(pyridin-2-yloxy)-piperidine-1-carbonyl]-phenyl}-propenyl)-pyridin-2-yl]-carbamic acid ethyl ester (Compound No. 251).

13. A method of treating diseases accompanying pathological stress in a living mammalian organism, including a human being, by inducing the expression of HSP-70 in the body cells of said living mammalian organism, comprising administering to said living mammalian organism in need thereof a therapeutically effective amount of a chemical compound, wherein said chemical compound is one or more of a 2-propene-1-one derivative represented by formula (I), as defined in claim 1.

14. The method as claimed in claim 13, wherein said diseases accompanying pathological stress are selected from stroke, myocardial infarction, inflammatory disorder, hepatotoxicity, sepsis, diseases of viral original, allograft rejection, tumourous diseases, gastric mucosal damage, brain haemorrhage, endothelial dysfunctions, diabetic complications, neuro-degenerative diseases, post-traumatic neuronal damage, acute renal failure, glaucoma and aging related skin degeneration.

15. The method as claimed in claim 14, wherein said disease accompanying pathological stress is stroke.

16. The method as claimed in claim 14, wherein said disease accompanying pathological stress is myocardial infarction.

17. The method as claimed in claim 14, wherein said disease accompanying pathological stress is an inflammatory disorder.

18. The method as claimed in claim 14, wherein said diabetic complications are selected from diabetic neuropathy, diabetic retinopathy and chronic wound healing.

19. The method as claimed in claim 14, wherein said neuro-degenerative diseases are selected from Alzheimer's disease, amyotrophic lateral sclerosis and Parkinson's disease.

20. A pharmaceutical composition comprising a therapeutically effective amount of one or more of a compound of formula (I) as defined in claim 1, in association with a pharmaceutically acceptable carrier, diluent or excipient.

21. The pharmaceutical composition as claimed in claim 20, in the form of an oral formulation or a parenteral formulation.

22. The pharmaceutical composition as claimed in claim 20 in the form of a tablet, capsule, powder, syrup, solution, infusion or suspension.

23. A pharmaceutical composition as claimed in claim 20, containing 1 to 1000 mg of a compound of general formula (I), at concentration levels ranging from about 0.5% to about 90% by weight of the total composition.

24. The pharmaceutical composition as claimed in claim 23, more preferably containing 20 to 500 mg of compound of general formula (I), at concentration levels ranging from about 10% to about 70% by weight of the total composition.

25. Use of one or more of a compound represented by the formula (I), as defined in claim 1, in the manufacture of a medicament for the treatment of diseases accompanying pathological stress in a living mammalian organism, including a human being, by inducing the expression of HSP-70 in the body cells of said living mammalian organism.

26. The use as claimed in claim 25, wherein said diseases accompanying pathological stress are selected from stroke, myocardial infarction, inflammatory disorder, hepatotoxicity, sepsis, diseases of viral original, allograft rejection, tumourous diseases, gastric mucosal damage, brain haemorrhage, endothelial dysfunctions, diabetic complications, neuro-degenerative diseases, post-traumatic neuronal damage, acute renal failure, glaucoma and aging related skin degeneration.

27. The use as claimed in claim 26, wherein said disease accompanying pathological stress is stroke.

28. The use as claimed in claim 26, wherein said disease accompanying pathological stress is myocardial infarction.

29. The use as claimed in claim 26, wherein said disease accompanying pathological stress is an inflammatory disorder.

30. The use as claimed in claim 26, wherein said diabetic complications are selected from diabetic neuropathy, diabetic retinopathy and chronic wound healing.

31. The use as claimed in claim 26, wherein said neuro-degenerative diseases are selected from Alzheimer's disease, amyotrophic lateral sclerosis and Parkinson's disease.
